SEQUENCE LISTING (45 sequences)

SEQ ID NO: 1 - DNA, 2496 bp, Arabidopsis thaliana
gaaaaaaaaaacgaattctaatgtgcccgctataaaatcttccgcaagagtgtaacagtgatgcagctgaatcaataagactgtcttcttctccgaatttgaaaattaaattccagttttttcagtttgactctgcttcttcttcctcgtgggtaacgacgatataccgttaaaattaggaaccaaattacccaatggtcgtcgtcaaatcatttttaatcccaatttggtatttttccacgtgggtcaaacaaaaaacaattttttacataaagagaagagagtagtgacgagaagattagcactactgaatcaaacttagccgccgccaccgtcacgttgaaaccttcatctctctatctctctgagacctctccttcaatggcgtcttcgcttctgagaaaccctaatctctactcatcaacaacaatcaccaccacttcttttcttcccaccttctcctctaaacccacacctatctcctcctctttccgtttccaaccatctcaccaccgttcaatctccctccgaagtcaaaccctccgtctctcatgctcaatctcagatccttctccactaccacctcacactcctcgccgtccccgtcctgaatacatccccaaccgcatttccgatccaaactacgtccgcgtcttcgatactactctccgtgacggtgaacaatctccaggagctacacttacttccaaggaaaaacttgacatcgctcgtcagctagctaaacttggtgttgacatcatcgaggctgggtttcctgctgcttccaaggatgattttgaagcggttaagactatagctgaaacagttggaaacactgttgatgagaatggttatgttcctgttatctgtggactctctagatgcaataagaaggatattgagagagcttgggatgctgtgaaatacgctaaacggcctaggattcatacttttatagctactagtgatatacatttggagtataaactaaagaaaaccaaagcagaggtcatcgaaatcgctaggagtatggttagattcgcgaggagcttggggtgcgaagatgttgagttcagtccagaagatgcaggaagatcggagagagagtacttatacgagattcttggtgaagtgataaaagcaggagcaacaactctcaacatacctgatactgttggtataactttgcctagtgagtttggtcaactgattactgatttaaaggccaatactccggggattgaaaatgttgtcatctcaacacattgtcagaatgatcttggactctctacggccaacactttatctggggcacatgcaggtgcgaggcagatggaagtgacgatcaatggaattggtgaaagagctggaaacgcttcactggaagaggttgtgatggccataaaatgccgtggagatcatgtattaggaggtctatttaccggaattgatactcggcacattgttatgacaagcaagatggtagaggagtacactgggatgcagacacaacctcataaggctattgtaggagcgaatgcctttgcgcatgaaagtggaattcaccaggatggaatgctgaaacacaagggtacatatgaaattatatgtcccgaagaaattggacttgaacgatcaaatgatgctggcattgtcttggggaagcttagtgggcgtcatgcgctgaaagaccgtttgactgagcttggttatcaattagatgatgaacagctaagtaccattttctggcgcttcaaaaccgtggctgagcagaaaaagagagttactgatgcggacataatagctttagtatctgatgaagttttccagccagaagccgtgtggaaactcctggacattcagataacttgtggaactctcgggctttcaacagcaactgtaaaacttgctgacgctgatggcaaagaacatgtcgcttgttctattggaactgggcctgtggattcagcttacaaggcagtagatcttatcgtaaaggaaccggctactctgcttgagtactcaatgaatgcagtaacagaaggcattgatgccatcgcaaccacaagagttcttatccgtggaagcaacaaatactcatctacaaacgcaataactggtgaagaggttcaaagaacctttagtggaactggagcaggaatggatattgtggtgtcaagcgtcaaagcttatgttggagctttgaacaaaatgatggacttcaaagaaaactccgccacaaaaatcccttcccaaaaaaacagagtcgctgcctgaattaaaaatctttccggcaaataccaaaaagtcagacagaagttaggttcttttattttcaagtacatagtttggtaataactggagtttcggagtttgcttgttgtttatcgaagttgcatgtcaaaagagtttggtgtactatatatatcttgatttaacttgaatctctatttttagaaataatggttttagaataaggaataaaaaccaaccgtt

SEQ ID NO: 2 - protein, 631 aa, Arabidopsis thaliana
MASSLLRNPNLYSSTTITTTSFLPTFSSKPTPISSSFRFQPSHHRSISLRSQTLRLSCSISDPSPLPPHTPRRPRPEYIPNRISDPNYVRVFDTTLRDGEQSPGATLTSKEKLDIARQLAKLGVDIIEAGFPAASKDDFEAVKTIAETVGNTVDENGYVPVICGLSRCNKKDIERAWDAVKYAKRPRIHTFIATSDIHLEYKLKKTKAEVIEIARSMVRFARSLGCEDVEFSPEDAGRSEREYLYEILGEVIKAGATTLNIPDTVGITLPSEFGQLITDLKANTPGIENVVISTHCQNDLGLSTANTLSGAHAGARQMEVTINGIGERAGNASLEEVVMAIKCRGDHVLGGLFTGIDTRHIVMTSKMVEEYTGMQTQPHKAIVGANAFAHESGIHQDGMLKHKGTYEIICPEEIGLERSNDAGIVLGKLSGRHALKDRLTELGYQLDDEQLSTIFWRFKTVAEQKKRVTDADIIALVSDEVFQPEAVWKLLDIQITCGTLGLSTATVKLADADGKEHVACSIGTGPVDSAYKAVDLIVKEPATLLEYSMNAVTEGIDAIATTRVLIRGSNKYSSTNAITGEEVQRTFSGTGAGMDIVVSSVKAYVGALNKMMDFKENSATKIPSQKNRVAA

SEQ ID NO: 3 - protein, 631 aa, Artificial Sequence
  A synthetic sequence
MASSLLRNPNLYSSTTITTTSFLPTFSSKPTPISSSFRFQPSHHRSISLRSQTLRLSCSISDPSPLPPHTPRRPRPEYIPNRISDPNYVRVFDTTLRDGEQSPGATLTSKEKLDIARQLAKLGVDIIEAGFPAASKDDFEAVKTIAETVGNTVDENGYVPVICGLSRCNKKDIERAWDAVKYAKRPRIHTFIATSDIHLEYKLKKTKAEVIEIARSMVRFARSLGCENVEFSPEDAGRSEREYLYEILGEVIKAGATTLNIPDTVGITLPSEFGQLITDLKANTPGIENVVISTHCQNDLGLSTANTLSGAHAGARQMEVTINGIGERAGNASLEEVVMAIKCRGDHVLGGLFTGIDTRHIVMTSKMVEEYTGMQTQPHKAIVGANAFAHESGIHQDGMLKHKGTYEIICPEEIGLERSNDAGIVLGKLSGRHALKDRLTELGYQLDDEQLSTIFWRFKTVAEQKKRVTDADIIALVSDEVFQPEAVWKLLDIQITCGTLGLSTATVKLADADGKEHVACSIGTGPVDSAYKAVDLIVKEPATLLEYSMNAVTEGIDAIATTRVLIRGSNKYSSTNAITGEEVQRTFSGTGAGMDIVVSSVKAYVGALNKMMDFKENSATKIPSQKNRVAA

SEQ ID NO: 4 - DNA, 1896 bp, Artificial Sequence
  A synthetic sequence
atggcgtcttcgcttctgagaaaccctaatctctactcatcaacaacaatcaccaccacttcttttcttcccaccttctcctctaaacccacacctatctcctcctctttccgtttccaaccatctcaccaccgttcaatctccctccgaagtcaaaccctccgtctctcatgctcaatctcagatccttctccactaccacctcacactcctcgccgtccccgtcctgaatacatccccaaccgcatttccgatccaaactacgtccgcgtcttcgatactactctccgtgacggtgaacaatctccaggagctacacttacttccaaggaaaaacttgacatcgctcgtcagctagctaaacttggtgttgacatcatcgaggctgggtttcctgctgcttccaaggatgattttgaagcggttaagactatagctgaaacagttggaaacactgttgatgagaatggttatgttcctgttatctgtggactctctagatgcaataagaaggatattgagagagcttgggatgctgtgaaatacgctaaacggcctaggattcatacttttatagctactagtgatatacatttggagtataaactaaagaaaaccaaagcagaggtcatcgaaatcgctaggagtatggttagattcgcgaggagcttggggtgcgaaaatgttgagttcagtccagaagatgcaggaagatcggagagagagtacttatacgagattcttggtgaagtgataaaagcaggagcaacaactctcaacatacctgatactgttggtataactttgcctagtgagtttggtcaactgattactgatttaaaggccaatactccggggattgaaaatgttgtcatctcaacacattgtcagaatgatcttggactctctacggccaacactttatctggggcacatgcaggtgcgaggcagatggaagtgacgatcaatggaattggtgaaagagctggaaacgcttcactggaagaggttgtgatggccataaaatgccgtggagatcatgtattaggaggtctatttaccggaattgatactcggcacattgttatgacaagcaagatggtagaggagtacactgggatgcagacacaacctcataaggctattgtaggagcgaatgcctttgcgcatgaaagtggaattcaccaggatggaatgctgaaacacaagggtacatatgaaattatatgtcccgaagaaattggacttgaacgatcaaatgatgctggcattgtcttggggaagcttagtgggcgtcatgcgctgaaagaccgtttgactgagcttggttatcaattagatgatgaacagctaagtaccattttctggcgcttcaaaaccgtggctgagcagaaaaagagagttactgatgcggacataatagctttagtatctgatgaagttttccagccagaagccgtgtggaaactcctggacattcagataacttgtggaactctcgggctttcaacagcaactgtaaaacttgctgacgctgatggcaaagaacatgtcgcttgttctattggaactgggcctgtggattcagcttacaaggcagtagatcttatcgtaaaggaaccggctactctgcttgagtactcaatgaatgcagtaacagaaggcattgatgccatcgcaaccacaagagttcttatccgtggaagcaacaaatactcatctacaaacgcaataactggtgaagaggttcaaagaacctttagtggaactggagcaggaatggatattgtggtgtcaagcgtcaaagcttatgttggagctttgaacaaaatgatggacttcaaagaaaactccgccacaaaaatcccttcccaaaaaaacagagtcgctgcctga

SEQ ID NO: 5 - protein, 631 aa, Artificial Sequence
  A synthetic sequence
MASSLLRNPNLYSSTTITTTSFLPTFSSKPTPISSSFRFQPSHHRSISLRSQTLRLSCSISDPSPLPPHTPRRPRPEYIPNRISDPNYVRVFDTTLRDGEQSPGATLTSKEKLDIARQLAKLGVDIIEAGFPAASKDDFEAVKTIAETVGNTVDENGYVPVICGLSRCNKKDIERAWDAVKYAKRPRIHTFIATSDIHLEYKLKKTKAEVIEIARSMVRFARSLGCEDVEFSPEDAGRSEREYLYEILGEVIKAGATTLNIPDTVGITLPSEFGQLITDLKANTPGIENVVISTHCQNDLGLSTANTLSGAHAGARQMEVTINGIGERAGNASLEEVVMAIKCRGDHVLGGLFTGIDTRHIVMTSKMVEEYTGMQTQPHKAIVGANAFAHESGIHQDGMLKHKGTYEIICPEEIGLERSNDAGIVLGKLSGRHALKDRLTELGYQLDDEQLSTIFWRFKTVAEQKKRVTDADIIALVSDEVFQPEAVWKLLDIQITCGTLGLSTATVKLADADGKEHVACSIGTGPVDSAYKAVDLIVKEPATLLEYSMNAVTEGIDAIATTRVLIRGSNKYSSTNAITGEEVQRTFSGTGAGMDIVVSSVKAYVEALNKMMDFKENSATKIPSQKNRVAA

SEQ ID NO: 6 - DNA, 1896 bp, Artificial Sequence
  A synthetic sequence
atggcgtcttcgcttctgagaaaccctaatctctactcatcaacaacaatcaccaccacttcttttcttcccaccttctcctctaaacccacacctatctcctcctctttccgtttccaaccatctcaccaccgttcaatctccctccgaagtcaaaccctccgtctctcatgctcaatctcagatccttctccactaccacctcacactcctcgccgtccccgtcctgaatacatccccaaccgcatttccgatccaaactacgtccgcgtcttcgatactactctccgtgacggtgaacaatctccaggagctacacttacttccaaggaaaaacttgacatcgctcgtcagctagctaaacttggtgttgacatcatcgaggctgggtttcctgctgcttccaaggatgattttgaagcggttaagactatagctgaaacagttggaaacactgttgatgagaatggttatgttcctgttatctgtggactctctagatgcaataagaaggatattgagagagcttgggatgctgtgaaatacgctaaacggcctaggattcatacttttatagctactagtgatatacatttggagtataaactaaagaaaaccaaagcagaggtcatcgaaatcgctaggagtatggttagattcgcgaggagcttggggtgcgaagatgttgagttcagtccagaagatgcaggaagatcggagagagagtacttatacgagattcttggtgaagtgataaaagcaggagcaacaactctcaacatacctgatactgttggtataactttgcctagtgagtttggtcaactgattactgatttaaaggccaatactccggggattgaaaatgttgtcatctcaacacattgtcagaatgatcttggactctctacggccaacactttatctggggcacatgcaggtgcgaggcagatggaagtgacgatcaatggaattggtgaaagagctggaaacgcttcactggaagaggttgtgatggccataaaatgccgtggagatcatgtattaggaggtctatttaccggaattgatactcggcacattgttatgacaagcaagatggtagaggagtacactgggatgcagacacaacctcataaggctattgtaggagcgaatgcctttgcgcatgaaagtggaattcaccaggatggaatgctgaaacacaagggtacatatgaaattatatgtcccgaagaaattggacttgaacgatcaaatgatgctggcattgtcttggggaagcttagtgggcgtcatgcgctgaaagaccgtttgactgagcttggttatcaattagatgatgaacagctaagtaccattttctggcgcttcaaaaccgtggctgagcagaaaaagagagttactgatgcggacataatagctttagtatctgatgaagttttccagccagaagccgtgtggaaactcctggacattcagataacttgtggaactctcgggctttcaacagcaactgtaaaacttgctgacgctgatggcaaagaacatgtcgcttgttctattggaactgggcctgtggattcagcttacaaggcagtagatcttatcgtaaaggaaccggctactctgcttgagtactcaatgaatgcagtaacagaaggcattgatgccatcgcaaccacaagagttcttatccgtggaagcaacaaatactcatctacaaacgcaataactggtgaagaggttcaaagaacctttagtggaactggagcaggaatggatattgtggtgtcaagcgtcaaagcttatgttgaagctttgaacaaaatgatggacttcaaagaaaactccgccacaaaaatcccttcccaaaaaaacagagtcgctgcctga

SEQ ID NO: 7 - protein, 631 aa, Arabidopsis thaliana
MESSILKSPNLSSPSFGVPSIPALSSSSTSPFSSLHLRSQNHRTISLTTAGKFRVSYSLSASSPLPPHAPRRRPNYIPNRISDPNYVRIFDTTLRDGEQSPGATLTSKEKLDIARQLAKLGVDIIEAGFPAASKDDFEAVKTIAETVGNTVDENGYVPVICGLSRCNKKDIETAWEAVKYAKRPRIHTFIATSDIHLKYKLKKSKEEVIEIARNMVRFARSLGCEDVEFSPEDAGRSEREYLYEILGEVIKAGATTLNIPDTVGITLPSEFGQLIADIKANTPGIQNVIISTHCQNDLGLSTANTLSGAHSGARQVEVTINGIGERAGNASLEEVVMAIKCRGDHVLGGLFTGIDTRHIVMTSKMVEEYTGMQTQPHKAIVGANAFAHESGIHQDGMLKHKGTYEIMSPEEIGLERSNDAGIVLGKLSGRHALKDRLNELGYVLDDGQLSNLFWRFKAVAEQKKRVTDADLIALVSDEVFQPEAVWKLLDMQITCGTLGLSTSTVKLADSDGKEHVACSVGTGPVDAAYKAVDLIVKEPATLLEYSMNAVTEGIDAIATTRVLIRGDNNYSSTNAVTGESVERTFSGTGAGMDIVVSSVKAYVGALNKMLGFKEHTSTLSKTPLETNEVPA

SEQ ID NO: 8 - DNA, 2261 bp, Arabidopsis thaliana
tttggttcggttcggttcggaacaattcaaataaataaaacaaatcaaaaatattcactagcaaagtagtaaccagagacactgtgccgtcgcccgtcgccgccgccgccacactatcatctctctcaggtttttgattttccacggcaatggagtcttcgattctcaaaagccctaatctctcttcaccatcgttcggtgtaccttcaattcccgccttatcctcctcctccacctcaccattttcatctcttcatctccgatcacagaaccaccgtaccatctctcttaccaccgccggaaaattccgtgtctcgtattctctctccgcttcttcacctctaccacctcatgctcctcgccgtcgtcccaattacatccctaaccgtatatccgatcccaattacgtcagaatcttcgatacaactctccgagacggtgaacagtctcccggagctacactaacctccaaggaaaagctcgatatcgctcgtcaattagccaagctcggagtcgacatcatcgaagctggatttcccgctgcttcaaaagacgatttcgaagctgttaaaaccatagctgagactgttggcaataccgtcgacgaaaatggctatgtccctgtaatctgtggtctctcgagatgtaacaagaaggatattgagacggcttgggaagctgtgaagtacgctaagcggccaagaatccatacgtttattgccactagtgatattcatctgaagtataagttgaagaagagtaaagaagaagttattgagatcgctaggaacatggttagattcgccagaagcttgggatgtgaagatgttgaatttagtccagaagatgccggaagatcggagagagagtacttatacgagattcttggtgaagtgatcaaagctggagcaaccactcttaacatacctgacactgttggtataaccttgcctagtgagtttggtcagttgattgctgatattaaagctaatactcctgggatccaaaatgttataatctctacacattgtcagaatgatcttggactctccaccgccaacactttatctggtgcacattcgggcgcgaggcaagtggaagtgactatcaatggaattggcgaaagagctggaaacgcttcattggaagaggttgtcatggccataaaatgccgtggagatcatgtcttaggaggcctatttactggaatcgatacccggcacattgttatgacaagcaagatggttgaggagtacactggtatgcaaacgcagccccataaggctattgtaggagcaaacgcctttgcgcatgaaagtggtattcatcaggatggaatgctgaagcacaagggtacctatgaaattatgtcccccgaagagattgggcttgagcgatcaaatgatgctggcatcgtgctgggaaagcttagtgggcgtcacgcactgaaagaccgtttaaatgagctcggttatgtcctggatgatgggcagctaagcaaccttttctggcgtttcaaagctgtggcagagcaaaaaaagagagttaccgatgctgacttaatagctttagtatctgatgaagtgtttcagccagaggctgtctggaaactcctggacatgcagataacttgtggaactctcggtctctcaacatctactgtaaaacttgctgactccgatggcaaagagcatgtagcttgttctgttggaaccggacctgtagatgcagcttacaaggcagttgatcttatcgttaaggaacctgcgactctgcttgagtactcgatgaatgcagtaacagaaggcattgatgctattgcaaccacacgggttctaatccgcggagacaacaactactcatcaacaaacgcggtaacgggtgaatctgttgaaagaacttttagtggaaccggagcaggaatggacattgttgtgtcgagcgttaaagcttatgttggagctttgaacaaaatgttgggtttcaaagaacacacctccactttaagtaaaacccctttggagaccaacgaagtccctgcctgaagaaaatcttacctgcaaatctcagagatcaaatcacaatttagatgagtaaacgtctaaaagatttttattttttgttaccgttattgtttgtataaaaagaatatgagttttggtttactaaaataactatgatataaaacagagtatttggttaaaacattgaaacaagaacaaattttctataaatagtgaaggcatttgccctaacaggaa

SEQ ID NO: 9 - protein, 631 aa, Artificial Sequence
  A synthetic sequence
MESSILKSPNLSSPSFGVPSIPALSSSSTSPFSSLHLRSQNHRTISLTTAGKFRVSYSLSASSPLPPHAPRRRPNYIPNRISDPNYVRIFDTTLRDGEQSPGATLTSKEKLDIARQLAKLGVDIIEAGFPAASKDDFEAVKTIAETVGNTVDENGYVPVICGLSRCNKKDIETAWEAVKYAKRPRIHTFIATSDIHLKYKLKKSKEEVIEIARNMVRFARSLGCENVEFSPEDAGRSEREYLYEILGEVIKAGATTLNIPDTVGITLPSEFGQLIADIKANTPGIQNVIISTHCQNDLGLSTANTLSGAHSGARQVEVTINGIGERAGNASLEEVVMAIKCRGDHVLGGLFTGIDTRHIVMTSKMVEEYTGMQTQPHKAIVGANAFAHESGIHQDGMLKHKGTYEIMSPEEIGLERSNDAGIVLGKLSGRHALKDRLNELGYVLDDGQLSNLFWRFKAVAEQKKRVTDADLIALVSDEVFQPEAVWKLLDMQITCGTLGLSTSTVKLADSDGKEHVACSVGTGPVDAAYKAVDLIVKEPATLLEYSMNAVTEGIDAIATTRVLIRGDNNYSSTNAVTGESVERTFSGTGAGMDIVVSSVKAYVGALNKMLGFKEHTSTLSKTPLETNEVPA

SEQ ID NO: 10 - protein, 631 aa, Artificial Sequence
  A synthetic sequence
MESSILKSPNLSSPSFGVPSIPALSSSSTSPFSSLHLRSQNHRTISLTTAGKFRVSYSLSASSPLPPHAPRRRPNYIPNRISDPNYVRIFDTTLRDGEQSPGATLTSKEKLDIARQLAKLGVDIIEAGFPAASKDDFEAVKTIAETVGNTVDENGYVPVICGLSRCNKKDIETAWEAVKYAKRPRIHTFIATSDIHLKYKLKKSKEEVIEIARNMVRFARSLGCEDVEFSPEDAGRSEREYLYEILGEVIKAGATTLNIPDTVGITLPSEFGQLIADIKANTPGIQNVIISTHCQNDLGLSTANTLSGAHSGARQVEVTINGIGERAGNASLEEVVMAIKCRGDHVLGGLFTGIDTRHIVMTSKMVEEYTGMQTQPHKAIVGANAFAHESGIHQDGMLKHKGTYEIMSPEEIGLERSNDAGIVLGKLSGRHALKDRLNELGYVLDDGQLSNLFWRFKAVAEQKKRVTDADLIALVSDEVFQPEAVWKLLDMQITCGTLGLSTSTVKLADSDGKEHVACSVGTGPVDAAYKAVDLIVKEPATLLEYSMNAVTEGIDAIATTRVLIRGDNNYSSTNAVTGESVERTFSGTGAGMDIVVSSVKAYVEALNKMLGFKEHTSTLSKTPLETNEVPA

SEQ ID NO: 11 - protein, 633 aa, Brachypodium distachyon
MAASPAKPCCFSSLNPASSTPLARRSRTLSSSAAAAKPHRFSHGLAAAAVAANPRAAALRRPVRACLAAGAAPRRPEYVPDRIDDPNYVRIFDTTLRDGEQSPGATMTSAEKLVVARQLARLGVDIIEAGFPASSPDDLDAVRSIAIEVGNTPLGGDGHVPVICGLSRCNKRDIDAAWEAVRHARKPRIHTFIATSEIHMQHKLRKTPEQVVAIAREMVAYARSLGCPDVEFSPEDAGRSNREFLYHILEEVIKAGATTLNIPDTVGYNLPHEFGKLIADIKANTPGIENAIISTHCQNDLGLASANTLAGAYAGARQLEVTINGIGERAGNASLEEVVMAIKCRRELLGGLYTGISTQHITMSSKMVQEHSGLHVQPHKAIVGANAFAHESGIHQDGMLKHKGTYEIISPEDIGLVRVNEFGIVLGKLSGRHAVKTKLVELGYEISDKEFEDFFKRYKEVAEKKKRVTDEDIEALLSDEIFQPKVIWSLGDVQATCGTLGLSTATVKLIAIDGEEKIGCSVGTGPVDAAYKAVDQIIQIPTVLREYSMTSVTEGIDAIATTRVVITGDVSNSKNALIGQNNRSFSGSGAALDVVVSSVRAYLSALNKMSSYVGAVKASSEAPESIRTVQTAE

SEQ ID NO: 12 - protein, 633 aa, Artificial Sequence
  A synthetic sequence
MAASPAKPCCFSSLNPASSTPLARRSRTLSSSAAAAKPHRFSHGLAAAAVAANPRAAALRRPVRACLAAGAAPRRPEYVPDRIDDPNYVRIFDTTLRDGEQSPGATMTSAEKLVVARQLARLGVDIIEAGFPASSPDDLDAVRSIAIEVGNTPLGGDGHVPVICGLSRCNKRDIDAAWEAVRHARKPRIHTFIATSEIHMQHKLRKTPEQVVAIAREMVAYARSLGCPNVEFSPEDAGRSNREFLYHILEEVIKAGATTLNIPDTVGYNLPHEFGKLIADIKANTPGIENAIISTHCQNDLGLASANTLAGAYAGARQLEVTINGIGERAGNASLEEVVMAIKCRRELLGGLYTGISTQHITMSSKMVQEHSGLHVQPHKAIVGANAFAHESGIHQDGMLKHKGTYEIISPEDIGLVRVNEFGIVLGKLSGRHAVKTKLVELGYEISDKEFEDFFKRYKEVAEKKKRVTDEDIEALLSDEIFQPKVIWSLGDVQATCGTLGLSTATVKLIAIDGEEKIGCSVGTGPVDAAYKAVDQIIQIPTVLREYSMTSVTEGIDAIATTRVVITGDVSNSKNALIGQNNRSFSGSGAALDVVVSSVRAYLSALNKMSSYVGAVKASSEAPESIRTVQTAE

SEQ ID NO: 13 - protein, 633 aa, Artificial Sequence
  A synthetic sequence
MAASPAKPCCFSSLNPASSTPLARRSRTLSSSAAAAKPHRFSHGLAAAAVAANPRAAALRRPVRACLAAGAAPRRPEYVPDRIDDPNYVRIFDTTLRDGEQSPGATMTSAEKLVVARQLARLGVDIIEAGFPASSPDDLDAVRSIAIEVGNTPLGGDGHVPVICGLSRCNKRDIDAAWEAVRHARKPRIHTFIATSEIHMQHKLRKTPEQVVAIAREMVAYARSLGCPDVEFSPEDAGRSNREFLYHILEEVIKAGATTLNIPDTVGYNLPHEFGKLIADIKANTPGIENAIISTHCQNDLGLASANTLAGAYAGARQLEVTINGIGERAGNASLEEVVMAIKCRRELLGGLYTGISTQHITMSSKMVQEHSGLHVQPHKAIVGANAFAHESGIHQDGMLKHKGTYEIISPEDIGLVRVNEFGIVLGKLSGRHAVKTKLVELGYEISDKEFEDFFKRYKEVAEKKKRVTDEDIEALLSDEIFQPKVIWSLGDVQATCGTLGLSTATVKLIAIDGEEKIGCSVGTGPVDAAYKAVDQIIQIPTVLREYSMTSVTEGIDAIATTRVVITGDVSNSKNALIGQNNRSFSGSGAALDVVVSSVRAYLEALNKMSSYVGAVKASSEAPESIRTVQTAE

SEQ ID NO: 14 - protein, 555 aa, Glycine max
MATKTSTNGTHHSLPEYIPNRIPDPHYVRILDTTLRDGEQAPGAAMTSDQKLQIARQLAKLGVDVIEGGFPSASQEDFNAVKMIAQEVGNNCDADGYVPVIAALCRCNERDITRAWEALKYAKRPRLMPFIAVSPIHMEYKLNKTKEEVLQIATDMIKFARGLGCTDIQFCSEDAARSDREFLYQILEEVIKAGATTLGIGDTVGITMPFEIRELVAGIKANVPGAENVIISIHCHNDLGHATANTIEAARAGAMQLEVTINGIGERAGNASLEEVVMALKCRGDHVLGGLYTGINTRHLLKTSKMVEEFSGMYLQPHKAVVGDNAFLHESGVHQAGLLKHRGTYEILSPEDIGHEKSNGVNMVLGKLSGRQALKSRLKELGYELRDEEVESVFRNFKAIAEKKKRVTDVDLKALVSDQASHAEPIWKLGGLQVTCGTMGSSTATIKLVTSDGSTHVACSVGVGPVDSAYKAINLIVKETVKVLEYSPSTVTGGTDAIATTRVVIRRENKQSPTPALNGNVIYPTFSGTGEGVDIVTSSVEAYITALNKMLDSKE

SEQ ID NO: 15 - protein, 555 aa, Artificial Sequence
  A synthetic sequence
MATKTSTNGTHHSLPEYIPNRIPDPHYVRILDTTLRDGEQAPGAAMTSDQKLQIARQLAKLGVDVIEGGFPSASQEDFNAVKMIAQEVGNNCDADGYVPVIAALCRCNERDITRAWEALKYAKRPRLMPFIAVSPIHMEYKLNKTKEEVLQIATDMIKFARGLGCTNIQFCSEDAARSDREFLYQILEEVIKAGATTLGIGDTVGITMPFEIRELVAGIKANVPGAENVIISIHCHNDLGHATANTIEAARAGAMQLEVTINGIGERAGNASLEEVVMALKCRGDHVLGGLYTGINTRHLLKTSKMVEEFSGMYLQPHKAVVGDNAFLHESGVHQAGLLKHRGTYEILSPEDIGHEKSNGVNMVLGKLSGRQALKSRLKELGYELRDEEVESVFRNFKAIAEKKKRVTDVDLKALVSDQASHAEPIWKLGGLQVTCGTMGSSTATIKLVTSDGSTHVACSVGVGPVDSAYKAINLIVKETVKVLEYSPSTVTGGTDAIATTRVVIRRENKQSPTPALNGNVIYPTFSGTGEGVDIVTSSVEAYITALNKMLDSKE

SEQ ID NO: 16 - protein, 555 aa, Artificial Sequence
  A synthetic sequence
MATKTSTNGTHHSLPEYIPNRIPDPHYVRILDTTLRDGEQAPGAAMTSDQKLQIARQLAKLGVDVIEGGFPSASQEDFNAVKMIAQEVGNNCDADGYVPVIAALCRCNERDITRAWEALKYAKRPRLMPFIAVSPIHMEYKLNKTKEEVLQIATDMIKFARGLGCTDIQFCSEDAARSDREFLYQILEEVIKAGATTLGIGDTVGITMPFEIRELVAGIKANVPGAENVIISIHCHNDLGHATANTIEAARAGAMQLEVTINGIGERAGNASLEEVVMALKCRGDHVLGGLYTGINTRHLLKTSKMVEEFSGMYLQPHKAVVGDNAFLHESGVHQAGLLKHRGTYEILSPEDIGHEKSNGVNMVLGKLSGRQALKSRLKELGYELRDEEVESVFRNFKAIAEKKKRVTDVDLKALVSDQASHAEPIWKLGGLQVTCGTMGSSTATIKLVTSDGSTHVACSVGVGPVDSAYKAINLIVKETVKVLEYSPSTVTGGTDAIATTRVVIRRENKQSPTPALNGNVIYPTFSGTGEGVDIVTSSVEAYIEALNKMLDSKE

SEQ ID NO: 17 - protein, 620 aa, Glycine max
MATVIRNPILFPSTSHHPNQNHTFLTLRFSQTLRSSLRSKSRFAVSCSQSEPPPPHPSSSRRRPPYIPNLIPDPSYVRIFDTTLRDGEQSPGASMTSKEKLDVARQLAKLGVDIIEAGFPAASKDDFEAVKMIAQAVGNAVENDGYVPVICGLSRCNEKDIRTAWEAVKYAKRPRIHTFIATSPIHMEYKLRMSKDKVVDIARNMVKFARSLGCDDVEFSPEDAGRSDREFLYEILGEVIKVGATTLNIPDTVGITMPSEFGKLIADIKANTPGIENVIISTHCQNDLGLSTANTIEGARAGARQLEVTINGIGERAGNASLEEVVMALRCGAHVNGNLYTGINTKHIFLTSKMVEEYTGLQIQPHKALVGANAFAHESGIHQDGMLKHKGTYEIISPEDIGLERTNEAGIVLGKLSGRHALRKRLEELGYELNDDQVQTLFWCFKAVAEQKKRVTDADLRALVSDEVFQAEPVWKLGDLQVTCGTLGLSTATVKLLSSDGSTHVACSIGTGPVDSAYKAVDLIVKEQVTLLEYSMNAVTEGIDAIATTRVVIRGESETSTITTHALTGETVIRTFSGTGAGMDVVVSSVKAYIAALNKMSGFKESSQSAEKISISSLKI

SEQ ID NO: 18 - protein, 620 aa, Artificial Sequence
  A synthetic sequence
MATVIRNPILFPSTSHHPNQNHTFLTLRFSQTLRSSLRSKSRFAVSCSQSEPPPPHPSSSRRRPPYIPNLIPDPSYVRIFDTTLRDGEQSPGASMTSKEKLDVARQLAKLGVDIIEAGFPAASKDDFEAVKMIAQAVGNAVENDGYVPVICGLSRCNEKDIRTAWEAVKYAKRPRIHTFIATSPIHMEYKLRMSKDKVVDIARNMVKFARSLGCDNVEFSPEDAGRSDREFLYEILGEVIKVGATTLNIPDTVGITMPSEFGKLIADIKANTPGIENVIISTHCQNDLGLSTANTIEGARAGARQLEVTINGIGERAGNASLEEVVMALRCGAHVNGNLYTGINTKHIFLTSKMVEEYTGLQIQPHKALVGANAFAHESGIHQDGMLKHKGTYEIISPEDIGLERTNEAGIVLGKLSGRHALRKRLEELGYELNDDQVQTLFWCFKAVAEQKKRVTDADLRALVSDEVFQAEPVWKLGDLQVTCGTLGLSTATVKLLSSDGSTHVACSIGTGPVDSAYKAVDLIVKEQVTLLEYSMNAVTEGIDAIATTRVVIRGESETSTITTHALTGETVIRTFSGTGAGMDVVVSSVKAYIAALNKMSGFKESSQSAEKISISSLKI

SEQ ID NO: 19 - protein, 620 aa, Artificial Sequence
  A synthetic sequence
MATVIRNPILFPSTSHHPNQNHTFLTLRFSQTLRSSLRSKSRFAVSCSQSEPPPPHPSSSRRRPPYIPNLIPDPSYVRIFDTTLRDGEQSPGASMTSKEKLDVARQLAKLGVDIIEAGFPAASKDDFEAVKMIAQAVGNAVENDGYVPVICGLSRCNEKDIRTAWEAVKYAKRPRIHTFIATSPIHMEYKLRMSKDKVVDIARNMVKFARSLGCDDVEFSPEDAGRSDREFLYEILGEVIKVGATTLNIPDTVGITMPSEFGKLIADIKANTPGIENVIISTHCQNDLGLSTANTIEGARAGARQLEVTINGIGERAGNASLEEVVMALRCGAHVNGNLYTGINTKHIFLTSKMVEEYTGLQIQPHKALVGANAFAHESGIHQDGMLKHKGTYEIISPEDIGLERTNEAGIVLGKLSGRHALRKRLEELGYELNDDQVQTLFWCFKAVAEQKKRVTDADLRALVSDEVFQAEPVWKLGDLQVTCGTLGLSTATVKLLSSDGSTHVACSIGTGPVDSAYKAVDLIVKEQVTLLEYSMNAVTEGIDAIATTRVVIRGESETSTITTHALTGETVIRTFSGTGAGMDVVVSSVKAYIAELNKMSGFKESSQSAEKISISSLKI

SEQ ID NO: 20 - protein, 641 aa, Zea mays
MAFNAKPYCSTTAKPPTPAAHRSPSGPSPCISVRAAAVSPRARHSAYGLSAAGNASSSTVRLRALAQRTRAQPQPPRRRPEYVPNRIDDPNYVRIFDTTLRDGEQSPGATMTSAEKLVVARQLARLGVDVIEAGFPASSPDDLDAVRSIAIEVGNPPPGDDGGAHVPVICGLSRCNRRDIDAAWEAVRHARRPRIHTFIATSEIHMQHKLRKTPDQVVAVAREMVAYARSLGCADVEFSPEDAGRSNREFLYHILEEVIKAGATTLNIPDTVGYTLPYEFGKLISDIKENTPGIENAIISTHCQNDLGLATANTLAGAHAGARQLEVTINGIGERAGNASLEEVVMAIKCRGELLDGLYTGINSQHITLTSKMVQEHSGLHVQPHKAIVGANAFAHESGIHQDGMLKYKGTYEIISPDDIGLARANEFGIVLGKLSGRHAVRSKLVELGYEISDKEFEDFFKRYKEVAEKKKRVTDEDIEALLSDEIFQPKVIWSLADVQATCGTLGLSTATVKLIGPDGEERIACSVGTGPVDAAYKAVDQIIQIPTVLREYGMTSVTEGIDAIATTRVVVTGDVANNSKHALTGHSFNRSFSGSGAAMDIVVSSVRAYLSALNKMCSFAGAARASSTEVPGSASVQRAE

SEQ ID NO: 21 - protein, 641 aa, Artificial Sequence
  A synthetic sequence
MAFNAKPYCSTTAKPPTPAAHRSPSGPSPCISVRAAAVSPRARHSAYGLSAAGNASSSTVRLRALAQRTRAQPQPPRRRPEYVPNRIDDPNYVRIFDTTLRDGEQSPGATMTSAEKLVVARQLARLGVDVIEAGFPASSPDDLDAVRSIAIEVGNPPPGDDGGAHVPVICGLSRCNRRDIDAAWEAVRHARRPRIHTFIATSEIHMQHKLRKTPDQVVAVAREMVAYARSLGCANVEFSPEDAGRSNREFLYHILEEVIKAGATTLNIPDTVGYTLPYEFGKLISDIKENTPGIENAIISTHCQNDLGLATANTLAGAHAGARQLEVTINGIGERAGNASLEEVVMAIKCRGELLDGLYTGINSQHITLTSKMVQEHSGLHVQPHKAIVGANAFAHESGIHQDGMLKYKGTYEIISPDDIGLARANEFGIVLGKLSGRHAVRSKLVELGYEISDKEFEDFFKRYKEVAEKKKRVTDEDIEALLSDEIFQPKVIWSLADVQATCGTLGLSTATVKLIGPDGEERIACSVGTGPVDAAYKAVDQIIQIPTVLREYGMTSVTEGIDAIATTRVVVTGDVANNSKHALTGHSFNRSFSGSGAAMDIVVSSVRAYLSALNKMCSFAGAARASSTEVPGSASVQRAE

SEQ ID NO: 22 - protein, 641 aa, Artificial Sequence
  A synthetic sequence
MAFNAKPYCSTTAKPPTPAAHRSPSGPSPCISVRAAAVSPRARHSAYGLSAAGNASSSTVRLRALAQRTRAQPQPPRRRPEYVPNRIDDPNYVRIFDTTLRDGEQSPGATMTSAEKLVVARQLARLGVDVIEAGFPASSPDDLDAVRSIAIEVGNPPPGDDGGAHVPVICGLSRCNRRDIDAAWEAVRHARRPRIHTFIATSEIHMQHKLRKTPDQVVAVAREMVAYARSLGCADVEFSPEDAGRSNREFLYHILEEVIKAGATTLNIPDTVGYTLPYEFGKLISDIKENTPGIENAIISTHCQNDLGLATANTLAGAHAGARQLEVTINGIGERAGNASLEEVVMAIKCRGELLDGLYTGINSQHITLTSKMVQEHSGLHVQPHKAIVGANAFAHESGIHQDGMLKYKGTYEIISPDDIGLARANEFGIVLGKLSGRHAVRSKLVELGYEISDKEFEDFFKRYKEVAEKKKRVTDEDIEALLSDEIFQPKVIWSLADVQATCGTLGLSTATVKLIGPDGEERIACSVGTGPVDAAYKAVDQIIQIPTVLREYGMTSVTEGIDAIATTRVVVTGDVANNSKHALTGHSFNRSFSGSGAAMDIVVSSVRAYLEALNKMCSFAGAARASSTEVPGSASVQRAE

SEQ ID NO: 23 - protein, 640 aa, Zea mays
MAFSAKPHCSCSTTTKPPTPVAHLSPPPSLSVRAAACAPRSAYGLSAAGVGGGKASPSTVRLRARAQRIRASQQQPRRRPEYVPNRIDDPNYVRIFDTTLRDGEQSPGATMTSAEKLVVARQLARLGVDIIEAGFPASSPDDLDAVRSIAIEVGNPPTASAGTVHVPVICGLSRCNRKDIDAAWEAVRHARRPRIHTFIATSEIHMQHKLRKTPEQVVAIAREMVAYARSLGCPDVEFSPEDAGRSNREFMYHILEEVIKAGATTLNIPDTVGYTLPYEFGKLIADIKANTSGIENAIISTHCQNDLGLATANTLAGARSGARQLEVTINGIGERAGNASLEEVVMAIKCRGELLDGLYTGINSQHITLTSKMVQEHSGLHVQPHKAIVGANAFAHESGIHQDGMLKYKGTYEIISPDDIGLTRANEFGIVLGKLSGRHAVRSKLVELGYEISDKEFEDFFKRYKEVAEKKKRVTDEDIEALLSDEIFQPKVIWSLADVQATCGTLGLSTATVKLIAPDGEERIACSVGTGPVDAAYKAIDQIIQIPTVLREYGMTSVTEGIDAIATTRVVVTGDVTNNSKHALTGRAFNRSFSGSGAAMDIVVSSVRAYLSALNKMCSFAGAAKASGEVPESASVQSAE

SEQ ID NO: 24 - protein, 640 aa, Artificial Sequence
  A synthetic sequence
MAFSAKPHCSCSTTTKPPTPVAHLSPPPSLSVRAAACAPRSAYGLSAAGVGGGKASPSTVRLRARAQRIRASQQQPRRRPEYVPNRIDDPNYVRIFDTTLRDGEQSPGATMTSAEKLVVARQLARLGVDIIEAGFPASSPDDLDAVRSIAIEVGNPPTASAGTVHVPVICGLSRCNRKDIDAAWEAVRHARRPRIHTFIATSEIHMQHKLRKTPEQVVAIAREMVAYARSLGCPNVEFSPEDAGRSNREFMYHILEEVIKAGATTLNIPDTVGYTLPYEFGKLIADIKANTSGIENAIISTHCQNDLGLATANTLAGARSGARQLEVTINGIGERAGNASLEEVVMAIKCRGELLDGLYTGINSQHITLTSKMVQEHSGLHVQPHKAIVGANAFAHESGIHQDGMLKYKGTYEIISPDDIGLTRANEFGIVLGKLSGRHAVRSKLVELGYEISDKEFEDFFKRYKEVAEKKKRVTDEDIEALLSDEIFQPKVIWSLADVQATCGTLGLSTATVKLIAPDGEERIACSVGTGPVDAAYKAIDQIIQIPTVLREYGMTSVTEGIDAIATTRVVVTGDVTNNSKHALTGRAFNRSFSGSGAAMDIVVSSVRAYLSALNKMCSFAGAAKASGEVPESASVQSAE

SEQ ID NO: 25 - protein, 640 aa, Artificial Sequence
  A synthetic sequence
MAFSAKPHCSCSTTTKPPTPVAHLSPPPSLSVRAAACAPRSAYGLSAAGVGGGKASPSTVRLRARAQRIRASQQQPRRRPEYVPNRIDDPNYVRIFDTTLRDGEQSPGATMTSAEKLVVARQLARLGVDIIEAGFPASSPDDLDAVRSIAIEVGNPPTASAGTVHVPVICGLSRCNRKDIDAAWEAVRHARRPRIHTFIATSEIHMQHKLRKTPEQVVAIAREMVAYARSLGCPDVEFSPEDAGRSNREFMYHILEEVIKAGATTLNIPDTVGYTLPYEFGKLIADIKANTSGIENAIISTHCQNDLGLATANTLAGARSGARQLEVTINGIGERAGNASLEEVVMAIKCRGELLDGLYTGINSQHITLTSKMVQEHSGLHVQPHKAIVGANAFAHESGIHQDGMLKYKGTYEIISPDDIGLTRANEFGIVLGKLSGRHAVRSKLVELGYEISDKEFEDFFKRYKEVAEKKKRVTDEDIEALLSDEIFQPKVIWSLADVQATCGTLGLSTATVKLIAPDGEERIACSVGTGPVDAAYKAIDQIIQIPTVLREYGMTSVTEGIDAIATTRVVVTGDVTNNSKHALTGRAFNRSFSGSGAAMDIVVSSVRAYLEALNKMCSFAGAAKASGEVPESASVQSAE

SEQ ID NO: 26 - protein, 612 aa, Solanum lycopersicum
MASITANHPISGKPLISFRPKNPLLQTQTLFNFKPSISKHSNSSFSIPVVRCSIRRRPEYTPSHIPDPNYVRIFDTTLRDGEQSPGATMTTKEKLDVARQLAKLGVDIIEAGFPASSEADLEAVKLIAKEVGNGVYEEGYVPVICGLARCNKKDIDKAWEAVKYAKKPRIHTFIATSEIHMNYKLKMSRDQVVEKARSMVAYARSIGCEDVEFSPEDAGRSDPEFLYHILGEVIKAGATTLNIPDTVGYTVPEEFGQLIAKIKANTPGVEDVIISTHCQNDLGLSTANTLAGACAGARQLEVTINGIGERAGNASLEEVVMALKCRGEQVLGGLYTGINTQHILMSSKMVEEYSGLHVQPHKAIVGANAFAHESGIHQDGMLKHKDTYEIISPEDIGLNRANESGIVLGKLSGRHALQAKMLELGYEIEGKELDDLFWRFKSVAEKKKKITDDDLVALMSDEVFQPQFVWQLQNVQVTSGSLGLSTATVKLIDADGREHISCSVGTGPVDAAYKAVDLIVKVPVTLLEYSMNAVTQGIDAIASTRVLIRGENGHTSTHAVTGETIHRTFSGTGADMDIVISSVRAYVGALNKMMSFRKLMAKNNKPESSAVV

SEQ ID NO: 27 - DNA, 2218 bp, Solanum lycopersicum
taacagtctcttacttgaattttcagtctcttctcccagccgccattaaaaccagcactcgcaaactccaatttctcttctccggcaatggcgtctatcaccgcaaatcatccaatctccggtaaaccattaatctcattccgtcccaaaaaccctttacttcaaacccaaactctcttcaatttcaaaccatcaatctccaagcactccaattcttcattttccattcccgttgtccgctgctcaatccgccgtagaccggaatatactccgagtcacattcccgatccaaactatgtccggatattcgacaccactctccgtgatggcgaacaatcaccaggggctacaatgactacaaaggagaaactggatgttgcacgtcagttagctaagcttggtgttgatataattgaggctggttttcctgcttcttctgaagctgatcttgaagctgtgaaattgatagctaaggaagttgggaatggtgtttatgaagagggatatgttccggttatttgtggattggcgaggtgtaataagaaggatattgataaggcgtgggaggctgtgaagtatgctaagaaaccgaggattcatacgtttattgctacaagtgagatacatatgaattataagctgaaaatgagtagagatcaagttgttgagaaagctaggagtatggtggcttatgcaaggagtattgggtgtgaggatgttgaatttagccctgaagatgctggaagatctgatccagagtttctttatcatatccttggagaggttatcaaagctggggcaacaacccttaacatccctgatactgttggatacactgtacccgaagaatttggacaattgattgctaaaataaaagcgaataccccaggagttgaagatgtgatcatttcaacacactgccagaacgatcttgggctttctactgccaacaccttagctggagcatgtgcaggtgcaagacaattggaagtgaccatcaatggaattggtgaaagagctggaaatgcttctttagaggaggttgtaatggccttaaagtgtcgtggagagcaagtactaggtggcctatatacagggattaatacacaacatatactcatgtcaagcaagatggtagaggagtattccggacttcatgtgcagccacacaaagccattgttggagctaatgcctttgctcatgaaagtggcatccatcaggatggaatgttaaaacacaaagatacatatgagattatatctcctgaagatattgggcttaatcgtgctaatgaatctggtattgtccttgggaaactcagtgggcgtcatgctttgcaagccaaaatgcttgagcttggatacgagattgagggcaaagaacttgatgacctgttctggcgattcaaatctgtggctgagaagaaaaagaaaattacagatgatgacctggtagcactgatgtcagatgaggttttccagcctcaatttgtgtggcaacttcaaaatgtacaggttacttctggaagtcttggcctttctacagcaactgttaagctcattgatgctgatggtcgagagcatatttcttgttctgttggaacggggccagttgacgcggcttataaggcagttgatctcattgttaaggtacctgtaacactccttgagtattccatgaatgcagtcacacaaggtatagatgctatagcttcaaccagagtcttaattcgtggagaaaatggccatacatcaacccatgccgtaactggagagactattcaccgtacatttagtggaaccggagcagatatggatattgtcatctccagtgtccgagcctatgttggtgcattgaataagatgatgagtttcagaaaactaatggcgaaaaataacaaacccgaaagcagtgcagtcgtataggtacttctgtgcaaatcaaggttatggaacttttgcaactgcactggagctttatcatttgtacaaaatgtaggagtctgttcaaagaatttgagcctgtagttttcaagaaaacaaagcttaatatgtctggtagtgcttgaaaatcatctaagtttatggtgtatcagttggaacattagacacattgtccatataactttgttcatgctcccgtttaactaatttatgaatctacataccacgcaagattttcgaaatgatttcagaaattatgaaaatctctgtatta

SEQ ID NO: 28 - protein, 614 aa, Solanum lycopersicum
MSSSSSLCSNSVFSYRNNFSIFQSKNVLLPPISSTNNFSFSIKKHYYSTFIRCSISNRRPEYVPSKISDPKYVRIFDTTLRDGEQSPGATMTTKEKLDVARQLAKLGVDIIEAGFPASSEADFESVKLIAEEIGNNTDENGFVPVICGLSRCNKSDIDKAWEAVKYAKKPRVHTFIATSEIHMKYKLKMSREQVVEKARSMVAYARSLGCEDVEFSPEDAGRSDREFLYDILGEVIKAGATTLNIPDTVGYTVPSEFGQLIADIKANTPGIENVIISTHCQNDLGLSTANTLAGACAGARQLEVTINGIGERAGNASLEEVVMALKCRGEQVLGGLYTGINTQHIVPSSKMVEEYSGLQVQPHKAIVGANAFAHESGIHQDGMLKHKDTYEIISPDDVGLSRSNEAGIVLGKLSGRHALKSKMLELGYDIDGKELEDLFWRFKSVAEKKKKITDDDLIALMSDEVLQPNVYWKLGDVQIMCGSLGLSTATVKLINTDGQEHIACSVGTGPVDAAYKAVDLIVKVPITLLEYSMNAVTEGIDAIASTRVSICSIDRHTIMNGSTGQTIHRTFSGTGADMDVVISSVRAYIGALNKMLSYEKLVSRYSKPEDSVVV

SEQ ID NO: 29 - DNA, 2282 bp, Solanum lycopersicum
ctctttgtgaaatgatctagaatcttttaacatatgagataaatgacttggtatccacttggttcaaataatttggagtacacttttttctttttattatataaatctccacactttgttagtattaaaaccccacttttccttttctcctctgtgtttgacccttttctgcacattttcagaatgtcttcttcttcttctctttgttcaaactctgtattttcttatagaaacaacttctcaatttttcaatccaaaaatgttcttcttcctccaatttctagtaccaataatttcagtttttcaatcaaaaaacactactactccacatttatccggtgttcgatttcgaatcgtcgaccggaatatgtacccagtaaaatctccgacccgaaatacgttcgcatattcgatactactcttcgtgacggtgagcaatctccaggtgctacaatgactacgaaagagaaactcgacgtcgctcgtcagctagcgaaactcggtgttgatataattgaagctggatttccagcttcatctgaagcagatttcgaatctgtgaaactaattgcagaggaaattggtaataatactgatgaaaatggatttgtgcctgtaatttgtgggttatctagatgtaataaaagtgatattgataaagcttgggaagcagtgaaatacgctaaaaaacctagggttcatacgtttattgctacgagtgaaatacatatgaagtataaattgaagatgagtagagaacaagtggtggaaaaagcaaggagtatggtagcttatgctagaagccttggatgtgaagatgttgaatttagtccagaagatgcaggaaggtctgatcgagagttcctttatgatatcctcggagaagttattaaagctggtgcaacaacacttaacatacctgatactgttggatacactgttccaagtgaatttggacaattaattgctgacataaaagccaatactccggggattgaaaatgtgataatttcaacacattgccagaacgatcttgggctttctactgccaacactttagctggagcttgtgcaggagcaagacaactagaggtgaccattaatggcattggtgaaagagctggaaatgcttctctggaggaggttgtaatggccttaaaatgtcgcggagagcaagtattaggtggcctctacacggggattaacactcaacatattgttccatcgagcaaaatggtggaggagtacagtgggctacaggtgcagccacataaggccattgttggagctaatgcatttgctcatgaaagtggcatccatcaggatggaatgttaaaacacaaggacacctatgagattatatctcctgatgatgttgggcttagtcgttctaatgaagcgggtattgtccttgggaaactcagtggtcgccatgcactgaaatccaaaatgcttgagcttggatatgacattgatggaaaagaactagaggacctcttttggcgttttaagtcagtagctgagaagaaaaagaaaattacagatgatgacttaatagcactgatgtcagatgaagttctccaacctaatgtttattggaagcttggagatgtacagattatgtgtggaagtcttggcctctctacagcaactgtgaagcttataaacactgatggtcaagagcatattgcttgttcagttggaacaggacctgttgatgcagcttacaaggcagtggacctcattgtgaaggtgcctattacgctccttgaatattccatgaatgcagtcacagaaggtatagatgccatagcatcaaccagagtgtcaatctgcagtattgataggcatactataatgaatggttcaactggacagactattcaccgcacatttagtggaaccggagcagatatggatgttgttatctctagtgtccgagcgtatattggtgcattgaacaaaatgttgagttacgaaaagctggtgtcaagatacagcaaacctgaagacagtgtggtggtataagaaaatgttcgtaatgttccagtttcttgtcatttctcttgtcaattgtatagaactaggggtgccttatcaacaaattacgacttgcctggaagaacgataaaaggcaaatttgagtcgtaatgcattttcattttctgcaggttgatgtacaagcttgtactaaatgtgttaaagtcattttaggctttgtgttgtaccaatcaaacacagatccttttatgtggtttagctttaaattgatttttgcttaa

SEQ ID NO: 30 - protein, 635 aa, Oryza sativa
MASSLLSSPKPSSFSSANPTSTPRPRAQTLSPFRAAAPRFSHGLATAAAAANPSASRRCYHRAFARPVRASMAQPRRPEYVPNRIDDPNYVRIFDTTLRDGEQSPGATMTSAEKLVVARQLARLGVDIIEAGFPASSPDDLDAVRSIAIEVGNTPVGEDGHVPVICGLSRCNKRDIDAAWEAVRHARRPRIHTFIATSEIHMQHKLRKTPEQVVAIAKEMVAYARSLGCPDVEFSPEDAGRSNREFLYHILEEVIKAGATTLNIPDTVGYTLPYEFGKLIADIKANTPGIENAIISTHCQNDLGLATANTLAGAHAGARQLEVTINGIGERAGNASLEEVVMAIKCRRELLGGLYTGINTQHITMSSKMVQEHSGLHVQPHKAIVGANAFAHESGIHQDGMLKYKGTYEIISPDDIGLTRANEFGIVLGKLSGRHAVRSKLVELGYEITDKEFEDFFKRYKEVAEKKKRVTDEDIEALLSDEIFQPKVFWSLADVQATCGTLGLSTATVKLIGPDGEEKIACAVGTGPVDAAYKAVDDIIQIPTVLREYSMTSVTEGIDAIATTRVVVTGDVSDSKHALTGHSFSRAFSGSGAALDIVVSSVRAYLSALNKMSSFVGAIKASSEVSESQRVQTTE

SEQ ID NO: 31 - DNA, 2395 bp, Oryza sativa
tcgtgcatcccacgaaacccccgttgtcatgtccagataagagctccccctctgactcacacacacatcaccgagaagcagagcaagtgcatctccctctcccaccatttgattcgattcccctaaacccccgagtcgtcgacgccacaaaccccggcgaccaccatggcctcctccctcctctcctccccaaaaccctcctccttctcctccgcaaaccccacctccactccacgcccacgcgcccaaactctctcccccttccgcgccgccgccccacgcttctcccatggcctcgccaccgccgccgccgccgcaaaccctagcgcctcccgccgctgctaccaccgcgccttcgcccgccccgtccgggcgtccatggcgcagccgcggcgcccggagtacgtccccaaccgcatcgacgaccccaactacgtccgcatcttcgacaccaccctccgcgacggggagcagtcccccggggccaccatgaccagcgccgagaagctcgtcgtcgcgcgccagctcgcccgcctcggcgtcgacatcatcgaggccgggttcccggcctcctcccccgacgacctcgacgccgtgcgctccatcgccatcgaggtcgggaacacgcccgtcggggaggacggccacgtgccggtcatctgtggcctctcgagatgcaataagcgagacattgatgctgcctgggaggccgtgcggcacgcgcggcggccgcgcatccacaccttcattgccaccagcgagatccatatgcagcacaagctaaggaagacgcccgagcaggtggtggccattgccaaggaaatggtggcctacgcccgcagcctaggctgccctgatgtcgagttcagccctgaagacgctggcaggtcaaacagagagtttctatatcatattctagaggaagtcataaaagctggagcaacaacactcaatatcccagacactgttggatacactcttccttatgaatttgggaagctaattgctgatataaaagcaaacactccaggaattgaaaatgctattatttctactcattgccagaatgaccttggtctagcaaccgccaatacattagcgggcgctcatgcaggagcacggcaattagaggtgactatcaacggtattggtgaaagggctggaaatgcttctttggaagaggttgtgatggcaattaaatgtcgccgagagctcttaggaggtctgtatactggaatcaatacccaacatatcactatgtcaagcaaaatggtacaagagcacagtggacttcatgtacaaccacataaagctattgtcggtgccaatgcctttgcacatgaaagtggaattcatcaggatgggatgcttaaatacaaaggaacttatgaaataatttctcctgatgatattggtctaacacgtgcaaacgagtttggtattgttcttgggaaactcagtggaaggcatgctgtgagatctaaactagtggagcttggatatgaaatcactgacaaggaatttgaggatttctttaaacgctacaaagaggttgcagagaagaaaaagcgtgtaactgatgaagacattgaggcgctgttgtctgatgagatatttcagcccaaggttttttggtcccttgctgatgtacaggcaacttgtggaacacttggtctgtctacagcaactgtcaaactgataggtccggatggagaggagaagattgcatgtgcagttggaacaggtccagttgatgcagcttacaaggctgttgatgatataatacagatcccaactgttcttcgagaatatagcatgacatcggtcacagaaggcattgatgcaattgcaactactagagtggttgtcactggagatgttagcgactctaaacatgctttgactggtcactccttcagccgggcattcagtgggagtggtgccgcactggatattgttgtttccagtgtgcgagcttacctgagtgccctgaacaagatgtccagttttgttggggctatcaaggctagtagtgaagtatctgaaagccaaagagttcaaaccacagaatgagtcttgacttccttttgggttttcatatccgatggttctatgtttcacattcccagcaaggagtatgtgcttgttgaaacatggtttttccgtccagaaaaaacatggttccgtttagtgcatctggaggatgttctgggtttcttggtggagcctgacttaaggttgaacatccaggacgttttgggatatgcagtgtataattcatatttgaaaaccgtatttacaataagacaataataaataattgtttgacatatgagtattgcaaaactattactgtaagaaattaatcgtgagaccaacctacggttgtacagta

SEQ ID NO: 32 - protein, 635 aa, Oryza sativa
MASSLLSSPKPSSFSSANPTSTPRPRAQTLSPFRAAAPRFSHGLATAAAAANPSASRRCYHRAFARPVRASMAQPRRPEYVPNRIDDPNYVRIFDTTLRDGEQSPGATMTSAEKLVVARQLARLGVDIIEAGFPASSPDDLDAVRSIAIEVGNTPVGEDGHVPVICGLSRCNKRDIDAAWEAVRHARRPRIHTFIATSEIHMQHKLRKTPEQVVAIAKEMVAYARSLGCPDVEFSPEDAGRSNREFLYHILEEVIKAGATTLNIPDTVGYTLPYEFGKLIADIKANTPGIENAIISTHCQNDLGLATANTLAGAHAGARQLEVTINGIGERAGNASLEEVVMAIKCRRELLGGLYTGINTQHITMSSKMVQEHSGLHVQPHKAIVGANAFAHESGIHQDGMLKYKGTYEIISPDDIGLTRANEFGIVLGKLSGRHAVRSKLVELGYEITDKEFEDFFKRYKEVAEKKKRVTDEDIEALLSDEIFQPKVFWSLADVQATCGTLGLSTATVKLIGPDGDEKIACAVGTGPVDAAYKAVDDIIQIPTVLREYSMTSVTEGIDAIATTRVVVTGDVSDSKHALTGHSFNRAFSGSGAALDIVVSSVRAYLSALNKMSSFVGAIKASSEVSESQRVQTTE

SEQ ID NO: 33 - protein, 620 aa, Sorghum bicolor
MAFTAKPYCSTPTKPPTLSARAPRSAHGLSAAAAATPSTVRLRAFAQRIRAQSQQQQQRRRRPEYVPHRIDDPNYVRIFDTTLRDGEQSPGATMTSAEKLVVARQLARLGVDIIEAGFPASSPDDLDAVRSIAIEVGNPVEEGAHVPVICGLSRCNKRDIDAAWEAVRNARRPRIHTFIATSEIHMQHKLRKTPEQVVAIAKEMVAYARSLGCPDVEFSPEDAGRSNREFLYHILEEVIKAGATTLNIPDTVGYTLPYEFGKLIADIKANTPGIENAIISTHCQNDLGLATANTLAGARAGARQLEVTINGIGERAGNASLEEVVMAIKCRGELLDGLYTGINSQHITLTSKMVQEHSGLHVQPHKAIVGANAFAHESGIHQDGMLKYKGTYEIISPDDIGLTRANEFGIVLGKLSGRHAVRSKLVELGYEISDKEFEDFFKRYKEVAEKKKRVTDEDIEALLSDEIFQPKVIWSLADVQATCGTLGLSTATVKLIAPDGEEKIGCSVGTGPVDAAYKAVDQIIQIPTVLREYGMTSVTEGIDAIATTRVVVTGDVTNNSKHALTGQSFNRSFSGSGAAMDIVVSSVRAYLSALNKMCSFAGAAKASSEVPESASVQRTE

SEQ ID NO: 34 - DNA, 2454 bp, Sorghum bicolor
cacggcacggcacctcaccgctctcgcgtgctcgccgcggcggccgccgtgctcgtgtcgtgtcacagccagcagctcgctccgacgacctccgatccgtgccgcctacgaaaccaccacccgtccggatcctccaggatatataccagccagctagtcatccccttgctggctgctgccttttccaaatccactcacattttccacatccaccgtcgatttaacacagtcccccgccgccgccgcccgcccgtccgtcccctcctaaaccccgcgacgaccctgagcgagcccgagccgagtccccggcgaccaaccaccatggccttcaccgctaaaccctactgctcaacccccaccaaaccccccaccctctccgcccgcgccccgcgctccgcccatggcctatccgccgccgccgccgcaaccccgagcaccgtccgcctccgcgcgttcgcccagcgcatccgggcgcagtcgcagcaacagcaacagcggcggcggcggcccgagtacgtgccgcaccgcatcgacgacccaaactacgtgcgcatcttcgacaccacgctccgcgacggggagcagtccccgggagccaccatgacgagcgccgagaagctggtggtcgcgcggcagctggcccggctcggcgtcgacatcatcgaggcggggttcccggcgtcctcccccgacgacctcgacgccgtgcgctccatcgccatcgaggtcggcaacccggtggaggaaggcgcccacgtgcccgtcatctgcggcctctcgcggtgcaacaagagggatattgatgccgcctgggaggccgtcaggaacgcgcgcaggccccggattcataccttcatcgccaccagcgagatccatatgcagcataagcttaggaagacgcctgagcaggtcgttgctattgctaaggagatggtggcgtatgcacgcagccttgggtgccctgatgtcgaattcagccctgaggatgccggcaggtcaaatcgagaattcctgtatcatatactggaggaagtcattaaagctggggcaactactcttaatatcccagacactgtcggatacactcttccttatgaatttgggaagttgattgctgacataaaggcaaacactcctggaattgaaaatgctatcatttccactcattgccagaatgaccttggtcttgcaactgccaacacattagcgggcgctcgtgcaggagcacgacagttagaggtgactattaatggtattggtgaaagagctggaaatgcttcgttggaagaggttgtcatggcaattaaatgtcgtggggagctcttagatggtctatatacgggaatcaattcccaacatattactttgacaagcaaaatggtacaagagcacagtggacttcatgtacaaccacataaagctattgttggtgccaatgcctttgctcatgaaagtggaattcatcaggatgggatgcttaagtacaagggaacatatgaaataatatcgcctgatgatattggtttaacacgtgcgaatgaatttggtattgttcttgggaaactcagcggaagacatgcagtgagatctaagctagtggagcttggatatgaaatcagtgacaaggaatttgaagatttctttaaacgctacaaagaggttgcagagaagaaaaagcgtgtaactgatgaagacatagaagcgttattgtcagatgagatattccagcctaaggttatttggtcccttgctgatgtacaggcaacatgtggaacacttggcttatctacagcaacagtgaaactgatagcaccagatggagaggagaaaataggatgttcagttggaacaggtccagttgatgcagcttacaaggctgttgaccaaataatccagattccaactgttctccgagaatatggtatgacttcagtcacagagggcattgacgctatcgcgacaactcgagtggttgtcactggagatgtgaccaacaactccaagcatgccttgactggtcaatctttcaaccgctccttcagtgggagcggggcagctatggacatcgttgtgtccagcgtcagagcttacctgagtgccctgaacaagatgtgcagctttgctggtgctgcgaaagccagcagcgaggtacctgagagcgcaagcgttcaacgcacagagtgagcttggcgctcctctttgttcccatgtgggcttggcgacgtaagagcttgagcaactgttatagagtgtatgtcgtttcagtaacaggctgttcaatattggggttttcccttgtcagtgtggagtgattgtgctgttctattttggaggatagtccctttagcttagaacatgcaggaaattttggccctatgtagtgtacaatttgtgccttatatgaaccatactttcaataatgaaataatattagggtccatccagccacccata

SEQ ID NO: 35 - protein, 623 aa, Brassica napus
MASSILRNPMLSSPTTITTPSLPSFSSKPSPLSFRFPPSHHRSSLRIKSLRLSCSLSDPSPPLRRRRPEYIPNRISDPNYVRVFDTTLRDGEQSPGATLTSKEKLDIARQLAKLGVDVIEAGFPAASKDDFEAVKTIAETVGNAVDGDGYVPVICGLSRCNKRDIETAWEAVKYAKRPRIHTFIATSDIHLEYKLKKSKDEVIEIARNMVKFARSLGCEDVEFSPEDAGRSEREFLYQILGEVIKAGATTLNIPDTVGITLPSEFGQLIADIKANTPGIENVIISTHCQNDLGLSTANTLSGAHSGARQLEVTINGIGERAGNASLEEVVMAIKCRGDHVLGGLYTGIDTRHIVMTSKMVEDYTGMQTQPHKAIVGANAFAHESGIHQDGMLKHKGTYEIICPEEIGLERSNDAGIVLGKLSGRHALKDRLTELGYVLDDEQLSSIFWRFKSVAERKKRVTDADIIALVSDEVFQPEALWKLLDIQITCGTLGLSTATVKLADADGKEHVACSMGTGPVDSAYKAVDLVVKEPATLLEYSMNAVTEGIDAIATTRVLIRGNNNYSTTNAITGEEVQRTFSGTGAGMDIVVSSVKAYVGALNKMLDFKENSTTKIPSQNNKVPA

SEQ ID NO: 36 - protein, 626 aa, Brassica napus
MASSILRNPMLSSPTTTIPTPSLPSSSSKPSPLSFRFPPSHHRSSVSLRSQSLRLSCSLSDPSPPLRRRRPEYIPNRISDPNYVRVFDTTLRDGEQSPGATLTSKEKLDIARQLAKLGVDVIEAGFPAASKDDFEAVKTIAETVGNAVDGDGYVPVICGLSRCNKRDIETAWEAVKYAKRPRIHTFIATSDIHLEYKLKKSKDEVIEIARNMVKFARSLGCEDVEFSPEDAGRSEREFLYEILGEVIKAGATTLNIPDTVGITLPSEFGQLIADIKANTPGIENVIISTHCQNDLGLSTANTLSGAHSGARQVEVTINGIGERAGNASLEEVVMAIKCRGDHVLGGLYTGIDTRHIVMTSKMVEDYTGMQTQPHKAIVGANAFAHESGIHQDGMLKHKGTYEIICPEEIGLERSNDAGIVLGKLSGRHALKDRLTELGYVLDDEQLSSIFWRFKSVAERKKRVTDADIIALVSDEVFQPEALWRLLDIQITCGTLGLSTATVKLVDADGKEHVACSMGAGPVDSAYKAIDLIVKEPATLLEYSMNAVTEGIDAIATTRVLIRGNNNYSTTNAITGEEVQRTFSGTGAGMDIVVSSVKAYVGALNKMLDFKENAPTKVPSQNNNVPA

SEQ ID NO: 37 - DNA, 2141 bp, Brassica napus
cgatgagacagagctggatcaagttaccgccgccacgttgaaccttcttctctatcgtcgtccccgtttaggtttaccactcttctttcaacaatggcgtcttcgattctcagaaaccctatgctctcatcaccaacaacaacaatccccaccccttctcttccctcctcctcctcaaaaccctcacctctctcattccgcttcccaccctcccaccaccgctcctccgtttccctccgcagccaatccctccgcctctcctgctccctctcagatccctctcctcccctccgccgccgccgcccggagtacatccccaaccgcatttccgaccccaactacgtccgagtcttcgacaccactctccgcgacggcgaacagtcccccggagccaccctcacctccaaggaaaagctcgacatcgcgcgccagctcgcgaagctaggcgtcgacgtaatcgaggccggcttccccgccgcctccaaggacgacttcgaagccgtcaaaaccatagccgaaaccgtgggaaacgccgtcgacggagacggttacgtccccgtcatctgcggactctcgagatgcaacaagagagatatagagacggcgtgggaggctgtgaagtacgccaaaaggccgaggatccataccttcatcgccacgagtgacattcacttggagtataagctgaagaagagcaaagacgaggtcatcgagatcgctaggaatatggttaagttcgcgaggagcttggggtgtgaggacgttgagtttagtcctgaagatgctggaagatcggagagagagtttttgtatgagattcttggggaagtgataaaagctggagcgacaacgcttaatatacctgacactgttggtataacgttgcctagtgagtttggtcagttgattgctgatattaaagccaatactcctgggatcgagaatgttatcatctcaacgcattgtcagaatgatcttgggctctccactgccaacactttatctggtgcacattcgggtgcaaggcaagtggaagtgactatcaatggcattggggaaagagctggaaacgcttcactagaagaggttgtgatggccataaaatgccgtggagatcatgtattaggaggcctatatactggaatcgatactcgccacattgttatgacaagcaagatggttgaggattacacaggaatgcaaacacagccccataaggctattgtaggagcgaatgcctttgcgcatgaaagtggtattcatcaggatggaatgctgaaacacaagggcacatatgaaattatatgccccgaagaaattggacttgaacgatctaatgatgctggcattgttttggggaagcttagtgggcgtcatgcgttgaaagaccgtttgactgagcttggttatgtactagatgatgaacagctaagttccattttctggcgcttcaaatctgtggctgagcggaaaaagagagttaccgacgcagatataatagctttggtttctgatgaggttttccagccagaagccttgtggagactcctggacattcagattacatgtgggactctcggactctcaacagcaaccgttaaacttgttgatgctgatggcaaagagcatgttgcctgttctatgggtgctgggcctgtcgattcagcttataaggcaatcgatcttattgtcaaggaaccagcgactttgcttgagtactcaatgaatgcggtaacagaaggcatcgatgccattgcaaccacacgagttcttatccgaggaaataacaattactcaactacaaatgcaatcactggtgaagaagttcaaaggacctttagtggaaccggagctggaatggacattgtggtgtcgagcgtcaaagcttatgtaggagctttgaacaaaatgctcgacttcaaagaaaacgccccaacgaaagtcccttctcaaaacaacaatgtacctgcctgaatcaaaattgtttctgagtcagaccagagttagtcttttctggtataggtacatagtttggtaataacgagagttcaaggcttgcatattgttttaatgaagtatctttgctgaaagagttcgtttactataaaatatttatatagaacttaaatctctttttattt

SEQ ID NO: 38 - protein, 626 aa, Brassica napus
MASSILRNPMLSSPTTTITTPSLPSSSSKPSPLSFRFPPSHHRSSLSLRIKSLRLSCSLSDPSPPLRRRRPEYIPNRISDPNYVRVFDTTLRDGEQSPGATLTSKEKLDIARQLAKLGVDVIEAGFPAASKDDFEAVKTIAETVGNAVDGDGYVPVICGLSRCNKRDIETAWEAVKYAKRPRIHTFIATSDIHLEYKLKKSKDEVIEIARNMVKFARSLGCEDVEFSPEDAGRSEREFLYEILGEVIKAGATTLNIPDTVGITLPSEFGQLIADIKANTPGIENVIISTHCQNDLGLSTANTLSGAHSGARQVEVTINGIGERAGNASLEEVVMAIKCRGDHVLGGLYTGIDTRHIVMTSKMVEDYTGMQTQPHKAIVGANAFAHESGIHQDGMLKHKGTYEIICPEEIGLERSNDAGIVLGKLSGRHALKDRLTELGYVLDDEQLSSIFWRFKSVAERKKRVTDADIIALVSDEVFQPEALWRLLDIQITCGTLGLSTATVKLVDADGKEHVACSMGAGPVDSAYKAIDLIVKEPATLLEYSMNAVTEGIDAIATTRVLIRGNNNYSTTNAITGEEVQRTFSGTGAGMDIVVSSVKAYVGALNKMLDFKENAPTKVPSQNNNVPA

SEQ ID NO: 39 - skipped

SEQ ID NO: 40 - protein, 26 aa, Artificial Sequence
  A synthetic sequence
RFARSLGCEDVEFSPEDAGRSEREYL

SEQ ID NO: 41 - protein, 26 aa, Artificial Sequence
  A synthetic sequence
RFAKSLGFNDIQFGCEDGGRSDKDFL

SEQ ID NO: 42 - protein, 26 aa, Artificial Sequence
  A synthetic sequence
KYAKSLGFKDIQFGCEDGGRTEKDFI

SEQ ID NO: 43 - protein, 26 aa, Artificial Sequence
  A synthetic sequence
AYARSIGCEDVEFSPEDAGRSDPEFL

SEQ ID NO: 44 - protein, 26 aa, Artificial Sequence
  A synthetic sequence
KHLRSLGCNDIEFSPEDAGRSDPKFL

SEQ ID NO: 45 - protein, 32 aa, Artificial Sequence
  A synthetic sequence
RKCVEQAAKYPGTQWRFEYSPESYTGTELEYA

(19) United States
(12) Patent Application Publication
(10) Pub. No.: US 2023/0136941 A1
(43) Pub. Date: May 4, 2023

(54) IMPROVING PLANT NUTRITIONAL VALUE AND GROWTH THROUGH ENHANCEMENT OF ESSENTIAL AMINO ACID LEVELS

(71) Applicant: Board of Trustees of Michigan State University, East Lansing, MI (US)

(72) Inventors: Federica Brandizzi, Okemos, MI (US); Pengfei Cao, East Lansing, MI (US); Anqi Xing, Lansing, MI (US)

(21) Appl. No.: 17/995,697

(22) PCT Filed: Apr. 7, 2021
(86) PCT No.: PCT/US2021/026166

§ 371 Date: Oct. 6, 2022

Related U.S. Application Data
(60) Provisional application No. 63/006,543, filed on Apr. 7, 2020.

(51) Int. Cl. A01H 1/00; C12N 9/10

(52) U.S. Cl. CPC A01H 1/108; A01H 1/121; C12N 9/1025

(57) ABSTRACT

Described herein are modified isopropylmalate synthase nucleic acids and proteins, as well as methods, plants, plant cells, and seeds that include the modified isopropylmalate synthase. As shown herein, plants and seeds with such modified isopropylmalate synthase nucleic acids and proteins have increased biomass and/or increased amino acid content.

This application claims the benefit of U.S. Provisional Application Ser. No. 63/006,543, filed Apr. 7, 2020, which application is incorporated by reference herein its entirety.

### GOVERNMENT FUNDING

This invention was made with government support under 1714561 awarded by the National Science Foundation. The government has certain rights in the invention.

### INCORPORATION BY REFERENCE OF SEQUENCE LISTING

A Sequence Listing is provided herewith as a text file, “2131055.txt” created on Apr. 6, 2021 and having a size of 172,032 bytes. The contents of the text file are incorporated by reference herein in their entirety.

### BACKGROUND OF THE INVENTION

Traditionally, agricultural scientists concentrated on breeding plants with high nutritional yield. Typically, these new varieties were richer in carbohydrates but usually poorer in essential proteins and amino acids than the wild type varieties from which they were derived. Previous attempts on fortifying crops with high yield of essential amino acids have not been successful, largely due to accompanying penalties in plant growth. Hence, it has been difficult to generate plants with a complete range and adequate amounts of essential amino acids.

### SUMMARY

Described herein are plants, plant cells, and seeds with increased essential amino acid content relative to wild type or in some cases relative to plants with a knockout IPMS gene. In particular, described herein are plants, plant cells, and seeds with a modified IPMS gene, having one or more mutations. The mutations can be in the IPMS catalytic domain or in the IPMS1 allosteric domain. For example, plants with a modified IPMS catalytic domain (e.g., those with eva1 mutations) can have a conserved aspartic acid replaced by another amino acid, and plants with a modified IPMS in the allosteric domain include plants with ipms1-1D mutations. Plants with such modifications or mutations can have significantly higher levels of any and all amino acids. In some cases, the plants with such modifications or mutations can have significantly higher levels of Gln, His, Ile, Leu. Lys, Met, Phe, Thr, Trp, Val, or any combination thereof. The increased amino acid levels can occur in vegetative tissues (e.g., leaves) and seeds. In some cases, various plant tissues can have an increased content of just one, or just two, or just three, or just four, or just five, just six, just seven, just eight, just nine, or just ten amino acids. For example, in seeds with ipms1-1D mutations in their allosteric domain Ile, Leu, His, Lys, Met, Phe, and Thr levels are significantly increased. In the vegetative tissues of plants with ipms1-1D mutations in their allosteric domain leucine and other amino acids are present in increased amounts. Similarly, plants with a modified IPMS1 in the catalytic domain include plants with mutations such as the eva1 mutations. These plants with catalytic domain mutations can, for example, have significantly higher levels of valine in their seeds than wild type seeds. In their vegetative tissues, plants with modified catalytic domains such as the eva1 mutations can higher levels of valine and other amino acids. Plants with knockout ipms1 mutations (e.g., ipms1-4 or ipms1-5) can have modified or even increased content of some amino acids compared to wild type, but modification of the catalytic or allosteric domains typically provide higher levels of key amino acids such as the branched amino acids.

In addition, plants with such modifications or mutations can have significant increases in their biomass compared to wild type or compared to plants with a knockout IPMS gene. For example, plants with modifications to their IPMS1 allosteric domain can have significantly increased biomass. In some cases, plants with modifications to their catalytic domains tend to be smaller and generally have lower biomass.

Hence, described herein are plant cells, plant seeds, and/or plants that include a modified isopropylmalate synthase (IPMS) protein. The isopropylmalate synthase (IPMS) protein can be encoded by a modified or mutant endogenous isopropylmalate synthase (IPMS) gene within the plant cells, plant seeds, and/or plants. In some cases, an expression cassette having a promoter operably linked to a nucleic acid segment encoding a modified isopropylmalate synthase (IPMS) protein can be used to introduce expression of the modified isopropylmalate synthase (IPMS) protein into the plant cell, plant seed, or plant.

The modified isopropylmalate synthase protein can have isopropylmalate synthase activity. In some cases, the modified isopropylmalate synthase protein has a modification within its catalytic domain, a modification within its allosteric domain, or a combination thereof. For example, the modified isopropylmalate synthase protein can have an aspartic acid within its catalytic domain that is replaced by another amino acid. Such an aspartic acid can be at a position within the isopropylmalate synthase protein that corresponds to position 228 of SEQ ID NO:2. In some cases, the modified isopropylmalate synthase protein has a glycine within its allosteric domain that is replaced by another amino acid. Such a glycine can be at a position corresponding to position 606 of SEQ ID NO:2 in the modified isopropylmalate synthase protein. The modified isopropylmalate synthase protein can have a sequence with at least one amino acid modification to any of SEQ ID NO: 2, 3, 5, 7, 9, 10, 12, 13, 14, 15, 16, 17, 18, 19, 20, 21, 22, 23, 24, 25, 26, 28, 30, 32, 33, 35, 36, or 38. The plant or a plant generated from the plant cell or the plant seed with the modified isopropylmalate synthase protein can have increased amino acid content.

The plant cell, plant seed, or plant with the modified isopropylmalate synthase protein can be a forage species, starch species, oil species, grain species, grass species, sugar producing species, vegetable species of plant, plant cell or plant seed. The modified isopropylmalate synthase (IPMS) protein can be generated from a forage species, starch species, oil species, grain species, grass species, sugar producing species, or vegetable species of isopropylmalate synthase (IPMS). In some cases, the plant cell, plant seed, plant, and/or the isopropylmalate synthase (IPMS) protein can be a canola, corn, soybean, sunflower, walnut, or olive species.

Methods are also described herein that involve cultivating one or more seeds or seedlings, where the seeds or seedlings have a modified isopropylmalate synthase (IPMS) nucleic acid that can express a modified isopropylmalate synthase (IPMS) protein, to generate one or more mature plants, and harvesting vegetative tissues and/or seeds from the one or more mature plants.

Methods are also described herein that involve modifying a plant cell by introducing a mutation or modification in an endogenous isopropylmalate synthase (IPMS) gene, generating a plant from the plant cell, cultivating the plant, analyzing the amino acid content and/or biomass of the plant, and selecting one or more plants that have increased biomass or increased amino acid content. The increased biomass or the increased amino acid content can be measured relative to an average amino acid content, or an average biomass, of wild type plants, or parental plants, or plants with a knockout IPMS gene that do not express the modified isopropylmalate synthase protein.

### DESCRIPTION OF THE FIGURES

FIG. 1A-1J illustrate identification of a plant mutant with defects in vacuole morphogenesis. FIG. 1A-1D are confocal images of cotyledon epidermal cells expressing tonoplast marker GFP-δTIP in wild type and eva1 at various times. GFP-δTIP is a fusion between green fluorescent protein fusion and delta-TIP, a vacuolar membrane channel protein. FIG. 1A shows a confocal image of cotyledon epidermal cells expressing tonoplast marker GFP-δTIP in 10-day old wild type cells. FIG. 1B shows a confocal image of cotyledon epidermal cells expressing tonoplast marker GFP-δTIP in 10-day old eva1 cells. FIG. 1C shows a confocal image of cotyledon epidermal cells expressing tonoplast marker GFP-δTIP in 20-day old wild type cells. FIG. 1D shows a confocal image of cotyledon epidermal cells expressing tonoplast marker GFP-δTIP in 20-day old eva1 cells. In FIG. 1A-1D the top panels present single images of the middle focal plane of the epidermal cells, while the bottom panels present Z-stack maximal projections, which is a stack of about 20 single images with 20 μm intervals that fully span the top-to-bottom Z-axis of the epidermal cells. Arrows point to trans-vacuolar strands and arrowheads indicate presumably small vacuoles, which are prominent in eva1. In FIG. 1A-1B the scale bars represent 20 μm. In FIG. 1C-1D the scale bars represent 50 μm. FIG. 1E illustrates the genomic structure of the isopropylmalate synthase 1 (IPMS1) (ATIG18500) chromosomal locus. Lighter gray boxes are untranslated regions; darker black boxes are exons; and lines are introns. FIG. 1F shows an amino acid sequence alignment of IPMS1 homologs using T-COFFEE in Jalview. Amino acids are grouped by color with ClustalX based on their similarity of physicochemical properties. Abbreviations: At, Arabidopsis thaliana; SI, Solanum lycopersicum; Cr, Chlamydomonas reinhardtii: Mt, Mycobacterium tuberculosis. The amino acid substitution of eva1 is outlined by a red box. The AtIPMS1 and AtIPMS2 sequence shown is RFARSLGCEDVEFSPEDAGRSEREYL (SEQ ID NO:40, where the hold, underlined residue (D) is the position of the eva1 mutation). The AtMAM1 sequence shown is RFAKSLGFNDIQFGCEDGGRSDKDFL (SEQ ID NO:41, where the bold, underlined residue (D) is the position of the eva1 mutation). The AtMAM3 sequence shown is KYAKSLGFKDIQFGCEDGGRTEKDFI (SEQ ID NO:42, where the bold, underlined residue (D) is the position of the eva1 mutation). The SIIPMS1 sequence shown is AYARSIGCEDVEFSPEDAGRSDPEFL (SEQ ID NO:43, where the bold, underlined residue (D) is the position of the eva1 mutation). The CrIPMS sequence shown is KHLRSLGCNDIEFSPEDAGRSDPKFL (SEQ ID NO:44, where the bold, underlined residue (D) is the position of the eva1 mutation). The MtLeuA sequence shown is RKCVEQAAKYPGTQWRFEYSPESYTGTELEYA (SEQ ID NO:45, where the bold, underlined residue (D) is the position of the eva1 mutation). FIG. 1G shows a photograph of 10-day old plants of the indicated genotypes. Scale bar represents 0.5 cm. FIG. 1H shows a photograph of 20-day old plants of the indicated genotypes. Scale bar represents 1 cm. FIG. 1I-1J illustrate quantification of eva1 vacuolar phenotypes. FIG. 1I graphically illustrates the number of unfused vacuoles. n=40 cells for each genotype. FIG. 1J graphically illustrates the length of trans-vacuolar strands. Notably, in 10 day old wild-type cotyledon epidermal cells, trans-vacuolar strands are rarely observed. Cells of eva1 background have numerous trans-vacuolar strands. Only the longest string in each cell type was measured. n=16. Values are mean±SD. The asterisks indicate significant differences compared to wild type (***p≤0.001, unpaired t test).

FIG. 2A-2C illustrate amino acid profiling of IPMS1 loss-of-function mutants. FIG. 2A is a schematic diagram of the branched-chain amino acid (BCAA) biosynthetic pathway in the chloroplast. Lines show known feedback inhibitions of enzymes by end products, where inhibition is indicated by a bar perpendicular to a line. Arrows indicate steps in the BCAA biosynthetic pathway. FIG. 2B graphically illustrates fold changes of each free amino acid in 10-day old eva1, ipms1-4 and ipms1-5 samplings compared to 10-day old wild type saplings (n=7 for WT, n=5 for eva1, n=8 for ipms1-4. n=7 for ipms1-5). FIG. 2C graphically illustrates fold changes of each free amino acid in 20-day old eva1, ipms1-4 and ipms1-5 samplings compared to 20-day old wild type samplings (n=6 for each genotype). Values are mean±SEM. The asterisks indicate significant differences compared to the wild type (*p≤0.05, **p≤0.01, ***p≤0.001, unpaired t test). Amino acids were extracted from aerial tissues of 10 days old seedlings and rosette leaves of 20 days old plants. Each value represents the mean±SEM. The asterisks indicate significant difference compared to wild type (WT) (*p≤0.05. **p≤0.01, ***p≤0.001, unpaired t test). FW, fresh weight; cFAA, total 19 free amino acids without cysteine.

FIG. 3A-3J illustrate that mutants of IPMS1 exhibit defects in cotyledon architecture and chloroplast ultrastructure. FIG. 3A show light microscopic images of cotyledon cross sections. Cotyledon thickness is denoted by red lines. Scale bar, 100 μm. FIG. 3B graphically illustrates cotyledon thickness of plants with the indicated genotypes. n=9 for WT, eva1 and ipms1-4; n=6 for ipms1-5. FIG. 3C graphically illustrates cotyledon size of plants with the indicated genotypes. n=20 for each genotype. FIG. 3D shows representative transmission electron microscopy images of chloroplasts. Arrows point to connecting stroma thylakoids that are existing in WT and absent in mutants. Scale bar, 0.5 μm. FIG. 3E graphically illustrates thylakoid lengths of cotyledons of the indicated genotype. Five cotyledons from each genotype were sampled for imaging, and at least 30 stroma thylakoids were measured in each sample (n≥150). For all graphical representations of data, columns are mean±SD. The asterisks indicate significant differences of each mutant compared to wild type (***p≤0.001, **p≤0.01, unpaired t test). FIG. 3F graphically illustrates fresh weights of wild type (WT) and five mutants of IPMS1 at the 10-day old stage. FIG. 3G graphically illustrates fresh weights of wild type (WT) and five mutants of IPMS1 at the 20-day old stage. FIG. 3H graphically illustrates primary root lengths of wild type (WT) and five mutants of IPMS1 at the 10-day old stage. FIG. 3I graphically illustrates primary root lengths of wild type (WT) and five mutants of IPMS1 at the 20-day old stage. Mutants tf1111 and tf1102 are two independent ethyl methanesulfonate (EMS) mutagenized lines of ipms1-1^{D}. Values are mean±SD. The asterisks indicate significant differences compared to wild type (n=30 for each genotype at 10-day old stage, n=20 for each genotype at 20-day old stage; ***p≤0.001, **p≤0.01, NS, p>0.05 and not significant, unpaired t test). FIG. 3J graphically illustrates total anthocyanins of wild type (WT) and IPMS1 mutants. The absorbance at 532 nm was measured of samples that contained 50 μL extraction buffer per 1 mg dry weight. Values are mean±SEM. The asterisk indicates significant differences between each mutant line and the wild type (n=5 for each genotype; *p≤0.05, unpaired t test).

FIG. 4A-4I illustrate that mutation of IPMS1 affects endoplasmic reticulum (ER) morphology and F-actin organization. FIG. 4A shows low magnification confocal (top) and high magnification planar (bottom) images of wild type cotyledon epidermal cells stained for the ER marker ERYK. FIG. 4B shows low magnification confocal (top) and high magnification planar (bottom) images of eva1 cotyledon epidermal cells stained for the ER marker ERYK. As illustrated by the Z-stack projection images, the ER morphology in eva1 cells is altered, featuring longer and more thickened ER strands, as the arrows indicate. FIG. 4C shows confocal images of the wild type and eva1 cotyledon epidermal cells stained for the F-actin marker YFP-ABD2. Scale bar, 50 μm. FIG. 4D graphically illustrates the percentage of ER occupancy, which is the percentage of the area occupied by ER in the total field of view (n=20 for each genotype). Single-plane images were used for the quantification. Columns show mean±SD. The asterisks indicate significant differences (**p≤0.01, unpaired t test). FIG. 4E graphically illustrates the quantity of F-actin binding as a measure of F-actin organization, where quantification of skewness was quantified as an indication of higher level of F-actin bundling in eva1 compared to wild type (n=32 for each genotype). FIG. 4F graphically illustrates the percentage of F-actin occupancy, which is the area occupied by F-actin in the total field of view. As illustrated, eva1 cells exhibit a lower occupancy of F-actin compared to wild type cells (n=28 for each genotype). Z-stack projection images were used for the quantification. Columns show mean±SD. The asterisks indicate significant differences (**p≤0.01 and ***p≤0.001, unpaired t test). FIG. 4G shows that the number of Golgi are altered in eva1 mutant cells. The numbers of Golgi in each genotype were measured in independent field of view squares (100 μm×100 μm; wild type, n=16, eva1, n=20). Columns show mean±SD. The asterisks indicate significant difference compared to the wild type (***p≤0.001, unpaired t test). FIG. 4H-4I illustrate that compared to wild type, IPMS1 loss-of-function mutants are less sensitive to latrunculin B (Lat B; an actin polymerization inhibitor). Ten day old wild type (Col-0), ipms1-4 and ipms1-5 seedlings were germinated and grown on ½ LS and 1% sucrose medium then transplanted to ½ LS and 1% sucrose medium containing DMSO, 50 nM Lat B or 100 nM Lat B. FIG. 4H graphically illustrates primary root length of the wild type, ipms1-4 and ipms1-5 seedlings on the day of transplant (0 day). FIG. 4I graphically illustrates primary root length of the wild type, ipms1-4 and ipms1-5 seedlings at 8 days after transplant. Values are mean±SEM. The asterisks indicate significant differences compared to the wild type (n=8 for each genotype on a specific medium; ***p≤0.001, **p≤0.01, NS, p>0.05 and not significant, unpaired t test).

FIG. 5A-5I illustrate that chemical interventions can fully or partially rescue the vacuolar mutant phenotypes of eva1. FIG. 5A shows a low magnification confocal image of cotyledon epidermal cells expressing GFP-δTIP from 10-day old eva1 plants after 2 hours treatment of DMSO. Scale bar of 50 μm. FIG. 5B shows a high magnification confocal image of cotyledon epidermal cells expressing GFP-δTIP from 10-day old eva1 plants after 2-hour treatment of DMSO. Scale bar of 10 μm. FIG. 5C shows a low magnification confocal image of cotyledon epidermal cells expressing GFP-δTIP from 10-day old eva1 plants after 2-hour treatment with wortmannin (Wm; a covalent inhibitor of phosphoinositide 3-kinases). Scale bar of 50 μm. FIG. 5D shows a high magnification confocal image of cotyledon epidermal cells expressing GFP-δTIP from 10-day old eva1 plants after 2-hour treatment with wortmannin (Wm). Scale bar of 10 μm. FIG. 5E shows a low magnification confocal image of cotyledon epidermal cells expressing GFP-δTIP from 10-day old eva1 plants after 2-hour treatment with latrunculin B (Lat B; an actin polymerization inhibitor). Scale bar of 50 sm. FIG. 5F shows a high magnification confocal image of cotyledon epidermal cells expressing GFP-δTIP from 10-day old eva1 plants after 2-hour treatment with latrunculin B (Lat B). Scale bar of 10 μm. FIG. 5G shows a low magnification confocal image of cotyledon epidermal cells expressing GFP-δTIP from 10-day old eva1 plants after 2-hour treatment with oryzalin. Scale bar of 50 μm. FIG. 5H shows a high magnification confocal image of cotyledon epidermal cells expressing GFP-δTIP from 10-day old eva1 plants after 2-hour treatment with oryzalin. Scale bar of 10 μm. Arrowheads suggest presumably unfused vacuolar structures and arrows pinpoint enhanced TVSs. All the images are Z-stack maximal projections. FIG. 5I graphically illustrates the number of presumably unfused small vacuoles in wild type and eva1 cotyledon epidermal cells of 10-day old wild type (WT) and eva1 plants before or after 3-hour treatment of DMSO or wortmannin (Wm). Values are mean±SD. The asterisks indicate significant differences (n=40 cells for each treatment: ***p≤0.001, NS, p>0.05 and not significant, unpaired t test).

FIG. 6A-6Q illustrate that vacuolar mutant phenotypes of eva1 are correlated with up-regulated TOR activity. FIGS. 6A-6F show that the TOR inhibitor AZD-8088 treatment rescues vacuolar mutant phenotypes of eva1. FIG. 6A is a confocal image acquired before 10-day old wild type seedlings were transferred to liquid growth medium containing 5 mM AZD-8055. FIG. 6B is a confocal image acquired before 10-day old eva1 seedlings were transferred to liquid growth medium containing 5 mM AZD-8055. FIG. 6C is a confocal image acquired 2 hours after 10-day old wild type seedlings were transferred to liquid growth medium containing 5 mM AZD-8055. FIG. 6D is a confocal image acquired 2 hours after 10-day old eva1 seedlings were transferred to liquid growth medium containing 5 mM AZD-8055. FIG. 6E is a confocal image acquired 4 hours after 10-day old wild type seedlings were transferred to liquid growth medium containing 5 mM AZD-8055. FIG. 6F is a confocal image acquired 4 hours after 10-day old eva1 seedlings were transferred to liquid growth medium containing 5 mM AZD-8055. The arrowhead indicates presumably unfused vacuolar structures and arrows point to enhanced trans-vacuolar strands. All the images are Z-stack maximal projections. Scale bar, 25 mm. FIG. 6G graphically illustrates the number of unfused vacuoles in eva1 before and after TOR inhibitor treatment. Values are mean±SD. The asterisks indicate significant differences (n=30 cells for each treatment; ***p≤−0.001, NS, p>0.05 and not significant, unpaired t test). FIG. 6H shows immunoblots that detect phosphorylation of S6K by TOR, as detected by staining with specific antisera against S6K-phosphorylated and S6K. S6K phosphorylates the 40S ribosomal protein S6 (S6) at five Ser residues. LC, loading control with Ponceau S staining. FIG. 6I graphically illustrates S6K phosphorylation status calculated by the ratio of S6K-p/S6K in fold change compared to wild type. n=3 and values are mean±SEM (***p≤0.05, unpaired t test). FIG. 6J shows images of 5-ethynyl-2′-deoxyuridine-stained (EdU-stained) root meristems of 10-day old seedlings. For each genotype, the lighter green spots with the dark background shows EdU-stained newly synthesized DNA and the bright-field image shows structure of root tip. Scale bar, 100 mm. FIG. 6K graphically illustrates EdU fluorescence intensity of EdU-stained root meristems shown in FIG. 6J. Values are mean±SD. The asterisks indicate significant differences compared to wild type (n=9 for each genotype; **p≤0.01, ***p≤0.001, unpaired t test). FIG. 6L-6M graphically illustrate that the effects of the TOR inhibitor AZD-8055 on ipms1 primary root elongation is dose-dependent. Ten-day old wild type (WT. Col-0), eva1, ipms1-4 and ipms1-5 seedlings were germinated and grown on ½ LS and 1% sucrose medium containing DMSO or increasing concentrations of TOR inhibitor AZD-8055. FIG. 6L graphically illustrates the primary root length at different AZD-8055 concentrations. Values are mean±SD. The asterisks indicate significant differences compared to the wild type. FIG. 6M graphically illustrates the change of root elongation, expressed as % of each AZD-8055 treatment versus control (DMSO solvent). Values are mean±SD. The asterisks in indicate significant differences compared to the DMSO control. n=50 for WT, n=100 for other genotypes on a specific medium; ***p≤0.001. *p≤0.05, unpaired t test. FIG. 6N-6Q illustrate the effects of the PI3K/TOR dual inhibitor wortmannin and the F-actin depolymerizer Lat B on ipms1 primary root elongation. Ten-day old wild type (WT, Col-0), eva1, ipms1-4 and ipms1-5 seedlings were germinated and growth on medium containing DMSO or increasing concentrations of wortmannin. FIG. 6N graphically illustrates the length of primary roots of wild type (WT, Col-0), eva1, ipms1-4 and ipms1-5 seedlings treated with various amounts of wortmannin. Values are mean±SD. The asterisks indicate significant differences compared to the wild type. FIG. 6O graphically illustrates the primary root length as a percent of control of wild type (WT, Col-0), eva1, ipms1-4 and ipms1-5 seedlings treated with various amounts of wortmannin. FIGS. 6N and 6O illustrate that wortmannin confers minimal impacts on ipms1 primary root elongation. FIG. 6P-6Q illustrate that compared to wild type, ipms1 primary root elongation is less sensitive to Lat B. Nine-day old wild type (WT, Col-0), eva1, ipms1-4 and ipms1-5 seedlings were germinated and grown on medium containing DMSO or increasing concentrations of Lat B. FIG. 6P graphically illustrates the length of primary roots of wild type (WT, Col-0), eva1, ipms1-4 and ipms1-5 seedlings treated with various amounts of Lat B. Values are mean±SD. The asterisks indicate significant differences compared to the wild type. FIG. 6Q graphically illustrates the primary root length as a percent of control of wild type (WT. Col-0), eva1, ipms1-4 and ipms1-5 seedlings treated with various amounts of Lat B. The asterisks in FIGS. 6P and 6Q indicate Lat B treatment leads to significant differences in wild type compared to the DMSO control, but that Lat B has less effects on ipms1 mutants. n=50 for WT, n=100 for other genotypes on a specific medium; ***p≤0.001, **p≤0.01, *p≤0.05, NS, p>0.05 and not significant, unpaired t test.

FIG. 7A-7L illustrate that feeding of exogenous branched-chain amino acids (BCAAs) and over aCcumulation of endogenous BCAAs induce actin bundling, which is dependent on functional TOR but not RAPTOR. FIG. 7A illustrates the organization of actin cytoskeleton in mock-treated wild type cotyledon epidermal cells expressing F-actin marker YFP-ABD2. FIG. 7B illustrates the organization of actin cytoskeleton in mock-treated tor-es (no induction) cotyledon epidermal cells expressing F-actin marker YFP-ABD2. FIG. 7C illustrates the organization of actin cytoskeleton in mock-treated for-es (with induction) cotyledon epidermal cells expressing F-actin marker YFP-ABD2. FIG. 7D illustrates the organization of actin cytoskeleton in mock-treated raptor1b cotyledon epidermal cells expressing F-actin marker YFP-ABD2. Higher fluorescence intensity of the actin marker suggests more bundling of actin filaments. Using ImageJ, a 50 μm red arrowed line was drawn to detect the pixel fluorescence intensity beneath such a line. In each image, the red arrowed line is positioned where the highest fluorescence intensity was detected using non-saturating imaging settings. A chart beneath the image presents plotted fluorescence intensity along the red arrowed line. Without actin bundling, fine actin filaments have fluorescence intensity about 1000 (relative unit). In contrast, induced actin bundling show fluorescence intensity peaks of 3000-4000 (relative unit) (FIG. 7A-7D). Without feeding of branched-chain amino acids (mock), wild type, for-es with or without silencing, and raptor/b did not show induced actin bundling. FIG. 7E illustrates the organization of actin cytoskeleton in wild type cotyledon epidermal cells expressing F-actin marker YFP-ABD2 treated with 1 mM branched-chain amino acids (BCAAs). FIG. 7F illustrates the organization of actin cytoskeleton in for-es (no induction) cotyledon epidermal cells expressing F-actin marker YFP-ABD2 treated with 1 mM BCAA. FIG. 7O illustrates the organization of actin cytoskeleton in tor-es (with induction) cotyledon epidermal cells expressing F-actin marker YFP-ABD2 treated with 1 mM BCAA. FIG. 7H illustrates the organization of actin cytoskeleton in raptor/b cotyledon epidermal cells expressing F-actin marker YFP-ABD2 treated with 1 mM BCAA. As illustrated in FIGS. 7E, 7F and 7H, feeding of 1 mM BCAAs induced striking actin bundling in wild type, tor-es without gene silencing and raptor1b, but not in for-es with induction of TOR silencing (FIG. 7G). FIG. 7I illustrates the organization of actin cytoskeleton in mock-treated ipms1-1^{D} cotyledon epidermal cells expressing F-actin marker YFP-ABD2. FIG. 7J illustrates the organization of actin cytoskeleton in mock-treated ahass1-1 cotyledon epidermal cells expressing F-actin marker YFP-ABD2. FIG. 7K illustrates the organization of actin cytoskeleton in mock-treated ipms1-5 cotyledon epidermal cells expressing F-actin marker YFP-ABD2. FIG. 7L illustrates the organization of actin cytoskeleton in mock-treated omr1-11^{D} cotyledon epidermal cells expressing F-actin marker YFP-ABD2. As shown in FIGS. 7I and J, without feeding of BCAAs (mock), mutants with small changes of BCAAs did not show induced actin bundling, however mutants with over-accumulation of endogenous BCAAs showed induced actin bundling (FIG. 7K-7L). All the images are Z-stack maximal projections. Scale bars, 50 μm.

FIG. 8 is a schematic diagram of TOR-regulated subcellular processes. Over-accumulation of BCAA Val, Leu and Ile stimulates TOR signaling. Except for the established downstream processes such as protein synthesis and cell proliferation, vacuole fusion, and actin reorganization are also regulated by TOR signaling, but the underlying mechanisms are unclear. Reorganization of the actin cytoskeleton is independent of TORC1, and prominent trans-vacuolar strands and ER strands are subsequently formed due to the strong interactions between the endomembranes and the F-actin in plant cells.

### DETAILED DESCRIPTION

Described herein are modified plants, plant cells, and plant seeds that provide improved amino acid content, for example, higher levels of branched-chain amino acids (BCAAs) and other amino acids. Examples of amino acids that can be at higher levels in the modified plants, plant cells, and plant seeds include Gln, His, Ile. Leu, Lys, Met, Phe. Thr. Trp, Val, or a combination thereof. In some cases, the BCAAs that are increased in the modified plant tissues are leucine, isoleucine, and valine. However, in some cases, one or two of leucine, isoleucine, or valine may not be increased in the modified plant tissues.

Methods for making and using such modified plants, plant cells, and plant seeds are also described herein. The modified nucleic acids, expression cassettes, plants, seeds and methods described herein can also be used to improve the growth and quantity of plant biomass even while having improved amino acid content. Methods of making and producing such plant seeds and plants can include, for example, cultivating seeds or seedlings, harvesting the plants, seeds, or the tissues of the plants. Such methods can also include isolating proteins and/or amino acids from the plants, seeds, or the tissues of the plants.

The plants, seeds, and plants cells described herein can have a modified or mutant isopropylmalate synthase (IPMS) gene. Surprisingly, the IPMS gene or IPMS nucleic acids that provide increased biomass and increased amino acid content can have a modification in its catalytic domain, in its allosteric domain, or in both domains. The modification in the catalytic domain can be in the acetyl-CoA binding surface near the pocket for the substrate. The modification in the allosteric domain can be located within about 20 amino acids of the IPMS protein C-terminus.

### IPMS

The IPMS1 and IPMS2 genes encode isopropylmalate synthase (IPMS, classified as EC 2.3.3.13) that catalyzes the first dedicated step in Leu biosynthesis. An alternate name for the IPMS1 enzyme is methylthioalkylmalate synthase-like 4 (MAML-4). The IPMS1 (MAML-4) protein is naturally expressed constitutively throughout the plant.

Examples of IPMS nucleic acids include the Arabidopsis thaliana IPMS1 (At1g18500) and IPMS2 (At1g74040) cDNAs. In Arabidopsis thaliana the IPMS1 gene is located on chromosome 1. A cDNA sequence that encodes an Arabidopsis thaliana isopropylmalate synthase IPMS1 protein is shown below as SEQ ID NO: 1.

| | |
|---|---|
| 1 | GAAAAAAAAA ACGAATTCTA ATGTGCCCGC TATAAAATCT TCCGCAAGAG |
| 51 | TGTAACAGTG ATGCAGCTGA ATCAATAAGA CTGTCTTCTT CTCCGAATTT |
| 101 | GAAAATTAAA TTCCAGTTTT TTCAGTTTGA CTCTGCTTCT TCTTCCTCGT |
| 151 | GGGTAACGAC GATATACCGT TAAAATTAGG AACCAAATTA CCCAATGGTC |
| 201 | GTCGTCAAAT CATTTTTAAT CCCAATTTGG TATTTTTCCA CGTGGGTCAA |
| 251 | ACAAAAAACA ATTTTTTACA TAAAGAGAAG AGAGTAGTGA CGAGAAGATT |
| 301 | AGCACTACTG AATCAAACTT AGCCGCCGCC ACCGTCACGT TGAAACCTTC |
| 351 | ATCTCTCTAT CTCTCTGAGA CCTCTCCTTC AATGGCGTCT TCGCTTCTGA |
| 401 | GAAACCCTAA TCTCTACTCA TCAACAACAA TCACCACCAC TTCTTTTCTT |
| 451 | CCCACCTTCT CCTCTAAACC CACACCTATC TCCTCCTCTT TCCGTTTCCA |
| 501 | ACCATCTCAC CACCGTTCAA TCTCCCTCCG AAGTCAAACC CTCCGTCTCT |
| 551 | CATGCTCAAT CTCAGATCCT TCTCCACTAC CACCTCACAC TCCTCGCCGT |
| 601 | CCCCGTCCTG AATACATCCC CAACCGCATT TCCGATCCAA ACTACGTCCG |
| 651 | CGTCTTCGAT ACTACTCTCC GTGACGGTGA ACAATCTCCA GGAGCTACAC |
| 701 | TTACTTCCAA GGAAAAACTT GACATCGCTC GTCAGCTAGC TAAACTTGGT |
| 751 | GTTGACATCA TCGAGGCTGG GTTTCCTGCT GCTTCCAAGG ATGATTTTGA |
| 801 | AGCGGTTAAG ACTATAGCTG AAACAGTTGG AAACACTGTT GATGAGAATG |
| 851 | GTTATGTTCC TGTTATCTGT GGACTCTCTA GATGCAATAA GAAGGATATT |
| 901 | GAGAGAGCTT GGGATGCTGT GAAATACGCT AAACGGCCTA GGATTCATAC |
| 951 | TTTTATAGCT ACTAGTGATA TACATTTGGA GTATAAATAA AAGAAAACCA |
| 1001 | AAGCAGAGGT CATCGAAATC GCTAGGAGTA TGGTTAGATT CGCGAGGAGC |
| 1051 | TTGGGGTGCG AAGATGTTGA GTTCAGTCCA GAAGATGCAG GAAGATCGGA |
| 1101 | GAGAGAGTAC TTATACGAGA TTCTTGGTGA AGTGATAAAA GCAGGAGCAA |
| 1151 | CAACTCTCAA CATACCTGAT ACTGTTGGTA TAACTTTGCC TAGTGAGTTT |
| 1201 | GGTCAACTGA TTACTGATTT AAAGGCCAAT ACTCCGGGGA TTGAAAATGT |
| 1251 | TGTCATCTCA ACACATTGTC AGAATGATCT TGGACTCTCT ACGGCCAACA |
| 1301 | CTTTATCTGG GGCACATGCA GGTGCGAGGC AGATGGAAGT GACGATGAAT |
| 1351 | GGAATTGGTG AAAGAGCTGG AAACGCTTCA CTGGAAGAGG TTGTGATGGC |
| 1401 | CATAAAATGC CGTGGAGATC ATGTATTAGG AGGTCTATTT ACCGGAATTG |
| 1451 | ATACTCGGCA CATTGTTATG ACAAGCAAGA TGGTAGAGGA GTACACTGGG |
| 1501 | ATGCAGACAC AACCTCATAA GGCTATTGTA GGAGCGAATG CCTTTGCGCA |
| 1551 | TGAAAGTGGA ATTCACCAGG ATGGAATGCT GAAACACAAG GGTACATATG |
| 1601 | AAATTATATG TCCCGAAGAA ATTGGACTTG AACGATCAAA TGATGCTGGC |
| 1651 | ATTGTCTTGG GGAAGCTTAG TGGGCGTCAT GCGCTGAAAG ACCGTTTGAC |
| 1701 | TGAGCTTGGT TATCAATTAG ATGATGAACA GCTAAGTACC ATTTTCTGGC |
| 1751 | GCTTCAAAAC CGTGGCTGAG CAGAAAAAGA GAGTTACTGA TGCGGACATA |
| 1801 | ATAGCTTTAG TATCTGATGA AGTTTTCCAG CCAGAAGCCG TGTGGAAACT |
| 1851 | CCTGGACATT CAGATAACTT GTGGAACTCT CGGGCTTTCA ACAGCAACTG |
| 1901 | TAAAACTTGC TGAGGCTGAT GGCAAAGAAC ATGTCGCTTG TTCTATTGGA |
| 1951 | ACTGGGCCTG TGGATTCAGC TTACAAGGCA GTAGATCTTA TCGTAAAGGA |
| 2001 | ACCGGCTACT CTGCTTGAGT ACTCAATGAA TGCAGTAACA GAAGGCATTG |
| 2051 | ATGCCATCGC AACCACAAGA GTTCTTATCC GTGAAGCAAC CAAATACTCA |
| 2101 | TCTACAAACG CAATAACTGG TGAAGAGGTT CAAAGAACCT TTAGTGGAAC |
| 2151 | TGGAGCAGGA ATGGATATTG TGGTGTCAAG CGTCAAAGCT TATGTTGGAG |
| 2201 | CTTTGAACAA AATGATGGAC TTCAAAGAAA ACTCCGCCAC AAAAATCCCT |
| 2251 | TCCCAAAAAA ACAGAGTCGC TGCCTGAATT AAAAATCTTT CCGGCAAATA |
| 2301 | CCAAAAAGTC AGACAGAAGT TAGGTTCTTT TATTTTCAAG TACATAGTTT |
| 2301 | CCAAAAAGTC AGACAGAAGT TAGGTTCTTT TATTTTCAAG TACATAGTTT |
| 2351 | GGTAATAACT GGAGTTTCGG AGTTTGCTTG TTGTTTATCG AAGTTGCATG |
| 2401 | TCAAAAGAGT TTGGTGTACT ATATATATCT TGATTTAACT TGAATCTCTA |
| 2451 | TTTTTAGAAA TAATGGTTTT AGAATAAGGA ATAAAAACCA ACCGTT |

The amino acid sequence for the Arabidopsis thaliana isopropylmalate synthase protein encoded by the SEQ ID NO:1 cDNA is shown below as SEQ ID NO:2.

| | |
|---|---|
| 1 | MASSLLRNPN LYSSTTITTT SFLPTFSSKP TPISSSFRFQ PSHHRSISLR |
| 51 | SQTLRLSCSI SDPSPLPPHT PRRPRPEYIP NRISDPNYVR VFDTTLRDGE |
| 101 | QSPGATLTSK EKLDIARQLA KLGVDIIEAG FPAASKDDFE AVKTIAETVG |
| 151 | NTVDENGYVP VICGLSRCNK KDIERAWDAV KYAKRPRIHT FIATSDIHLE |
| 201 | YKLKKTKAEV IEIARSMVRF ARSLGCEDVE FSPEDAGRSE REYLYEILGE |
| 251 | VIKAGATTLN IPDTVGITLP SEFGQLITDL KANTPGIENV VISTHCQNDL |
| 301 | GLSTANTLSG AHAGARQMEV TINGIGERAG NASLEEVVMA IKCRGDHVLG |
| 351 | GLFTGIDTRH IVMTSKMVEE YTGMQTQPHK AIVGANAFAH ESGIHQDGML |
| 401 | KHKGTYEIIC PEEIGLERSN DAGIVLGKLS GRHALKDRLT ELGYQLDDEQ |
| 451 | LSTIFWRFKT VAEQKKRVTD ADIIALVSDE VFQPEAVWKL LDIQITCGTL |
| 501 | GLSTATVKLA DADGKEHVAC SIGTGPVDSA YKAVDLIVKE PATLLEYSMN |
| 551 | AVTEGIDAIA TTRVLIRGSN KYSSTNAITG EEVQRTFSGT GAGMDIVVSS |
| 601 | VKAYVGALNK MMDFKENSAT KIPSQKNRVA A |

One example of a modified IPMS1 protein that provides plants with significantly higher levels of Gln, His, Ile, Leu, Lys. Met, Phe, Thr, Trp, Val, or a combination thereof in their leaves and seeds, and significant increases in their biomass or amino acid content compared to wild type, parental, or IPMS knockout leaves and/or seeds, can have a mutation located in the acetyl-CoA binding surface near the pocket for 2-oxoisovalerate substrate. One example of a modified IPMS1 protein that has an altered amino acid content is the eva1 protein, which has a point mutation at position 228 of SEQ ID NO:2, where the aspartic acid (D) can, for example, be an asparagine (N). This modification is identified above in SEQ ID NO:2 in bold and with underlining. The eva1 protein with the asparagine (N) substitution for aspartic acid (D) at position 228 (D228N) has the sequence shown below as SEQ ID NO:3.

| | |
|---|---|
| 1 | MASSLLRNPN LYSSTTITTT SFLPTFSSKP TPISSSFRFQ PSHHRSISLR |
| 51 | SQTLRLSCSI SDPSPLPPHT PRRPRPEYIP NRISDPNYVR VFDTTLRDGE |
| 101 | QSPGATLTSK EKLDIARQLA KLGVDIIEAG FPAASKDDFE AVKTIAETVG |
| 151 | NTVDENGYVP VICGLSRCNK KDIERAWDAV KYAKRPRIHT FIATSDIHLE |
| 201 | YKLKKTKAEV IEIARSMVRF ARSLGCENVE FSPEDAGRSE REYLYEILGE |
| 251 | VIKAGATTLN IPDTVGITLP SEFGQLITDL KANTPGIENV VISTHCQNDL |
| 301 | GLSTANTLSG AHAGARQMEV TINGIGERAG NASLEEVVMA IKCRGDHVLG |
| 351 | GLFTGIDTRH IVMTSKMVEE YTGMQTQPHK AIVGANAFAH ESGIHQDGML |
| 401 | KHKGTYEIIC PEEIGLERSN DAGIVLGKLS GRHALKDRLT ELGYQLDDEQ |
| 451 | LSTIFWRFKT VAEQKKRVTD ADIIALVSDE VFQPEAVWKL LDIQITCGTL |
| 501 | GLSTATVKLA DADGKEHVAC SIGTGPVDSA YKAVDLIVKE PATLLEYSMN |
| 551 | AVTEGIDAIA TTRVLIRGSN KYSSTNAITG EEVQRTFSGT GAGMDIVVSS |
| 601 | VKAYVGALNK MMDFKENSAT KIPSQKNRVA A |

Compared to the SEQ ID NO:1 IPMS11 cDNA, the cDNA encoding the SEQ ID NO:3 eva1 protein can have the following sequence (SEQ ID NO:4), where the guanine at position 682 is an adenine (highlighted in bold with underlining).

| | |
|---|---|
| 1 | ATGGCGTCTT CGCTTCTGAG AAACCCTAAT CTCTACTCAT CAACAACAAT |
| 51 | CACCACCACT TCTTTTCTTC CCACCTTCTC CTCTAAACCC ACACCTATCT |
| 101 | CCTCCTCTTT CCGTTTCCAA CCATCTCACC ACCGTTCAAT CTCCCTCCGA |
| 151 | AGTCAAACCC TCCGTCTCTC ATGCTCAATC TCAGATCCTT CTCCACTACC |
| 201 | ACCTCACACT CCTCGCCGTC CCCGTCCTGA ATACATCCCC AACCGCATTT |
| 251 | CCGATCCAAA CTACGTCCGC GTCTTCCATA CTACTCTCCG TGACGGTGAA |
| 301 | CAATCTCCAG GAGCTACACT TACTTCCAAG GAAAAACTTG ACATCGCTCG |
| 351 | TCAGCTAGCT AAACTTGGTG TTGACATCAT CGAGGCTGGG TTTCCTGCTG |
| 401 | CTTCCAAGGA TGATTTTGAA GCGGTTAAGA CTATAGCTGA AACAGTTGGA |
| 451 | AACACTGTTG ATGAGAATGG TTATGTTCCT GTTATCTGTG GACTCTCTAG |
| 501 | ATGCAATAAG AAGGATATTG AGAGAGCTTG GGATGCTGTG AAATACGCTA |
| 551 | AACGGCCTAG GATTCATACT TTTATAGCTA CTAGTGATAT ACATTTGGAG |
| 601 | TATAAACTAA AGAAAACCAA AGCAGAGGTC ATCGAAATCG CTAGGAGTAT |
| 651 | GGTTAGATTC GCGAGGAGCT TGGGGTGCGA AAATGTTGAG TTCAGTCCAG |
| 701 | AAGATGCAGG AAGATCGGAG AGAGAGTACT TATACGAGAT TCTTGGTGAA |
| 751 | GTGATAAAAG CAGGAGCAAC AACTCTCAAC ATACCTGATA CTGTTGGTAT |
| 801 | AACTTTGCCT AGTGAGTTTG GTCAACTGAT TACTGATTTA AAGGCCAATA |
| 851 | CTCCGGGGAT TGAAAATGTT GTCATCTCAA CACATTGTCA GAATGATCTT |
| 901 | GGACTCTCTA CGGCCAACAC TTTATCTGGG GCACATGCAG GTGCGAGGCA |
| 951 | GATGGAAGTG ACGATCAATG GAATTGGTGA AAGAGCTGGA AACGCTTCAC |
| 1001 | TGGAAGAGGT TGTGATGGCC ATAAAATGCC GTGGAGATCA TGTATTAGGA |
| 1051 | GGTCTATTTA CCGGAATTGA TACTCGGCAC ATTGTTATGA CAAGCAAGAT |
| 1101 | GGTAGAGGAG TACACTGGGA TGCAGACACA ACCTCATAAG GCTATTGTAG |
| 1151 | GAGCGAATGC CTTTGCGCAT GAAAGTGGAA TTCACCAGGA TGGAATGCTG |
| 1201 | AAACACAAGG GTACATATGA AATTATATGT CCCGAAGAAA TTGGACTTGA |
| 1251 | ACGATCAAAT GATGCTGGCA TTGTCTTGGG GAAGCTTAGT GGGCGTCATG |
| 1301 | CGCTGAAAGA CCGTTTGACT GAGCTTGGTT ATCAATTAGA TGATGAACAG |
| 1351 | CTAAGTACCA TTTTCTGGCG CTTCAAAACC GTGGCTGAGC AGAAAAAGAG |
| 1401 | AGTTACTGAT GCGGACATAA TAGCTTTAGT ATCTGATGAA GTTTTCCAGC |
| 1451 | CAGAAGCCGT GTGGAAACTC CTGGACATTC AGATAACTTG TGGAACTCTC |
| 1501 | GGGCTTTCAA CAGCAACTGT AAAACTTGCT GACGCTGATG GCAAAGAACA |
| 1551 | TGTCGCTTGT TCTATTGGAA CTGGGCCTGT GGATTCAGCT TACAAGGCAG |
| 1601 | TAGATCTTAT CGTAAAGGAA CCGGCTACTC TGCTTGAGTA CTCAATGAAT |
| 1651 | GCAGTAACAG AAGGCATTGA TGCCATCGCA ACCACAAGAG TTCTTATCCG |
| 1701 | TGGAAGCAAC AAATACTCAT CTACAAACGC AATAACTGGT GAAGAGGTTC |
| 1751 | AAAGAACCTT TAGTGGAACT GGAGCAGGAA TGGATATTGT GGTGTCAAGC |
| 1801 | GTCAAAGCTT ATGTTGGAGC TTTGAACAAA ATGATGGACT TCAAAGAAAA |
| 1851 | CTCCGCCACA AAAATCCCTT CCCAAAAAAA CAGAGTCGCT GCCTGA |

Another example of a modified IPMS1 protein that provides plants with significantly higher levels of Gln, His, Ile, Leu, Lys, Met, Phe, Thr, Trp. Val, or a combination thereof in their leaves and seeds, and significant increases in their amino acid content or biomass compared to wild type, parental, or IPMS knockout leaves and/or seeds, is a modified IPMS1 protein with a modification in the IPMS1 allosteric domain, which can be located within about 20 amino acids of the C-terminus. One example, of an IPMS1 protein with a modification in the allosteric domain is dominant ipms1-1D feedback-insensitive mutant, which can provide small Val decreases but increases in Leu. In Arabidopsis, the ipms1-1D protein can, for example, have a point mutation at position 606 where the glycine (G) can be substituted with another amino acid. For example, the glycine at position 606 can be a glutamic acid (E). The position of this modification is identified in SEQ ID NO:2 in bold and with underlining. The SEQ ID NO:2 with the glutamic acid (E) substitution for glycine (G) at position 606 (G606E) has the sequence shown below as SEQ ID NO:5.

| | |
|---|---|
| 1 | MASSLLRNPN LYSSTTITTT SFLPTFSSKP TPISSSFRFQ PSHHRSISLR |
| 51 | SQTLRLSCSI SDPSPLPPHT PRRPRPEYIP NRISDPNYVR VFDTTLRDGE |
| 101 | QSPGATLTSK EKLDIARQLA KLGVDIIEAG FPAASKDDFE AVKTIAETVG |
| 151 | NTVDENGYVP VICGLSRCNK KDIERAWDAV KYAKRPRIHT FIATSDIHLE |
| 201 | YKLKKTKAEV IEIARSMVRF ARSLGCEDVE FSPEDAGRSE REYLYEILGE |
| 251 | VIKAGATTLN IPDTVGITLP SEFGQLITDL KANTPGIENV VISTHCQNDL |
| 301 | GLSTANTLSG AHAGARQMEV TINGIGERAG NASLEEVVMA IKCRGDHVLG |
| 351 | GLFTGIDTRH IVMTSKMVEE YTGMQTQPHK AIVGANAFAH ESGIHQDGML |
| 401 | KHKGTYEIIC PEEIGLERSN DAGIVLGKLS GRHALKDRLT ELGYQLDDEQ |
| 451 | LSTIFWRFKT VAEQKKRVTD ADIIALVSDE VFQPEAVWKL LDIQITCGTL |
| 501 | GLSTATVKLA DADGKEHVAC SIGTGPVDSA YKAVDLIVKE PATLLEYSMN |
| 551 | AVTEGIDAIA TTRVLIRGSN KYSSTNAITG EEVQRTFSGT GAGMDIVVSS |
| 601 | VKAYVEALNK MMDFKENSAT KIPSQKNRVA A |

Compared to the SEQ ID NO:1 IPMS1 cDNA, the coding region DNA for IPMS1-1^{D} can have the following sequence (SEQ ID NO:6), where the guanine at position 1817 is an adenine (highlighted in bold with underlining).

| | |
|---|---|
| 1 | ATGGCGTCTT CGCTTCTGAG AAACCCTAAT CTCTACTCAT CAACAACAAT |
| 51 | CACCACCACT TCTTTTCTTC CCACCTTCTC CTCTAAACCC ACACCTATCT |
| 101 | CCTCCTCTTT CCGTTTCCAA CCATCTCACC ACCGTTCAAT CTCCCTCCGA |
| 151 | AGTCAAACCC TCCGTCTCTC ATGCTCAATC TCAGATCCTT CTCCACTACC |
| 201 | ACCTCACACT CCTCGCCGTC CCCGTCCTGA ATACATCCCC AACCGCATTT |
| 251 | CCGATCCAAA CTACGTCCGC GTCTTCGATA CTACTCTCCG TGACGGTGAA |
| 301 | CAATCTCCAG GAGCTACACT TACTTCCAAG GAAAAACTTG ACATCGCTCG |
| 351 | TCAGCTAGCT AAACTTGGTG TTGACATCAT CGAGGCTGGG TTTCCTGCTG |
| 401 | CTTCCAAGGA TGATTTTGAA GCGGTTAAGA CTATAGCTGA AACAGTTGGA |
| 451 | AACACTGTTG ATGAGAATGG TTATGTTCCT GTTATCTGTG GACTCTCTAG |
| 501 | ATGCAATAAG AAGGATATTG AGAGAGCTTG GGATGCTGTG AAATACGCTA |
| 551 | AACGGCCTAG GATTCATACT TTTATAGCTA CTAGTGATAT ACATTTGGAG |
| 601 | TATAAACTAA AGAAAACCAA AGCAGAGGTC ATCGAAATCG CTAGGAGTAT |
| 651 | GGTTAGATTC GCGAGGAGCT TGGGGTGCGA AGATGTTGAG TTCAGTCCAG |
| 701 | AAGATGCAGG AAGATCGGAG AGAGAGTACT TATACGAGAT TCTTGGTGAA |
| 751 | GTGATAAAAG CAGGAGCAAC AACTCTCAAC ATACCTGATA CTGTTGGTAT |
| 801 | AACTTTGCCT AGTGAGTTTG GTCAACTGAT TACTGATTTA AAGGCCAATA |
| 851 | CTCCGGGGAT TGAAAATGTT GTCATCTCAA CACATTGTCA GAATGATCTT |
| 901 | GGACTCTCTA CGGCCAACAC TTTATCTGGG GCACATGCAG GTGCGAGGCA |
| 951 | GATGGAAGTG ACGATCAATG GAATTGGTGA AAGAGCTGGA AACGCTTCAC |
| 1001 | TGGAAGAGGT TGTGATGGCC ATAAAATGCC GTGGAGATCA TGTATTAGGA |
| 1051 | GGTCTATTTA CCGGAATTGA TACTCGGCAC ATTGTTATGA CAAGCAAGAT |
| 1101 | GGTAGAGGAG TACACTGGGA TGCAGACACA ACCTCATAAG GCTATTGTAG |
| 1151 | GAGCGAATGC CTTTGCGCAT GAAAGTGGAA TTCACCAGGA TGGAATGCTG |
| 1201 | AAACACAAGG GTACATATGA AATTATATGT CCCGAAGAAA TTGGACTTGA |
| 1251 | ACGATCAAAT GATGCTGGCA TTGTCTTGGG GAAGCTTAGT GGGCGTCATG |
| 1301 | CGCTGAAAGA CCGTTTGACT GAGCTTGGTT ATCAATTAGA TGATGAACAG |
| 1351 | CTAAGTACCA TTTTCTGGCG CTTCAAAACC GTGGCTGAGC AGAAAAAGAG |
| 1401 | AGTTACTGAT GCGGAGATAA TAGCTTTAGT ATCTGATGAA GTTTTCCAGC |
| 1451 | CAGAAGCCGT GTGGAAACTC CTGGACATTC AGATAACTTG TGGAACTCTC |
| 1501 | GGGCTTTCAA CAGCAACTGT AAAACTTGCT GACGCTGATG GCAAAGAACA |
| 1551 | TGTCGCTTGT TCTATTGGAA CTGGGCCTGT GGATTCAGCT TACAAGGCAG |
| 1601 | TAGATCTTAT CGTAAAGGAA CCGGCTACTC TGCTTGAGTA CTCAATGAAT |
| 1651 | GCAGTAACAG AAGGCATTGA TGCCATCGCA ACCACAAGAG TTCTTATCCG |
| 1701 | TGGAAGCAAC AAATACTCAT CTACAAACGC AATAACTGGT GAAGAGGTTC |
| 1751 | AAAGAACCTT TAGTGGAACT GGAGCAGGAA TGGATATTGT GGTGTCAAGC |
| 1801 | GTCAAAGCTT ATGTTGAAGC TTTGAACAAA ATGATGGACT TCAAAGAAAA |
| 1851 | CTCCGCCACA AAAATCCCTT CCCAAAAAAA CAGAGTCGCT GCCTGA |

Another Arabidopsis thaliana isopropylmalate synthase IPMS1 protein sequence is shown below as SEQ ID NO:7, where two positions (226 and 604) are highlighted that can be modified to increase the production of various amino acids.

```
                10 20 30 40 50
MESSILKSPN LSSPSFGVPS IPALSSSSTS PFSSLHLRSQ NHRTISLTTA
                60 70 80 90 100
GKFRVSYSLS ASSPLPPHAP RRRPNYIPNR ISDPNYVRIF DTTLRDGEQS
              110 120 130 140 150
PGATLTSKEK LDIARQLAKL GVDIIEAGFP AASKDDFEAV KTIAETVGNT
              160 170 180 190 200
VDENGYVPVI CGLSRCNKKD IETAWEAVKY AKRPRIHTFI ATSDIHLKYK
              210 220 230 240 250
LKKSKEEVIE IARNMVRFAR SLGCEDVEFS PEDAGRSERE YLYEILGEVI
              260 270 280 290 300
KAGATTLNIP DTVGITLPSE FGQLIADIKA NTPGIQNVII STHCQNDLGL
              310 320 330 340 350
STANTLSGAH SGARQVEVTI NGIGERAGNA SLEEVVMAIK CRGDHVLGGL
              360 370 380 390 400
FTGIDTRHIV MTSKMVEEYT GMQTQPHKAI VGANAFAHES GIHQDGMLKH
              410 420 430 440 450
KGTYEIMSPE EIGLERSNDA GIVLGKLSGR HALKDRLNEL GYVLDDGQLS
              460 470 480 490 500
NLFWRFKAVA EQKKRVTDAD LIALVSDEVF QPEAVWKLLD MQITCGTLGL
              510 520 530 540 550
STSTVKLADS DGKEHVACSV GTGPVDAAYK AVDLIVKEPA TLLEYSMNAV
              560 570 580 590 600
TEGIDAIATT RVLIRGDNNY SSTNAVTGES VERTFSGTGA GMDIVVSSVK
              610 620 630
AYVGALNKML GFKEHTSTLS KTPLETNEVP A
```

A cDNA encoding the Arabidopsis thaliana IPMS1 protein sequence with SEQ ID NO:7 is shown below as SEQ ID NO:8, where modification of the guanine at position 825 (highlighted in bold with underlining) to an adenine can provide a protein like the eva1 protein, and/or modification of the guanine at position 1960 (highlighted in bold with underlining) to an adenine can provide a protein like the IPMS1-1D protein.

| | |
|---|---|
| 1 | TTTGGTTCGG TTCGGTTCGG AACAATTCAA ATAAATAAAA |
| 41 | CAAATCAAAA ATATTCACTA GCAAAGTAGT AACCAGAGAC |
| 81 | ACTGTGCCGT CGCCCGTCGC CGCCGCCGCC ACACTATCAT |
| 121 | CTCTCTCAGG TTTTTGATTT TCCACGGCAA TGGAGTCTTC |
| 161 | GATTCTCAAA AGCCCTAATC TCTCTTCACC ATCGTTCGGT |
| 201 | GTACCTTCAA TTCCCGCCTT ATCCTCCTCC TCCACCTCAC |
| 241 | CATTTTCATC TCTTCATCTC CGATCACAGA ACCACCGTAC |
| 281 | CATCTCTCTT ACCACCGCCG GAAAATTCCG TGTCTCGTAT |
| 321 | TCTCTCTCCG CTTCTTCACC TCTACCACCT CATCCTCCTC |
| 361 | GCCGTCGTCC CAATTACATC CCTAACCGTA TATCCGATCC |
| 401 | CAATTACGTC AGAATCTTCG ATACAACTCT CCGAGACGGT |
| 441 | GAACAGTCTC CCGGAGCTAC ACTAACCTCC AAGGAAAAGC |
| 481 | TCGATATCGC TCGTCAATTA GCCAAGCTCG GAGTCGACAT |
| 521 | CATCGAAGCT GGATTTCCCG CTGCTTCAAA AGACGATTTC |
| 561 | GAAGCTGTTA AAACCATAGC TGAGACTGTT GGCAATACCG |
| 601 | TCGACGAAAA TGGCTATGTC CCTGTAATCT GTGGTCTCTC |
| 641 | GAGATGTAAC AAGAAGGATA TTGAGACGGC TTGGGAAGCT |
| 681 | GTGAAGTACG CTAAGCGGCC AAGAATCCAT ACGTTTATTG |
| 721 | CCACTAGTGA TATTCATCTG AAGTATAAGT TGAAGAAGAG |
| 761 | TAAAGAAGAA GTTATTGAGA TCGCTAGGAA CATGGTTAGA |
| 801 | TTCGCCAGAA GCTTGGGATG TGAAGATGTT GAATTTAGTC |
| 841 | CAGAAGATGC CGGAAGATCG GAGAGAGAGT ACTTATACGA |
| 881 | GATTCTTGGT GAAGTGATCA AAGCTGGAGC AACCACTCTT |
| 921 | AACATACCTG ACACTGTTGG TATAACCTTG CCTAGTGAGT |
| 961 | TTGGTCAGTT GATTGCTGAT ATTAAAGCTA ATACTCCTGG |
| 1001 | GATCCAAAAT GTTATAATCT CTACACATTG TCAGAATGAT |
| 1041 | CTTGGACTCT CCACCGCCAA CACTTTATCT GGTGCACATT |
| 1081 | CGGGCGCGAG GCAAGTGGAA GTGACTATCA ATGGAATTGG |
| 1121 | CGAAAGAGCT GGAAACGCTT CATTGGAAGA GGTTGTCATG |
| 1161 | GCCATAAAAT GCCGTGGAGA TCATGTCTTA GGAGGCCTAT |
| 1201 | TTACTGGAAT CGATACCCGG CACATTGTTA TGACAAGCAA |
| 1241 | GATGGTTGAG GAGTACACTG GTATGCAAAC GCAGCCCCAT |
| 1281 | AAGGCTATTG TAGGAGCAAA CGCCTTTGCG CATGAAAGTG |
| 1321 | GTATTCATCA GGATGGAATG CTGAAGCACA AGGGTACCTA |
| 1361 | TGAAATTATG TCCCCCGAAG AGATTGGGCT TGAGCGATCA |
| 1401 | AATGATGCTG GCATCGTGCT GGGAAAGCTT AGTGGGCGTC |
| 1441 | ACGCACTGAA AGACCGTTTA AATGAGCTCG GTTATGTCCT |
| 1481 | GGATGATGGG CAGCTAAGCA ACCTTTTCTG GCGTTTCAAA |
| 1521 | GCTGTGGCAG AGCAAAAAAA GAGAGTTACC GATGCTGACT |
| 1561 | TAATAGCTTT AGTATCTGAT GAAGTGTTTC AGCCAGAGGC |
| 1601 | TGTCTGGAAA CTCCTGGACA TCCAGATAAC TTGTGGAACT |
| 1641 | CTCGGTCTCT CAACATCTAC TGTAAAACTT GCTGACTCCG |
| 1681 | ATGGCAAAGA GCATGTAGCT TGTTCTGTTG GAACCGGACC |
| 1721 | TGTAGATGCA GCTTACAAGG CAGTTGATCT TATCGTTAAG |
| 1761 | GAACCTGCGA CTCTGCTTGA GTACTCGATG AATGCAGTAA |
| 1801 | CAGAAGGCAT TGATGCTATT GCAACCACAC GGGTTCTAAT |
| 1841 | CCGCGGAGAC AACAACTACT CATCAACAAA CGCGGTAACG |
| 1881 | GGTGAATCTG TTGAAAGAAC TTTTAGTGGA ACCGGAGCAG |
| 1921 | GAATGGACAT TGTTGTGTCG AGCGTTAAAG CTTATGTTGG |
| 1961 | AGCTTTGAAC AAAATGTTGG GTTTCAAAGA ACACACCTCC |
| 2001 | ACTTTAAGTA AAACCCCTTT GGAGACCAAC GAAGTCCCTG |
| 2041 | CCTGAAGAAA ATCTTACCTG CAAATCTCAG AGATCAAATC |
| 2081 | ACAATTTAGA TGAGTAAACG TCTAAAAGAT TTTTATTTTT |
| 2121 | TGTTACCGTT ATTGTTTGTA TAAAAAGAAT ATGAGTTTTG |
| 2161 | GTTTACTAAA ATAACTATGA TATAAAACAG AGTATTTGGT |
| 2201 | TAAAACATTG AAACAAGAAC AAATTTTCTA TAAATAGTGA |
| 2241 | AGGCATTTGC CCTAACAGGA A |

Hence, a modified IPMS1 protein that provides plant with significantly higher levels of Gln, His, Ile, Leu, Lys, Met, Phe, Thr, Tim, Val, or a combination thereof in their leaves and seeds, and significant increases in their amino acid content or biomass compared to wild type, parental, or IPMS knockout leaves and/or seeds, the SEQ ID NO:7 protein can have a substitution at position 226. For example, the sequence of the SEQ ID NO:7 IPMS1 protein can be modified to have an asparagine at position 226 instead of an aspartic acid (D226N), which has the following sequence (SEQ ID NO:9).

```
                10 20 30 40 50
MESSILKSPN LSSPSFGVPS IPALSSSSTS PFSSLHLRSQ NHRTISLTTA
                60 70 80 90 100
GKFRVSYSLS ASSPLPPHAP RRRPNYIPNR ISDPNYVRIF DTTLRDGEQS
              110 120 130 140 150
PGATLTSKEK LDIARQLAKL GVDIIEAGFP AASKDDFEAV KTIAETVGNT
              160 170 180 190 200
VDENGYVPVI CGLSRCNKKD IETAWEAVKY AKRPRIHTFI ATSDIHLKYK
              210 220 230 240 250
LKKSKEEVIE IARNMVRFAR SLGCENVEFS PEDAGRSERE YLYEILGEVI
              260 270 280 290 300
KAGATTLNIP DTVGITLPSE FGQLIADIKA NTPGIQNVII STHCQNDLGL
              310 320 330 340 350
STANTLSGAH SGARQVEVTI NGIGERAGNA SLEEVVMAIK CRGDHVLGGL
              360 370 380 390 400
FTGIDTRHIV MTSKMVEEYT GMQTQPHKAI VGANAFAHES GIHQDGMLKH
              410 420 430 440 450
KGTYEIMSPE EIGLERSNDA GIVLGKLSGR HALKDRLNEL GYVLDDGQLS
              460 470 480 490 500
NLFWRFKAVA EQKKRVTDAD LIALVSDEVF QPEAVWKLLD MQITCGTLGL
              510 520 530 540 550
STSTVKLADS DGKEHVACSV GTGPVDAAYK AVDLIVKEPA TLLEYSMNAV
              560 570 580 590 600
TEGIDAIATT RVLIRGDNNY SSTNAVTGES VERTFSGTGA GMDIVVSSVK
              610 620 630
AYVGALNKML GFKEHTSTLS KTPLETNEVP A
```

Also, a modified IPMS1 protein that provides plants with significantly higher levels of Gln, His, Ile, Leu, Lys, Met, Phe, Thr, Trp, Val, or a combination thereof in their leaves and seeds, and significant increases in their biomass or amino acid content compared to wild type, parental, or IPMS knockout leaves and/or seeds, the SEQ ID NO:7 protein can have a substitution at position 604. For example, the sequence of the SEQ ID NO:7 IPMS1 protein can be modified to have a glutamic acid at position 604 instead of a glycine (G604E), which has the following sequence (SEQ ID NO:10).

| | |
|---|---|
| 1 | MESSILKSPN LSSPSFGVPS IPALSSSSTS PFSSLHLRSQ |
| 41 | NHRTISLTTA GKFRVSYSLS ASSPLPPHAP RRRPNYIPNR |
| 81 | ISDPNYVRIF DTTLRDGEOS PGATLTSKEK LDIARQLAKL |
| 121 | GVDIIEAGFP AASKDDFEAV KTIAETVGNT VDENGYVPVI |
| 161 | CGLSRCNKKD IETAWEAVKY AKRPRIHTFI ATSDIHLKYK |
| 201 | LKKSKEEVIE IARNMVRFAR SLGCEDVEFS PEDAGRSERE |
| 241 | YLYEILGEVI KAGATTLNIP DTVGITLPSE FGQLIADIKA |
| 281 | NTPGIONVII STHCQNDLGL STANTLSGAH SGARQVEVTI |
| 321 | NGIGERAGNA SLEEVVMAIK CRGDHVLGGL FTGIDTRHIV |
| 361 | MTSKMVEEYT GMQTQPHKAI VGANAFAHES GIHQDGMLKH |
| 401 | KGTYEIMSPE EIGLERSNDA GIVLGKLSGR HALKDRLNEL |
| 441 | GYVLDDGQLS NLFWRFKAVA EQKKRVIDAD LIALVSDEVF |
| 481 | QPEAVWKLLD MQITCGTLGL STSTVKLADS DGKEHVACSV |
| 521 | GTGPVDAAYK AVDLIVKEPA TLLEYSMNAV TEGIDAIATI |
| 561 | RVLIRGDNNY SSTNAVTGES VERTFSGTGA GMDIVVSSVK |
| 601 | AYVEALNKML GFKEHTSTLS KTPLETNEVP A |

Various plant species have IPMS1 genes that can be modified to provide those plant species with significantly higher levels of Gln, His, Ile, Leu, Lys, Met, Phe, Thr, Trp, Val, or a combination thereof, in their leaves and seeds, and significant increases in their biomass or amino acid content compared to wild type, parental, or IPMS knockout leaves and/or seeds. Examples of sequences from various plant species are described below.

For example, a sequence for a Brachypodium distachyon (Purple false brome) IPMS protein is shown below as SEQ ID NO:11, where two positions (229 and 604) are highlighted that can be modified to increase the production of various amino acids.

```
                10 20 30 40 50
MAASPAKPCC FSSLNPASST PLARRSRTLS SSAAAAKPHR FSHGLAAAAV
                60 70 80 90 100
AANPRAAALR RPVRACLAAG AAPRRPEYVP DRIDDPNYVR IFDTTLRDGE
              110 120 130 140 150
QSPGATMTSA EKLVVARQLA RLGVDIIEAG FPASSPDDLD AVRSIAIEVG
              160 170 180 190 200
NTPLGGDGHV PVICGLSRCN KRDIDAAWEA VRHARKPRIH TFIATSEIHM
              210 220 230 240 250
QHKLRKTPEQ VVAIAREMVA YARSLGCPDV EFSPEDAGRS NREFLYHILE
              260 270 280 290 300
EVIKAGATTL NIPDTVGYNL PHEFGKLIAD IKANTPGIEN AIISTHCQND
              310 320 330 340 350
LGLASANTLA GAYAGARQLE VTINGIGERA GNASLEEVVM AIKCRRELLG
              360 370 380 390 400
GLYTGISTOH ITMSSKMVQE HSGLHVQPHK AIVGANAFAH ESGIHQDGML
              410 420 430 440 450
KHKGTYEIIS PEDIGLVRVN EFGIVLGKLS GRHAVKTKLV ELGYEISDKE
              460 470 480 490 500
FEDFFKRYKE VAEKKKRVTD EDIEALLSDE IFQPKVIWSL GDVQATCGTI
              510 520 530 540 550
GLSTATVKLI AIDGEEKIGC SVGTGPVDAA YKAVDQIIQI PTVLREYSMT
              560 570 580 590 600
SVTEGIDAIA TTRVVITGDV SNSKNALIGQ NNRSFSGSGA ALDVVVSSVR
              610 620 630
AYLSALNKMS SYVGAVKASS EAPESIRTVQ TAE
```

The Brachypodium distachyon (Purple false brome) IPMS protein with SEQ ID NO:11 is encoded by the 2-isopropylmalate synthase A gene on chromosome 4 (LOC100832390; locus tag BRADI_4g43130; see NCBI webpage at ncbi.nlm.nih.gov/gene/100832390).

To generate a modified Brachypodium distachyon IPMS protein that provides plants with significantly higher levels of Gln, His, Ile, Leu, Lys, Met. Phe, Thr, Trp, Val, or a combination thereof in their leaves and seeds, and significant increases in their biomass or amino acid content compared to wild type, parental, or IPMS knockout leaves and/or seeds, the SEQ ID NO: 11 protein can be modified to have a substitution at position 229. For example, the sequence of the SEQ ID NO:11 IPMS protein can be modified to have an asparagine at position 229 instead of an aspartic acid (D229N), which has the following sequence (SEQ ID NO:12).

```
                10 20 30 40 50
MAASPAKPCC FSSLNPASST PLARRSRTLS SSAAAAKPHR FSHGLAAAAV
                60 70 80 90 100
AANPRAAALR RPVRACLAAG AAPRRPEYVP DRIDDPNYVR IFDTTLRDGE
              110 120 130 140 150
QSPGATMTSA EKLVVARQLA RLGVDIIEAG FPASSPDDLD AVRSIAIEVG
              160 170 180 190 200
NTPLGGDGHV PVICGLSRCN KRDIDAAWEA VRHARKPRIH TFIATSEIHM
              210 220 230 240 250
QHKLRKTPEQ VVAIAREMVA YARSLGCPNV EFSPEDAGRS NREFLYHILE
              260 270 280 290 300
EVIKAGATTL NIPDTVGYNL PHEFGKLIAD IKANTPGIEN AIISTHCQND
              310 320 330 340 350
LGLASANTLA GAYAGARQLE VYINGIGERA GNASLEEVVM AIKCRRELLG
              360 370 380 390 400
GLYTGISTQH ITMSSKMVQE ASGLHVQPHK AIVGANAFAH ESGIHQDGML
              410 420 430 440 450
KHKGTYEIIS PEDIGLVRVN EFGIVLGKLS GRHAVKTKLV ELGYEISDKE
              460 470 480 490 500
FEDFFKRYKE VAEKKKRVTD EDIEALLSDE IFQPKVIWSL GDVQATCGTL
              510 520 530 540 550
GLSTATVKLI AIDGEEKIGC SVGTGPVDAA YKAVDQIIQI PTVLREYSMT
              560 570 580 590 600
SVTEGIDAIA TTRVVITGDV SNSKNALIGQ NNRSFSGSGA ALDVVVSSVR
              610 620 630
AYLSALNKMS SYVGAVKASS EAPESIRTVQ TAE
```

To generate a modified Brachypodium distachyon IPMS protein that provides plants with significantly higher levels of Gln, His. Ile, Leu, Lys. Met, Phe, Thr. Trp. Val, or a combination thereof in their leaves and seeds, and significant increases in their biomass or amino acid content compared to wild type, parental, or IPMS knock-out leaves and/or seeds, the SEQ ID NO:11 protein can have a substitution at position 604. For example, the sequence of the SEQ ID NO:11 IPMS1 protein can be modified to have a glutamic acid at position 604 instead of a serine (S604E), which has the following sequence (SEQ ID NO: 13).

```
                10 20 30 40 50
MAASPAKPCC FSSLNPASST PLARRSRTLS SSAAAAKPHR FSHGLAAAAV
                60 70 80 90 100
AANPRAAALR RPVRACLAAG AAPRRPEYVP DRIDDPNYVR IFDTTLRDGE
              110 120 130 140 150
QSPGATMTSA EKLVVARQLA RLGVDIIEAG FPASSPDDLD AVRSIAIEVG
              160 170 180 190 200
NTPLGGDGHV PVICGLSRCN KRDIDAAWEA VRHARKPRIH TFIATSEIHM
              210 220 230 240 250
QHKLRKTPEQ VVAIAREMVA YARSLGCPDV EFSPEDAGRS NREFLYHILE
              260 270 280 290 300
EVIKAGATTL NIPDTVGYNL PHEFGKLIAD IKANTPGIEN AIISTHCQND
              310 320 330 340 350
LGLASANTLA GAYAGARQLE VTINGIGERA GNASLEEVVM AIKCRRELLG
              360 370 380 390 400
GLYTGISTQH TTMSSKMVQE HSGLHVQPHK AIVGANAFAH ESGIHQDGML
              410 420 430 440 450
KHKGTYEIIS PEDIGLVRVN EFGIVLGKLS GRHAVKTKLV ELGYEISDKE
              460 470 480 490 500
FEDFFKRYKE VAEKKKRVTD EDIEALLSDE IFQPKVIWSL GDVQATCGTL
              510 520 530 540 550
GLSTATVKLI AIDGEEKIGC SVGTGPVDAA YKAVDQIIQI PTVLREYSMT
              560 570 580 590 600
SVTEGIDAIA TTRVVITGDV SNSKNALIGQ NNRSFSGSGA ALDVVVSSVR
              610 620 630
AYLEALNKMS SYVGAVKASS EAPESIRTVQ
```

A sequence for a Glycine max (soybean) IPMS1 protein is shown below as SEQ ID NO: 14, where two positions (167 and 545) are highlighted that can be modified to increase the production of various amino acids.

```
                10 20 30 40 50
MATKTSTNGT HHSLPEYIPN RIPDPHYVR1 LDTTLRDGEQ APGAAMTSDQ
                60 70 80 90 100
KLQIARQLAK LGVDVIEGGE PSASQEDFNA VKMIAQEVGN NCDADGYVPV
              110 120 130 140 150
IAALCRCNER DITRAWEALK YAKRPRLMPF IAVSPIHMEY KLNKTKEEVL
              160 170 180 190 200
QIATDMIKFA RGLGCTDIQF CSEDAARSDR EFLYQILEEV IKAGATTLGI
              210 220 230 240 250
GDTVGITMPF EIRELVAGIK ANVPGAENVI ISIHCHNDLG HATANTIEAA
              260 270 280 290 300
RAGAMQLEVT 1NGIGERAGN ASLEEVVMAL KCRGDHVLGG LYTGINTRHL
              310 320 330 340 350
LKTSKMVEEE SGMYLQPHKA VVGDNAFLHE SGVHQAGLLK HRGTYEILSP
              360 370 380 390 400
EDIGHEKSNG VNMVLGKLSG RQALKSRLKE LGYELRDEEV ESVFRNFKAI
              410 420 430 440 450
AEKKKRVTDV DLKALVSDQA SHAEPIWKLG GLQVTCGTMG SSTATIKLVT
              460 470 480 490 500
SDGSTHVACS VGVGPVDSAY KAINLIVKET VKVLEYSPST VTGGTDAIAT
              510 520 530 540 550
TRVVIRRENK QSPTPALNGN VIYPTFSGTG EGVDIVTSSV EAYITALNKM
LDSKE
```

The Glycine max (soybean) IPMS1 protein IPMS protein with SEQ ID NO: 14 is encoded by the 2-isopropylmalate synthase gene on chromosome 3 (LOC100816439; locus tag GLYMA_03(3005700: see NCBI website).

The SEQ ID NO: 14 IPMS1 protein has about 67% sequence identity with the SEQ ID NO:2 IPMS1 protein as illustrated below.

| | | |
|---|---|---|
| Sq2 | 76 | PEYIPNRISDPNYVRVFDTTLRDGEQSPGATLTSKEKLDIARQLAKLGVDIIEAGFPAAS |
| Sq14 | 15 | PEYIPNRIPDPHYVRILDTTLRDGEQAPGAAMTSDQKLQIARQLAKLGVDVIEGGFPSAS |
| | | ******** ** *** ********* *** ** ** *********** ** *** ** |
| Sq2 | 136 | KDDFEAVKTIAETVGNTVDENGYVPVICGLSRCNKKDIERAWDAVKYAKRPRIHTFIATS |
| Sq14 | 75 | QEDFNAVKMIAQEVGNNCDADGYVPVIAALCRCNERDITRAWEALKYAKRPRLMPFIAVS |
| | | ** *** ** *** * ****** * *** ** *** * ******* *** * |
| Sq2 | 196 | DIHLEYKLKKTKAEVIEIARSMVRFARSLGCEDVEFSPEDAGRSEREYLYEILGEVIKAG |
| Sq14 | 135 | PIHMEYKLNKTKEEVLQIATDMIKFARGLGCTDIQFCSEDAARSDREFLYQILEEVIKAG |
| | | ** **** *** ** ** * *** *** * * *** ** ** ** ** ****** |
| Sq2 | 256 | ATTLNIPDTVGITLPSEFGQLITDLKANTPGIENVVISTHCQNDLGLSTANTLSGAHAGA |
| Sq14 | 195 | ATTLGIGDTVGITMPFEIRELVAGIKANVPGAENVIISIHCHNDLGHATANTIEAARAGA |
| | | **** * ****** * * * *** ** *** ** ** **** **** * *** |
| Sq2 | 316 | RQMEVTINGIGERAGNASLEEVVMAIKCRGDHVLGGLFTGIDTRHIVMTSKMVEEYTGMQ |
| Sq14 | 255 | MQLEVTINGIGERAGNASLEEVVMALKCRGDHVLGGLYTGINTRHLLKTSKMVEEFSGMY |
| | | * ********************** *********** *** *** ******* ** |
| Sq2 | 376 | TQPHKAIVGANAFAHESGIHQDGMLKHKGTYEIICPEEIGLERSNDAGIVLGKLSGRHAL |
| Sq14 | 315 | LQPHKAVVGDNAFLHESGVHQAGLLKHRGTYEILSPEDIGHEKSNGVNMVLGKLSGRQAL |
| | | ***** ** *** **** ** * *** ***** ** ** * ** ******** ** |
| Sq2 | 436 | KDRLTELGYQLDDEQLSTIFWRFKTVAEQKKRVTDADIIALVSDEVFQPEAVWKLLDIQI |
| Sq14 | 375 | KSRLKELGYELRDEEVESVFRNFKAIAEKKKRVTDVDLKALVSDQASHAEPIWKLGGLQV |
| | | * ** **** * ** * ** ** ****** * ***** * *** * |
| Sq2 | 496 | TCGTLGLSTATVKLADADGKEHVACSIGTGPVDSAYKAVDLIVKEPATLLEYSMNAVTEG |
| Sq14 | 435 | TCGTMGSSTATIKLVTSGDSTHVACSVGVGPVDSAYKAINLIVKETVKVLEYSPSTVTGG |
| | | **** * **** ** ** ***** * ********* ***** **** ** * |
| Sq2 | 556 | IDAIATTRVLIRGSNKYSSTNAITGEEVQRTFSGTGAGMDIVVSSVKAYVGALNKMMDFK |
| Sq14 | 495 | TDAIATTRVVIRRENKQSPTPALNGNVIYPTFSGTGEGVDIVTSSVEAYITALKKMLDSK |
| | | ******** ** ** * * * * ****** * *** *** ** ***** * * |
| Sq2 | 616 | E |
| Sq14 | 555 | E |
| | | * |

To generate a modified IPMS1 protein that provides Glycine max (soybean) plants with significantly higher levels of Gln, His Ile Leu Lys, Met, Phe, Thr, Trp Val, or a combination thereof in their leaves and seeds, and significant increases in their biomass or amino acid content compared to wild type, parental, or IPMS knockout leaves and/or seeds, the SEQ ID NO: 14 protein can have a substitution at position 167. For example, the sequence of the SEQ ID NO:14 IPMS1 protein can be modified to have an asparagine at position 167 instead of an aspartic acid (D167N′), which has the following sequence (SEQ ID NO: 15).

```
                10 20 30 40 50
MATKTSTNGT HHSLPEYIPN RIPDPHYVRI LDTTLRDGEQ APGAAMTSDQ
                60 70 80 90 100
KLQIARQLAK LGVDVIEGGF PSASQEDFNA VKMIAQEVGN NCDADGYVPV
              110 120 130 140 150
IAALCRCNER DITRAWEALK YAKRPRLMPF IAVSPIHMEY KLNKTKEEVL
              160 170 180 190 200
QIATDMIKFA RGLGCTNIQF CSEDAARSDR EFLYQILEEV IKAGATTLGI
              210 220 230 240 250
GDTVGITMPF EIRELVAGIK ANVPGAENVI ISIHCHNDLG HATANTIEAA
              260 270 280 290 300
RAGAMQLEVT INGIGERAGN ASLEEVVMAL KCRGDHVLGG LYTGINTRHL
              310 320 330 340 350
LKTSKMVEEF SGMYLQPHKA VVGDNAFLHE SGVHQAGLLK HRGTYEILSP
              360 370 380 390 400
EDIGHEKSNG VNMVLGKLSG RQALKSRLKE LGYELRDEEV ESVFRNFKAI
              410 420 430 440 450
AEKKKRVTDV DLKALVSDQA SHAEPIWKLG GLQVTCGTMG SSTATIKLVT
              460 470 480 490 500
SDGSTHVACS VGVGPVDSAY KAINLIVKET VKVLEYSPST VTGGTDAIAT
              510 520 530 540 550
TRVVIRRENK QSPTPALNGN VIYPTFSGTG EGVDIVTSSV EAYITALNKM
LDSKE
```

To generate a modified IPMS1 protein that provides Glycine max (soybean) plant with significantly higher levels of Gln, His, Ile, Leu, Lys, Met, Phe, Thr, Trp, Vat, or a combination thereof in their leaves and seeds, and significant increases in their biomass or amino acid content compared to wild type, parental, or IPMS knockout leaves and/or seeds, the SEQ ID NO: 14 protein can have a substitution at position 545. For example, the sequence of the SEQ ID NO: 14 IPMS1 protein can be modified to have a glutamic acid at position 545 instead of a threonine (T545E), which has the following sequence (SEQ ID NO: 16).

```
                10 20 30 40 50
MATKTSTNGT HHSLPEYIPN RIPDPHYVRI LDTTLRDGEQ APGAAMTSDQ
                60 70 80 90 100
KLQIARQLAK LGVDVIEGGF PSASQEDFNA VKMIAQEVGN NCDADGYVPV
              110 120 130 140 150
IAALCRCNER DITRAWEALK YAKRPRLMPF IAVSPIHMEY KLNKTKEEVL
              160 170 180 190 200
QTATDMIKFA RGLGCTDIQF CSEDAARSDR EFLYQILEEV IKAGATTLGI
              210 220 230 240 250
GDTVGITMPF EIRELVAGIK ANVPGAENVI ISIHCHNDLG HATANTIEAA
              260 270 280 290 300
RAGAMQLEVT INGIGERAGN ASLEEVVMAL KCRGDHVLGG LYTGINTRHL
              310 320 330 340 350
LKTSKMVEEF SGMYLQPHKA VVGDNAFLHE SGVHQAGLLK HRGTYEILSP
              360 370 380 390 400
EDIGHEKSNG VNMVLGKLSG RQALKSRLKE LGYELRDEEV ESVFRNEKAI
              410 420 430 440 450
AEKKKRVTDV DLKALVSDQA SHAEPIWKLG GLQVTCGTMG SSTATIKLVT
              460 470 480 490 500
SDGSTHVACS VGVGPVDSAY KAINLIVKET VKVLEYSPST VTGGTDAIAT
              510 520 530 540 550
TRVVIRRENK QSPTPALNGN VIYPTFSGTG EGVDIVTSSV EAYIEALNKM
LDSKE
```

Another example of a Glycine max (soybean) IPMS1 protein is shown below as SEQ ID NO: 17, where 2-3 positions (225, 226, and 595) are highlighted as amino acids that can be modified to increase the production of various amino acids.

```
                10 20 30 40 50
MATVIRNPIL FPSTSHHPNQ NHTFLTLRFS QTLRSSLRSK SRFAVSCSQS
                60 70 80 90 100
EPPPPHPSSS RRRPPYIPNL IPDPSYVRIF DTTLRDGEQS PGASMTSKEK
              110 120 130 140 150
LDVARQLAKL GVDIIEAGFP AASKDDFEAV KMIAQAVGNA VENDGYVPVI
              160 170 180 190 200
CGLSRCNEKD IRTAWEAVKY AKRPRIHTFI ATSPIHMEYK LRMSKDKVVD
              210 220 230 240 250
IARNMVKFAR SLGCDDVEFS PEDAGRSDRE FLYEILGEVI KVGATTLNIP
              260 270 280 290 300
DTVGITMPSE FGKLIADIKA NTPGIENVII STHCQNDLGL STANTIEGAR
              310 320 330 340 350
AGARQLEVTI NGIGERAGNA SLEEVVMALR CGAHVNGNLY TGINTKHIFL
              360 370 380 390 400
TSKMVEEYTG LQIQPHKALV GANAFAHESG IHQDGMLKHK GTYEIISPED
              410 420 430 440 450
IGLERTNEAG IVLGKLSGRH ALRKRLEELG YELNDDQVQT LFWCFKAVAE
              460 470 480 490 500
QKKRVTDADL RALVSDEVFQ AEPVWKLGDL QVTCGTLGLS TATVKLLSSD
              510 520 530 540 550
GSTHVACSIG TGPVDSAYKA VDLIVKEQVT LLEYSMNAVT EGIDAIATTR
              560 570 580 590 600
VVIRGESETS TITTHALTGE TVIRTFSGTG AGMDVVVSSV KAYIAALNKM
              610 620
SGFKESSQSA EKISISSLKI
```

The Glycine max (soybean) IPMS1 protein IPMS protein with SEQ ID NO: 17 is encoded by the 2-isopropylmalate synthase gene on chromosome 10 (LOC100788955; locus tag GLYMA_10G295400; see NCBI website).

The SEQ ID NO: 17 IPMS1 protein has about 75% sequence identity with the SEQ ID NO:2 IPMS1 protein as illustrated below.

| | | |
|---|---|---|
| Sq2 | 14 | STTITTTSFLPTFSSKPTPISSSFRFQPSHHRSISLRSQT-LRLSCSISDPSPLPPHTPR |
| Sq17 | 2 | ATVIRNPILFPSTSHHPNQNHTFLTLRFSQTLRSSLRSKSRFAVSCSQSEPPPPHPSSSR |
| | | * * * * * * **** *** * * * * * |
| Sq2 | 73 | RPRPEYIPNRISDPNYVRVFDTTLRDGEQSPGATLTSKEKLDIARQLAKLGVDIIEAGFP |
| Sq17 | 62 | R-RPPYIPNLIPDPSYVRIFDTTLRDGEQSPGASMTSKEKLDVARQLAKLGVDIIEAGFP |
| | | * ** **** * ** *** ************** ******* ***************** |
| Sq2 | 133 | AASKDDFEAVKTIAETVGNTVDENGYVPVICGLSRCNKKDIERAWDAVKYAKRPRIHTFI |
| Sq17 | 121 | AASKDDFEAVKMIAQAVGNAVENDGYVPVICGLSRCNEKDIRTAWEAVKYAKRPRIHTFI |
| | | *********** ** *** * ************* *** ** ************** |
| Sq2 | 193 | ATSDTHLEYKLKKTKAEVIEIARSMVRFARSLGCEDVEFSPEDAGRSEREYLYEILGEVI |
| Sq17 | 181 | ATSPIHMEYKLRMSKDKVVDIARNMVKFARSLGCDDVEFSPEDAGRSDREFLYEILGEVI |
| | | *** ** **** * * *** ** ******* ************ ** ********* |
| Sq2 | 253 | KAGATTLNIPDTVGITLPSEFGQLITDLKANTPGIENVVISTHCQNDLGLSTANTLSGAH |
| Sql7 | 241 | KVGATTLNIPDTVGITMPSEFGKLIADIKANTPGIENVIISTHCQNDLGLSTANTIEGAR |
| | | * ************** ***** ** * ********** **************** ** |
| Sq2 | 313 | AGARQMEVTINGIGERAGNASLEEVVMAIKCRGDHVLGGLFTGIDTRHIVMTSKMVEEYT |
| Sql7 | 301 | AGARQLEVTINGIGERAGNASLEEVVMALRC-GAHVNGNLYTGINTKHIFLTSKMVEEYT |
| | | ***** ********************** * * ** * * *** * ** ********* |
| Sq2 | 373 | GMQTQPHKAIVGANAFAHESGIHQDGMLKHKGTYEIICPEEIGLERSNDAGIVLGKLSGR |
| Sq17 | 360 | GLQIQPHKALVGANAFAHESGIHQDGMLKHKGTYEIISPEDIGLERTNEAGIVLGKLSGR |
| | | * * ***** *************************** ** ***** * *********** |
| Sq2 | 433 | HALKDRLTELGYQLDDEQLSTIFWRFKTVAEQKKRVTDADIIALVSDEVFQPEAVWKLLD |
| Sq17 | 420 | HALRKRLEELGYELNDDQVQTLFWCFKAVAEQKKRVTDADLRALVSDEVFQAEPVWKLGD |
| | | *** ** **** * * * * ** ** ************ ********* * **** * |
| Sq2 | 493 | IQITCGTLGLSTATVKLADADGKEHVACSIGTGPVDSAYKAVDLIVKEPATLLEYSMNAV |
| Sq17 | 480 | LQVTCGTLGLSTATVKLLSSDGSTHVACSIGTGPVDSAYKAVDLIVKEQVTLLEYSMNAV |
| | | * ************** ** ************************ ********** |
| Sq2 | 553 | TEGIDAIATTRVLIRGSNKYSS--TNAITGEEVQRTFSGTGAGMDIVVSSVKAYVGALNK |
| Sq17 | 540 | TEGIDAIATTRVVIRGESETSTITTHALTGETVIRTFSGTGAGMDVVVSSVKAYIAALNK |
| | | ************ *** * * * *** * *********** ******** **** |
| Sq2 | 611 | MMDFKENS |
| Sq1 | 600 | MSGFKESS |
| | | * *** * |

To generate a modified IPMS protein that provides Glycine max (soybean) plants with significantly higher levels of Gln, His, Ile, Leu, Lys, Met, Phe, Thr, Trp, Val, or a combination thereof in their leaves and seeds, and significant increases in their biomass or amino acid content compared to wild type, parental, or IPMS knockout leaves and/or seeds, the SEQ ID NO:17 protein can have a substitution at position 225 and/or 226. For example, the sequence of the SEQ ID NO:17 IPMS protein can be modified to have an asparagine at 216 instead of an aspartic acid (D216N), which has the following sequence (SEQ ID NO:18).

```
                10 20 30 40
MATVIRNPIL FPSTSHHPNQ NHTFLTLRFS QTLRSSLRSK
                50 60 70 80
SRFAVSCSQS EPPPPHPSSS RRRPPYIPNL IPDPSYVRIF
                90 100 110 120
DTTLRDGEQS PGASMTSKEK LDVARQLAKL GVDIIEAGFP
              130 140 150 160
AASKDDFEAV KMIAQAVGNA VENDGYVPVI CGLSRCNEKD
              170 180 190 200
IRTAWEAVKY AKRPRIHTFI ATSPIHMEYK LRMSRDKVVD
              210 220 230 240
IARNMVKFAR SLGCDNVEFS PEDAGRSDRE FLYEILGEVI
              250 260 270 280
KVGATTLNIP DTVGITMPSE FGKLIADIKA NTPGIENVII
              290 300 310 320
STHCQNDLGL STANTIEGAR AGARQLEVTI NGIGERAGNA
              330 340 350 360
SLEEVVMALR CGAHVNGNLY TGINTKHIFL TSKMVEEYTG
              370 380 390 400
LQIQPHKALV GANAFAHESG IHQDGMLKHK GTYEIISPED
              410 420 430 440
IGLERTNEAG IVLGKLSGRH ALRKRLEELG YELNDDQVQT
              450 460 470 480
LFWCFKAVAE QKKRVTDADL RALVSDEVFQ AEPVWKLGDL
              490 500 510 520
QVTCGTLGLS TATVKLLSSD GSTHVACSIG TGPVDSAYKA
              530 540 550 560
VDLIVKEQVT LLEYSMNAVT EGIDAIATTR VVIRGESETS
              570 580 590 600
TITTHALTGE TVIRTFSGTG AGMDVVVSSV KAYIAALNKM
              610 620
SGFKESSQSA EKISISSLKI
```

To generate a modified IPMS protein that provides Glycine max (soybean) plants with significantly higher levels of Gln, His, Ile, Leu, Lys, Met, Phe, Thr, Trp, Vat, or a combination thereof in their leaves and seeds, and significant increases in their biomass or amino acid content compared to wild type, parental, or IPMS knock-out leaves and/or seeds, the SEQ ID NO: 17 protein can have a substitution at position 595. For example, the sequence of the SEQ ID NO:17 IPMS1 protein can be modified to have a glutamic acid at position 595 instead of an alanine (A595E), which has the following sequence (SEQ ID NO: 19).

```
                10 20 30 40
MATVIRNPIL FPSTSHHPNQ NHTFLTLRFS QTLRSSLRSK
                50 60 70 80
SRFAVSCSQS EPPPPHPSSS RRRPPYIPNL IPDPSYVRIF
                90 100 110 120
DTTLRDGEQS PGASMTSKEK LDVARQLAKL GVDIIEAGFP
              130 140 150 160
AASKDDFEAV KMIAQAVGNA VENDGYVPVI CGLSRCNEKD
              170 180 190 200
IRTAWEAVKY AKRPRIHTFI ATSPIHMEYK LRMSKDKVVD
              210 220 230 240
IARNMVKFAR SLGCDDVEFS PEDAGRSDRE FLYEILGEVI
              250 260 270 280
KVGATTLNIP DTVGITMPSE FGKLIADIKA NTPGIENVII
              290 300 310 320
STHCQNDLGL STANTIEGAR AGARQLEVTI NGIGERAGNA
              330 340 350 360
SLEEVVMALR CGAHVNGNLY TGINTKHIFL TSKMVEEYTG
              370 380 390 400
LQIQPHKALV GANAFAHESG IHQDGMLKHK GTYEIISPED
              410 420 430 440
IGLERTNEAG IVLGKLSGRH ALRKRLEELG YELNDDQVQT
              450 460 470 480
LFWCFKAVAE QKKRVTDADL RALVSDEVFQ AEPVWKLGDL
              490 500 510 520
QVTCGTLGLS TATVKLLSSD GSTHVACSIG TGPVDSAYKA
              530 540 550 560
VDLIVKEQVT LLEYSMNAVT EGIDAIATTR VVIRGESETS
              570 580 590 600
TITTHALTGE TVIRTFSGTG AGMDVVVSSV KAYIAELNKM
              610 620
SGFKESSQSA EKISISSLKI
```

An example of Zea mays (corn) IPMS1 protein is shown below as SEQ ID NO:20, where two positions (235 and 612) are highlighted that can be modified to increase the production of various amino acids.

| | |
|---|---|
| 1 | MAFNAKPYCS TTAKPPTPAA HRSPSGPSPC ISVRAAAVSP |
| 41 | RARHSAYGLS AAGNASSSTV RLRALAQRTR AQPQPPRRRP |
| 81 | EYVPNRIDDP NYVRIFDTTL RDGEQSPGAT MTSAEKLVVA |
| 121 | RQLARLGVDV IEAGFPASSP DDLDAVRSIA IEVGNPPPGD |
| 161 | DGGAHVPVIC GLSRCNRRDI DAAWEAVRHA RRPRIHTFIA |
| 201 | TSEIHMQHKL RKTPDQVVAV AREMVAYARS LGCADVEFSP |
| 241 | EDAGRSNREF LYHILEEVIK AGATTLNIPD TVGYTLPYEF |
| 281 | GKLISDIKEN TPGIENAIIS THCQNDLGLA TANTLAGAHA |
| 321 | GARQLEVTIN GIGERAGNAS LEEVVMAIKC RGELLDGLYT |
| 361 | GINSQHITLT SKMVQEHSGL HVQPHKAIVG ANAFAHESGI |
| 401 | HQDGMLKYKG TYEIISPDDI GLARANEFGI VLGKLSGRHA |
| 441 | VRSKLVELGY EISDKEFEDF FKRYKEVAEK KKRVTDEDIE |
| 481 | ALLSDEIFQP KVIWSLADVQ ATCGTLGLST ATVKLIGPDG |
| 521 | EERIACSVGT GPVDAAYKAV DQIIQIPTVL REYGMTSVTE |
| 561 | GIDAIATTRV VVTGDVANNS KHALTGHSFN RSFSGSGAAM |
| 601 | DIVVSSVRAY LSALNKMCSF AGAARASSTE VPGSASVQRA |
| 641 | E |

The Zea mays (corn) IPMS1 protein IPMS protein with SEQ ID NO:20 is encoded by the 2-isopropylmalate synthase gene on chromosome 4 (LOC100280189; locus tag ZEAMMB73_Zm00001d052472; see NCBI website).

The SEQ ID NO:20 corm IPMS1 protein has about 75% sequence identity with the SEQ ID NO:2 Arabidopsis IPMS1 protein as illustrated below.

| | | |
|---|---|---|
| Sq2 | 71 | PRRPRPEYIPNRISDPNYVRVFDTTLRDGEQSPGATLTSKEKLDIARQLAKLGVDIIEAG |
| Sq20 | 75 | PPRRRPEYVPNRIDDPNYVRIFDTTLRDGEQSPGATMTSAEKLVVARQLARLGVDVIEAG |
| | | * * **** **** ****** *************** ** *** ***** **** **** |
| Sq2 | 131 | FPAASKDDFEAVKTIAETVGNTV---DENGYVPVICGLSRCNKKDIERAWDAVKYAKRPR |
| Sq20 | 135 | FPASSPDDLDAVRSIAIEVGNPPPGDDGGAHVPVICGLSRCNRRDIDAAWEAVRHARRPR |
| | | *** * ** ** ** *** * *********** ** ** ** * *** |
| Sq2 | 188 | IHTFIATSDIHLEYKLKKTKAEVIEIARSMVRFARSLGCEDVEFSPEDAGRSEREYLYEI |
| Sq20 | 195 | IHTFIATSEIHMQHKLRKTPDQVVAVAREMVAYARSLGCADVEFSPEDAGRSNREFLYHI |
| | | ******** ** ** ** * ** ** ****** ************ ** ** * |
| Sq2 | 248 | LGEVIKAGATTLNIPDTVGITLPSEFGQLITDLKANTPGIENVVISTHCQNDLGLSTANT |
| Sq20 | 255 | LEEVIKAGATTLNIPDTVGYTLPYEFGKLISDIKENTPGIENAIISTHCQNDLGLATANT |
| | | * ***************** *** *** ** * * ******* *********** **** |
| Sq2 | 308 | LSGAHAGARQMEVTINGIGERAGNASLEEVVMAIKCRGDHVLGGLFTGIDTRHIVMTSKM |
| Sq20 | 315 | LAGAHAGARQLEVTINGIGERAGNASLEEVVMAIKCRGE-LLDGLYTGINSQHITLTSKM |
| | | * ******** *************************** * ** *** ** **** |
| Sq2 | 368 | VEEYTGMQTQPHKAIVGANAFAHESGIHQDGMLKHKGTYEIICPEEIGLERSNDAGIVLG |
| Sq20 | 374 | VQEHSGLHVQPHKAIVGANAFAHESGIHQDGMLKYKGTYEIISPDDIGLARANEFGIVLG |
| | | * * * ************************* ******* * *** * * ***** |
| Sq2 | 428 | KLSGRHALKDRLTELGYQLDDEQLSTIFWRFKTVAEQKKRVTDADIIALVSDEVFQPEAV |
| Sq20 | 434 | KLSGRHAVRSKLVELGYEISDKEFEDFFKRYKEVAEKKKRVTDEDIEALLSDELFQPKVI |
| | | ******* * **** * * * * *** ****** ** ** *** *** |
| Sq2 | 488 | WKLLDIQITCGTLGLSTATVKLADADGKEHVACSIGTGPVDSAYKAVDLIVKEPATLLEY |
| Sq20 | 494 | WSLADVQATCGTLGLSTATVKLIGPDGEERIACSVGTGPVDAAYKAVDQIIQIPTVLREY |
| | | * * * * ************** ** * *** ****** ****** * * * ** |
| Sq2 | 548 | SMNAVTEGIDAIATTRVLIRGSNKYSSTNAITGEEVQRTFSGTGAGMDIVVSSVKAYVGA |
| Sq20 | 554 | GMTSVTEGIDAIATTRVVVTGDVANNSKHALTGHSFNRSFSGSGAAMDIVVSSVRAYLSA |
| | | * ************* * * * ** * *** ** ******** ** * |
| Sq2 | 608 | LNKMMDF |
| Sq20 | 614 | LNKMCSF |
| | | **** * |

To generate a modified IPMS1 protein that provides corn plants with significantly higher levels of Gln, His, Ile, Leu, Lys, Met, Phe, Thr, Trp, Val, or a combination thereof in their leaves and seeds, and significant increases in their biomass compared to wild type, parental, or IPMS knockout leaves and/or seeds, the SEQ ID NO:20 protein can have a substitution at position 235. For example, the sequence of the SEQ ID NO:20 IPMS11 protein can be modified to have an asparagine at 235 instead of an aspartic acid (D235N), which has the following sequence (SEQ ID NO:21).

| | |
|---|---|
| 1 | MAFNAKPYCS TTAKPPTPAA HRSPSGPSPC ISVRAAAVSP |
| 41 | RARHSAYGLS AAGNASSSTV RLRALAQRTR AQPQPPRRRP |
| 81 | EYVPNRIDDP NYVRIFDTTL RDGEQSPGAT MTSAEKLVVA |
| 121 | RQLARLGVDV IEAGFPASSP DDLDAVRSIA IEVGNPPPGD |
| 161 | DGGAHVPVIC GLSRCNRRDI DAAWEAVRHA RRPRIHTFIA |
| 201 | TSEIHMQHKL RKTPDQVVAV AREMVAYARS LGCANVEFSP |
| 241 | EDAGRSNREF LYHILEEVIK AGATTLNIPD TVGYTLPYEF |
| 281 | GKLISDIKEN TPGIENAIIS THCQNDLGLA TANTLAGAHA |
| 321 | GARQLEVTIN GIGERAGNAS LEEVVMAIKC RGELLDGLYT |
| 361 | GINSQHITLT SKMVQEHSGL HVQPHKAIVG ANAFAHESGI |
| 401 | HQDGMLKYKG TYEIISPDDI GLARANEFGI VLGKLSGRHA |
| 441 | VRSKLVELGY EISDKEFEDF FKRYKEVAEK KKRVTDEDIE |
| 481 | ALLSDEIFQP KVIWSLADVQ ATCGTLGLST ATVKLIGPDG |
| 521 | EERIACSVGT GPVDAAYKAV DQIIQIPTVL REYGMTSVTE |
| 561 | GIDAIATTRV VVTGDVANNS KHALTGHSFN RSFSGSGAAM |
| 601 | DIVVSSVRAY LSALNKMCSF AGAARASSTE VPGSASVQRA |
| 641 | E |

To generate a modified IPMS1 protein that provides corn plants with significantly higher levels of Gln, His, Ile, Leu, Lys, Met, Phe, Thr, Trp. Val. or a combination thereof in (heir leaves and seeds, and significant increases in their biomass compared to wild type, parental, or IPMS knock-out leaves and/or seeds, the SEQ ID NO:20 protein can have a substitution at position 612. For example, the sequence of the SEQ ID NO:20 IPMS1 protein can be modified to have a glutamic acid at position 612 instead of a serine (S612E), which has the following sequence (SEQ ID NO:22).

| | |
|---|---|
| 1 | MAFNAKPYCS TTAKPPTPAA HRSPSGPSPC ISVRAAAVSP |
| 41 | RARHSAYGLS AAGNASSSTV RLRALAQRTR AQPQPPRRRP |
| 81 | EYVPNRIDDP NYVRIFDTTL RDGEQSPGAT MTSAEKLVVA |
| 121 | RQLARLGVDV IEAGFPASSP DDLDAVRSIA IEVGNPPPGD |
| 161 | DGGAHVPVIC GLSRCNRRDI DAAWEAVRHA RRPRIHTFIA |
| 201 | TSEIHMQHKL RKTPDQVVAV AREMVAYARS LGCADVEFSP |
| 241 | EDAGRSNREF LYHILEEVIK AGATTLNIPD TVGYTLPYEF |
| 281 | GKLISDIKEN TPGIENAIIS THCQNDLGLA TANTLAGAHA |
| 321 | GARQLEVTIN GIGERAGNAS LEEVVMAIKC RGELLDGLYT |
| 361 | GINSQHITLI SKMVQEHSGL HVQPHKAIVG ANAFAHESGI |
| 401 | HQDGMLKYKG TYEIISPDDI GLARANEFGI VLGKLSGRHA |
| 441 | VRSKLVELGY EISDKEFEDF FKRYKEVAEK KKRVTDEDIE |
| 481 | ALLSDEIFQP KVIWSLADVQ ATCGTLGLST ATVKLIGPDG |
| 521 | EERIACSVGT GPVDAAYKAV DQIIQIPTVL REYGMTSVTE |
| 561 | GIDAIATTRV VVTGDVANNS KHALTGHSFN RSFSGSGAAM |
| 601 | DIVVSSVRAY LEALNKMCSF AGAARASSTE VPGSASVQRA |
| 641 | E |

Another Zea mays (corn) IPMS1 protein is shown below as SEQ ID NO:23, where two positions (235 and 612) are highlighted that can be modified to increase the production of various amino acids.

| | |
|---|---|
| 1 | MAFSAKPHCS CSTTTKPPTP VAHLSPPPSL SVRAAACAPR |
| 41 | SAYGLSAAGV GGGKASPSTV RLRARAQRIR ASQQQPRRRP |
| 81 | EYVPNRIDDP NYVRIFDTTL RDGEQSPGAT MTSAEKLVVA |
| 121 | RQLARLGVDI IEAGFPASSP DDLDAVRSIA IEVGNPPTAS |
| 161 | AGTVHVPVIC GLSRCNRKDI DAAWEAVRHA RRPRIHTFIA |
| 201 | TSEIHMQHKL RKTPEQVVAI AREMVAYARS LGCPDVEFSP |
| 241 | EDAGRSNREF MYHILEEVIK AGATTLNIPD TVGYTLPYEF |
| 281 | GKLIADIKAN TSGIENAIIS THCQNDLGLA TANTLAGARS |
| 321 | GARQLEVTIN GIGERAGNAS LEEVVMAIKC RGELLDGLYT |
| 361 | GINSQHITLT SKMVQEHSGL HVQPHKAIVG ANAFAHESGI |
| 401 | HQDGMLKYKG TYEIISPDDI GLTRANEFGI VLGKLSGRHA |
| 441 | VRSKLVELGY EISDKEFEDF FKRYKEVAEK KKRVTDEDIE |
| 481 | ALLSDEIFQP KVIWSLADVQ ATCGTLGLST ATVKLIAPDG |
| 521 | EERIACSVGT GPVDAAYKAI DQIIQIPTVL REYGMTSVTE |
| 561 | GIDAIATTRV VVTGDVTNNS KHALTGRAFN RSFSGSGAAM |
| 601 | DIVVSSVRAY LSALNKMCSF AGAAKASGEV PESASVQSAE |

The Zea mays (corn) IPMS1 protein IPMS protein with SEQ ID NO:23 is encoded by the 2-isopropylmalate synthase B gene on chromosome 2 (LOC100281571; locus tag ZEAMMB73_Zm00001d004960; see NCBI website).

The SEQ ID NO:23 corn IPMS1 protein has about 70% sequence identity with the SEQ ID NO:2 Arabidopsis IPMS1 protein as illustrated below.

| | | |
|---|---|---|
| Sq2 | 73 | RPRPEYIPNRISDPNYVRVFDTTLRDGEQSPGATLTSKEKLDIARQLAKLGVDIIEAGFP |
| Sq23 | 77 | RRRPEYVPNRIDDPNYVRIFDTTLRDGEQSPGATMTSAEKLVVARQLARLGVDIIEAGFP |
| | | * **** **** ****** *************** ** *** ***** *********** |
| Sq2 | 133 | AASKDDFEAVKTIAETVGNTVDENG---YVPVICGLSRCNKKDIERAWDAVKYAKRPRIH |
| Sq23 | 137 | ASSPDDLDAVRSIAIEVGNPPTASAGTVHVPVICGLSRCNRKDIDAAWEAVRHARRPRIH |
| | | * * ** ** ** *** *********** *** ** ** * ***** |
| Sq2 | 190 | TFIATSDIHLEYKLKKTKAEVIEIARSMVRFARSLGCEDVEFSPEDAGRSEREYLYEILG |
| Sq23 | 197 | TFIATSEIHMQHKLRKTPEQVVAIAREMVAYARSLGCPDVEFSPEDAGRSNREFMYHILE |
| | | ****** ** ** ** * *** ** ****** ************ ** * ** |
| Sq2 | 250 | EVIKAGATTLNIPDTVGITLPSEFGQLITDLKANTPGIENVVISTHCQNDLGLSTANTLS |
| Sq23 | 257 | EVIKAGATTLNIPDTVGYTLPYEFGKLIADIKANTSGIENAIISTHCQNDLGLATANTLA |
| | | ***************** *** *** ** * **** **** *********** ***** |
| Sq2 | 310 | GAHAGARQMEVTINGIGERAGNASLEEVVMAIKCRGDHVLGGLFTGIDTRHIVMTSKMVE |
| Sq23 | 317 | GARSGARQLEVTINGIGERAGNASLEEVVMAIKCRGE-LLDGLYTGINSQHITLTSKMVQ |
| | | ** **** *************************** * ** *** ** ***** |
| Sq2 | 370 | EYTGMQTQPHKAIVGANAFAHESGIHQDGMLKHKGTYEIICPEEIGLERSNDAGIVLGKL |
| Sq23 | 376 | EHSGLHVQPHKAIVGANAFAHESGIHQDGMLKYKGTYEIISPDDIGLTRANEFGIVLGKL |
| | | * * ************************* ******* * *** * * ******* |
| Sq2 | 430 | SGRHALKDRLTELGYQLDDEQLSTIFWRFKTVAEQKKRVTDADIIALVSDEVFQPEAVWK |
| Sq23 | 436 | SGRHAVRSKLVELGYEISDKEFEDFFKRYKEVAEKKKRVTDEDIEALLSDEIFQPKVIWS |
| | | ***** * **** * * * * *** ****** ** ** *** *** * |
| Sq2 | 490 | LLDIQITCGTLGLSTATVKLADADGKEHVACSIGTGPVDSAYKAVDLIVKEPATLLEYSM |
| Sq23 | 496 | LADVQATCGTLGLSTATVKLIAPDGEERIACSVGTGPVDAAYKAIDQIIQIPTVLREYGM |
| | | * * * ************** ** * *** ****** **** * * * * ** * |
| Sq2 | 550 | NAVTEGIDAIATTRVLIRGSNKYSSTNAITGEEVQRTFSGTGAGMDIVVSSVKAYVGALN |
| Sq23 | 556 | TSVTEGIDAIATTRVVVTGDVTNNSKHALTGRAFNRSFSGSGAAMDIVVSSVRAYLSALN |
| | | ************* * * * ** * *** ** ******** ** *** |
| Sq2 | 610 | KMMDF |
| Sq23 | 616 | KMCSF |
| | | ** * |

To generate a modified IPMS1 protein that provides corn plants with significantly higher levels of Gln, His, Ile, Leu, Lys, Met, Phe, Thr, Trp, Val, or a combination thereof in their leaves and seeds, and significant increases in their biomass compared to wild type, parental, or IPMS knockout leaves and/or seeds, the SEQ ID NO:23 protein can have a substitution at position 235. For example, the sequence of the SEQ ID NO:23 IPMS1 protein can be modified to have an asparagine at 235 instead of an aspartic acid (D235N), which has the following sequence (SEQ ID NO:24).

| | |
|---|---|
| 1 | MAFSAKPHCS CSTTTKPPTP VAHLSPPPSL SVRAAACAPR |
| 41 | SAYGLSAAGV GGGKASPSTV RLRARAQRIR ASQQQPRRRP |
| 81 | EYVPNRIDDP NYVRIFDTTL RDGEQSPGAT MTSAEKLVVA |
| 121 | RQLARLGVDI IEAGFPASSP DDLDAVRSIA IEVGNPPTAS |
| 161 | AGTVHVPVIC GLSRCNRKDI DAAWEAVRHA RRPRIHTFIA |
| 201 | TSEIHMQHKL RKTPEQVVAI AREMVAYARS LGCPNVEFSP |
| 241 | EDAGRSNREF MYHILEEVIK AGATTLNIPD TVGYTLPYEF |
| 281 | GKLIADIKAN TSGIENAIIS THCQNDLGLA TANTLAGARS |
| 321 | GARQLEVTIN GIGERAGNAS LEEVVMAIKC RGELLDGLYT |
| 361 | GINSQHITLT SKMVQEHSGL HVQPHKAIVG ANAFAHESGI |
| 401 | HQDGMLKYKG TYEIISPDDI GLTRANEFGI VLGKLSGRHA |
| 441 | VRSKLVELGY EISDKEFEDF FKRYKEVAEK KKRVTDEDIE |
| 481 | ALLSDEIFQP KVIWSLADVQ ATCGTLGLST ATVKLIAPDG |
| 521 | EERIACSVGT GPVDAAYKAI DQIIQIPTVL REYGMTSVTE |
| 561 | GIDAIATTRV VVTGDVTNNS KHALTGRAFN RSFSGSGAAM |
| 601 | DIVVSSVRAY LSALNKMCSF AGAAKASGEV PESASVQSAE |

To generate a modified IPMS1 protein that provides corn plants with significantly higher levels of Gln, His, Ile, Leu, Lys, Met, Phe, Thr, Trp, Val, or a combination thereof in their leaves and seeds, and significant increases in their biomass compared to wild type, parental, or IPMS knockout leaves and/or seeds, the SEQ ID NO:23 protein can have a substitution at position 612. For example, the sequence of the SEQ ID NO:23 IPMS1 protein can be modified to have a glutamic acid at position 612 instead of a serine (S612E), which has the following sequence (SEQ ID NO:25).

| | |
|---|---|
| 1 | MAFSAKPHCS CSTTTKPPTP VAHLSPPPSL SVRAAACAPR |
| 41 | SAYGLSAAGV GGGKASPSTV RLRARAQRIR ASQQQPRRRP |
| 81 | EYVPNRIDDP NYVRIFDTTL RDGEQSPGAT MTSAEKLVVA |
| 121 | RQLARLGVDI IEAGFPASSP DDLDAVRSIA IEVGNPPTAS |
| 161 | AGTVHVPVIC GLSRCNRKDI DAAWEAVRHA RRPRIHTFIA |
| 201 | TSEIHMQHKL RKTPEQVVAI AREMVAYARS LGCPDVEFSP |
| 241 | EDAGRSNREF MYHILEEVIK AGATTLNIPD TVGYTLPYEF |
| 281 | GKLIADIKAN TSGIENAIIS THCQNDLGLA TANTLAGARS |
| 321 | GARQLEVTIN GIGERAGNAS LEEVVMAIKC RGELLDGLYT |
| 361 | GINSQHITLT SKMVQEHSGL HVQPHKAIVG ANAFAHESGI |
| 401 | HQDGMLKYKG TYEIISPDDI GLTRANEFGI VLGKLSGRHA |
| 441 | VRSKLVELGY EISDKEFEDF FKRYKEVAEK KKRVTDEDIE |
| 481 | ALLSDEIFQP KVIWSLADVQ ATCGTLGLST ATVKLIAPDG |
| 521 | EERIACSVGT GPVDAAYKAI DQIIQIPTVL REYGMTSVTE |
| 561 | GIDAIATTRV VVTGDVTNNS KHALTGRAFN RSFSGSGAAM |
| 601 | DIVVSSVRAY LEALNKMCSF AGAAKASGEV PESASVQSAE |

A Solanum lycopersicum (tomato) IPMS1 protein is shown below as SEQ ID NO:26, where two positions (210 and 588) are highlighted that can be modified to increase the production of various amino acids.

| | |
|---|---|
| 1 | MASITANHPI SGKPLISFRP KNPLLQTQTL FNFKPSISKH |
| 41 | SNSSFSIPVV RCSIRRRPEY TPSHIPDPNY VRIFDTTLRD |
| 81 | GEQSPGATMT TKEKLDVARQ LAKLGVDIIE AGFPASSEAD |
| 121 | LEAVKLIAKE VGNGVYEEGY VPVICGLARC NKKDIDKAWE |
| 161 | AVKYAKKPRI HTFIATSEIH MNYKLKMSRD QVVEKARSMV |
| 201 | AYARSIGCED VEFSPEDAGR SDPEFLYHIL GEVIKAGATT |
| 241 | LNIPDTVGYT VPEEFGQLIA KIKANTPGVE DVIISTHCQN |
| 281 | DLGLSTANTL AGACAGARQL EVTINGIGER AGNASLEEVV |
| 321 | MALKCRGEQV LGGLYTGINT QHILMSSKMV EEYSGLHVQP |
| 361 | HKAIVGANAF AHESGIHQDG MLKHKDTYEI ISPEDIGLNR |
| 401 | ANESGIVLGK LSGRHALQAK MLELGYEIEG KELDDLFWRF |
| 441 | KSVAEKKKKI TDDDLVALMS DEVFQPQFVW QLQNVQVTSG |
| 481 | SLGLSTATVK LIDADGREHI SCSVGTGPVD AAYKAVDLIV |
| 521 | KVPVTLLEYS MNAVTQGIDA IASTRVLIRG ENGHTSTHAV |
| 561 | TGETIHRTFS GTGADMDIVI SSVRAYVGAL NKMMSFRKLM |
| 601 | AKNNKPESSA VV |

The Solanum lycopersicum (tomato) IPMS protein with SEQ ID NO:26 is encoded by the 2-isopropylmalate synthase B gene on chromosome 6 (LOC101245066, see NCBI website).

As illustrated below the SEQ ID NO:26 Solanum lycopersicum IPMS1 sequence has about 75% sequence identity with the Arabidopsis IPMS1 SEQ ID NO: 2 sequence.

| | | |
|---|---|---|
| Sq2 | 73 | RPRPEYIPNRISDPNYVRVFDTTLRDGEQSPGATLTSKEKLDIARQLAKLGVDIIEAGFP |
| Sq26 | 55 | RRRPEYTPSHIPDPNYVRIFDTTLRDGEQSPGATMTTKEKLDVARQLAKLGVDIIEAGFP |
| | | * **** * * ****** *************** * ***** ***************** |
| Sq2 | 133 | AASKDDFEAVKTIAETVGNTVDENGYVPVICGLSRCNKKDIERAWDAVKYAKRPRIHTFT |
| Sq26 | 115 | ASSEADLEAVKLIAKEVGNGVYEEGYVPVICGLARCNKKDIDKAWEAVKYAKKPRIHTFI |
| | | * * * **** ** *** * * ********* ******* ** ****** ******* |
| Sq2 | 193 | ATSDIHLEYKLKKTKAEVIEIARSMVRFARSLGCEDVEFSPEDAGRSEREYLYEILGEVI |
| Sq26 | 175 | ATSEIHMNYKLKMSRDQVVEKARSMVAYARSIGCEDVEFSPEDAGRSDPEFLYHILGEVI |
| | | *** ** **** * * ***** *** *************** * ** ****** |
| Sq2 | 253 | KAGATTLNIPDTVGITLPSEFGQLITDLKANTPGIENVVISTHCQNDLGLSTANTLSGAH |
| Sq26 | 235 | KAGATTLNIPDTVGYTVPEEFGQLIAKIKANTPGVEDVIISTHCQNDLGLSTANTLAGAC |
| | | ************** * * ****** ****** * * ***************** ** |
| Sq2 | 313 | AGARQMEVTINGIGERAGNASLEEVVMAIKCRGDHVLGGLFTGIDTRHIVMTSKMVEEYT |
| Sq26 | 295 | AGARQLEVTINGIGERAGNASLEEVVMALKCRGEQVLGGLYTGINTQHILMSSKMVEEYS |
| | | ***** ********************** **** ***** *** * ** * ******* |
| Sq2 | 373 | GMQTQPHKAIVGANAFAHESGIHQDGMLKHKGTYEIICPEEIGLERSNDAGIVLGKLSGR |
| Sq26 | 355 | GLHVQPHKAIVGANAFAHESGIHQDGMLKHKDTYEIISPEDIGLNRANESGIVLGKLSGR |
| | | * *************************** ***** ** *** * * ********** |
| Sq2 | 433 | HALKDRLTELGYQLDDEQLSTIFWRFKTVAEQKKRVTDADIIALVSDEVFQPEAVWKLLD |
| Sq26 | 415 | HALQAKMLELGYEIEGKELDDLFWRFKSVAEKKKKITDDDLVALMSDEVFQPQFVWQLQN |
| | | *** **** * ***** *** ** ** * ** ******* ** * |
| Sq2 | 493 | IQITCGTLGLSTATVKLADADGKEHVACSIGTGPVDSAYKAVDLIVKEPATLLEYSMNAV |
| Sq26 | 475 | VQVTSGSLGLSTATVKLIDADGREHISCSVGTGPVDAAYKAVDLIVKVPVTLLEYSMNAV |
| | | * * * ********** **** ** ** ****** ********** * ********** |
| Sq2 | 553 | TEGIDAIATTRVLIRGSNKYSSTNAITGEEVQRTFSGTGAGMDIVVSSVKAYVGALNKMM |
| Sq26 | 535 | TQGIDAIASTRVLIRGENGHTSTHAVTGETIHRTFSGTGADMDIVISSVRAYVGALNKMM |
| | | * ****** ******* * ** * *** ******** **** *** ********** |
| Sq2 | 613 | DFKE |
| Sq26 | 595 | SFRK |
| | | * |

A cDNA encoding the Solanum lycopersicum (tomato) IPMS1 protein with SEQ ID NO:26 is shown below as SEQ ID NO:27.

| | |
|---|---|
| 1 | TAACAGTCTC TTACTTGAAT TTTCAGTCTC TTCTCCCAGC |
| 41 | CGCCATTAAA ACCAGCACTC GCAAACTCCA ATTTCTCTTC |
| 81 | TCCGGCAATG GCGTCTATCA CCGCAAATCA TCCAATCTCC |
| 121 | GGTAAACCAT TAATCTCATT CCGTCCCAAA AACCCTTTAC |
| 161 | TTCAAACCCA AACTCTCTTC AATTTCAAAC CATCAATCTC |
| 201 | CAAGCACTCC AATTCTTCAT TTTCCATTCC CGTTGTCCGC |
| 241 | TGCTCAATCC GCCGTAGACC GGAATATACT CCGAGTCACA |
| 281 | TTCCCGATCC AAACTATGTC CGGATATTCG ACACCACTCT |
| 321 | CCGTGATGGC GAACAATGAG CAGGGGCTAC AATGACTACA |
| 361 | AAGGAGAAAC TGGATGTTGC ACGTCAGTTA GCTAAGCTTG |
| 401 | GTGTTGATAT AATTGAGGCT GGTTTTCCTG CTTCTTCTGA |
| 441 | AGCTGATCTT GAAGCTGTGA AATTGATAGC TAAGGAAGTT |
| 481 | GGGAATGGTG TTTATGAAGA GGGATATGTT CCGGTTATTT |
| 521 | GTGGATTGGC GAGGTGTAAT AAGAAGGATA TTGATAAGGC |
| 561 | GTGGGAGGCT GTGAAGTATG CTAAGAAACC GAGGATTCAT |
| 601 | ACGTTTATTG CTACAAGTGA GATACATATG AATTATAAGC |
| 641 | TGAAAATGAG TAGAGATCAA GTTGTTGAGA AAGCTAGGAG |
| 681 | TATGGTGGCT TATGCAAGGA GTATTGGGTG TGAGGATGTT |
| 721 | GAATTTAGCC CTGAAGATGC TGGAAGATCT GATCCAGAGT |
| 761 | TTCTTTATCA TATCCTTGGA GAGGTTATCA AAGCTGGGGC |
| 801 | AACAACCCTT AACATCCCTG ATACTGTTGG ATACACTGTA |
| 841 | CCCGAAGAAT TTGGACAATT GATTGCTAAA ATAAAAGCGA |
| 881 | ATACCCCAGG AGTTGAAGAT GTGATCATTT CAACACACTG |
| 921 | CCAGAACGAT CTTGGGCTTT CTACTGCCAA CACCTTAGCT |
| 961 | GGAGCATGTG CAGGTGCAAG ACAATTGGAA GTGACCATCA |
| 1001 | ATGGAATTGG TGAAAGAGCT GGAAATGCTT CTTTAGAGGA |
| 1041 | GGTTGTAATG GCCTTAAAGT GTCGTGGAGA GCAAGTACTA |
| 1081 | GGTGGCCTAT ATACAGGGAT TAATACACAA CATATACTCA |
| 1121 | TGTCAAGCAA GATGGTAGAG GAGTATTCCG GACTTCATGT |
| 1161 | GCAGCCACAC AAAGCCATTG TTGGAGCTAA TGCCTTTGCT |
| 1201 | CATGAAAGTG GCATCCATCA GGATGGAATG TTAAAACACA |
| 1241 | AAGATACATA TGAGATTATA TCTCCTGAAG ATATTGGGCT |
| 1281 | TAATCGTGCT AATGAATCTG GTATTGTCCT TGGGAAACTC |
| 1321 | AGTGGGCGTC ATGCTTTGCA AGCCAAAATG CTTGAGCTTG |
| 1361 | GATACGAGAT TGAGGGCAAA GAACTTGATG ACCTGTTCTG |
| 1401 | GCGATTCAAA TCTGTGGCTG AGAAGAAAAA GAAAATTACA |
| 1441 | GATGATGACC TGGTAGCACT GATGTCAGAT GAGGTTTTCC |
| 1481 | AGCCTCAATT TGTGTGGCAA CTTCAAAATG TACAGGTTAC |
| 1521 | TTCTGGAAGT CTTGGGCTTT CTACAGCAAC TGTTAAGCTC |
| 1561 | ATTGATGCTG ATGGTCGAGA GCATATTTCT TGTTCTGTTG |
| 1601 | GAACGGGGCC AGTTGACGCG GCTTATAAGG CAGTTGATCT |
| 1641 | CATTGTTAAG GTACCTGTAA CACTCCTTGA GTATTCCATG |
| 1681 | AATGCAGTCA CACAAGGTAT AGATGCTATA GCTTCAACCA |
| 1721 | GAGTCTTAAT TCGTGGAGAA AATGGCCATA CATCAACCCA |
| 1761 | TGCCGTAACT GGAGAGACTA TTCACCGTAC ATTTAGTGGA |
| 1801 | ACCGGAGCAG ATATGGATAT TGTCATCTCC AGTGTCCGAG |
| 1841 | CCTATGTTGG TGCATTGAAT AAGATGATGA GTTTCAGAAA |
| 1881 | ACTAATGGCG AAAAATAACA AACCCGAAAG CAGTGCAGTC |
| 1921 | GTATAGGTAC TTCTGTGCAA ATCAAGGTTA TGGAACTTTT |
| 1961 | GCAACTGCAC TGGAGCTTTA TCATTTGTAC AAAATGTAGG |
| 2001 | AGTCTGTTCA AAGAATTTGA GCCTGTAGTT TTCAAGAAAA |
| 2041 | CAAAGCTTAA TATGTCTGGT AGTGCTTGAA AATCATCTAA |
| 2081 | GTTTATGGTC TATCAGTTGG AACATTAGAC ACATTGTCCA |
| 2121 | TATAACTTTG TTCATGCTCC CGTTTAACTA ATTTATGAAT |
| 2161 | CTACATACCA CGCAAGATTT TCGAAATGAT TTCAGAAATT |
| 2201 | ATGAAAATCT CTGTATTA |

Another example of a Solanum lycopersicum (tomato) IPMS1 protein sequence is shown below as SEQ ID NO:28, where two positions (212 and 590) are highlighted that can be modified to increase the production of various amino acids.

| | |
|---|---|
| 1 | MSSSSSLCSN SVFSYRNNFS IFQSKNVLLP PISSTNNFSF |
| 41 | SIKKHYYSTF IRCSISNRRP EYVPSKISDP KYVRIFDTTL |
| 81 | RDGEQSPGAT MTTKEKLDVA RQLAKLGVDI IEAGFPASSE |
| 121 | ADFESVKLIA EEIGNNTDEN GFVPVICGLS RCNKSDIDKA |
| 161 | WEAVKYAKKP RVHTFIATSE IHMKYKLKMS REQVVEKARS |
| 201 | MVAYARSLGC EDVEFSPEDA GRSDREFLYD ILGEVIKAGA |
| 241 | TTLNIPDTVG YTVPSEFGQL IADIKANTPG IENVIISTHC |
| 281 | QNDLGLSTAN TLAGACAGAR QLEVTINGIG ERAGNASLEE |
| 321 | VVMALKCRGE QVLGGLYTGI NTQHIVPSSK MVEEYSGLQV |
| 361 | QPHKAIVGAN AFAHESGIHQ DGMLKHKDTY EIISPDDVGL |
| 401 | SRSNEAGIVL GKLSGRHALK SKMLELGYDI DGKELEDLFW |
| 441 | RFKSVAEKKK KITDDDLIAL MSDEVLQPNV YWKLGDVQIM |
| 481 | CGSLGLSTAT VKLINTDGQE HIACSVGTGP VDAAYKAVDL |
| 521 | IVKVPITLLE YSMNAVTEGI DAIASTRVSI CSIDRHTIMN |
| 561 | GSTGQTIHRT FSGTGADMDV VISSVRAYIG ALNKMLSYEK |
| 601 | LVSRYSKPED SVVV |

The Solanum lycopersicum (tomato) IPMS protein with SEQ ID NO:28 is encoded by the 2-isopropylmalate synthase B gene on chromosome 8 (Gene ID: 101251907; see NCBI website).

As illustrated below the SEQ ID NO:28 Solanum lycopersicum IPMS1 sequence has about 76% sequence identity with the Arabidopsis IPMS1 SEQ ID NO: 2 sequence.

| | | |
|---|---|---|
| Sq2 | 75 | RPEYIPNRISDPNYVRVFDTTLRDGEQSPGATLTSKEKLDIARQLAKLGVDIIEAGFPAA |
| Sq28 | 59 | RPEYVPSKISDPKYVRIFDTTLRDGEQSPGATMTTKEKLDVARQLAKLGVDIIEAGFPAS |
| | | **** * **** *** *************** * ***** ****************** |
| Sq2 | 135 | SKDDFEAVKTIAETVGNTVDENGYVPVICGLSRCNKKDIERAWDAVKYAKRPRIHTFIAT |
| Sq28 | 119 | SEADFESVKLIAEEIGNNTDENGFVPVICGLSRCNKSDIDKAWEAVKYAKKPRVHTFIAT |
| | | * *** ** *** ** **** ************ ** ** ****** ** ****** |
| Sq2 | 195 | SDIHLEYKLKKTKAEVIEIARSMVRFARSLGCEDVEFSPEDAGRSEREYLYEILGEVIKA |
| Sq28 | 179 | SEIHMKYKLKMSREQVVEKARSMVAYARSLGCEDVEFSPEDAGRSDREFLYDILGEVIKA |
| | | * ** **** * * ***** ******************* ** ** ******** |
| Sq2 | 255 | GATTLNIPDTVGITLPSEFGQLITDLKANTPGIENVVISTHCQNDLGLSTANTLSGAHAG |
| Sq28 | 239 | GATTLNIPDTVGYTVPSEFGQLIADIKANTPGIENVIISTHCQNDLGLSTANTLAGACAG |
| | | ************ * ******** * ********** ***************** ** ** |
| Sq2 | 315 | ARQMEVTINGIGERAGNASLEEVVMAIKCRGDHVLGGLFTGIDTRHIVMTSKMVEEYTGM |
| Sq28 | 299 | ARQLEVTINGIGERAGNASLEEVVMALKCRGEQVLGGLYTGINTQHIVPSSKMVEEYSGL |
| | | *** ********************** **** ***** *** * *** ******* * |
| Sq2 | 375 | QTQPHKAIVGANAFAHESGIHQDGMLKHKGTYEIICPEEIGLERSNDAGIVLGKLSGRHA |
| Sq28 | 359 | QVQPHKAIVGANAFAHESGIHQDGMLKHKDTYEIISPDDVGLSRSNEAGIVLGKLSGRHA |
| | | * *************************** ***** * ** *** ************* |
| Sq2 | 435 | LKDRLTELGYQLDDEQLSTIFWRFKTVAEQKKRVTDADIIALVSDEVFQPEAVWKLLDIQ |
| Sq28 | 419 | LKSKMLELGYDIDGKELEDLFWRFKSVAEKKKKITDDDLIALMSDEVLQPNVYWKLGDVQ |
| | | ** **** * * ***** *** ** ** * *** **** ** *** * * |
| Sq2 | 495 | ITCGTLGLSTATVKLADADGKEHVACSIGTGPVDSAYKAVDLIVKEPATLLEYSMNAVTE |
| Sq28 | 479 | IMCGSLGLSTATVKLINTDGQEHIACSVGTGPVDAAYKAVDLIVKVPITLLEYSMNAVTE |
| | | * ** ********** ** ** *** ****** ********** * ************ |
| Sq2 | 555 | GIDAIATTRVLIRGSNKYSSTNAITGEEVQRTFSGTGAGMDIVVSSVKAYVGALNKMMDE |
| Sq28 | 539 | GIDAIASTRVSICSIDRHTIMNGSTGQTIHRTFSGTGADMDVVISSVRAYIGALNKMLSY |
| | | ****** *** * * ** ******** ** * *** ** ****** |
| Sq2 | 615 | KE |
| Sq28 | 599 | EK |

A cDNA encoding the SEQ ID NO:28 tomato IPMS1 protein is shown below as SEQ ID NO:29.

| | |
|---|---|
| 1 | CTCTTTGTGA AATGATCTAG AATCTTTTAA CATATGAGAT |
| 41 | AAATGACTTG GTATCCACTT GGTTCAAATA ATTTGGAGTA |
| 81 | CACTTTTTTC TTTTTATTAT ATAAATCICC ACACTTTGTT |
| 121 | AGTATTAAAA CCCCACTTTT CCTTTTCTCC TCTGTGTTTG |
| 161 | ACCCTTTTCT GCACATTTTC AGAATGTCTT CTTCTTCTTC |
| 201 | TCTTTGTTCA AACTCTGTAT TTTCTTATAG AAACAACTTC |
| 241 | TCAATTTTTC AATCCAAAAA TGTTCTTCTT CCTCCAATTT |
| 281 | CTAGTACCAA TAATTTCAGT TTTTCAATCA AAAAACACTA |
| 321 | CTACTCCACA TTTATCCGGT GTTCGATTTC GAATCGTCGA |
| 361 | CCGGAATATG TACCCAGTAA AATCTCCGAC CCGAAATACG |
| 401 | TTCGCATATT CGATACTACT CTTCGTGACG GTGAGCAATC |
| 441 | TCCAGGTGCT ACAATGACTA CGAAAGAGAA ACTCGACGTC |
| 481 | GCTCGTCAGC TAGCGAAACT CGGTGTTGAT ATAATTGAAG |
| 521 | CTGGATTTCC AGCTTCATCT GAAGCAGATT TCGAATCTGT |
| 561 | GAAACTAATT GCAGAGGAAA TTGGTAATAA TACTGATGAA |
| 601 | AATGGATTTG TGCCTGTAAT TTGTGGGTTA TCTAGATGTA |
| 641 | ATAAAAGTGA TATTGATAAA GCTTGGGAAG CAGTGAAATA |
| 681 | CGCTAAAAAA CCTAGGGTTC ATACGTTTAT TGCTACGAGT |
| 721 | GAAATACATA TGAAGTATAA ATTGAAGATG AGTAGAGAAC |
| 761 | AAGTGGTGGA AAAAGCAAGG AGTATGGTAG CTTATGCTAG |
| 801 | AAGCCTTGGA TGTGAAGATG TTGAATTTAG TCCAGAAGAT |
| 841 | GCAGGAAGGT CTGATCGAGA GTTCCTTTAT GATATCCTCG |
| 881 | GAGAAGTTAT TAAAGCTGGT GCAACAACAC TTAACATACC |
| 921 | TGATACTGTT GGATACACTG TTCCAAGTGA ATTTGGACAA |
| 961 | TTAATTGCTG ACATAAAAGC CAATACTCCG GGGATTGAAA |
| 1001 | ATGTGATAAT TTCAACACAT TGCCAGAACG ATCTTGGGCT |
| 1041 | TTCTACTGCC AACACTTTAG CTGGAGCTTG TGCAGGAGCA |
| 1081 | AGACAACTAG AGGTGACCAT TAATGGCATT GGTGAAAGAG |
| 1121 | CTGGAAATGC TTCTCTGGAG GAGGTTGTAA TGGCCTTAAA |
| 1161 | ATGTCGCGGA GAGCAAGTAT TAGGCTGGCC CTACACGGGG |
| 1201 | ATTAACACTC AACATATTGT TCCATCGAGC AAAATGGTGG |
| 1241 | AGGAGTACAG TGGGCTACAG GTGCAGCCAC ATAAGGCCAT |
| 1281 | TGTTGGAGCT AATGCATTTG CTCATGAAAG TGGCATCCAT |
| 1321 | CAGGATGGAA TGTTAAAACA CAAGGACACC TATGAGATTA |
| 1361 | TATCTCCTGA TGATGTTGGG CTTAGTCGTT CTAATGAAGC |
| 1401 | GGGTATTGTC CTTGGGAAAC TCAGTGGTCG CCATGCACTG |
| 1441 | AAATCCAAAA TGCTTGAGCT TGGATATGAC ATTGATGGAA |
| 1481 | AAGAACTAGA GGACCTCTTT TGGCGTTTTA AGTCAGTAGC |
| 1521 | TGAGAAGAAA AAGAAAATTA CAGATGATGA CTTAATAGCA |
| 1561 | CTGATGTCAG ATGAAGTTCT CCAACCTAAT GTTTATTGGA |
| 1601 | AGCTTGGAGA TGTACAGATT ATGTGTGGAA GTCTTGGCCT |
| 1641 | CTCTACAGCA ACTGTGAAGC TTATAAACAC TGATGGTCAA |
| 1681 | GAGCATATTG CTTGTTCAGT TGGAACAGGA CCTGTTGATG |
| 1721 | CAGCTTAGAA GGCAGTGGAC CTCATTGTGA AGGTGCCTAT |
| 1761 | TACGCTCCTT GAATATTCCA TGAATGCAGT CACAGAAGGT |
| 1801 | ATAGATGCCA TAGCATCAAC CAGAGTGTCA ATCTGCAGTA |
| 1841 | TTGATAGGCA TAGTATAATG AATGGTTCAA CTGGACAGAC |
| 1881 | TATTCACCGC ACATTTAGTG GAACCGGAGC AGATATGGAT |
| 1921 | GTTGTTATCT CTAGTGTCCG AGCGTATATT GGTGCATTGA |
| 1961 | ACAAAATGTT GAGTTACGAA AAGCTGGTGT CAAGATACAG |
| 2001 | CAAACCTGAA GACAGTGTGG TGGTATAAGA AAATGTTCGT |
| 2041 | AATGTTCCAG TTTCTTGTCA TTTCTCTTGT CAATTGTATA |
| 2081 | GAACTAGGGG TGCCTTATCA ACAAATTACG ACTTGCCTGG |
| 2121 | AAGAACGATA AAAGGCAAAT TTGAGTCGTA ATGCATTTTC |
| 2161 | ATTTTCTGCA GGTTGATGTA CAAGCTTGTA CTAAATGTGT |
| 2201 | TAAAGTCATT TTAGGCTTTG TGTTGTACCA ATCAAACACA |
| 2241 | GATCCTTTTA TGTGGTTTAG CTTTAAATTG ATTTTTGGTT |
| 2281 | AA |

An Oryza sativa (rice) IPMS1 protein is shown below as SEQ ID NO:30, where two positions (231 and 607) are highlighted that can be modified to increase the production of various amino acids.

| | |
|---|---|
| 1 | MASSLLSSPK PSSFSSANPT STPRPRAQTL SPFRAAAPRF |
| 41 | SHGLATAAAA ANPSASRRCY HRAFARPVRA SMAQPRRPEY |
| 81 | VPNRIDDPNY VRIFDTTLRD GEQSPGATMT SAEKLVVARQ |
| 121 | LARLGVDIIE AGFPASSPDD LDAVRSIAIE VGNTPVGEDG |
| 161 | HVPVICGLSR CNKRDIDAAW EAVRKARRPR IHTFIATSEI |
| 201 | HMQHKLRKTP EQVVAIAKEM VAYARSLGCP DVEFSPEDAG |
| 241 | RSNREFLYHI LEEVIKAGAT TLNIPDTVGY TLPYEFGKLI |
| 281 | ADIKANTPGI ENAIISTHCQ NDLGLATANT LAGAHAGARQ |
| 321 | LEVTINGIGE RAGNASLEEV VMAIKCRREL LGGLYTGINT |
| 361 | QHITMSSKMV QEHSGLHVQP HKAIVGANAF AHESGIHQDG |
| 401 | MLKYKGTYEI ISPDDIGLTR ANEFGIVLGK LSGRHAVRSK |
| 441 | LVELGYEITD KEFEDFFKRY KEVAEKKKRV TDEDIRALLS |
| 481 | DEIFQPKVFW SLADVOATCG TLGLSTATVK LIGPDGEEKI |
| 521 | ACAVGTGPVD AAYKAVDDII QIPTVLREYS MTSVTEGIDA |
| 561 | IATTRVVVTG DVSDSKHALT GHSFSRAFSG SGAALDIVVS |
| 601 | SVRAYLSALN KMSSFVGAIK ASSEVSESQR VQTTE |

The Oryza sativa (rice) IPMS protein with SEQ ID NO:30 is encoded by the 2-isopropylmalate synthase A gene on chromosome 11 (LOC4349745; locus tag OSNPB_110142500; see NCBI website).

As illustrated below the SEQ ID NO:30 Oryza sativa IPMS1 sequence has about 72% sequence identity with the Arabidopsis IPMS1 SEQ ID NO:2 sequence.

| | | |
|---|---|---|
| Sq2 | 75 | RPEYIPNRISDPNYVRVFDTTLRDGEQSPGATLTSKEKLDIARQLAKLGVDIIEAGFPAA |
| Sq30 | 77 | RPEYVPNRIDDPNYVRIFDTTLRDGEQSPGATMTSAEKLVVARQLARLGVDIIEAGFPAS |
| | | **** **** ****** ************** ** *** ***** ************* |
| Sq2 | 135 | SKDDFEAVKTIAETVGNT-VDENGYVPVICGLSRCNKKDIERAWDAVKYAKRPRIHTFIA |
| Sq30 | 137 | SPDDLDAVRSIAIEVGNTPVGEDGHVPVICGLSRCNKRDIDAAWEAVRHARRPRIHTFIA |
| | | * ** ** ** **** * * * ************ ** ** ** * ********* |
| Sq2 | 194 | TSDIHLEYKLKKTKAEVIEIARSMVRFARSLGCEDVEFSPEDAGRSEREYLYEILGEVIK |
| Sq30 | 197 | TSEIHMQHKLRKTPEQVVAIAKEMVAYARSLGCPDVEFSPEDAGRSNREFLYHILEEVIK |
| | | ** ** ** * ** ** ***** ************ ** ** ** **** |
| Sq2 | 254 | AGATTLNTPDTVGITLPSEFGQLITDLKANTPGIENVVISTHCQNDLGLSTANTLSGAHA |
| Sq30 | 257 | AGATTLNIPDTVGYTLPYEFGKLIADIKANTPGIENAIISTHCQNDLGLATANTLAGAHA |
| | | ************* *** *** ** * ********* *********** ***** **** |
| Sq2 | 314 | GARQMEVTINGIGERAGNASLEEVVMAIKCRGDHVLGGLFTGIDTRHIVMTSKMVEEYIG |
| Sq30 | 317 | GARQLEVTINGIGERAGNASLEEVVMAIKCRRE-LLGGLYTGINTQHITMSSKMVQERSG |
| | | **** ************************** **** *** * ** * **** * * |
| Sq2 | 374 | MQTQPHKAIVGANAFAHESGIHQDGMLKHKGTYEIICPEEIGLERSNDAGIVLGKLSGRH |
| Sq30 | 376 | LHVQPHKAIVGANAFAHESGIHQDGMLKYKGTYEIISPDDIGLTRANEFGIVLGKLSGRH |
| | | ************************* ******* * *** * * *********** |
| Sq2 | 434 | ALKDRLTELGYQLDDEQLSTTFWRFKTVAEQKKRVTDAD1IALVSDEVFQPEAVWKLLDI |
| Sq30 | 436 | AVRSKLVELGYEITDKEFEDFFKRYKEVAEKKKRVTDEDIEALLSDEIFQPKVFWSLADV |
| | | * * **** * * * * *** ****** ** ** *** *** * * * |
| Sq2 | 494 | QTTCGTLGLSTATVKLADADGKEHVACSIGTGPVDSAYKAVDLIVKEPATLLEYSMNAVT |
| Sq30 | 496 | QATCGTLGLSTATVKLIGPDGEEKIACAVGIGPVDAAYKAVDDIIQIPTVLREYSMTSVT |
| | | * ************** ** * ** ****** ****** * * * **** ** |
| Sq2 | 554 | EGIDAIATTRVLIRGSNKYSSTNAITGEEVQRTFSGTGAGMDIVVSSVKAYVGALNKMMD |
| Sq30 | 556 | EGIDAIATTRVVVTGDVS-DSKHALTGHSFSRAFSGSGAALDIVVSSVRAYLSALNKMSS |
| | | *********** * * * ** * *** ** ******* ** ***** |

A cDNA encoding the SEQ ID NO:30 rice IPMS1 protein is shown below as SEQ ID NO:31.

| | |
|---|---|
| 1 | TCGTGCATCC CACGAAACCC CCGTTGTCAT GTCCAGATAA |
| 41 | GAGCTCCCCC TCTGACTCAC ACACACATCA CCGAGAAGCA |
| 81 | GAGCAAGTGC ATCTCCCTCT CCCACCATTT GATTCGATTC |
| 121 | CCCTAAACCC CCGAGTCGTC GACGCCACAA ACCCCGGCGA |
| 161 | CCACCATGGC CTCCTCCCTC CTCTCCTCCC CAAAACCCTC |
| 201 | CTCCTTCTCC TCCGCAAACC CCACCTCCAC TCCACGCCCA |
| 241 | CGCGCCCAAA CTCTCTCCCC CTTCCGCGCC GCCGCCCCAC |
| 281 | GCTTCTCCCA TGGCCTCGCC ACCGCCGCCG CCGCCGCAAA |
| 321 | CCCTAGCGCC TCCCGCCGCT GCTACCACCG CGCCTTCGCC |
| 361 | CGCCCCGTCC GGGCGTCCAT GGCGCAGCCG CGGCGCCCGG |
| 401 | AGTACGTCCC CAACCGCATC GACGACCCCA ACTACGTCCG |
| 441 | CATCTTCGAC ACCACCCTCC GCGACGGGGA GCAGTCCCCC |
| 481 | GGGGCCACCA TGACCAGCGC CGAGAAGCTC GTCGTCGCGC |
| 521 | GCCAGCTCGC CCGCCTCGGC GTCGACATCA TCGAGGCCGG |
| 561 | GTTCCCGGCC TCCTCCCCCG ACGACCTCGA CGCCGTGCGC |
| 601 | TCCATCGCCA TCGAGGTCGG GAACACGCCC GTCGGGGAGG |
| 641 | ACGGCCACGT GCCGGTCATC TGTGGCCTCT CGAGATGCAA |
| 681 | TAAGCGAGAC ATTGATGCTG CCTGGGAGGC CGTGCGGCAC |
| 721 | GCGCGGCGGC CGCGCATCCA CACCTTCATT GCCACCAGCG |
| 761 | AGATCCATAT GCAGCACAAG CTAAGGAAGA CGCCCGAGCA |
| 801 | GGTGGTGGCC ATTGCCAAGG AAATGGTGGC CTACGCCCGC |
| 841 | AGCCTAGGCT GCCCTGATGT CGAGTTCAGC CCTGAAGACG |
| 881 | CTGGCAGGTC AAACAGAGAG TTTCTATATC ATATTCTAGA |
| 921 | GGAAGTCATA AAAGCTGGAG CAACAACACT CAATATCCCA |
| 961 | GACACTGTTG GATACACTCT TCCTTATGAA TTTGGGAAGC |
| 1001 | TAATTGCTGA TATAAAAGCA AACACTCCAG GAATTGAAAA |
| 1041 | TGCTATTATT TCTACTCATT GCCAGAATGA CCTTGGTCTA |
| 1081 | GCAACCGCCA ATACATTAGC GGGCGCTCAT GCAGGAGCAC |
| 1121 | GGCAATTAGA GGTGACTATC AACGGTATTG GTGAAAGGGC |
| 1161 | TGGAAATGCT TCTTTGGAAG AGGTTGTGAT GGCAATTAAA |
| 1201 | TGTCGCCGAG AGCTCTTAGG AGGTCTGTAT ACTGGAATCA |
| 1241 | ATACCCAACA TATCACTATG TCAAGCAAAA TGGTACAAGA |
| 1281 | GCACAGTGGA CTTCATGTAC AACCACATAA AGCTATTGTC |
| 1321 | GGTGCCAATG CCTTTGCACA TGAAAGTGGA ATTCATCAGG |
| 1361 | ATGGGATGCT TAAATACAAA GGAACTTATG AAATAATTTC |
| 1401 | TCCTGATGAT ATTGGTCTAA CACGTGCAAA CGAGTTTGGT |
| 1441 | ATTGTTCTTG GGAAACTCAG TGGAAGGCAT GCTGTGAGAT |
| 1481 | CTAAACTAGT GGAGCTTGGA TATGAAATCA CTGACAAGGA |
| 1521 | ATTTGAGGAT TTCTTTAAAC GCTACAAAGA GGTTGCAGAG |
| 1561 | AAGAAAAAGC GTGTAACTGA TGAAGACATT GAGGCGCTGT |
| 1601 | TGTCTGATGA GATATTTCAG CCCAAGGTTT TTTGGTCCCT |
| 1641 | TGCTGATGTA CAGGCAACTT GTGGAACACT TGGTCTGTCT |
| 1681 | ACAGCAACTG TCAAACTGAT AGGTCCGGAT GGAGAGGAGA |
| 1721 | AGATTGCATG TGCAGTTGGA ACAGGTCCAG TTGATGCAGC |
| 1761 | TTACAAGGCT GTTGATGATA TAATACAGAT CCCAACTGTT |
| 1801 | CTTCGAGAAT ATAGCATGAC ATCGGTCACA GAAGGCATTG |
| 1841 | ATGCAATTGC AACTACTAGA GTGGTTGTCA CTGGAGATGT |
| 1881 | TAGCGACTCT AAACATGCTT TGACTGGTCA CTCCTTCAGC |
| 1921 | CGGGCATTCA GTGGGAGTGG TGCCGCACTG GATATTGTTG |
| 1961 | TTTCCAGTGT GCGAGCTTAC CTGAGTGCCC TGAACAAGAT |
| 2001 | GTCCAGTTTT GTTGGGGCTA TCAAGGCTAG TAGTGAAGTA |
| 2041 | TCTGAAAGCC AAAGAGTTCA AACCACAGAA TGAGTCTTGA |
| 2081 | CTTCCTTTTG GGTTTTCATA TCCGATGGTT CTATGTTTCA |
| 2121 | CATTCCCAGC AAGGAGTATG TGCTTGTTGA AACATGGTTT |
| 2161 | TTCCGTCCAG AAAAAACATG GTTCCGTTTA GTGCATCTGG |
| 2201 | AGGATGTTCT GGGTTTCTTG GTGGAGCCTG ACTTAAGGTT |
| 2241 | GAACATCCAG GACGTTTTGG GATATGCAGT GTATAATTCA |
| 2281 | TATTTGAAAA CCGTATTTAC AATAAGACAA TAATAAATAA |
| 2321 | TTGTTTGACA TATGAGTATT GCAAAACTAT TACTGTAAGA |
| 2361 | AATTAATCGT GAGACCAACC TAGGGTTGTA CAGTA |

Another Oryza sativa IPMS1 protein is shown below as SEQ ID NO: 32, where two positions (231 and 607) are highlighted that can be modified to increase the production of various amino acids.

| | |
|---|---|
| 1 | MASSLLSSPK PSSFSSANPT STPRPRAQTL SPFRAAAPRF |
| 41 | SHGLATAAAA ANPSASRRCY HRAFARPVRA SMAQPRRPEY |
| 81 | VPNRIDDPNY VRIFDTTLRD GEQSPGATMI SAEKLVVARQ |
| 121 | LARLGVDIIE AGFPASSPDD LDAVRSIAIE VGNTPVGEDG |
| 161 | HVPVICGLSR CNKRDIDAAW EAVRHARRPR IHTFIATSEI |
| 201 | HMQHKLRKTP EOVVAIAKEM VAYARSLGCP DVEFSPEDAG |
| 241 | RSNREFLYHI LEEVIKAGAT TLNIPDTVGY TLPYEFGKLI |
| 281 | ADIKANTPGI ENAIISTHCQ NDLGLATANT LAGAHAGARQ |
| 321 | LEVTINGIGE RAGNASLEEV VMAIKCRREL LGGLYTGINT |
| 361 | QHITMSSKMV QEHSGLHVQP HKAIVGANAF AHESGIHQDG |
| 401 | MLKYKGTYEI ISPDDIGLTR ANEFGIVLGK LSGRHAVRSK |
| 441 | LVELGYEITD KEFEOFFKRY KEVAEKKKRV TDEDIEALLS |
| 481 | DEIFQPKVFW SLADVQATCG TLGLSTATVK LIGPDGDEKI |
| 521 | ACAVGTGPVD AAYKAVDDII QIPTVLREYS MTSVTEGIDA |
| 561 | IATTRVVVTG DVSDSKHALT GHSFNRAFSG SGAALDIVVS |
| 601 | SVRAYLSALN KMSSFVGAIK ASSEVSESQR VQTTE |

The Oryza sativa (rice) IPMS protein with SEQ ID NO:32 is encoded by the 2-isopropylmalate synthase A gene on chromosome 12 (LOC4351460; locus tag OSNPB_120138900; see NCBI website).

As illustrated below the SEQ ID NO:32 Oryza sativa IPMS1 sequence has about 72% sequence identity with the Arabidopsis IPMS1 SEQ ID NO:2 sequence.

| | | |
|---|---|---|
| Sq2 | 75 | RPEYIPNRISDPNYVRVEDLILRDGEQSPGATLLSKEKLDIARQLAKLGVDIIEAGFPAA |
| Sq32 | 77 | RPEYVPNRIDDPKYVPIFDTTLRDGEQSPGATMTSAEKLVVARQLARLGVDIIEAGRPAS |
| | | **** **** ****** *************** ** *** ****************** |
| Sq2 | 135 | SKDDFEAVKTIAETVGNT-VDENGYVPVICGLSRCNKKDIERAWDAVKYAKRPRIHTFIA |
| Sq32 | 137 | SPDDLDAVRSIAIEVGNTPVGEDGHVPVTCGLSRCNKRDIDAAWEAVRHARRPRIHTFIA |
| | | * ** ** ** **** * * * ************ ** ** ** * ********* |
| Sq2 | 194 | TSDIHLEYKLKKTKAEVIEIARSMVRFARSLGCEDVEFSPEDAGRSEREYLYEILGEVIK |
| Sq32 | 197 | TSEIHMQHKLRKTPEQVVAIAKEMVAYARSLGCPDVEFSPEDAGRSNREFLYHILEEVIK |
| | | ** ** ** ** * ** ** ****** *********** ** ** ** **** |
| Sq2 | 254 | AGATTLNIPDTVGITLPSEFGQLIIDLKANTPGIENVVISTHCQNDLGLSTANTLSGAHA |
| Sq32 | 257 | AGATTLNIPDTVGYTLPYEFGKLIADIKANTPGIENAIISTHCQNDLGLATANTLAGAHA |
| | | ************* *** *** ** * ********* *********** ***** **** |
| Sq2 | 314 | GARQMEVTINGIGERAGNASLEEVVMAIKCRGDHVLGGLFTGIDTRHIVMTSKMVEEYTG |
| Sq32 | 317 | GARQLEVTINGIGERAGNASLEEVVMAIKCRRE-LLGGLYTGINTQHITMSSKMVQEHSG |
| | | **** ************************** **** *** * ** * **** * * |
| Sq2 | 374 | MQTQPHKAIVGANAFAHESGIHQDGMLKHKGTYEIICPEEIGLERSNDAGIVLGKLSGRH |
| Sq32 | 376 | LHVQPHKAIVGANAFAHESGIHQDGMLKYKGTYEIISPDDIGLTRANEFGIVLGKLSGRE |
| | | ************************* ******* * *** * * *********** |
| Sq2 | 434 | ALKDRLTELGYQLDDEQLSTIFWRFKTVAEQKKRVTDADTIALVSDEVFQPEAVWKLLDT |
| Sq32 | 436 | AVRSKLVELGYEITDKEFEDFFKRYKEVAEKKKRVTDEDIEALLSDEIFQPKVFWSLADV |
| | | * * **** * * * * *** ****** ** ** *** *** * * * |
| Sq2 | 494 | QTTCGTLGLSTATVKLADADGKEHVACSIGTGPVDSAYKAVDLIVKEPATLLEYSMNAVT |
| Sq32 | 496 | QATCGTLGLSTATVKLIGPDGDEKIACAVGTGPVDAAYKAVDDIIQIPTVLREYSMTSVT |
| | | * ************** ** * ** ****** ****** * * * **** ** |
| Sq2 | 554 | EGIDAIATTRVLIRGSNKYSSTNAITGEEVQRTFSGTGAGMDIVVSSVKAYVGALNKMMD |
| Sq32 | 556 | EGIDAIATTRVVVTGDVS-DSKHALTGHSFNRAFSGSGAALDIVVSSVRAYLSALNKMSS |
| | | *********** * * * ** * *** ** ******* ** ***** |
| Sq2 | 614 | F |
| Sq32 | 615 | F |
| | | * |

A Sorghum bicolor IPMS1 protein is shown below as SEQ ID NO:33, where two positions (215 and 592) are highlighted that can be modified to increase the production of various amino acids.

| | |
|---|---|
| 1 | MAFTAKPYCS TPTKPPTLSA RAPRSAHGLS AAAAATPSTV |
| 41 | RLRAFAQRIR AQSQQQQQRR RRPEYVPHRI DDPNYVRIFD |
| 81 | TTLRDGEQSP GATMTSAEKL VVARQLARLG VDIIEAGFPA |
| 121 | SSPDDLDAVR SIAIEVGNPV EEGAHVPVIC GLSRCNKRDI |
| 161 | DAAWEAVRNA RRPRIHTFIA TSEIHMQHKL RKTPEQVVAI |
| 201 | AKEMVAYARS LGCPDVEFSP EDAGRSNREF LYHILEEVIK |
| 241 | AGATTLNIPD TVGYTLPYEF GKLIADIKAN TPGIENAIIS |
| 281 | THCQNDLGLA TANTLAGARA GARQLEVTIN GIGERAGNAS |
| 321 | LEEVVMAIKC RGELLDGLYT GINSQHITLT SKMVQEHSGL |
| 361 | HVQPHKAIVG ANAFAHESGI HQDGMLKYKG TYEIISPDDI |
| 401 | GLTRANEFGI VLGKLSGRHA VRSKLVELGY EISDKEFEDE |
| 441 | FKRYKEVAEK KKRVTDEDIE ALLSDEIFQP KVIWSLADVQ |
| 481 | ATCGTLGLST ATVKLIAPDG EEKIGCSVGT GPVDAAYKAV |
| 521 | DQIIQIPTVL REYGMTSVTE GIDAIATTRV VVTGDVTNNS |
| 561 | KHALTGQSFN RSFSGSGAAM DIVVSSVRAY LSALNKMCSF |
| 601 | AGAAKASSEV PESASVQRTE |

The Sorghum bicolor IPMS protein with SEQ ID NO:33 is encoded by the 2-isopropylmalate synthase A gene on chromosome 5 (LOC8085635; locus tag SORBI_-3005G030100; see NCBI website).

As illustrated below the Sorghum bicolor IPMS11 protein with SEQ ID NO:33 has about 71%-7-1% sequence identity with the Arabidopsis IPMS1 SEQ ID NO:2 sequence.

| | | |
|---|---|---|
| Sq2 | 72 | RRPRPEYTPNRTSDPNYVRVFDTTLRDGEQSPGATLTSKERLDIARQLAKLGVDITEAGE |
| Sq33 | 59 | RRRRPEYVPHRIDDPNYVRIFDTTLRDGEQSPGATMTSAEKLVVARQLARLGVDIIEAGE |
| | | ** **** * ** ****** *************** ** *** ***** ********** |
| Sq2 | 132 | PAASKDDPEAVKTIAETVGNTVDENGYVPVICGLSRCNKKDIERAWDAVKYAKRPRIHTE |
| Sq33 | 119 | PASSPDDLDAVRSIAIEVGNPVEEGAHVPVICGLSRCNKRDIDAAWEAVRNARRPRIHTE |
| | | ** * ** ** ** *** * * ************ ** ** ** * ******* |
| Sq2 | 192 | IATSDIHLEYKLKKTKAEVIEIARSMVRFARSLGCEDVEPSPEDAGRSEREYLYEILGEV |
| Sq33 | 179 | IATSEIHMQHKLRKTPEQVVAIAKEMVAYARSLGCPDVEFSPEDAGRSNREFLYHILEEV |
| | | **** ** ** ** * ** ** ****** ************ ** ** ** ** |
| Sq2 | 252 | IKAGATTLNIPDTVGITLPSEFGQLITDLKANTPGIENVVISTHCQNDLGLSTANTLSGA |
| Sq33 | 239 | IKAGATTLNIPDTVGYTLPYEFGKLIADIKANTPGIENAIISTHCQNDLGLATANTLAGA |
| | | *************** *** *** ** * ********* *********** ***** ** |
| Sq2 | 312 | HAGARQMEVTINGIGERAGNASLEEVVMAIKCRGDHVLGGLFTGIDTRHIVMTSKMVEEY |
| Sq33 | 299 | RAGARQLEVTINGIGERAGNASLEEVVMAIKCRGE-LLDGLYTGINSQHITLTSKMVQEH |
| | | ***** *************************** * ** *** ** ***** * |
| Sq2 | 372 | TGMQTQPHKAIVGANAFAHESGIHQDGMLKHKGTYEIICPEEIGLERSNDAGIVLGKLSG |
| Sq33 | 358 | SGLHVQPHKAIVGANAFAHESGIHQDGMLKYKGTYEIISPDDIGLTRANEFGIVLGKLSG |
| | | * ************************* ******* * *** * * ********* |
| Sq2 | 432 | RHALKDRLTELGYQLDDEQLSTIPWRFKTVAEQKKRVTDADIIALVSDEVFQPEAVWKLL |
| Sq33 | 418 | RHAVRSKLVELGYEISDKEFEDFFKRYKEVAEKKKRVTDEDIEALLSDEIFQPKVIWSLA |
| | | *** * **** * * * * *** ****** ** ** *** *** * * |
| Sq2 | 492 | DIQITCGTLGLSTATVKLADADGKEHVACSIGTGPVDSAYKAVDLIVKEPATLLEYSMNA |
| Sq33 | 478 | DVQATCGTLGLSTATVKLIAPDGEEKIGCSVGTGPVDAAYKAVDQIIQIPTVLREYGMTS |
| | | * * ************* ** * ** ****** ****** * * * ** * |
| Sq2 | 552 | VTEGTDATATTRVLIRGSNKYSSTNAITGEEVQRTFSGTGAGMDIVVSSVKAYVGALNKM |
| Sq33 | 538 | VTEGIDAIATTRVVVTGDVTNNSKHALTGQSFNRSFSGSGAAMDIVVSSVRAYLSALNKM |
| | | ************* * * * ** * *** ** ****** ** ***** |
| Sq2 | 612 | MDF |
| Sq33 | 598 | CSF |
| | | * |

A cDNA encoding the Sorghum bicolor IPMS1 protein with SEQ ID NO:33 is shown below as SEQ ID NO:34.

```
      1 CACGGCACGG CACCTCACCG CTCTCGCGTG CTCGCCGCGG
    41 CGGCCGCCGT GCTCGTGTCG TGTCACAGCC AGCAGCTCGC
    81 TCCCACGACC TCCGATCCGT GCCGCCTACG AAACCACCAC
  121 CCGTCCGGAT CCTCCAGGAT ATATACCAGC CAGCTAGTCA
  161 TCCCCTTGCT GGCTGCTGCC TTTTCCAAAT CCACTCACAT
  201 TTTCCACATC CACCGTCGAT TTAACACAGT CCCCCGCCGC
  241 CGCCGCCCGC CCGTCCGTCC CCTCCTAAAC CCCGCGACGA
  281 CCCTGAGCGA GCCCGAGCCG AGTCCCCGGC GACCAACCAC
  321 CATGGCCTTC ACCGCTAAAC CCTACTGCTC AACCCCCACC
  361 AAACCCCCCA CCCTCTCCGC CCGCGCCCCG CGCTCCGCCC
  401 ATGGCCTATC CGCCGCCGCC GCCGCAACCC CGAGCACCGT
  441 CCGCCTCCGC GCGTTCGCCC AGCGCATCCG GGCGCAGTCG
  481 CAGCAACAGC AACAGCGGCG GCGGCGGCCC GAGTACGTGC
  521 CGCACCGCAT CGACGACCCA AACTACGTGC GCATCTTCGA
  561 CACCACGCTC CGCGACGGGG AGCAGTCCCC GGGAGCCACC
  601 ATGACGAGCG CCGAGAAGCT GGTGGTCGCG CGGCAGCTGG
  641 CCCGGCTCGG CGTCGACATC ATCGAGGCGG GGTTCCCGGC
  681 GTCCTCCCCC GACGACCTCG ACGCCGTGCG CTCCATCGCC
  721 ATCGAGGTCG GCAACCCGGT GGAGGAAGGC GCCCACGTGC
  761 CCGTCATCTG CGGCCTCTCG CGGTGCAACA AGAGGGATAT
  801 TGATGCCGCC TGGGAGGCCG TCAGGAACGC GCGCAGGCCC
  841 CGGATTCATA CCTTCATCGC CACCAGCGAG ATCCATATGC
  881 AGCATAAGCT TAGGAAGACG CCTGAGCAGG TCGTTGCTAT
  921 TGCTAAGGAG ATGGTGGCGT ATGCACGCAG CCTTGGGTGC
  961 CCTGATGTCG AATTCAGCCC TGAGGATGCC GGCAGGTCAA
1001 ATCGAGAATT CCTGTATCAT ATACTGGAGG AAGTCATTAA
1041 AGCTGGGGCA ACTACTCTTA ATATCCCAGA CACTGTCGGA
1081 TACACTCTTC CTTATGAATT TGGGAAGTTG ATTGCTGACA
1121 TAAAGGCAAA CACTCCTGGA ATTGAAAATG CTATCATTTC
1161 CACTCATTGC CAGAATGACC TTGGTCTTGC AACTGCCAAC
1201 ACATTAGCGG GCGCTCGTGC AGGAGCACGA CAGTTAGAGG
1241 TGACTATTAA TGGTATTGGT GAAAGAGCTG GAAATGCTTC
1281 GTTGGAAGAG GTTGTCATGG CAATTAAATG TCGTGGGGAG
1321 CTCTTAGATG GTCTATATAC GGGAATCAAT TCCCAACATA
1361 TTACTTTGAC AAGCAAAATG GTACAAGAGC ACAGTGGACT
1401 TCATGTACAA CCACATAAAG CTATTGTTGG TGCCAATGCC
1441 TTTGCTCATG AAAGTGGAAT TCATCAGGAT GGGATGCTTA
1481 AGTACAAGGG AACATATGAA ATAATATCGC CTGATGATAT
1521 TGGTTTAACA CGTGCGAATG AATTTGGTAT TGTTCTTGGG
1561 AAACTCAGCG GAAGACATGC AGTGAGATCT AAGCTAGTGG
1601 AGCTTGGATA TGAAATCAGT GACAAGGAAT TTGAAGATTT
1641 CTTTAAACGC TACAAAGAGG TTGCAGAGAA GAAAAAGCGT
1681 GTAACTGATG AAGACATAGA AGCGTTATTG TCAGATGAGA
1721 TATTCCAGCC TAAGGTTATT TGGTCCCTTG CTGATGTACA
1761 GGCAACATGT GGAACACTTG GCTTATCTAC AGCAACAGTG
1801 AAACTGATAG CACCAGATGG AGAGGAGAAA ATAGGATGTT
1841 CAGTTGGAAC AGGTCCAGTT GATGCAGCTT ACAAGGCTGT
1881 TGACCAAATA ATCCAGATTC CAACTGTTCT CCGAGAATAT
1921 GGTATGACTI CAGTCACAGA GGGCATTGAC GCTATCGCGA
1961 CAACTCGAGT GGTTGTCACT GGAGATGTGA CCAACAACTC
2001 CAAGCATGCC TTGACTGGTC AATCTTTCAA CCGCTCCTTC
2041 AGTGGGAGCG GGGCAGCTAT GGACATCGTT GTGTCCAGCG
2081 TCAGAGCTTA CCTGAGTGCC CTGAACAAGA TGTGCAGCTT
2121 TGCTGGTGCT GCGAAAGCCA GCAGCGAGGT ACCTGAGAGC
2161 GCAAGCGTTC AACGCACAGA GTGAGCTTGG CGCTCCTCTT
2201 TGTTCCCATG TGGGCTTGGC GACGTAAGAG CTTGAGCAAC
2241 TGTTATAGAG TGTATGTCGT TTCAGTAACA GGCTGTTCAA
2281 TATTGGGGTT TTCCCTTGTC AGTGTGGAGT GATTGTGCTG
2321 TTCTATTTTG GAGGATAGTC CCTTTAGCTT AGAACATGCA
2361 GGAAATTTTG GCCCTATGTA GTGTACAATT TGTGCCTTAT
2401 ATGAACCATA CTTTCAATAA TGAAATAATA TTAGGGTCCA
2441 TCCAGCCACC CATA
```

A Brassica napus IPMS protein is shown below as SEQ ID NO: 35, where two positions (219 and 598) are highlighted that can be modified to increase the production of various amino acids.

| | |
|---|---|
| 1 | MASSILRNPM LSSPTTITTP SLPSFSSKPS PLSFRFPPSH |
| 41 | HRSSLRIKSL RLSCSLSDPS PPLRRRRPEY IPNRISDPNY |
| 81 | VRVFDTTLRD GEQSPGATLT SKEKLDIARQ LAKLGVDVIE |
| 121 | AGFPAASKDD FEAVKTIAET VGNAVDGDGY VPVICGLSRC |
| 161 | NKRDIETAWE AVKYAKRPRI HTFIATSDIH LEYKLKKSKD |
| 201 | EVIEIARNMV KFARSLGCED VEFSPEDAGR SEREFLYQIL |
| 241 | GEVIKAGATI LNIPDTVGIT LPSEFGQLIA DIKANTPGIE |
| 281 | NVIISTHCQN DLGLSTANTL SGAHSGARQL EVTINGIGER |
| 321 | AGNASLEEVV MAIKCRGDHV LGGLYTGIDT RHIVMTSKMV |
| 361 | EDYTGMQTQP HKAIVGANAF AHESGIHQDG MLKHKGTYEI |
| 401 | ICPEEIGLER SNDAGIVLGK LSGRHALKDR LTELGYVLDD |
| 441 | EQLSSIFWRF KSVAERKKRV TDADIIALVS DEVFQPEALW |
| 481 | KLLDIQITCG TLGLSTATVK LADADGKEHV ACSMGTGPVD |
| 521 | SAYKAVDLVV KEPATLLEYS MNAVTEGIDA IATTRVLIRG |
| 561 | NNNYSTTNAI TGEEVQRTFS GTGAGMDIVV SSVKAYVGAL |
| 601 | NKMLDFKENS TTKIPSQNNK VPA |

As illustrated below the Brassica napus IPMS protein with SEQ ID NO:35 has about 90%-91% sequence identity with the Arabidopsis IPMS1 SEQ ID NO:2 sequence.

| | | |
|---|---|---|
| Sq2 | 1 | MASSLLRNPNLYSSTTITTTSFLPTFSSKPTPISSSFRFQPSHHRSISLRSQTLRLSCSI |
| Sq35 | 1 | MASSILRNPMLSSPTTITTPS-LPSFSSKPSPLS--FRFPPSHHRS-SLRIKSLRLSCSL |
| | | **** **** * * ***** * ** ***** * * *** ****** *** ******* |
| Sq2 | 61 | SDPSPLPPHTPRRPRPEYIPNRISDPNYVRVFDTTLRDGEQSPGATLTSKEKLDIARQLA |
| Sq35 | 57 | SDPSP----PLRRRRPEYTPNRISDPNYVRVFDTTLRDGEQSPGATLTSKEKLDIARQLA |
| | | ****** ** ********************************************** |
| Sq2 | 121 | KLGVDIIEAGFPAASKDDFEAVKTIAETVGNTVDENGYVPVICGLSRCNKKDIERAWDAV |
| Sq35 | 113 | KLGVDVIEAGFPAASKDDFEAVKTIAETVGNAVDGDGYVPVICGLSRCNKRDIETAWEAV |
| | | ***** ************************* ** *************** *** ** ** |
| Sq2 | 181 | KYAKRPRIHTFIATSDIHLEYKLKKTKAEVIEIARSMVRFARSLGCEDVEFSPEDAGRSE |
| Sq35 | 173 | KYAKRPRIHTFIATSDIHLEYKLKKSKDEVIEIARNMVKFARSLGCEDVEFSPEDAGRSE |
| | | ************************* ******* ** ********************* |
| Sq2 | 241 | REYLYEILGEVIKAGATTLNIPDTVGITLPSEFGQLITDLKANTPGIENVVISTHCQNDL |
| Sq35 | 435 | REYLYQILGEVIKAGATTLNIPDTVGITLPSEFGQLIADIKANTPGIENVIISTHCQNDL |
| | | ** ** ******************************* * ********** ******** |
| Sq2 | 301 | GLSTANTLSGAHAGARQMEVTINGIGERAGNASLEEVVMAIKCRGDHVLGGLFTGIDTRH |
| Sq35 | 293 | GLSTANTLSGAHSGARQLEVTINGIGERAGNASLEEVVMAIKCRGDHVLGGLYTGIDTRH |
| | | ************ **** ********************************** ******* |
| Sq2 | 361 | IVMTSKMVEEYTGMQTQPHKAIVGANAFAHESGIHQDGMLKHKGTYEIICPEEIGLERSN |
| Sq35 | | IVMTSKMVEDYTGMQTQPHKAIVGANAFAHESGIHQDGMLKHKGTYEIICPEEIGLERSN |
| | | ********* ************************************************** |
| Sq2 | 421 | DAGIVLGKLSGRHALKDRLTELGYQLDDEQLSTIFWRFKTVAEQKKRVTDADIIALVSDE |
| Sq35 | 413 | DAGIVLGKLSGRHALKDRLTELGYVLDDEQLSSIFWRFKSVAERKKRVTDADIIALVSDE |
| | | ************************ ******* ***** *** **************** |
| Sq2 | 481 | VFQPEAVWKLLDIQITCGTLGLSTATVKLADADGKEHVACSIGTGPVDSAYKAVDLIVKE |
| Sq35 | 473 | VFQPEALWKLLDIQITCGTLGLSTATVKLADADGKEHVACSMGTGPVDSAYKAVDLVVKE |
| | | ***************************************** ****************** |
| Sq2 | 541 | PATLLEYSMNAVTEGIDAIATTRVLIRGSNKYSSTNAITGEEVQRTFSGTGAGMDIVVSS |
| Sq35 | 533 | PATLLEYSMNAVTEGIDAIATTRVLIRGNNNYSTTNAITGEEVQRTFSGTGAGMDIVVSS |
| | | **************************** * ** ************************** |
| Sq2 | 601 | VKAYVGALNKMMDPKENSATKIPSQKNRVAA |
| Sq35 | 593 | VKAYVGALNKMLDFKENSTTKIPSQNNKVPA |
| | | *********** ****** ****** * * * |

Another Brassica napus IPMS protein sequence is shown below as SEQ ID NO:36, where two positions (223 and 601) are highlighted that can be modified to increase the production of various amino acids.

| | |
|---|---|
| 1 | MASSILRNPM LSSPTTTIPT PSLPSSSSKP SPLSFREPPS |
| 41 | HHRSSVSLRS QSLRLSCSLS DPSPPLRRRR PEYIPNRISD |
| 81 | PNYVRVFDTT LRDGEQSPGA TLTSKEKLDI ARQLAKLGVD |
| 121 | VIEAGFPAAS KDDFEAVKTI AETVGNAVDG DGYVPVICGL |
| 161 | SRCNKRDIET AWEAVKYAKR PRIHTFIATS DIHLEYKLKK |
| 201 | SKDEVIEIAR NMVKFARSLG CEDVEFSPED AGRSEREFLY |
| 241 | EILGEVIKAG ATTLNIPDTV GITLPSEFGQ LIADIKANTP |
| 281 | GIENVIISTH CQNDLGLSTA NTLSGAHSGA RQVEVTINGI |
| 321 | GERAGNASLE EVVMAIKCRG DHVLGGLYTG IDTRHIVMTS |
| 361 | KMVEDYTGMQ TQPHKAIVGA NAFARESGIH QDGMLKHKGT |
| 401 | YEIICPEEIG LERSNDAGIV LGKLSGRHAL KDRLTELGYV |
| 441 | LDDEQLSSIF WRFKSVAERK KRVTDADIIA LVSDEVFQPE |
| 481 | ALWRLLDIQI TCGTLGLSTA TVKLVDADGK ERVACSMGAG |
| 521 | PVDSAYKAID LIVKEPATLL EYSMNAVTEG IDAIATTRVL |
| 561 | IRGNNNYSTT NAITGEEVQR TFSGTGAGMD IVVSSVKAYV |
| 601 | GALNKMLDFK ENAPTKVPSQ NNNVPA |

The Brassica napus IPMS protein with SEQ ID NO:36 is encoded by the 2-isopropylmalate synthase 1 gene on chromosome A6 (LOC106346910; locus tag SORBI_30050030100; see NCBI website). A cDNA that encodes the Brassica napus IPMS protein with SEQ ID NO:36 is shown below as SEQ ID NO:37.

| 1 | CGATGAGACA GAGCTGGATC AAGTTACCGC CGCCACGTTG |
|---|---|
| 41 | AACCTTCTTC TCTATCGTCG TCCCCGTTTA GGTTTACCAC |
| 81 | TCTTCTTTCA ACAATGGCGT CTTCGATTCT CAGAAACCCT |
| 121 | ATGCTCTCAT CACCAACAAC AACAATCCCC ACCCCTTCTC |
| 161 | TTCCCTCCTC CTCCTCAAAA CCCTCACCTC TCTCATTCCG |
| 201 | CTTCCCACCC TCCCACCACC GCTCCTCCGT TTCCCTCCGC |
| 241 | AGCCAATCCC TCCGCCTCTC CTGCTCCCTC TCAGATCCCT |
| 281 | CTCCTCCCCT CCGCCGCCGC CGCCCGGAGT ACATCCCCAA |
| 321 | CCGCATTTCC GACCCCAACT ACGTCCGAGT CTTCGACACC |
| 361 | ACTCTCCGCG ACGGCGAACA GTCCCCCGGA GCCACCCTCA |
| 401 | CCTCCAAGGA AAAGCTCGAC ATCGCGCGCC AGCTCGCGAA |
| 441 | GCTAGGCGTC GACGTAATCG AGGCCGGCTT CCCCGCCGCC |
| 481 | TCCAAGGACG ACTTCGAAGC CGTCAAAACC ATAGCCGAAA |
| 521 | CCGTGGGAAA CGCCGTCGAC GGAGACGGTT ACGTCCCCGT |
| 561 | CATCTGCGGA CTCTCGAGAT GCAACAAGAG AGATATAgAG |
| 601 | ACGGCGTGGG AGGCTGTGAA GTACGCCAAA AGGCCGAGGA |
| 641 | TCCATACCTI CATCGCCACG AGTGACATTC ACTTGGAGTA |
| 681 | TAAGCTGAAG AAGAGCAAAG ACGAGGTCAT CGAGATCGCT |
| 721 | AGGAATATGG TTAAGTTCGC GAGGAGCTTG GGGTGTGAGG |
| 761 | ACGTTGAGTT TAGTCCTGAA GATGCTGGAA GATCGGAGAG |
| 801 | AGAGTTTTTG TATGAGATTC TTGGGGAAGT GATAAAAGCT |
| 841 | GGAGCGACAA CGCTTAATAT ACCTGACACT GTTGGTATAA |
| 881 | CGTTGCCTAG TGAGTTTGGT CAGTTGATTG CTGATATTAA |
| 921 | AGCCAATACT CCTGGGATCG AGAATGTTAT CATCTCAACG |
| 961 | CATTGTCAGA ATGATCTTGG GCTCTCCACT GCGAACACTT |
| 1001 | TATCTGGTGC ACATTCGGGT GCAAGGCAAG TGGAAGTGAC |
| 1041 | TATCAATGGC ATTGGGGAAA GAGCTGGAAA CGCTTCACTA |
| 1081 | GAAGAGGTTG TGATGGCCAT AAAATGCCGI GGAGATCATG |
| 1121 | TATTAGGAGG CCTATATACT GGAATCGATA CTCGCCACAT |
| 1161 | TGTTATGAGA AGCAAGATGG TTGAGGATTA CACAGGAATG |
| 1201 | CAAACACAGC CCCATAAGGC TATTGTAGGA GCGAATGCCT |
| 1241 | TTGCGCATGA AAGTGGTATT CATCAGGATG GAATGCTGAA |
| 1281 | ACACAAGGGC ACATATGAAA TTATATGCCC CGAAGAAATT |
| 1321 | GGACTTGAAC GATCTAATGA TGCTGGCATT GTTTTGGGGA |
| 1361 | AGCTTAGTGG GCGTCATGCG TTGAAAGACC GTTTGACTGA |
| 1401 | GCTTGGTTAT GTACTAGATG ATGAACAGCT AAGTTCCATT |
| 1441 | TTCTGGCGCT TCAAATCTGT GGCTGAGCGG AAAAAGAGAG |
| 1481 | TTACCGACGC AGATATAATA GCTTTGGTTT CTGATGAGGT |
| 1521 | TTTCCAGCCA GAAGCCTTGT GGAGACTCCT GGACATTCAG |
| 1561 | ATTACATGTG GGACTCTCGG ACTCTCAACA GCAACCGTTA |
| 1601 | AACTTGTTGA TGCTGATGGC AAAGAGCATG TTGCCTGTTC |
| 1641 | TATGGGTGCT GGGCCTGTCG ATTCAGCTTA TAAGGCAATC |
| 1681 | GATCTTATTG TCAAGGAACC AGCGACTTTG CTTGAGTACT |
| 1721 | CAATGAATGC GGTAACAGAA GGCATCGATG CCATTGCAAC |
| 1761 | CACACGAGTT CTTATCCGAG GAAATAACAA TTACTCAACT |
| 1801 | ACAAATGCAA TCACTGGTGA AGAAGTTCAA AGGACCTTTA |
| 1841 | GTGGAACCGG AGCTGGAATG GACATTGTGG TGTCGAGCGT |
| 1881 | CAAAGCTTAT GTAGGAGCTT TGAACAAAAT GCTCGACTTC |
| 1921 | AAAGAAAACG CCCCAACGAA AGTCCCTTCT CAAAACAACA |
| 1961 | ATGTACCTGC CTGAATCAAA ATTGTTTCTG AGTCAGACCA |
| 2001 | GAGTTAGTCT TTTCTGGTAT AGGTACATAG TTTGGTAATA |
| 2041 | ACGAGAGTTC AAGGCTTGCA TATTGTTTTA ATGAAGTATC |
| 2081 | TTTGCTGAAA GAGTTCGTTT ACTATAAAAT ATTTATATAG |
| 2121 | AACTTAAATC TCTTTTTATT T |

As illustrated below the Brassica napus IPMS protein with SEQ ID NO:36 has about 89-90% sequence identity with the Arabidopsis IPMS1 SEQ ID NO:2 sequence.

| | | |
|---|---|---|
| Sq2 | 1 | MASSLLRNPNLYSSTTITTTSFLPTESSKPIPISSSFRFQPSHHRS-ISLRSQTLRLSCS |
| Sq36 | 1 | MASSILRNPMLSSPTTTIPTPSLPSSSSKPSPLS--FRFPPSHHRSSVSLRSQSLRLSCS |
| | | **** **** * * ** * ** **** * * *** ****** ***** ****** |
| Sq2 | 60 | ISDPSPLPPHTPRRPRPEYIPNRISDPNYVRVFDTTLRDGEQSPGATLTSKEKLDIARQL |
| Sq36 | 60 | LSDPSP----PLRRRRPEYIPNRISDPNYVRVFDTTLRDGEQSPGATLTSKEKLDIARQL |
| | | ****** ** ********************************************* |
| Sq2 | 120 | AKLGVDIIEAGFPAASKDDFEAVKTIAETVGNTVDENGYVPVICGLSRCNKKDIERAWDA |
| Sq36 | 115 | AKLGVDVIEAGFPAASKDDPEAVKTIAETVGNAVDGDGYVPVICGLSRCNKRDIETAWEA |
| | | ****** ************************* ** ************** *** ** * |
| Sq2 | 180 | VKYAKRPRIHTFIATSDTHLEYKLKRTKAEVTEIARSMVRFARSLGCEDVEFSPEDAGRS |
| Sq36 | 175 | VKYAKRPRIHTFIATSDIHLEYKLKKSKDEVIEIARNMVKFARSLGCEDVEFSPEDAGRS |
| | | ************************** * ******* ** ******************** |
| Sq2 | 240 | EREYLYEILGEVIKAGATTLNIPDTVGITLPSEFGQLITDLKANTPGIENVVISTHCQND |
| Sq36 | 235 | EREFLYEILGEVIKAGATTLNIPDTVGITLPSEFGQLIADIKANTPGIENVIISTHCQND |
| | | *** ********************************** * ********** ******** |
| Sq2 | 300 | LGLSTANTLSGAHAGARQMEVTINGIGERAGNASLEEVVMAIKCRGDHVLGGLFTGIDIR |
| Sq36 | 295 | LGLSTANTLSGAHSGARQVEVTINGIGERAGNASLEEVVMATKCRGDHVLGGLYTGIDTR |
| | | ************* **** ********************************* ****** |
| Sq2 | 360 | HIVMTSKMVEEYIGMQTQPHKAIVGANAFAHESGIHQDGMLKHKGTYEIICPEEIGLERS |
| Sq36 | 355 | HIVMTSKMVEDYTGMQTQPHKAIVGANAFAHESGIHQDGMLKHKGTYEIICPEEIGLERS |
| | | ********** ************************************************* |
| Sq2 | 420 | NDAGIVLGKLSGRHALKDRLTELGYQLDDEQLSTIFWRFKTVAEQKKRVTDADIIALVSD |
| Sq36 | 415 | NDAGIVLGKLSGRHALKDRLTELGYVLDDEQLSSIFWRFKSVAERKKRVTDADIIALVSD |
| | | ************************* ******* ****** *** ************** |
| Sq2 | 480 | EVFQPEAVWKLLDIQITCGTLGLSTATVKLADADGKEHVACSIGTGPVDSAYKAVDLIVK |
| Sq36 | 475 | EVFQPEALWRLLDIQITCGTLGLSTATVKLVDADGKEHVACSMGAGPVDSAYKAIDLIVK |
| | | ******* * ******************** *********** * ********* ***** |
| Sq2 | 540 | EPATLLEYSMNAVTEGIDAIATTRVLIRGSNKYSSTNAITGEEVQRTFSGTGAGMDIVVS |
| Sq36 | 535 | EPATLLEYSMNAVTEGIDAIATTRVLIRGNNNYSTTNAITGEEVQRTESGTGAGMDIVVS |
| | | ***************************** * ** ************************* |
| Sq2 | 600 | SVKAYVGALNKMMDFKENSATKIPSQKNRVAA |
| Sq36 | 595 | SVKAYVGALNKMLDFKENAPTKVPSQNNNVPA |
| | | ************ ***** ** *** * * * |

Another Brassica napus IPMS protein sequence is shown below as SEQ ID NO:38, where two positions (223 and 601) are highlighted that can be modified to increase the production of various amino acids.

| | |
|---|---|
| 1 | MASSILRNPM LSSPTTTITT PSLPSSSSKD SPLSFREPPS |
| 41 | HHRSSLSLRL KSLRLSCSLS DPSPPLRRRR PEYIPNRISD |
| 81 | PNYVRVFDTT LRDGEQSPGA TLTSKEKLDI ARQLAKLGVD |
| 121 | VIEAGFPAAS KDDFEAVKTI AETVGNAVDG DGYVPVICGL |
| 161 | SRCNKRDIET AWEAVKYAKR PRIHTFIATS DIHLEYKLKK |
| 201 | SKDEVIEIAR NMVKFARSLG CEDVEFSPED AGRSEREFLY |
| 241 | EILGEVIKAG ATTLNIPDTV GITLPSEFGQ LEAD1KANTP |
| 281 | GIENVIISTH CQNDLGLSTA NTLSGAHSGA RQVEVTINGI |
| 321 | GERAGNASLE EVVMAIKCRG DHVLGGLYTG IDTRHIVMTS |
| 361 | KMVEDYTGMQ TQPHKAIVGA NAFAHESGIH QDGMLKHKGT |
| 401 | YEIICPEEIG LERSNDAGIV LGKLSGRHAL KDRLTELGYV |
| 441 | LDDEQLSSIF WRFKSVAERK KRVTDADIIA LVSDEVEQPE |
| 481 | ALWRLLDIQI TCGTLGLSTA TVKLVDADGK EHVACSMGAG |
| 521 | PVDSAYKAID LIVKEPATLL EYSMNAVTEG IDAIATTRVL |
| 561 | IRGNNNYSTT NAITGEEVQR TFSGTGAGMD IVVSSVKAYV |
| 601 | GALNKMLDFK ENAPTKVPSQ NNNVPA |

Table 1 lists some of accession numbers for IPMS homologs.

TABLE 1
IPMS Homologs

| | UniProt | |
|---|---|---|
| Organism | Accession | Gene |
| Arabidopsis thaliana | Q9C550-1 | AT1G74040.1 |
| (Thale cress) | | |
| Arabidopsis thaliana | Q9FG67-1 | AT5G23010.1 |
| (Thale cress) | | |
| Arabidopsis thaliana | Q9FN52-1 | AT5G23020.1 |
| (Thale cress) | | |
| Brachypodium distachyon | I1IUJ8 | BRADI4G43130 |
| (Purple false brome) | | |
| Chlamydomonas reinhardtii | A8HXS9 | LEU2 |
| (Chlamydomonas) | | |
| Glycine max (Soybean) | I1JK46 | GLYMA03G00800 |
| Glycine max (Soybean) | K7LM62 | GLYMA10G44180 |
| Glycine max (Soybean) | K7LQ10 | GLYMA11G17781 |
| Glycine max (Soybean) | I1LKJ2 | GLYMA11G17790 |
| Glycine max (Soybean) | K7LQ11 | GLYMA11G17804 |
| Glycine max (Soybean) | K7LYP6 | GLYMA13G12400 |
| Glycine max (Soybean) | K7LYP8 | GLYMA13G12470 |
| Glycine max (Soybean) | K7LYP9 | GLYMA13G12484 |
| Glycine max (Soybean) | K7LYQ0 | GLYMA13G12498 |
| Glycine max (Soybean) | K7LYQ4 | GLYMA13G12565 |

The sequences used in the plans, plant cells, seeds and methods described herein can have less than 100% sequence identity to any of SEQ ID NO:1-38. For example, the sequences can have about at least 40% sequence identity, or at least 50% sequence identity, or at least 60% sequence identity, or at least 70% sequence identity, or at least 80% sequence identity, or at least 90% sequence identity, or at least 95% sequence identity, or at least 96% sequence identity, or at least 97% sequence identity, or at least 98% sequence identity, or at least 99% sequence identity, or at least 99.5% sequence identity, or 60-99%, sequence identity, or 70-99% sequence identity, or 80-99% sequence identity, or 90-95% sequence identity, or 90-99% sequence identity, or 95-97% sequence identity, or 97-99% sequence identity, or 100% sequence identity with any of SEQ ID N A1-38.

The modified IPMS proteins described herein can have a variety of amino acids, and a variety of mutations. For example, the modified IPMS1 proteins described herein can have any of the amino acids listed in Table 2.

TABLE 2

| | One-Letter | Common |
|---|---|---|
| Amino Acid | Symbol | Abbreviation |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |
| β-Alanine | | bAla |
| N-Methylglycine | | MeGly |
| (sarcosine) | | |
| Ornithine | | Orn |
| Norleucine | | Nle |
| Penicillamine | | Pen |
| Homoarginine | | hArg |
| N-methylvaline | | MeVal |
| Homocysteine | | hCys |
| Homoserine | | hSer |

The modified plant cells, plants, and seeds described herein can have genomic mutations that alter one or more amino acids in the encoded IPMS protein. For example, one or more amino acids in the IPMS polypeptide can be replaced or deleted. In some cases, one or more amino acids in the IPMS can have be replaced by a conservative amino acid. In other cases, one or more amino acids having physical and/or chemical properties that are different from the amino acid(s) that are present in the parental or wild type plant cells, plants, or seeds. For example, to change the physical and/or chemical properties of amino acids, the amino acids can be deleted or replaced by amino acids of another class, where the classes are identified in the following Table 3.

TABLE 3

| | |
|---|---|
| | Genetically |
| Classification | Encoded |

Hydrophobic

| | |
|---|---|
| Aromatic | F, Y, W |
| Apolar | M, G, P |
| Aliphatic | A, V, L, I |

Hydrophilic

| | |
|---|---|
| Acidic | D, E |
| Basic | H, K, R |
| Polar | Q, N, S, T, Y |
| Cysteine-Like | C |

Different types of amino acids can therefore be employed in the modified IPMS proteins.

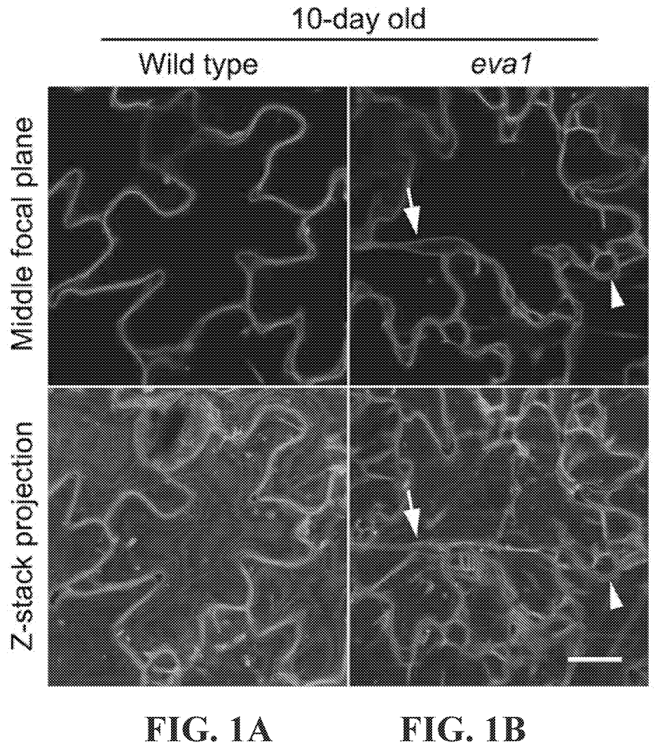

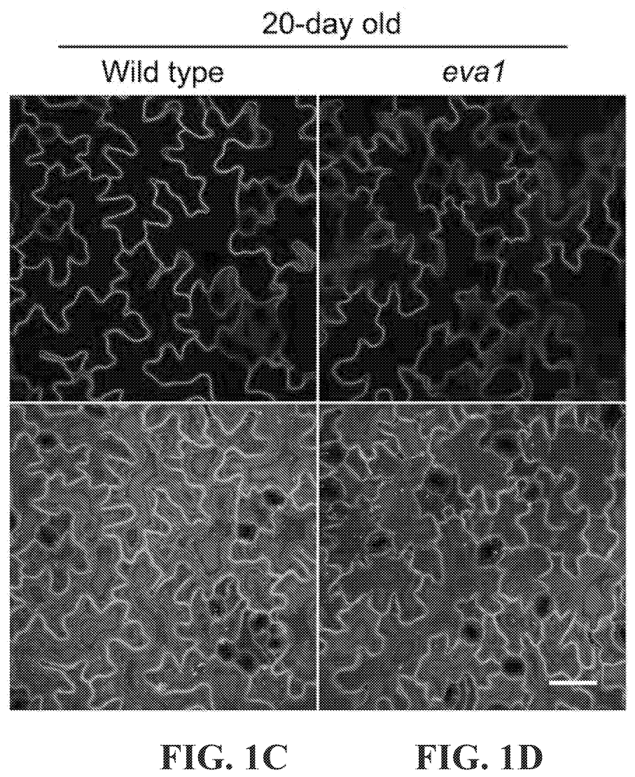

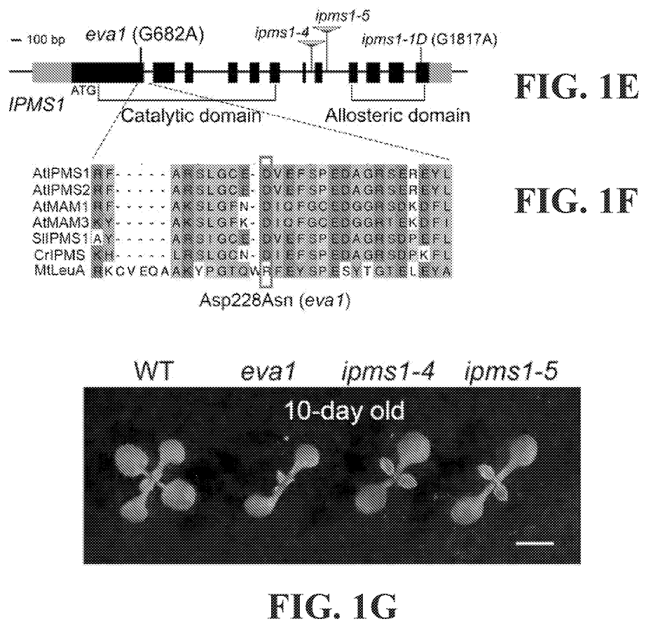

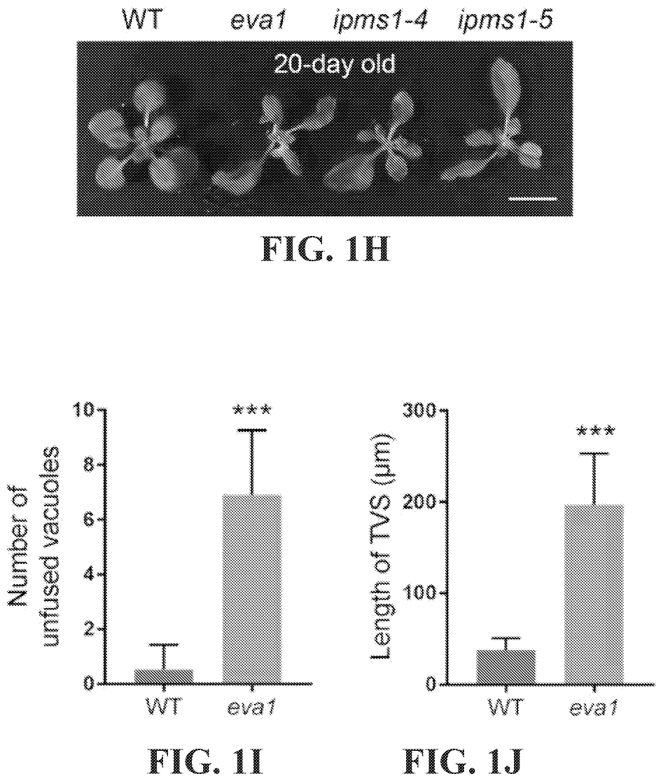

For example, in some cases an acidic amino acid in a catalytic domain of an IPMS protein can be replaced with a polar amino acid. Such acidic amino acids can, for example, be aspartic acid (D) or glutamic acid (E). In some cases, the acidic amino acid in a catalytic domain of an IPMS protein that is modified, mutated, or replaced can be aspartic acid, for example, in the catalytic domain sequence shown in FIG. 1E-1F. Such an aspartic acid can be within the catalytic domain at a position that corresponds to position 228 of the SEQ ID NO:2 sequence. The polar amino acid that replaces the acidic amino acid can be an asparagine, glutamine, serine, threonine, or tyrosine. In some cases, the acidic amino acid is replaced by an asparagine. For example, the catalytic domain aspartic acid at a position that corresponds to position 228 of the SEQ ID NO:2 sequence that is modified in any IPMS can be replaced with an asparagine.

In another example, an apolar amino acid in the allosteric domain can be modified. Such an apolar amino acid can be a glycine, methionine or proline amino acid. In some cases, the apolar amino acid in an allosteric domain of an IPMS protein that is modified, mutated, or replaced can be glycine, for example, in the allosteric domain shown in FIG. 1E-1F. Such an apolar amino acid can be within the allosteric domain at a position that corresponds to position 606 of the SEQ ID NO:2 sequence. The apolar amino acid in the allosteric domain can be modified, mutated, or replaced with an acidic amino acid such as an aspartic acid or a glutamic acid. For example, a glycine in any IPMS at a position corresponding to position 606 of the SEQ ID NO:2 sequence can be a glutamic acid (E).

### Modifying Plant Cells, Plants, and Seeds

Modified IPMS1 nucleic acids and/or modified IPMS1 proteins are introduced into plant cells, plants, and seeds to provide higher levels of Gln, His, Ile, Leu, Lys, Met. Phe, Thr, Trp, Val. or a combination thereof in their vegetative tissues (e.g., leaves, roots, stems, branches) and seeds. In some cases, at least one native IPMS1 gene is modified or mutated to induce expression of one or more modified IPMS1 proteins. In other cases, and one or more expression cassettes is introduced that includes an expression cassette for expressing a modified IPMS1 protein, where the expression cassette encodes a modified IPMS1 coding region under the control of a promoter. One of skill in the art can generate genetically modified plant cells, plants, and/or seeds that contain nucleic acids encoding a modified IPMS1 within their somatic and/or germ cells. Such genetic modification can be accomplished by various procedures.

Non-limiting examples of methods of introducing a modification into the genome of a cell can include use of microinjection, viral delivery, recombinase technologies, homologous recombination, TALENS, CRISPR, and/or ZFN, see, e.g. Clark and Whitelaw Nature Reviews Genetics 4:825-833 (2003); which is incorporated by reference herein in its entirety.

For example, nucleases such as zinc finger nucleases (ZFNs), transcription activator like effector nucleases (TALENs), and/or meganucleases can be employed with a guide nucleic acid that allows the nuclease to target the genomic IPMS1 site(s). In some cases, a targeting vector can be used to introduce a deletion or modification of one or more genomic IPMS1 site(s).

A “targeting vector” is a vector generally has a 5′ flanking region and a 3′ flanking region homologous to segments of the gene of interest. The 5′ flanking region and a 3′ flanking region can surround a DNA sequence comprising a modification and/or a foreign DNA sequence to be inserted into the gene. For example, the foreign DNA sequence may encode a selectable marker. In some cases, the targeting vector does not comprise a selectable marker, but such a selectable marker can facilitate identification and selection of cells with desirable mutations. Examples of suitable selectable markers include antibiotics resistance genes such as chloramphenicol resistance, gentamycin resistance, kanamycin resistance, spectinomycin resistance (SpecR), neomycin resistance gene (NEO), and/or the hygromycin p-phosphotransferase genes. The 5′ flanking region and the 3′ flanking region can be homologous to regions within the gene, or to regions flanking the gene to be deleted, modified, or replaced with the unrelated DNA sequence.

The targeting vector is contacted with the native gene of interest in vivo (e.g., within the cell) under conditions that favor homologous recombination. For example, the cell can be contacted with the targeting vector under conditions that result in transformation of the cyanobacterial cell(s) with the targeting vector.

A typical targeting vector contains nucleic acid fragments of not less than about 0.1 kb nor more than about 10.0 kb from both the 5′ and the 3′ ends of the genomic locus which encodes the gene to be modified (e.g. the genomic IPMS site(s)). These two fragments are separated by an intervening fragment of nucleic acid which encodes the modification to be introduced. When the resulting construct recombines homologously with the chromosome at this locus, it results in the introduction of the modification, e.g. a deletion of a portion of the genomic IPMS site(s), replacement of one or more amino acids in the genomic IPMS coding region site(s), or the insertion of non-conserved codon into the IPMS coding region.

In some cases, a Cas9/CRISPR system can be used to create a modification in genomic IPMS1 site(s). Clustered regularly interspaced short palindromic repeats (CRISPR)/CRISPR-associated (Cas) systems are useful for, e.g. RNA-programmable genome editing (see e.g., Marraffini and Sontheimer. Nature Reviews Genetics 11: 181-190 (2010); Sorek et al. Nature Reviews Microbiology 2008 6: 181-6; Karginov and Hannon. Mol Cell 2010 1:7-19; Hale et al. Mol Cell 2010:45:292-302; Jinek et al. Science 2012 337:815-820; Bikard and Marraffini Curr Opin Immunol 2012 24:15-20; Bikard et al. Cell Host & Microbe 2012 12: 177-186; all of which are incorporated by reference herein in their entireties). A CRISPR guide RNA can be used that can target a Cas enzyme to the desired location in the genome, where it generates a double strand break. This technique is described, for example, by Mali et al. Science 2013 339:823-6; which is incorporated by reference herein in its entirety. Kits for the design and use of CRISPR-mediated genome editing are commercially available, e.g. the PRECISION X CAS9 SMART NUCLEASE™ System (Cat No. CAS900A-1) from System Biosciences, Mountain View, Calif.

In other cases, a cre-lox recombination system of bacteriophage P1, described by Abremski et al. 1983. Cell 32:1301 (1983). Sternberg et al., Cold Spring Harbor Symposia on Quantitative Biology, Vol. XLV 297 (1981) and others, can be used to promote recombination and alteration of the genomic MinC and/or MinD site(s). The cre-lox system utilizes the cre recombinase isolated from bacteriophage P1 in conjunction with the DNA sequences that the recombinase recognizes (termed lox sites). This recombination system has been effective for achieving recombination in plant cells (see, e.g., U.S. Pat. No. 5,658,772), animal cells (U.S. Pat. Nos. 4,959,317 and 5,801,030), and in viral vectors (Hardy et al., J. Virology 71:1842 (1997).

Another method for generating modified plant cells, plants and/or seeds include introducing an expression cassette or expression vector that can express modified IPMS1 polypeptides. Plant cells can be transformed by the expression cassette or expression vector, and whole plants (and their seeds) can be generated from the plant cells that were successfully transformed with the IPMS nucleic acids. Some procedures for making such genetically modified plants and their seeds are described below.

Promoters: The modified IPMS nucleic acids described herein can include a modified IPMS1 coding region operably linked to a promoter, which provides for expression of mRNA from the modified IPMS coding region. The promoter is typically a promoter functional in plants and/or seeds and can be a promoter functional during plant growth and development. A modified IPMS nucleic acid is operably linked to the promoter when it is located downstream from the promoter, to thereby form an expression cassette.

Most endogenous genes have regions of DNA that are known as promoters, which regulate gene expression. Promoter regions are typically found in the flanking DNA upstream from the coding sequence in both prokaryotic and eukaryotic cells. A promoter sequence provides for regulation of transcription of the downstream gene sequence and typically includes from about 50 to about 2,000 nucleotide base pairs. Promoter sequences also contain regulatory sequences such as enhancer sequences that can influence the level of gene expression. Some isolated promoter sequences can provide for gene expression of heterologous DNAs, that is a DNA different from the native or homologous DNA.

Promoter sequences are also known to be strong or weak, or inducible. A strong promoter provides for a high level of gene expression, whereas a weak promoter provides for a very low level of gene expression. An inducible promoter is a promoter that allows gene expression to be turned on and off in response to an exogenously added agent, or to an environmental or developmental stimulus. For example, a bacterial promoter such as the P_{tac} promoter can be induced to vary levels of gene expression depending on the level of isothiopropylgalactoside added to the transformed cells. Promoters can also provide for tissue specific or developmental regulation. An isolated promoter sequence that is a strong promoter for heterologous DNAs is advantageous because it provides for a sufficient level of gene expression for easy detection and selection of transformed cells and provides for a high level of gene expression when desired.

Expression cassettes generally include, but are not limited to, a plant promoter such as the CaMV 35S promoter (Odell et al., Nature, 313:810-812 (1985)), or others such as CaMV 19S (Lawton et al., Plant Molecular Biology, 9:315-324 (1987)), nos (Ebert et al., Proc. Natl. Acad. Sci. USA. 84:5745-5749 (1987)), Adh1 (Walker et al., Proc. Natl. Acad. Sci. USA. 84:6624-6628 (1987)), sucrose synthase (Yang et al., Proc. Natl. Acad. Sci. USA. 87:4144-4148 (1990)), α-tubulin, ubiquitin, actin (Wang et al., Mol. Cell. Biol. 12:3399 (1992)), cab (Sullivan et al., Mol. Gen. Genet. 215:431 (1989)), PEPCase (Hudspeth et al., Plant Molecular Biology. 12:579-589 (1989)) or those associated with the R gene complex (Chandler et al., The Plant Cell. 1:1175-1183 (1989)). Further suitable promoters include the poplar xylem-specific secondary cell wall specific cellulose synthase 8 promoter, cauliflower mosaic virus promoter, the Z10 promoter from a gene encoding a 10 kDa zein protein, a Z27 promoter from a gene encoding a 27 kDa zein protein, inducible promoters, such as the light inducible promoter derived from the pea rbcS gene (Coruzzi et al., EMBO J. 3:1671 (1971)) and the actin promoter from rice (McElroy et al., The Plant Cell. 2:163-171 (1990)). Seed specific promoters, such as the phaseolin promoter from beans, may also be used (Sengupta-Gopalan, Proc. Natl. Acad. Sci. USA. 83:3320-3324 (1985). Other examples of seed specific promoters that can be used include the P1, P3, P4, P6, P7, P9, P13, P14, P15, P16, P17, and P19 promoters described in U.S. Pat. No. 7,081,565 (which information about the P1, P3, P4, P6, P7, P9, P13. P14, P15, P16, P17, and P19 promoters is incorporated by reference herein in its entirety).

A modified IPMS1 nucleic acid can be combined with the promoter by standard methods to yield an expression cassette, for example, as described in Sambrook et al. (MOLECULAR CLONING: A LABORATORY MANUAL. Second Edition (Cold Spring Harbor, N.Y.: Cold Spring Harbor Press (1989); MOLECULAR CLONING: A LABORATORY MANUAL. Third Edition (Cold Spring Harbor, N.Y.: Cold Spring Harbor Press (2000)). Briefly, a plasmid containing a promoter such as the 35S CaMV promoter can be constructed as described in Jefferson (Plant Molecular Biology Reporter 5:387-405 (1987)) or obtained from Clontech Lab in Palo Alto, Calif. (e.g., pBI121 or pBI221). Typically, these plasmids are constructed to have multiple cloning sites having specificity for different restriction enzymes downstream from the promoter. The IPMS1 nucleic acid segment can be subcloned downstream from the promoter using restriction enzymes and positioned to ensure that the DNA is inserted in proper orientation with respect to the promoter so that the DNA can be expressed as sense RNA. Once the IPMS1 nucleic acid segment is operably linked to a promoter, the expression cassette so formed can be subcloned into a plasmid or other vector (e.g., an expression vector).

In some embodiments, a cDNA clone encoding a modified IPMS1 protein is isolated from plant tissue, for example, a root, stem, leaf, seed, or flower tissue. For example, cDNA clones from selected species (that encode an IPMS1 protein with homology to any of those described herein) are made from isolated mRNA from selected plant tissues. In another example, a nucleic acid encoding a mutant or modified IPMS1 protein can be prepared by available methods or as described herein. For example, the nucleic acid encoding a mutant or modified IPMS1 protein can be any nucleic acid with a coding region that hybridizes to a segment of a SEQ ID NO:1, 4, 6, 8, 27, 29, 31, 34, or 37 nucleic acid. For example, any of the modified IPMS1 nucleic acids can have one or more nucleotide differences to any of the SEQ ID NO:1, 4, 6, 8, 27, 29, 31, 34, or 37 nucleic acid sequences. Such a nucleic acid can encode an enzyme with isopropylmalate synthase activity and/or protein folding activity. Using restriction endonucleases, the entire coding sequence for the modified IPMS1 nucleic acid segment is subcloned downstream of the promoter in a 5′ to 3′ sense orientation.

Targeting Sequences: Additionally, expression cassettes can be constructed and employed to target the modified IPMS1 proteins to an intracellular compartment within plant cells, into a membrane, or to direct an encoded protein to the extracellular environment. This can generally be achieved by joining a DNA sequence encoding a transit or signal peptide sequence to the coding sequence of the modified IPMS1 nucleic acid. The resultant transit, or signal, peptide will transport the protein to a particular intracellular, or extracellular destination, respectively, and can then be posttranslational removed. Transit peptides act by facilitating the transport of proteins through intracellular membranes, e.g., vacuole, vesicle, plastid and mitochondrial membranes, whereas signal peptides direct proteins through the extracellular membrane. By facilitating transport of the protein into compartments inside or outside the cell, these sequences can increase the accumulation of a particular gene product in a particular location. For example, see U.S. Pat. No. 5,258,300.

3′Sequences: When the expression cassette is to be introduced into a plant cell, the expression cassette can also optionally include 3′ nontranslated plant regulatory DNA sequences that act as a signal to terminate transcription and allow for the polyadenylation of the resultant mRNA. The 3′ nontranslated regulatory DNA sequence preferably includes from about 300 to 1.000 nucleotide base pairs and contains plant transcriptional and translational termination sequences. For example, 3′ elements that can be used include those derived from the nopaline synthase gene of Agrobacterium tumefaciens (Bevan et al., Nucleic Acid Research. 11:369-385 (1983)), or the terminator sequences for the T7 transcript from the octopine synthase gene of Agrobacterium tumefaciens, and/or the 3′ end of the protease inhibitor I or II genes from potato or tomato. Other 3′ elements known to those of skill in the art can also be employed. These 3′ nontranslated regulatory sequences can be obtained as described in An (Methods in Enzymology. 153:292 (1987)). Many such 3′ nontranslated regulatory sequences are already present in plasmids available from commercial sources such as Clontech, Palo Alto, Calif. The 3′ nontranslated regulatory sequences can be operably linked to the 3′ terminus of the IPMS1 nucleic acids by standard methods.

Selectable and Screenable Marker Sequences: To improve identification of transformants, a selectable or screenable marker gene can be employed with the expressible IPMS1 nucleic acids. “Marker genes” are genes that impart a distinct phenotype to cells expressing the marker gene and thus allow such transformed cells to be distinguished from cells that do not have the marker. Such genes may encode either a selectable or screenable marker, depending on whether the marker confers a trait which one can ‘select’ for by chemical means, e.g., by use of a selective agent (e.g., an herbicide, antibiotic, or the like), or whether it is simply a trait that one can identify through observation or testing. i.e., by ‘screening’ (e.g., the R-locus trait). Of course, many examples of suitable marker genes are known to the art and can be employed in the practice of the invention.

Included within the terms selectable or screenable marker genes are also genes which encode a “secretable marker” whose secretion can be detected as a means of identifying or selecting for transformed cells. Examples include markers which encode a secretable antigen that can be identified by antibody interaction, or secretable enzymes that can be detected by their catalytic activity. Secretable proteins fall into a number of classes, including small, diffusible proteins detectable. e.g., by ELISA; and proteins that are inserted or trapped in the cell wall (e.g., proteins that include a leader sequence such as that found in the expression unit of extensin or tobacco PR-S).

With regard to selectable secretable markers, the use of a gene that encodes a polypeptide that becomes sequestered in the cell wall, where the polypeptide includes a unique epitope may be advantageous. Such a secreted antigen marker can employ an epitope sequence that would provide low background in plant tissue, a promoter-leader sequence that imparts efficient expression and targeting across the plasma membrane and can produce protein that is bound in the cell wall and yet is accessible to antibodies. A normally secreted wall protein modified to include a unique epitope would satisfy such requirements.

Examples of proteins suitable for modification in this manner include extensin or hydroxyproline rich glycoprotein (HPRG). For example, the maize HPRG (Stiefel et al., The Plant Cell. 2:785-793 (1990)) is well characterized in terms of molecular biology, expression, and protein structure and therefore can readily be employed. However, any one of a variety of extensins and/or glycine-rich wall proteins (Keller et al., EMBO J. 8:1309-1314 (1989)) could be modified by the addition of an antigenic site to create a screenable marker.

Numerous other possible selectable and/or screenable marker genes will be apparent to those of skill in the art in addition to those forth herein below. Therefore, it will be understood that the discussion herein is exemplary rather than exhaustive. In light of the techniques disclosed herein and the general recombinant techniques that are known in the art, the present invention readily allows the introduction of any gene, including marker genes, into a recipient cell to generate a transformed plant cell, e.g., a monocot cell or dicot cell.

Possible selectable markers for use in connection with the present invention include, but are not limited to, a neo gene (Potrykus et al., Mol. Gen. Genet. 199:183-188 (1985)) which codes for kanamycin resistance and can be selected for using kanamycin, G418, and the like; a bar gene which codes for bialaphos resistance; a gene which encodes an altered EPSP synthase protein (Hinchee et al., Bio/Technology. 6:915-922 (1988)) thus conferring glyphosate resistance; a nitrilase gene such as brn from Klebsiella ozaenae which confers resistance to bromoxynil (Stalker et al., Science. 242:419-423 (1988)); a mutant acetolactate synthase gene (ALS) which confers resistance to imidazolinone, sulfonylurea or other ALS-inhibiting chemicals (European Patent Application 154.204 (1985)); a methotrexate-resistant DHFR gene (Thillet et al., J. Biol. Chem. 263:12500-12508 (1988)); a dalapon dehalogenase gene that confers resistance to the herbicide dalapon; or a mutated anthranilate synthase gene that confers resistance to 5-methyl tryptophan. Where a mutant EPSP synthase gene is employed, additional benefit may be realized through the incorporation of a suitable chloroplast transit peptide, CTP (European Patent Application 0 218 571 (1987)).

An illustrative embodiment of a selectable marker gene capable of being used in systems to select transformants is the gene that encode the enzyme phosphinothricin acetyltransferase, such as the bar gene from Streptomyces hygroscopicus or the pat gene from Streptomyces viridochromogenes (U.S. Pat. No. 5,550,318). The enzyme phosphinothricin acetyl transferase (PAT) inactivates the active ingredient in the herbicide bialaphos, phosphinothricin (PPT). PPT inhibits glutamine synthetase, (Murakami et al., Mol. Gen. Genet. 205:42-50 (1986); Twell et al., Plant Physiol. 91:1270-1274 (1989)) causing rapid accumulation of ammonia and cell death. The success in using this selective system in conjunction with monocots was surprising because of the major difficulties that have been reported in transformation of cereals (Potrykus, Trends Biotech. 7:269-273 (1989)).

Screenable markers that may be employed include, but are not limited to, a p-glucuronidase or uidA gene (GUS) that encodes an enzyme for which various chromogenic substrates are known; an R-locus gene, which encodes a product that regulates the production of anthocyanin pigments (red color) in plant tissues (Dellaporta et al., In: Chromosome Structure and Function: Impact of New Concepts, 18^{th} Stadler Genetics Symposium. J. P. Gustafson and R. Appels, eds. (New York: Plenum Press) pp. 263-282 (1988)); a β-lactamase gene (Sutcliffe, Proc. Natl. Acad. Sci. USA. 75:3737-3741 (1978)), which encodes an enzyme for which various chromogenic substrates are known (e.g., PADAC, a chromogenic cephalosporin); a xvlE gene (Zukowsky et al., Proc. Natl. Acad. Sri. USA. 80:1101 (1983)) which encodes a catechol dioxygenase that can convert chromogenic catechols; an α-amylase gene (Ikuta et al., Bio/technology 8:241-242 (1990)); a tyrosinase gene (Katz et al., J. Gen. Microbiol. 129:2703-2714 (1983)) which encodes an enzyme capable of oxidizing tyrosine to DOPA and dopaquinone which in turn condenses to form the easily detectable compound melanin; a β-galactosidase gene, which encodes an enzyme for which there are chromogenic substrates; a luciferase (lux) gene (Ow et al., Science. 234:856-859.1986), which allows for bioluminescence detection; or an aequorin gene (Prasher et al., Biochem. Biophys. Res. Comm. 126:1259-1268 (1985)), which may be employed in calcium-sensitive bioluminescence detection, or a green or yellow fluorescent protein gene (Niedz et al., Plant Cell Repons. 14:403 (1995)).

For example, genes from the maize R gene complex can be used as screenable markers. The R gene complex in maize encodes a protein that acts to regulate the production of anthocyanin pigments in most seed and plant tissue. Maize strains can have one, or as many as four. R alleles that combine to regulate pigmentation in a developmental and tissue specific manner. A gene from the R gene complex does not harm the transformed cells. Thus, an R gene introduced into such cells will cause the expression of a red pigment and, if stably incorporated, can be visually scored as a red sector. If a maize line carries dominant alleles for genes encoding the enzymatic intermediates in the anthocyanin biosynthetic pathway (C2, A1, A2, Bz1 and Bz2), but carries a recessive allele at the R locus, transformation of any cell from that line with R will result in red pigment formation. Exemplary lines include Wisconsin 22 that contains the rg-Stadler allele and TR112, a K55 derivative that is r-g, b, Pl. Alternatively any genotype of maize can be utilized if the C1 and R alleles are introduced together.

The R gene regulatory regions may be employed in chimeric constructs to provide mechanisms for controlling the expression of chimeric genes. More diversity of phenotypic expression is known at the R locus than at any other locus (Coe et al., in Corn and Corn Improvement, eds. Sprague, G. F. & Dudley, J. W. (Am. Soc. Agron., Madison, Wis.), pp. 81-258 (1988)). It is contemplated that regulatory regions obtained from regions 5′ to the structural R gene can be useful in directing the expression of genes, e.g., insect resistance, drought resistance, herbicide tolerance or other protein coding regions. For the purposes of the present invention, it is believed that any of the various R gene family members may be successfully employed (e.g., P, S, Lc, etc.). However, one that can be used is Sn (particularly Sn:bol3). Sn is a dominant member of the R gene complex and is functionally similar to the R and B loci in that Sn controls the tissue specific deposition of anthocyanin pigments in certain seedling and plant cells, therefore, its phenotype is similar to R.

A further screenable marker contemplated for use in the present invention is firefly luciferase, encoded by the lux gene. The presence of the lux gene in transformed cells may be detected using, for example, X-ray film, scintillation counting, fluorescent spectrophotometry, low-light video cameras, photon counting cameras or multiwell luminometry. It is also envisioned that this system may be developed for population screening for bioluminescence, such as on tissue culture plates, or even for whole plant screening.

Other Optional Sequences: An expression cassette of the invention can also further comprise plasmid DNA. Plasmid vectors include additional DNA sequences that provide for easy selection, amplification, and transformation of the expression cassette in prokaryotic and eukaryotic cells, e.g., pUC-derived vectors such as pUC8, pUC9, pUC18, pUC19, pUC23, pUC19, and pUC120, pSK-derived vectors, pGEM-derived vectors, pSP-derived vectors, or pBS-derived vectors. The additional DNA sequences include origins of replication to provide for autonomous replication of the vector, additional selectable marker genes, preferably encoding antibiotic or herbicide resistance, unique multiple cloning sites providing for multiple sites to insert DNA sequences or genes encoded in the expression cassette and sequences that enhance transformation of prokaryotic and eukaryotic cells.

Another vector that is useful for expression in both plant and prokaryotic cells is the binary Ti plasmid (as disclosed in Schilperoort et al., U.S. Pat. No. 4,940,838) as exemplified by vector pGA582. This binary Ti plasmid vector has been previously characterized by An (Methods in Enzymology. 153:292 (1987)) and is available from Dr. An. This binary Ti vector can be replicated in prokaryotic bacteria such as E. coli and Agrobacterium. The Agrobacterium plasmid vectors can be used to transfer the expression cassette to dicot plant cells, and under certain conditions to monocot cells, such as barley, corn, rice, or wheat cells. The binary Ti vectors preferably include the nopaline T DNA right and left borders to provide for efficient plant cell transformation, a selectable marker gene, unique multiple cloning sites in the T border regions, the colE1 replication of origin and a wide host range replicon. The binary Ti vectors carrying an expression cassette of the invention can be used to transform both prokaryotic and eukaryotic cells but is preferably used to transform dicot plant cells.

In Vitro Screening of Expression Cassettes: Once the expression cassette is constructed and subcloned into a suitable plasmid, it can be screened for the ability to substantially inhibit the translation of an mRNA coding for a seed storage protein by standard methods such as hybrid arrested translation. For example, for hybrid selection or arrested translation, a preselected antisense DNA sequence is subcloned into an SP6MT containing plasmids (as supplied by ProMega Corp.). For transformation of plants cells, suitable vectors include plasmids such as described herein. Typically, hybrid arrest translation is an in vitro assay that measures the inhibition of translation of an mRNA encoding a particular seed storage protein. This screening method can also be used to select and identify preselected antisense DNA sequences that inhibit translation of a family or subfamily of zein protein genes. As a control, the corresponding sense expression cassette is introduced into plants and the phenotype assayed.

DNA Delivery of the DNA Molecules into Host Cells: The present invention generally includes steps directed to introducing IPMS1 nucleic acids, such as a preselected cDNA encoding the modified IPMS1 enzyme, into a recipient cell to create a transformed cell. In some instances, the frequency of occurrence of cells taking up exogenous (foreign) DNA may be low. Moreover, it is most likely that not all recipient cells receiving DNA segments or sequences will result in a transformed cell wherein the DNA is stably integrated into the plant genome and/or expressed. Some may show only initial and transient gene expression. However, certain cells from virtually any dicot or monocot species may be stably transformed, and these cells regenerated into transgenic plants, through the application of the techniques disclosed herein.

Another aspect of the invention is a plant with isopropylmalate synthase activity, increased amino acid content, normal to increased biomass, wherein the plant has a modified IPMS nucleic acid. The modified IPMS nucleic acid can be from any species. This application provides examples of modified IPMS nucleic acids and proteins that can be used.

The plants and seeds can be monocotyledon or dicotyledon plants and seeds. Another aspect of the invention includes plant cells (e.g., embryonic cells or other cell lines) that can regenerate fertile transgenic plants and/or seeds. The cells can be derived from either monocotyledons or dicotyledons.

Suitable examples of plant and IPMS species include grasses, softwoods, hardwoods, or agricultural crop species. For example, the species of the IPMS nucleic acids and proteins employed as well as the species of modified plant cells, plants, and seeds can be a species of alfalfa, canola, corn, wheat, rice, maize, barley, rye, Brachypodium, Arabidopsis, oats, sorghum, millet, miscanthus, switchgrass, poplar, eucalyptus, sugarcane, bamboo, bean, tobacco, cucumber, tomato, lettuce, pea, soybean, and the like. In some embodiments, the IPMS, plant or cell is a monocotyledon IPMS, plant, seed, or cell. For example, the IPMS, plant or cell can be a grass IPMS, plant, seed, or cell. In some embodiments, the IPMS, plant, seed, or cell is a dicotyledon IPMS, plant, seed, or cell. For example, IPMS, plant, seed, or cell can be a hardwood IPMS, plant, seed, or cell. The cell(s) may be in a suspension cell culture or may be in an intact plant part, such as an immature embryo, or in a specialized plant tissue, such as callus, such as Type I or Type II callus.

Transformation of the cells of the plant tissue source can be conducted by any one of a number of methods can be used. Examples are: Transformation by direct DNA transfer into plant cells by electroporation (U.S. Pat. Nos. 5,384,253 and 5,472,869, Dekeyser et al., The Plant Cell. 2:591-602 (1990)); direct DNA transfer to plant cells by PEG precipitation (Hayashimoto et al., Plant Physiol. 93:857-863 (1990)); direct DNA transfer to plant cells by microprojectile bombardment (McCabe et al., Bio/Technology. 6:923-926 (1988); Gordon-Kamm et al., The Plant Cell. 2:603-618 (1990); U.S. Pat. Nos. 5,489,520; 5,538,877; and 5,538,880) and DNA transfer to plant cells via infection with Agrobacterium. Methods such as microprojectile bombardment or electroporation can be carried out with “naked” DNA where the expression cassette may be simply carried on any E. coli-derived plasmid cloning vector. In the case of viral vectors, it is desirable that the system retain replication functions, but lack functions for disease induction.

One method for dicot transformation, for example, involves infection of plant cells with Agrobacterium tumefaciens using the leaf-disk protocol (Horsch et al., Science 227:1229-1231 (1985). Monocots such as Zea mays can be transformed via microprojectile bombardment of embryogenic callus tissue or immature embryos, or by electroporation following partial enzymatic degradation of the cell wall with a pectinase-containing enzyme (U.S. Pat. Nos. 5,384,253; and 5,472,869). For example, embryogenic cell lines derived from immature Zea mays embryos can be transformed by accelerated particle treatment as described by Gordon-Kamm et al. (The Plant Cell. 2:603-618 (1990)) or U.S. Pat. Nos. 5,489,520; 5,538,877 and 5,538,880, cited above. Excised immature embryos can also be used as the target for transformation prior to tissue culture induction, selection and regeneration as described in U.S. application Ser. No. 08/112,245 and PCT publication WO 95/06128. Furthermore, methods for transformation of monocotyledonous plants utilizing Agrobacterium tumefaciens have been described by Hiei et al. (European Patent 0 604 662, 1994) and Saito et al. (European Patent 0 672 752, 1995).

Methods such as microprojectile bombardment or electroporation are carried out with “naked” DNA where the expression cassette may be simply carried on any E. coli-derived plasmid cloning vector. In the case of viral vectors, it is desirable that the system retain replication functions, but lack functions for disease induction.

The choice of plant tissue source for transformation will depend on the nature of the host plant and the transformation protocol. Useful tissue sources include callus, suspension culture cells, protoplasts, leaf segments, stem segments, tassels, pollen, embryos, hypocotyls, tuber segments, meristematic regions, and the like. The tissue source is selected and transformed so that it retains the ability to regenerate whole, fertile plants following transformation, i.e., contains totipotent cells. Type I or Type II embryonic maize callus and immature embryos are preferred Zea mays tissue sources. Similar tissues can be transformed for softwood or hardwood species. Selection of tissue sources for transformation of monocos is described in detail in U.S. application Ser. No. 08/112,245 and PCT publication WO 95/06128.

The transformation is carried out under conditions directed to the plant tissue of choice. The plant cells or tissue are exposed to the DNA or RNA carrying the IPMS1 nucleic acids for an effective period of time. This may range from a less than one second pulse of electricity for electroporation to a 2-3 days co-cultivation in the presence of plasmid-bearing Agrobacterium cells. Buffers and media used will also vary with the plant tissue source and transformation protocol. Many transformation protocols employ a feeder layer of suspended culture cells (tobacco or Black Mexican Sweet corn, for example) on the surface of solid media plates, separated by a sterile filter paper disk from the plant cells or tissues being transformed.

An Example of Production and Characterization of Stable Transgenic Maize: After effecting delivery of a modified IPMS1 nucleic acid to recipient cells by any of the methods discussed above, the transformed cells can be identified for further culturing and plant regeneration. As mentioned above, to improve the ability to identify transformants, one may desire to employ a selectable or screenable marker gene as, or in addition to, the expressible IPMS1 nucleic acids. In this case, one would then generally assay the potentially transformed cell population by exposing the cells to a selective agent or agents, or one would screen the cells for the desired marker gene trait.

Selection: An exemplary embodiment of methods for identifying transformed cells involves exposing the bombarded cultures to a selective agent, such as a metabolic inhibitor, an antibiotic, herbicide or the like. Cells which have been transformed and have stably integrated a marker gene conferring resistance to the selective agent used, will grow and divide in culture. Sensitive cells will not be amenable to further culturing.

To use the bar-bialaphos or the EPSPS-glyphosate selective system, bombarded tissue is cultured for about 0-28 days on nonselective medium and subsequently transferred to medium containing from about 1-3 mg/l bialaphos or about 1-3 mM glyphosate, as appropriate. While ranges of about 1-3 mg/l bialaphos or about 1-3 mM glyphosate can be employed, it is proposed that ranges of at least about 0.1-50 mg/l bialaphos or at least about 0.1-50 mM glyphosate will find utility in the practice of the invention. Tissue can be placed on any porous, inert, solid or semi-solid support for bombardment, including but not limited to filters and solid culture medium. Bialaphos and glyphosate are provided as examples of agents suitable for selection of transformants, but the technique of this invention is not limited to them.

An example of a screenable marker trait is the red pigment produced under the control of the R-locus in maize. This pigment may be detected by culturing cells on a solid support containing nutrient media capable of supporting growth at this stage and selecting cells from colonies (visible aggregates of cells) that are pigmented. These cells may be cultured further, either in suspension or on solid media. The R-locus is useful for selection of transformants from bombarded immature embryos. In a similar fashion, the introduction of the C1 and B genes will result in pigmented cells and/or tissues.

The enzyme luciferase is also useful as a screenable marker in the context of the present invention. In the presence of the substrate luciferin, cells expressing luciferase emit light which can be detected on photographic or X-ray film, in a luminometer (or liquid scintillation counter), by devices that enhance night vision, or by a highly light sensitive video camera, such as a photon counting camera. All of these assays are nondestructive and transformed cells may be cultured further following identification. The photon counting camera is especially valuable as it allows one to identify specific cells or groups of cells which are expressing luciferase and manipulate those in real time.

It is further contemplated that combinations of screenable and selectable markers may be useful for identification of transformed cells. For example, selection with a growth inhibiting compound, such as bialaphos or glyphosate at concentrations below those providing 100% inhibition followed by screening of growing tissue for expression of a screenable marker gene such as luciferase would allow one to recover transformants from cell or tissue types that are not amenable to selection alone. In an illustrative embodiment embryogenic Type II callus of Zea mays L, can be selected with sub-lethal levels of bialaphos. Slowly growing tissue was subsequently screened for expression of the luciferase gene and transformants can be identified.

Regeneration and Seed Production: Cells that survive the exposure to the selective agent, or cells that have been scored positive in a screening assay, are cultured in media that supports regeneration of plants. One example of a growth regulator that can be used for such purposes is dicamba or 2,4-D. However, other growth regulators may be employed, including NAA, NAA+2,4-D or perhaps even picloram. Media improvement in these and like ways can facilitate the growth of cells at specific developmental stages. Tissue can be maintained on a basic media with growth regulators until sufficient tissue is available to begin plant regeneration efforts, or following repeated rounds of manual selection, until the morphology of the tissue is suitable for regeneration, at least two weeks, then transferred to media conducive to maturation of embryoids. Cultures are typically transferred every two weeks on this medium. Shoot development signals the time to transfer to medium lacking growth regulators.

The transformed cells, identified by selection or screening and cultured in an appropriate medium that supports regeneration, can then be allowed to mature into plants. Developing plantlets are transferred to soilless plant growth mix, and hardened, e.g., in an environmentally controlled chamber at about 85% relative humidity, about 600 ppm CO₂, and at about 25-250 microeinsteins/sec·m² of light. Plants can be matured either in a growth chamber or greenhouse. Plants are regenerated from about 6 weeks to 10 months after a transformant is identified, depending on the initial tissue. During regeneration, cells are grown on solid media in tissue culture vessels. Illustrative embodiments of such vessels are petri dishes and Plant Con™. Regenerating plants can be grown at about 19° C. to 28° C. After the regenerating plants have reached the stage of shoot and root development, they may be transferred to a greenhouse for further growth and testing.

Mature plants are then obtained from cell lines that are known to express the trait. In some embodiments, the regenerated plants are self-pollinated. In addition, pollen obtained from the regenerated plants can be crossed to seed grown plants of agronomically important inbred lines. In some cases, pollen from plants of these inbred lines is used to pollinate regenerated plants. The trait is genetically characterized by evaluating the segregation of the trait in first and later generation progeny. The heritability and expression in plants of traits selected in tissue culture are of interest if the traits are to be commercially useful.

Regenerated plants can be repeatedly crossed to inbred plants to introgress the IPMS1 nucleic acids into the genome of the inbred plants. This process is referred to as backcross conversion. When a sufficient number of crosses to the recurrent inbred parent have been completed to produce a product of the backcross conversion process that is substantially isogenic with the recurrent inbred parent except for the presence of the introduced IPMS1 nucleic acids, the plant is self-pollinated at least once to produce a homozygous backcross converted inbred containing the modified IPMS1 nucleic acids. Progeny of these plants are true breeding.

Alternatively, seed from transformed monocot plants regenerated from transformed tissue cultures is grown in the field and self-pollinated to generate true breeding plants.

Seed from the fertile transgenic plants can then be evaluated for the presence and/or expression of the IPMS1 nucleic acids (or IPMS1 proteins). Transgenic plant and/or seed tissue can be analyzed for modified IPMS1 expression using standard methods such as SDS polyacrylamide gel electrophoresis, liquid chromatography (e.g., HPLC) or other means of detecting a product of IPMS1 activity (e.g., increased amino acid content and/or biomass).

Once a transgenic seed expressing the modified IPMS1 sequence and having an increase in amino acid content in the plant is identified, the seed can be used to develop true breeding plants. The true breeding plants are used to develop a line of plants with an increase in the percent of amino acid content (e.g., of various amino acids) and growth or biomass of the plant while still maintaining other desirable functional agronomic traits. Adding the trait of increased amino acid content (with or without normal to improved biomass) of the plant can be accomplished by back-crossing with this trait and with plants that do not exhibit this trait and studying the pattern of inheritance in segregating generations. Those plants expressing the target trait in a dominant fashion are preferably selected. Back-crossing is carried out by crossing the original fertile transgenic plants with a plant from an inbred line exhibiting desirable functional agronomic characteristics while not necessarily expressing the trait of an increased percent of isopropylmalate synthase activity, normal to improved growth, and/or protein folding in the plant. The resulting progeny are then crossed back to the parent that expresses the increased IPMS1 trait (increased amino acids, with or without normal to improved biomass). The progeny from this cross will also segregate so that some of the progeny carry the trait and some do not. This back-crossing is repeated until an inbred line with the desirable functional agronomic traits, and with expression of the trait involving an increase in amino acid content with or without normal to improved biomass of the plant. Such expression of the increased amino acid content with or without normal to improved biomass of a plant can be expressed in a dominant fashion.

Subsequent to back-crossing, the new transgenic plants can be evaluated for an increase in the weight percent of various amino acids, increased modified isopropylmalate synthase activity, and/or normal to improved biomass of the plant. This can be done, for example, by immunofluorescence analysis of whole plant cell walls (e.g., by microscopy), isopropylmalate synthase activity assays, amino acid content analyses, biomass measurements, and any of the assays described herein or available to those of skill in the art.

The new transgenic plants can also be evaluated for a battery of functional agronomic characteristics such as lodging, kernel hardness, yield, resistance to disease, resistance to insect pests, drought resistance, and/or herbicide resistance.

As described herein, expression of IPMS1 can not only increase the amino acid content of plant tissues but such expression can also increase the biomass, growth or height of plants. Hence it is useful to modify a variety of plant types to express IPMS1.

Plants that can be improved include but are not limited to forage plants (e.g., alfalfa, clover, soybeans, turnips, bromegrass, bluestem, and fescue), starch or oil plants (e.g., canola, potatoes, lupins, sunflower, soybean, and cottonseed), grains (maize, wheat, barley, oats, rice, sorghum, millet and rye), grasses (switchgrass, prairie grass, wheat grass, sudangrass, sorghum, straw-producing plants, miscanthus, switchgrass), sugar producing plants (sugarcane, beets), vegetable plants (e.g., cucumber, lettuce, tomato), Brachypodium, Arabidopsis, bamboo, softwood, hardwood and other woody plants (e.g., those used for paper production such as poplar species, pine species, and eucalyptus). In some embodiments the plant is an agricultural crop species, or a species useful for foraging by agricultural animals. Plants useful for generating dairy forage include legumes such as alfalfa, as well as clover, soybeans, turnips, Brachypodium, Arabidopsis, and forage grasses such as bromegrass, and bluestem. In some cases, the plant is an oil producing plant such as canola, corn, soybean, sunflower, walnut, olive, or the like.

The IPMS nucleic acids or IPMS proteins that are modified can be from the same species as the plant, plant cell, or plant seed that is modified to include the modified IPMS1 nucleic acids or modified IPMS1 proteins. In other cases, the IPMS nucleic acids or IPMS proteins that are modified can be from a different species from the plant, plant cell, or plant seed that is modified to include the modified IPMS1 nucleic acids or the modified IPMS1 proteins.

Determination of Stably Transformed Plant Tissues: To confirm the presence of the IPMS1 nucleic acids in the regenerating plants, or seeds or progeny derived from the regenerated plant, a variety of assays may be performed. Such assays include, for example, molecular biological assays available to those of skill in the art, such as Southern and Northern blotting and PCR; biochemical assays, such as detecting the presence of a protein product. e.g., by immunological means (ELISAs and Western blots) or by enzymatic function; plant part assays, such as leaf, seed or root assays; and also, by analyzing the phenotype of the whole regenerated plant.

Whereas DNA analysis techniques may be conducted using DNA isolated from any part of a plant. RNA may only be expressed in particular cells or tissue types and so RNA for analysis can be obtained from those tissues. PCR techniques may also be used for detection and quantification of RNA produced from introduced IPMS1 nucleic acids. PCR also be used to reverse transcribe RNA into DNA, using enzymes such as reverse transcriptase, and then this DNA can be amplified by use of conventional PCR techniques. Further information about the nature of the RNA product may be obtained by Northern blotting. This technique will demonstrate the presence of an RNA species and give information about the integrity of that RNA. The presence or absence of an RNA species can also be determined using dot or slot blot Northern hybridizations. These techniques are modifications of Northern blotting and also demonstrate the presence or absence of an RNA species.

While Southern blotting and PCR may be used to detect the IPMS1 nucleic acid in question, they do not provide information as to whether the preselected DNA segment is being expressed. Expression may be evaluated by specifically identifying the protein products of the introduced modified IPMS1 nucleic acids or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange, liquid chromatography or gel exclusion chromatography. The unique structures of individual proteins offer opportunities for use of specific antibodies to detect their presence in formats such as an ELISA assay. Combinations of approaches may be employed with even greater specificity such as Western blotting in which antibodies are used to locate individual gene products that have been separated by electrophoretic techniques. Additional techniques may be employed to absolutely confirm the identity of the modified IPMS1 sequences such as by amino acid sequencing following purification of IPMS1 nucleic acid or IPMS1 protein. The Examples of this application also provide assay procedures for detecting and quantifying IPMS1 activity. Other procedures may be additionally used.

The expression of a gene product can also be determined by evaluating the phenotypic results of its expression. These assays also may take many forms including but not limited to analyzing changes in the chemical composition, morphology, or physiological properties of the plant. Chemical composition may be altered by expression of preselected DNA segments encoding storage proteins which change amino acid composition and may be detected by amino acid analysis.

### Definitions

As used herein, the term “plant” is used in its broadest sense. It includes, but is not limited to, any species of grass (e.g. forage, grain-producing, turf grass species), ornamental or decorative, crop or cereal, fodder or forage, fruit or vegetable, fruit plant or vegetable plant, herb plant, woody plant, flower plant or tree. It is not meant to limit a plant to any particular structure. It also refers to a unicellular plant (e.g. microalga) and a plurality of plant cells that are largely differentiated into a colony (e.g. volvox) or a structure that is present at any stage of a plant's development. Such structures include, but are not limited to, a seed, a tiller, a sprig, a stolen, a plug, a rhizome, a shoot, a stem, a leaf, a flower petal, a fruit, et cetera.

As used herein, “isolated” means a nucleic acid or polypeptide has been removed from its natural or native cell. Thus, the nucleic acid or polypeptide can be physically isolated from the cell or the nucleic acid or polypeptide can be present or maintained in another cell where it is not naturally present or synthesized.

The term “transgenic” when used in reference to a plant or leaf or fruit or seed or plant biomass, for example a “transgenic plant.” transgenic leaf,” “transgenic fruit.” “transgenic fruit,” “transgenic seed,” “transgenic biomass,” or a “transgenic host cell” refers to a plant or leaf or fruit or seed or biomass that contains at least one heterologous or foreign gene in one or more of its cells. The term “transgenic plant material” refers broadly to a plant, a plant structure, a plant tissue, a plant seed or a plant cell that contains at least one heterologous gene in one or more of its cells.

The term “transgene” refers to a foreign gene that is placed into an organism (e.g. a plant) or host cell by the process of transfection. The term “foreign gene” or heterologous gene refers to any nucleic acid (e.g., gene sequence) that is introduced into the genome of an organism or tissue of an organism or a host cell by experimental manipulations, such as those described herein, and may include gene sequences found in that organism so long as the introduced gene does not reside in the same location, as does the naturally occurring gene.

As used herein, a “native” nucleic acid or polypeptide means a DNA. RNA or amino acid sequence or segment that has not been manipulated in vitro, i.e., has not been isolated, purified, and/or amplified.

As used herein, the term “wild-type” when made in reference to a gene refers to a functional gene common throughout an outbred population. As used herein, the term “wild-type” when made in reference to a gene product refers to a functional gene product common throughout an outbred population. A functional wild-type gene is that which is most frequently observed in a population and is thus arbitrarily designated the “normal” or “wild-type” form of the gene. As used herein, the term “wild-type” when made in reference to a plant refers to the plant type common throughout an outbred population that has not been genetically manipulated to contain an expression cassette, e.g., any of the expression cassettes described herein.

The following Examples illustrate some of the experimental work performed and materials used in the development of the invention. Appendix A may provide further information.

### Example 1: Materials and Methods

This Example illustrates some of the materials and methods used in the development of the invention.

### Resources

Table 4 below lists some of the resources used in the experiments described herein.

TABLE 4

| Reagent type | | | | |
|---|---|---|---|---|
| (species) or | | Source or | | Additional |
| resource | Designation | reference | Identifiers | information |
| Gene | AtIPMS1 | | TAIR: AT1G18500 | |
| (Arabidopsis thaliana) | | | | |
| Gene | AtOMR1 | | TAIR: AT3G10050 | |
| (Arabidopsis thaliana) | | | | |
| Gene | AtAHASS1 | | TAIR: AT2G31810 | |
| (Arabidopsis thaliana) | | | | |
| Gene | AtAHASS2 | | TAIR: AT5G16290 | |
| (Arabidopsis thaliana) | | | | |
| Gene | AtTOR | | TAIR: AT1G50030 | |
| (Arabidopsis thaliana) | | | | |
| Gene | AtRaptor1B | | TATR: AT3G08850 | |
| (Arabidopsis thaliana) | | | | |
| Genetic reagent | eva1 | this work | | EMS line with |
| (Arabidopsis thaliana) | | | | a mutation of |
| | | | | IPMS1 |
| Genetic reagent | ipms1-4 | Xing and | SALK_101771 | |
| (Arabidopsis thaliana) | | Last, 2017 | | |
| Genetic reagent | ipms1-5 | Xing and | WiseDsLoxHs221_05F | |
| (Arabidopsis thaliana) | | Last, 2017 | | |
| Genetic reagent | tfl111 | Xing and | TAIR: CS69734 | |
| (Arabidopsis thaliana) | (ipms1-1^{D}) | Last, 2017 | | |
| Genetic reagent | tfl102 | Xing and | TAIR: CS69733 | |
| (Arabidopsis thaliana) | (ipms1-1^{D}) | Last, 2017 | | |
| Genetic reagent | ahass1-1 | Xing and | SALK_096207 | |
| (Arabidopsis thaliana) | | Last, 2017 | | |
| Genetic reagent | ahass2-7 | Xing and | WiseDsLoxHs009_02G | |
| (Arabidopsis thaliana) | | Last, 2017 | | |
| Genetic reagent | ahass2-1^{D} | Xing and | TAIR: CS69724 | |
| (Arabidopsis thaliana) | | Last, 2017 | | |
| Genetic reagent | omr1-11^{D} | Xing and | TAIR: CS69720 | |
| (Arabidopsis thaliana) | | Last, 2017 | | |
| Genetic reagent | tor-es | Xiong and | TAIR: CS69829 | |
| (Arabidopsis thaliana) | | Sheen, 2012 | | |
| Genetic reagent | raptor1b | Salem et | SALK_022096 | |
| (Arabidopsis thaliana) | | al., 2017 | | |
| Antibody | Anti-S6K (Rabbit | Agrisera | AS12 1855 | Western |
| | polyclonal) | | | blotting |
| | | | | (1:1000 |
| | | | | dilution) |
| Antibody | Anti-S6K- | Abcam | ab207399 | Western |
| | phosphorylated | | | blotting |
| | (Rabbit | | | (1:1000 |
| | polyclonal) | | | dilution) |
| Antibody | HRP conjugated | Sigma- | A0545 | Western |
| | anti-rabbit (Goat | Aldrich | | blotting |
| | polyclonal) | | | (1:10000 |
| | | | | dilution) |
| Commercial | Click-iT EdU | Invitrogen | C10337 | |
| assay or kit | Alexa Fluor 488 | | | |
| | Imaging Kit | | | |

The T-DNA insertional mutants ipms1-4 (SALK_101771), ipms1-5 (WiscDsLoxHs221_05F), ipms2-1 (WiscDsLox426A07), ipms2-2 (SALK_046876), ahass2-7 (WiscDsLoxHs009_02G), ahass2-8 (WiscDsLoxHs110_12G), ahass1-1 (SALK_096207), and ahass1-2 (SALK_108628) can be obtained from ABRC (see website at abrc.osu.edu).

An EMS mutant line was first identified in a screen for vacuolar phenotypes (Avila et al., 2003) was crossed with wild type (Col-0) for three times to obtain a progeny with consistently inherited vacuolar and growth phenotypes, which was designated as eva1.

Except for chronical treatments with specified chemicals, Arabidopsis seeds were stratified and grown on medium containing half-strength Linsmaier and Skoog nutrients (½ LS; Caisson Labs, LSP03), 1% sucrose and 0.4% phytagel (Sigma-Aldrich, P8169) in chambers configured with 21° C. and 16-hour light: 8-hour dark cycle.

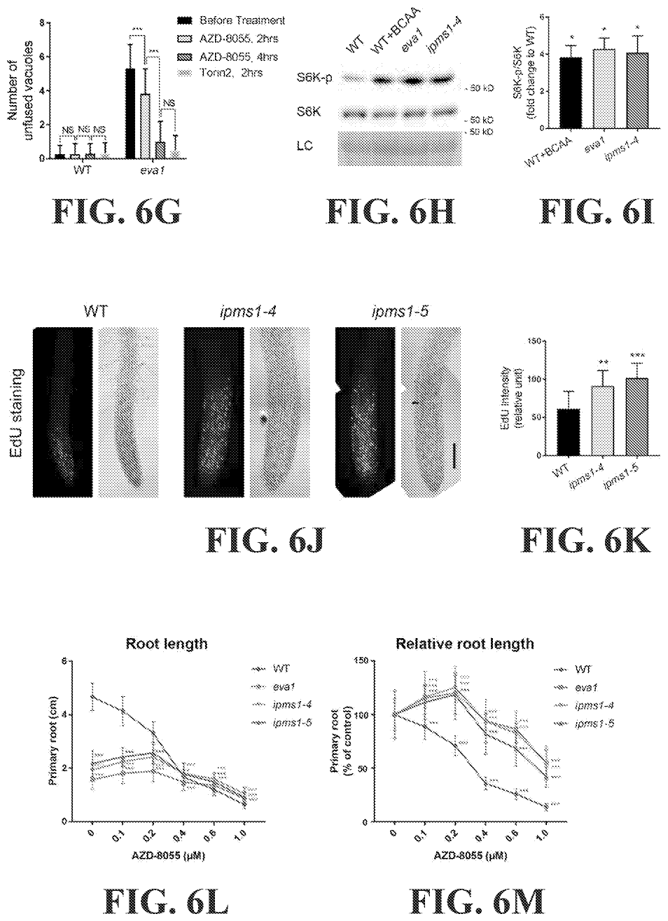

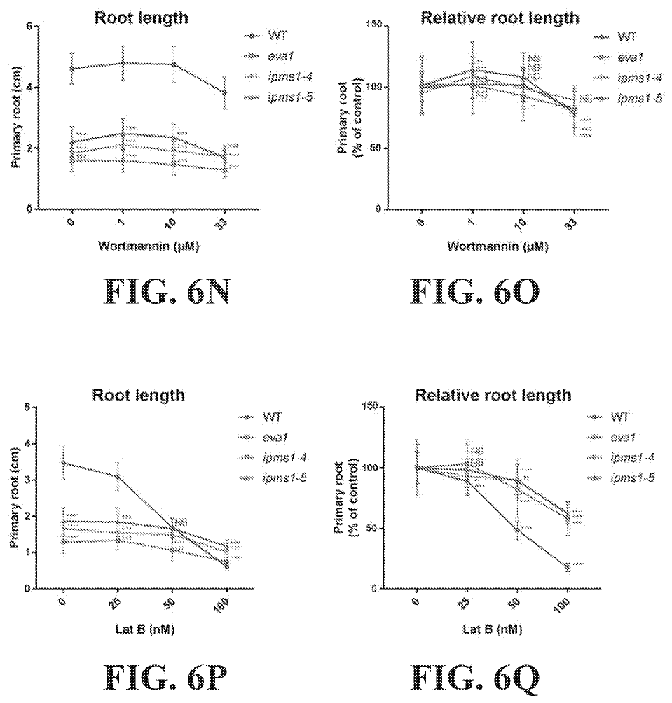

To examine the effect of latrunculin B (Lat B) on root elongation (FIG. 6L-6Q), wild type (Col-0), ipms1-4 and ipms1-5 lines germinated and grew on horizontally staged Petri dishes containing Arabidopsis growth medium (½ LS, 1% sucrose and 0.4% phytagel). 10-day old seedlings were transplanted to Petri dishes containing ½ LS, 1% sucrose and 1% Agar (Acumedia, 7558A) medium containing DMSO or 50 nM Lat B or 100 nM Lat B. Photographs were acquired immediately after the transplant and the Petri dishes were vertically staged in a Percival chamber.

Photographs were also acquired 8 days after the transplant.

In another pharmaceutical examination using AZD-8055, wortmannin and Lat B (FIG. 6A-6G, 6L-6Q), wild type (Col-0), eva1, ipms1-4 and ipms1-5 lines germinated and grew on vertically staged Petri dishes containing Arabidopsis growth medium containing specific chemical inhibitors.

Exogenous feeding of 1 mM BCAA was performed by stratification and germination of seeds on ½ LS, 1% sucrose and 1% Agar medium containing 1 mM equal concentrations of Ile, Val and Leu. L-Isoleucine (Sigma-Aldrich, I2752), L-Valine (Sigma-Aldrich, V0500) and L-Leucine (Sigma-Aldrich, L8000) were dissolved in water to prepare 1 M stock solutions, which were filtered by Millex-GS 0.22 μm filter units (Millipore, SLGS033SS).

### Confocal Microscopy

A Zeiss LSM 510 META and a Nikon A1Rsi laser scanning confocal microscope were used for imaging. Acquired images were handled by NIS-Elements Advanced Research (Nikon), ZEN (Zeiss) and Fiji (ImageJ) (Schindelin et al., 2012). The fluorescent protein fusions used in this study are GFP-δTIP (Cutler et al., 2000), ERYK (Nelson et al., 2007), YFP-ABD2 (Sheahan et al., 2004), GFP-CASP (Renna et al., 2005), SEC-RFP (Faso et al., 2009) and -TIP-YFP (Nelson et al., 2007). Transformation of Arabidopsis plants were conducted using floral dip method (Clough and Bent, 1998).

### Quantitative Analysis of ER Morphology and Actin Cytoskeletal Organization

Image acquisition and further evaluation of the ER cisternae was conducted using a previously described method (Cao et al., 2016) that measures the occupancy of ER area in a region of interest. Analyses of the actin cytoskeletal organization were performed following a previously described procedure (Lu and Day, 2017). Briefly, Z-stack images with 0.5 μm intervals were acquired to cover the whole epidermal cell. The Z-stack series were converted to maximal projection images using NIS-Elements Advanced Research (Nikon) and Fiji (ImageJ) (Schindelin et al., 2012). Utilizing two ImageJ macros that were previously generated (Lu and Day, 2017), skewness was measured to present the distribution of YFP-ABD2 fluorescence intensity and occupancy was measured for the density of skeletonized YFP-ABD2 fluorescence signal.

### Chemical Stocks and Treatments

All temporal chemical treatments were performed using 10-day old seedlings. Each of the following chemicals was first dissolved in DMSO to prepare a stock solution, and then diluted in Arabidopsis growth medium (½ LS and 1% sucrose) to reach the specific working concentration. 33 μM Wortmannin (Sigma-Aldrich, W1628) and 100 μM LY294002 (MedChemExpress, HY-10108) were used to treat seedlings for 2 hr. Latrunculin B (Sigma-Aldrich, L5288) and Oryzalin (Chem Service Inc, N-12729) were diluted to 25 μM and 40 μM, respectively, for 2-hour treatments. For TOR inhibition, seedlings were incubated with 5 μM AZD-8055 (MedChemExpress, HY-10422) or 1 μM Torin2 (MedChemExpress, HY-13002) for 2 or 4 hours as the figure legends indicated. 10 μM solution of β-estradiol (Sigma-Aldrich, E8875) was used to induce gene silencing.

### Amino Acid Extraction and LC-MS/MS Analysis

Plants used for amino acid extraction were grown under standard conditions for 10 or 20 days. The aerial tissue (fresh weight around 10 mg) was harvested into a 2 mL tube with two 3 mm steel beads and flash frozen in liquid N2. Tissue was either used immediately or stored at −80° C. until extraction. Tissue was pulverized using a mixer mill (Retsch Mill, MM400) for 1 min at 30 times per second. Amino acids were extracted as previously reported (Xing and Last, 2017; Angelovici et al., 2013). Briefly, an amino acid extraction buffer was prepared with ˜2 μM heavy labeled amino acids standards (13C, 15N, Sigma-Aldrich), 10 μM 1,4-dithiothreitol (DTT, Sigma-Aldrich), and 10 mM perfluoroheptanoic acid (PFHA, Sigma-Aldrich). To the ground tissue, 350 μL of extraction buffer was added, vortexed for 10 s and heated at 90° C. for 10 min. Tubes were cooled on ice for 5 min and centrifuged for 10 min at 4° C. at 13,000×g. The supernatant was applied to a low-binding hydrophilic 0.2 μm centrifugal polytetrafluoroethylene (PTFE) filter (Millipore, UFC30LG25) and centrifuged for 5 min at 3,500×g. 150 μL flow through was transferred to 2 mL glass vials with glass insert for LC-MS analysis.

Amino acid detection and quantification by LC-MS/MS was performed as previously reported (Xing and Last, 2017; Angelovici et al., 2013). Briefly, a dilution series (12.2 nM to 250 μM) of each individual amino acid standard was made containing the same concentration of the heavy standards as was in the amino acid extraction buffer. Samples were injected into a Quattro micro API LC/MS/MS (Waters) equipped with an Acquity UHPLC HSS T3 1.8 μm column (Waters) using a three-function method. A 13 min LC method was used with solvent A (10 mM PFHA) and solvent B (acetonitrile) at a flow rate of 0.3 mL/minute. Amino acids were quantified by comparison to their standard curves using QuanLynx.

### EdU Staining

EdU (5-ethynyl-2′-deoxyuridine) staining of root apical meristem was performed using Click-iT EdU Alexa Fluor 488 Imaging Kit (Invitrogen, C10337), following a protocol that was adapted for plant tissues (Kotogdny et al., 2010). Labeling was performed by incubating 10-day old Arabidopsis seedlings in 10 μM EdU in Arabidopsis growth medium (½ LS and 1% sucrose) for 30 min in a Percival chamber. All samples were then incubated with a fixation buffer (4% formaldehyde, 0.1% Triton X-100, 1×PBS) for 30 min. All samples were washed for three times, 10 min each, with 1×PBS after fixation. The EdU detection was conducted by 30 min incubation in dark with the Click-iT cocktail, which was prepared according to the manual of Click-iT EdU Alexa Fluor 488 Imaging Kit. Each sample was immediately washed for three times, 10 min each, with 1×PBS before imaging.

### PI Staining of Root Tip and Measurement

PI (propidium iodide) staining was conducted by 3 min incubation of Arabidopsis seedlings in propidium iodide (Invitrogen. P3566) diluted to 1 μg/mL using Arabidopsis growth medium (½ LS and 1% sucrose). After staining, all samples were immediately washed for 1 min and then subjected to imaging. Confocal images of propidium iodide stained root tips were analyzed using Cell-O-Tape (French et al., 2012), which is a plugin of ImageJ that automatically segments three zones in a root tip (the meristem, the transition zone and the mature zone) by comparing the lengths of adjacent cells in the same cortical layer. Adjacent cells with significant increase in cell length belong to the transition zone. Cells before and after the transition zone are categorized as cells in the meristem and the mature zone respectively. The program records the length of each cell and the cell number in each zone.

### Protein Preparation and Immunoblotting

To detect the phosphorylation status of S6K, 50 mg plant aerial tissue was used for protein extraction using 1.5 mL extraction buffer of 1×PBS, pH 7.4, containing 250 mM sucrose. Protease Inhibitor Cocktail (Sigma-Aldrich, P9599) and PhosSTOP phosphatase inhibitor (Roche. 4906845001). Three times of centrifugation, 1 k×g for 5 min, 14 k×g for 5 min and 135 k×g for 30 min, were conducted to separate the soluble proteins. The supernatant from the last centrifugation was separated, concentrated to 200 μL using an Amicon Ultra centrifugal unit (Millipore, UFC501024), and then mixed with 40 μL 6× Laemmli buffer. Proteins were denatured by incubation at 95° C. for 10 min. Protein samples were separated on 15% SDS-PAGE with 8M urea and blotted to PVDF membranes (Bio-Rad, 1620177). Blots were blocked with 5% milk for 1 hour at room temperature. Blots were incubated with primary antibodies of either anti-S6K (Agrisera, AS12 1855) or anti-S6K-phosphorylated (Abcam, ab207399) overnight at 4° C. and subsequently with secondary HRP conjugated goat anti-rabbit antibody (Sigma-Aldrich, A0545) for 1 hour at room temperature.

### Extraction and Measurement of Anthocyanins

The aerial parts of 10-day old Arabidopsis seedlings were collected, and then lyophilized and measured for dry weight. Total anthocyanins were extracted using 1 μL extraction buffer (50% methanol containing 3% formic acid) per 50 μg dry weight. After overnight incubation with extraction buffer at room temperature, the supernatant was collected and measured absorbance of 532 nm.

### TEM and Measurement of Leaf Thickness

The electron microscopic imaging of the endomembrane structures and chloroplasts were performed following an established protocol (Kim et al., 2018). In brief, 1 mm×1 mm pieces of cotyledon samples were cut and fixed in TEM fixative buffer (2.5% paraformaldehyde and 2.5% glutaraldehyde in 0.1 M cacodylate buffer, pH 7.4) with vacuum infiltration. The fixed samples were stained with 1% osmium tetroxide overnight at 4° C. After series of dehydration with acetone, the samples were infiltrated and embedded in Spurr's Resin. Sections with 50 nm thickness were cut and mounted on the copper grid and 10 well slides. For TEM, the grids were post-stained in 2% uranyl-acetate for 30 min and then treated with 1% lead citrate for 15 min. JEOL 100CX TEM (JEOL USA) was used to observe the ultrastructure of cotyledon.

The thickness of cotyledons was measured as previously described (Weraduwage et al., 2016). Briefly, 2 mm xl mm samples cut from the center of the cotyledons were fixed in fixative buffer (4% paraformaldehyde and 0.5% glutaraldehyde in 1×PBS, pH 7.4) with vacuum infiltration. The fixed samples were stained with 1% osmium tetroxide overnight at 4° C. After series of dehydration with acetone, the samples were infiltrated and embedded in Spurr's Resin. Sections with 500 nm thickness were cut and mounted on the copper grid and 10 well slides. For leaf thickness analysis, the sections were stained with 1% toluidine blue for 1 min and washed with running water. Images were taken using Axio Imager M2 (Zeiss), and measurement of leaf thickness was performed using AxioVision SE64 Rel. 4.9.1 (Zeiss) software. Three biological samples with three technical replicates were used to measure leaf thickness.

### Example 2: Identification of a Mutant with Defects in Vacuole Morphogenesis

A confocal microscopy-based screen was performed on an EMS-mutagenized population to identify mutants with defects in the subcellular distribution of a GFP-tagged tonoplast intrinsic protein (TIP), GFP-δTIP (Avila et al., 2003; Cutler et al., 2000). The inventors identified mutant line eva1, a mutant characterized by severe defects in vacuole morphology early in development. During the first 10 days after germination, in wild-type (WT) cotyledon epidermal cells, small vacuoles undergo membrane fusion to form a single large central vacuole (Zhang et al., 2014) (FIG. 1A). In contrast, 10-day old eva1 cotyledon epidermal cells displayed numerous additional vacuolar structures that vary in shape and size (FIG. 1B). To further characterize eva1 vacuolar phenotypes, two prominent vacuolar structures were identified that are rarely observed in wild type: trans-vacuolar strands and presumably unfused vacuoles. Trans-vacuolar strands are strands formed upon association between vacuolar membrane and bundled actin filaments (Ueda et al., 2010), which were greatly enhanced in number, length and thickness in eva1 (FIG. 1). The inventors defined presumably unfused vacuoles as spherical structures that are isolated from the large central vacuole and have diameter >5 μm. Both two vacuolar phenotypes were attenuated in 20-day old eva1 cotyledons, which closely resembled WT (FIG. 1C-1D). The eva1 vacuole phenotypes were verified in 10-day old eva1 cotyledons expressing γTIP-YFP (Nelson et al., 2007), which labels the large central vacuole and other vacuolar structures not marked by GFP-δTIP (Gattolin et al., 2010) (FIG. 1). These results support the conclusion that the tonoplast organization and vacuolar morphology are compromised in eva1 in early stages of growth independently from the tonoplast marker used for the analyses.

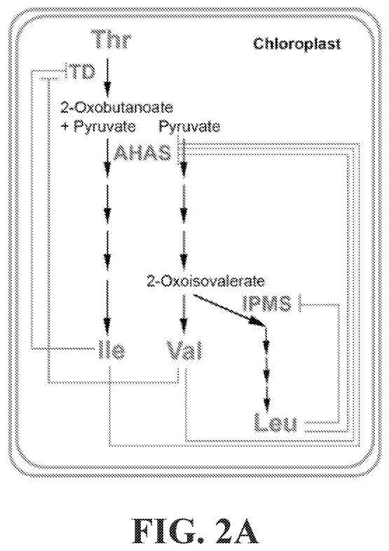

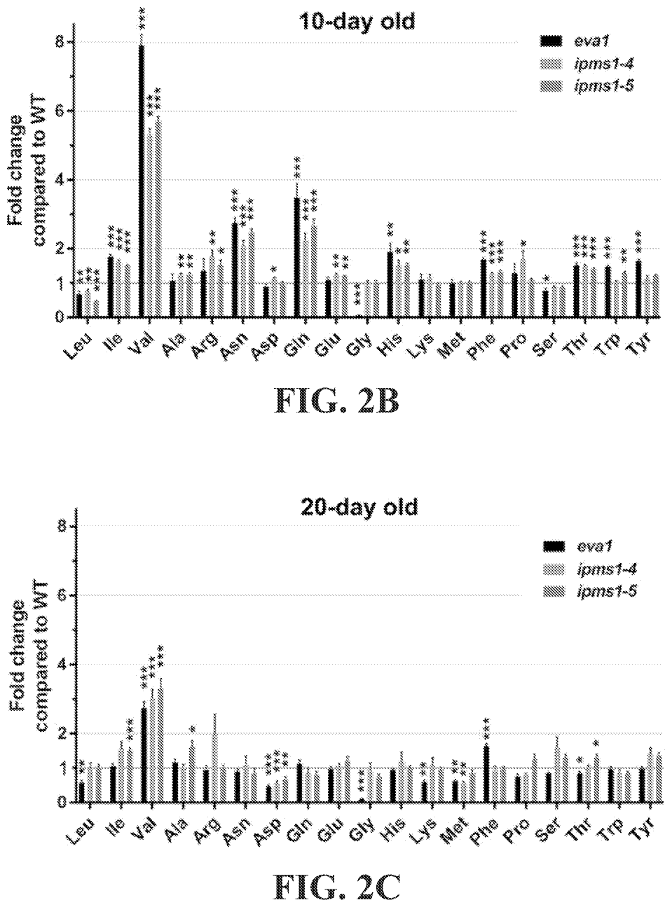

The inventors next identified the causative mutation in eva1. Bulked segregant analysis and whole-genome resequencing narrowed down the eva1 mutation to a G-to-A transition in IPMS1 (ATIG18500) causing an aspartate (Asp)-to-asparagine (Asn) residue substitution (FIG. 1E-1F). IPMS1 catalyzes condensation of 2-oxoisovalerate and acetyl-CoA into 2-isopropylmalate, the committed step for Leu biosynthesis (de Kraker et al., 2007; Field et al., 2004) (FIG. 2A). Homology modeling of IPMS1 predicted that the mutated Asp228 is located in the acetyl-CoA binding surface near the pocket for 2-oxoisovalerate substrate. In addition to eva1, we used three other IPMS1 alleles that had been characterized: two recessive loss-of-function mutants, ipms1-4 and ipms1-5, and a gain-of-function ipms1-1D, with a point mutation that impairs allosteric regulation (Xing and Last, 2017; de Kraker et al., 2007) (FIG. 1E). 10-day old eva1, ipms1-4 and ipms1-5 seedlings exhibited similar delay in emergence of true leaves (FIG. 1G). These growth and developmental phenotypes, as well as the subcellular phenotypes, were attenuated by 20 days of growth (FIG. 1H). The presence of the eva1 phenotypes in the eva1×ipms1-5 F1 progeny confirmed allelism of eva1 to IPMS1 (FIG. 1). Together, these results support that the eva1 vacuole and plant growth phenotypes are correlated to a loss of functional IPMS1, which has a consistent subcellular impact on early stages of growth.

### Example 3: Eva1 Plants Over-Accumulate Valine

The role of IPMS1 in BCAA biosynthesis has been characterized as directing flux towards Leu biosynthesis, and away from the competing product Val (Xing and Last, 2017; de Kraker et al., 2007; Field et al., 2004) (FIG. 2A). The Arabidopsis genome encodes two IPMS isoforms: IPMS1 mRNA accumulates to higher levels than IPMS2 mRNA through most stages of plant growth (de Kraker et al., 2007). An earlier work examined two-week old plants and found that Val and Ile were increased in both ipms1-4 and ipms1-5 but Leu was decreased in ipms1-4 and increased in ipms1-5 (Xing and Last. 2017). To determine the impact of the eva1 mutation on amino acid homeostasis at earlier stages of growth, we conducted free amino acid analysis of 10-day and 20-day old WT and ipms1 mutants. Notably, the eva1, ipms1-4 and ipms1-5 plants had similar increases in Val and total BCAAs and decreases in Leu, consistent with the data that eva1 is a loss-of-function allele of IPMS1 (FIG. 2B-2C). In addition, in these mutants exhibited similar changes in Asp-derived amino acids (threonine-Thr, methionine-Met, lysine-Lys and Ile) and aromatic amino acids (phenylalanine-Phe, tryptophan-Trp and tyrosine-Tyr) (FIG. 2B-2C). Consistent with a disappearance of the subcellular phenotypes of the mutants during growth (FIG. 1A-ID), the impact of ipms1 mutations on amino acid homeostasis was mitigated at 20 days of growth, with the fold change of Val becoming smaller in the mutants versus WT, and the types of amino acids significantly changed in the mutants compared to WT becoming fewer (FIG. 2B-2C). Taken together, these data indicate that eva1 is a loss-of-function mutant of IPMS1 equivalent to ipms1-4 and ipms1-5 and that the alteration in BCAA levels is most notable for increased in Val levels.

### Example 4: Disruption of BCAA Homeostasis Leads to Pleiotropic Defects in Plant Growth and Development

This Example describes experiments designed to evaluate whether the transient changes in BCAA accumulation and vacuole morphology affected early plant growth.

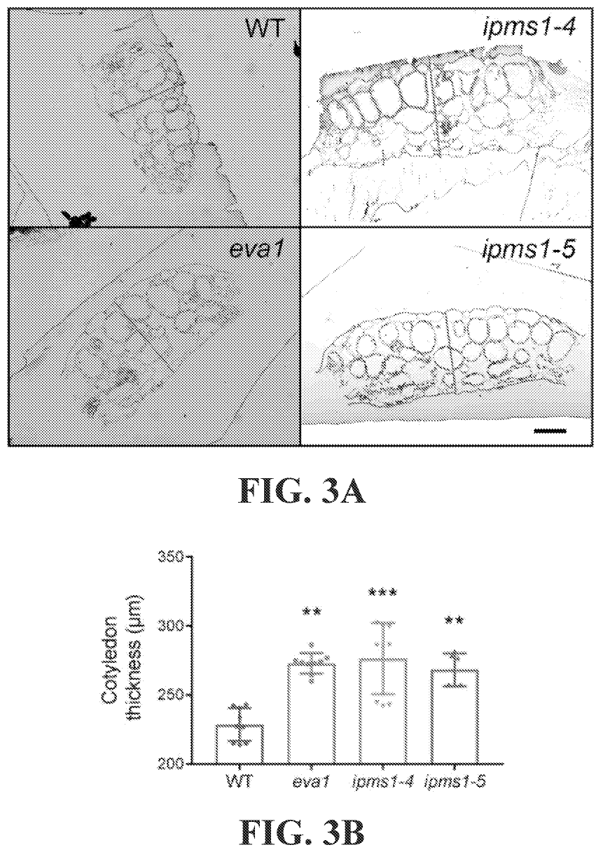

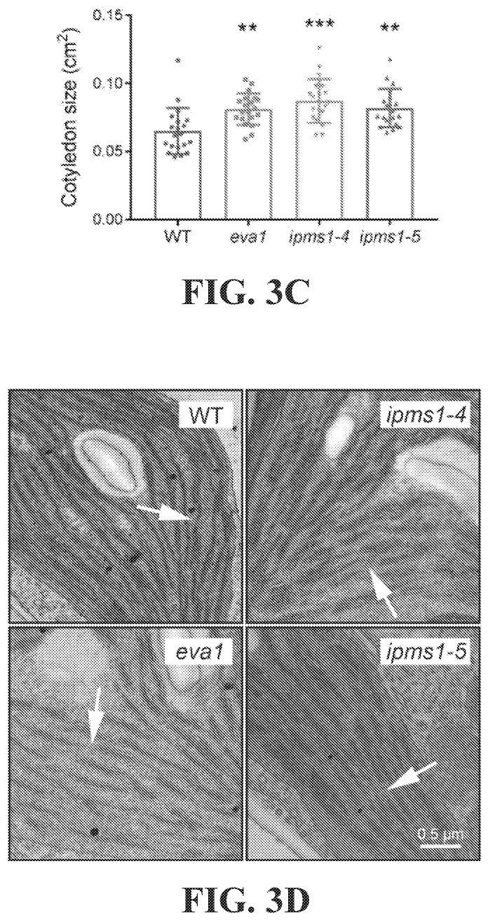

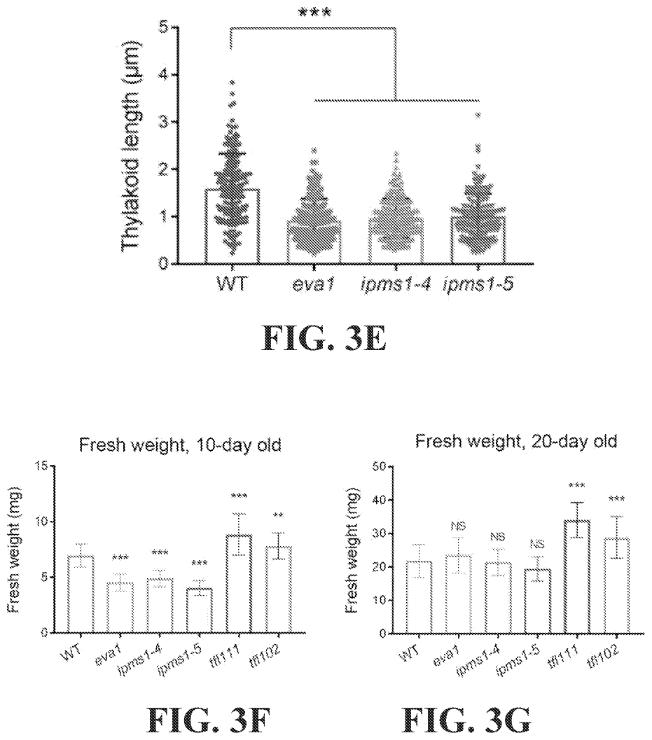

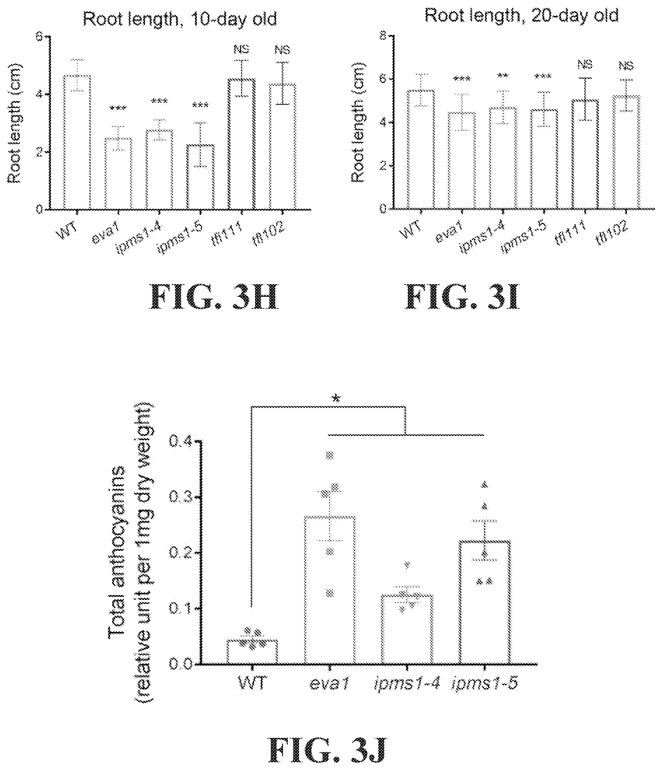

At 10 days following germination, the IPMS1 loss-of-function mutants displayed retardation of growth and development (FIG. 1G-1H), exhibiting approximately 30-40% decreases in aerial tissue fresh weight and 40-50% decreases in primary root length compared to WT (FIG. 3). Propidium iodide staining showed a strikingly delayed formation of root hairs in ipms1 mutants compared to WT, which was accompanied by increases in both cell length and number in the elongation zone. Meanwhile, in ipms1 meristem has increased cell number but reduced cell length. At 20 days of growth, the difference in fresh weight between ipms1 alleles and WT became not significant, though the primary roots of the ipms1 mutants were still slightly shorter than WT (FIG. 3H-3I). In contrast, two independent lines of dominant ipms1-1D feedback-insensitive mutant, which have small Val decrease and Leu increase, exhibited indistinguishable primary root elongation, but increased fresh weight compared to WT (FIG. 3F-3I). Additionally, notable differences were not observed between six-week old WT and IPMS1 loss-of-function mutant plants growing in soil. The transient retardation of overall plant growth of IPMS1 loss-of-function mutants correlated with the emergence-and-disappearance period of both vacuole morphology and BCAA homeostasis perturbation phenotypes (FIG. 1A-1D; FIG. 2B-2C).

The inventors then examined the development of cotyledons by confocal microscopy. The cotyledons constituted most of the aerial tissue for amino acid profiling and were used for analyses. Cotyledons of ipms1 mutants were thicker and larger than WT (FIG. 3A-3C). Despite a delay of true leaf emergence (FIG. 1G), the expanded first pair of true leaves in these mutants were larger than WT (FIG. 1H). Analyses of chloroplast ultrastructure revealed an absence of connecting stroma thylakoids and a reduction of thylakoid length ipms1 alleles compared to WT (FIG. 3D-3E). Additionally, purple pigmentation appeared in 10-day old IPMS1 loss-of-function mutants, particularly in cotyledon petioles and emerging true leaves. Anthocyanin extraction and measurement confirmed that these mutants contained higher levels of total anthocyanins compared to WT (FIG. 3J). These results indicate that the growth of certain tissues of the ipms1 mutants is particularly promoted but the overall plant growth and development are temporarily inhibited.

### Example 5: The Organization of ER Network and Actin Cytoskeleton is Altered in Eva1

This Example describes analysis of other endomembrane compartments by experiments designed to provide more insights into the eva1 vacuolar phenotypes.

The endoplasmic reticulum (ER) is the most extensively distributed organelle of the plant secretory pathway, and it is closely associated with several other membrane-bound organelles, including the vacuole (Ueda et al., 2010).

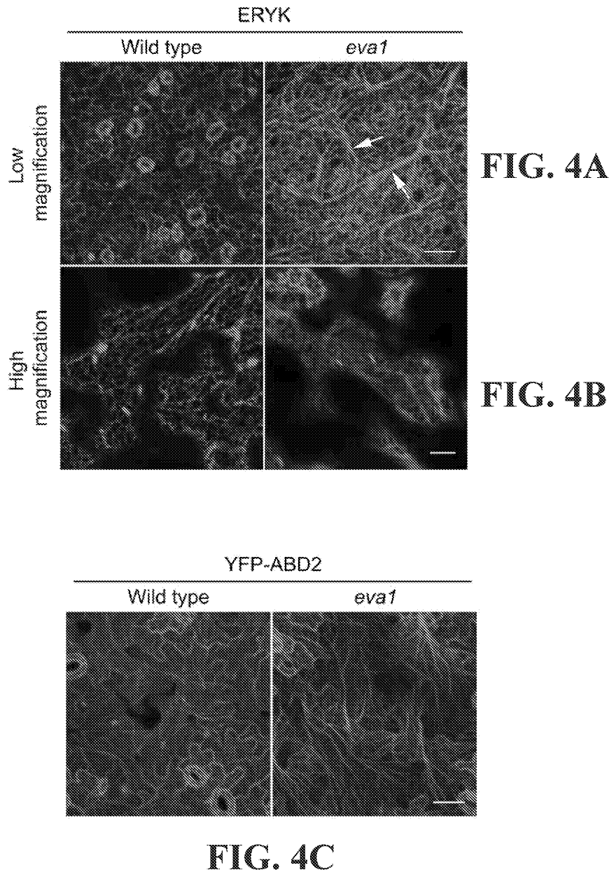

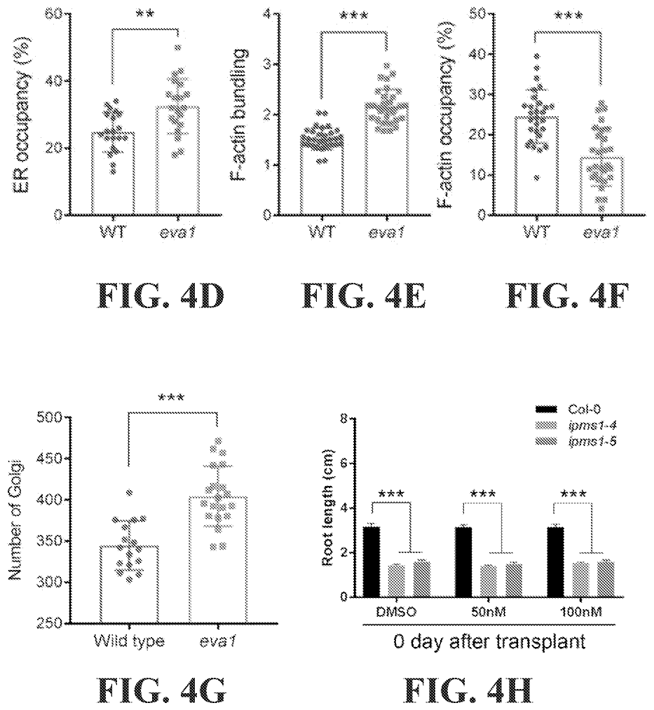

In the eva1 mutant, the ER luminal marker ERYK (Nelson et al., 2007) revealed a pronounced appearance of the cortical ER network with strikingly thickened strands compared to WT (FIG. 4A, see arrows). The thickened ER strands did not completely overlap with the trans-vacuolar strands. High-magnification confocal microscopy images of the cortical ER revealed a pronounced cisternation in eva1 compared to WT (FIG. 4B). Quantitative analyses of the surface area occupancy of the ER in the total field of view confirmed these observations (i.e., larger ER-occupied area in eva1 compared to WT) (FIG. 4D). The appearance of the Golgi apparatus, which in plant cells is organized in disperse stacks of cisternae in close association with the ER (Brandizzi and Barlowe, 2013), also was abnormal. Indeed, the Golgi marker GFP-CASP (Renna et al., 2005) revealed increased clustering and higher abundance of Golgi stacks at the cell cortex in eva1 compared to WT (FIG. 4G). The apoplast with the bulk flow marker SEC-RFP was also examined using methods described by Faso et al. (2009) to assess secretion, which is an important function of the endomembrane system (Renna et al., 2013; Renna et al., 2018). No intracellular retention of the SEC-RFP marker was observed in eva1 cotyledon epidermal cells.

These results and the absence of retention of the vacuolar marker in the ER document that the morphology of the vacuole, organization of the Golgi and the ER network are markedly affected by the eva1 mutation, while bulk-flow secretion is unaffected.

Collectively, the root-related defects of the ipms1 mutants, including delayed formation of root hairs and reduced number of lateral roots, are reminiscent of mutants with impaired actin depolymerization or promoted actin bundling (Ketelaar et al., 2004; Deeks et al., 2005), consistent with the possibility that reorganization of actin cytoskeleton may be causative of the observed developmental phenotypes. The establishment and maintenance of the trans-vacuolar strands, ER network and Golgi subcellular distribution are dependent on the actin cytoskeleton. The inventors hypothesized that the organization of actin cytoskeleton may be altered in eva1.

Confocal microscopy in cells expressing the actin filament (F-actin) marker YFP-ABD2 (Sheahan et al., 2004) revealed coalescence of actin cables compared to WT (FIG. 4C). Quantitative analyses of actin organization identified higher skewness, suggesting enhanced bundling, and lower density, suggesting decreased occupancy of F-actin in the cytoplasm in eva1 compared to WT (FIG. 4E-4F). These results indicate that the prominent phenotypes of the endomembranes in eva1 may be due to their connections with F-actin, whose organization is largely altered in the eva1 mutant.

Experiments were designed to validate this hypothesis by testing the sensitivity of the ipms1 alleles to the F-actin depolymerizing reagent latrunculin B (Lat B) (Cao et al., 2016). The primary root length of 10-day old ipms1-4 and ipms1-5 was approximately 50% of WT (FIG. 4H-4I). Seedlings of all genotypes were then transferred to medium containing DMSO or 50 nM or 100 nM Lat B in DMSO. After another 8 days, Lat B treatment promoted the formation of lateral roots in WT seedlings, but not in ipms1 alleles. Additionally, the primary root length of ipms1-4 and ipms1-5 was approximately 65% compared to WT on DMSO medium; however, this difference was reduced in the presence of increasing levels of Lat B in the growth medium (i.e., 80% to WT on 50 nM Lat B, and not significantly different from WT on 100 nM Lat B) (FIG. 4I). These results demonstrate that the ipms1 alleles are less sensitive to F-actin depolymerization compared to WT, supporting a functional connection between the disruption of IPMS1 and altered organization of the actin cytoskeleton.

### Example 6: The Eva1 Vacuolar Phenotypes are Rescued by PI3K/TOR Dual Inhibitors and Partially Recovered by Disruption of F-Actin

This Example describes experiments involving chemicals that alter the vacuolar morphogenesis and cytoskeleton integrity to provide insights into the mechanisms by which eva1 defects in BCAA biosynthesis led to alteration of the organization of subcellular structures. The inventors hypothesized that the persistence of small vacuoles in eva1 could be due to delayed vacuole membrane fusion during vacuole morphogenesis. To test this, wortmannin (Wm), an inhibitor of phosphoinositide 3-kinases (PI3Ks), was first employed to disrupt the balance of phosphoinositides and promotes homotypic tonoplast fusion (Zheng et al., 2014; Wang et al., 2009; Mayer et al., 2000).

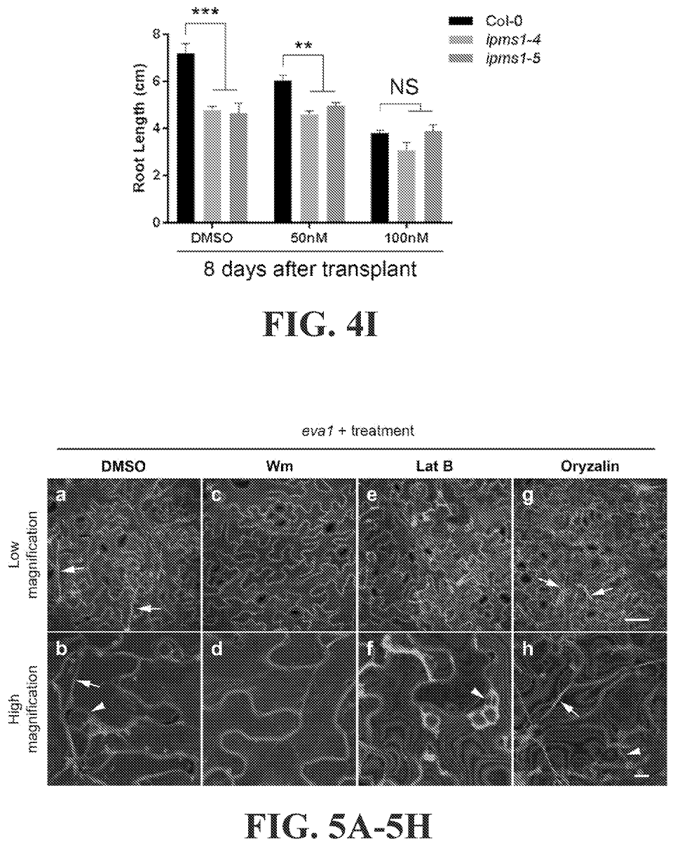

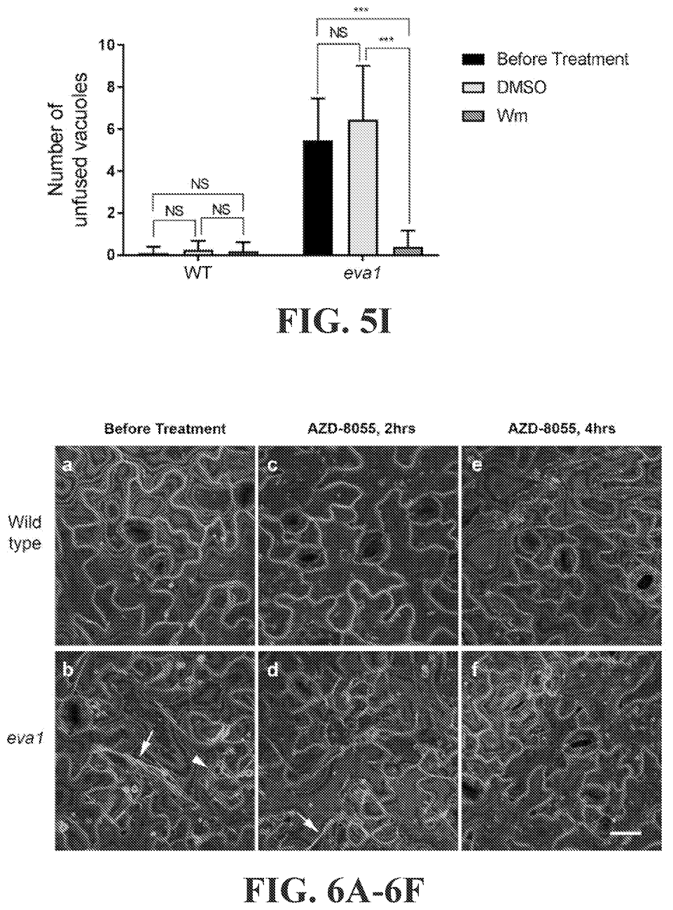

As shown in FIGS. 5A-5D and 5I, treatment of 10-day old WT and eva1 seedlings for two-three hours with wortmannin suppressed the eva1 phenotypes. The effects of wortmannin were mirrored by treatment with another PI3K inhibitor, LY294002 (Zheng et al., 2014) (FIG. 5E-5H).

The relationship between trans-vacuolar strands and integrity of the cytoskeleton in eva1 was then investigated. After a two-hour treatment with Lat B, the trans-vacuolar strands disappeared but the small vacuoles persisted in eva1 cotyledon epidermal cells (FIG. 5E-5F). By contrast, a two-hour treatment with oryzalin, a microtubule disrupting reagent (Zheng et al., 2014), did not lead to discernable change of vacuole morphology (FIG. 5G-5H). Together these results indicate that the presumably unfused vacuole and enhanced trans-vacuolar strand phenotypes in eva1 are both responsive to wortmannin and LY294002, but only the enhanced trans-vacuolar strand phenotype is related to the verified reorganization of F-actin.

### Example 7: Loss of Function of IPMS1 Leads to Up-Regulation of TOR Activity

As described above, homotypic membrane fusion and F-actin bundling are two processes directly involved in the eva1 Leu biosynthetic mutant phenotypes (FIG. 5)). This creates a quandary given that the role of IPMS1 in chloroplast BCAA biosynthesis is both functionally disconnected with—and spatially isolated from—the endomembrane compartments and actin cytoskeleton. Although the functions of wortmannin and LY294002 in inhibiting PI3Ks and promoting homotypic vacuolar membrane fusion have been established in plant cells (Cui et al., 2019; Zheng et al., 2014; Wang et al., 2009; Marshall and Vierstra, 2018), in mammalian cell studies these chemicals have been used to inhibit TOR signaling (Sarbassov et al., 2004; Brunn et al., 1996). This is because TOR belongs to the phosphoinositide kinase-related kinase (PIKK) family, whose members share similar kinase domains with PI3Ks (Andrs et al., 2015). Indeed, wortmannin and LY294002 are effective inhibitors of mammalian TOR (Brunn et al., 1996), and thus are considered as PI3K/TOR dual inhibitors (Benjamin et al., 2011). These foregoing results led the inventors to hypothesize that the effects of wortmannin and LY294002 in suppressing the eva1 vacuole phenotypes could be related to TOR inhibition.

To test this hypothesis, two TOR inhibitors with high selectivity for TOR over PI3Ks were employed: AZD-8055 and Torin2 (Benjamin et al., 2011; Liu et al., 2011; Chresta et al., 2010), which also effectively inhibit plant TOR (Li et al., 2017; Wang et al., 2018; Dong et al., 2017; Pu et al., 2017).

Ten-day old wild type and eva1 seedlings were transferred to liquid growth medium containing 5 μM AZD-8055. Compared to untreated samples, wild type cells did not exhibit significant changes in the morphology of the central vacuole and the few thin trans-vacuolar strands after 2 or 4 hours of incubation, although numerous fluorescent punctae appeared (FIGS. 6A, 6C, and 6E). TOR is the major negative regulator of autophagy (Pu et al., 2017). The punctae could then be autophagic structures resulting from the TOR inhibition by the chemicals. Untreated eva1 cells contained numerous small vacuoles and conspicuous trans-vacuolar strands (FIG. 6B); however, by 2 hours of treatment with AZD-8055, these structures were reduced in appearance (FIG. 6D). By 4 hours of treatment, the eva1 cells were indistinguishable from wild type, including the appearance of the small punctae (FIG. 6E-6F). These results were mirrored by Torin2 treatment: presumably unfused vacuoles and trans-vacuolar strands were no longer present in the eva1 cells by 2 hours of 1 μM Torin2 treatment. This result is consistent with the higher in vitro TOR inhibitory activity of Torin2 compared to AZD-8055 (Liu et al., 2011; Chresta et al., 2010).

In addition to the effects of temporal treatment on vacuolar phenotype (FIG. 6G), the effects of chronic inhibition of TOR were tested. As shown in FIG. 6L-6M, ipms1 primary root elongation was promoted by lower concentrations (0.1 and 0.2 μM), but inhibited by higher concentrations (0.4, 0.6 and 1.0 μM) of AZD-8055. These results indicate that moderate TOR inhibition led to optimized plant growth of ipms1. Similarly, a low concentration of wortmannin caused minimal but significant promotion of ipms1 root elongation (FIG. 6N-6O). By comparison, promotion of root elongation was not observed upon Lat B treatment (FIG. 6P-6Q). Together these results indicate that both subcellular and growth defects of ipms1 are associated with up-regulated TOR and are suppressed by TOR inhibition.

The inventors next sought to confirm these results by testing the activation status of TOR in ipms1 mutants. Based on the evidence that TOR inhibition rescued the ipms1 subcellular phenotypes, we predicted to find an increased level of TOR activity in ipms1 mutants compared to wild type. S6K is a conserved substrate of TOR protein kinase and its phosphorylation status has been adopted as an indicator of TOR activity in plants (Pfeiffer et al., 2016; Wang et al., 2018; Dong et al., 2017; Xiong and Sheen, 2012). Indeed, immunoblot analyses with specific antisera for either phosphorylated or total S6K (Pfeiffer et al., 2016; Wang et al., 2018; Dong et al., 2017; Xiong and Sheen, 2012) revealed increased levels of TOR-phosphorylated S6K in eva1 and ipms1-4 compared to WT, despite similar levels of total S6K in three genotypes (FIG. 6H-6I). The foregoing data support the conclusion that TOR signaling is upregulated in the ipms1 background.

To validate this conclusion, DNA synthesis was monitored in root tips because stimulated TOR signaling promotes cell proliferation in the root apical meristem. Such cellular proliferation can be detected by EdU staining of newly synthesized DNA (Li et al., 2017; Dong et al., 2017; Xiong et al., 2013). Consistent with the inventors' hypothesis, EdU staining displayed enhanced labeling in the root apical meristem of ipms1-4 and ipms1-5 compared to wild type (FIG. 6J-6K). This result was further confirmed by propidium iodide staining and morphometric analyses of root tips showing increased cell numbers in the root apical meristem of eva1, ipms1-4 and ipms1-5 compared to wild type.

The foregoing results show suppression of vacuole phenotypes by TOR inhibition, increased levels of S6K phosphorylation, and root apical meristem activity (i.e., increased DNA synthesis and cell number) in the ipms1 mutants. These results support the hypothesis that TOR signaling is up-regulated in the IPMS1 loss-of-function mutants.

### Example 8: Over-Accumulation of BCAAs Alters the Subcellular Organization of the Actin Cytoskeleton and Endomembranes

This Example describes testing of the role of TOR signaling and its specificity in the verified BCAA over-accumulation-induced phenotypes.

An estradiol-inducible TOR mutant (tor-es) (Xiong and Sheen, 2012) and a loss-of-function mutant of AtRAPTOR1B (raptor1b, SALK_022096) (Salem et al., 2017; a locus encoding the functional TORC1 component RAPTOR in Arabidopsis (Salem et al., 2018: Anderson et al., 2005)) were used in these experiments.

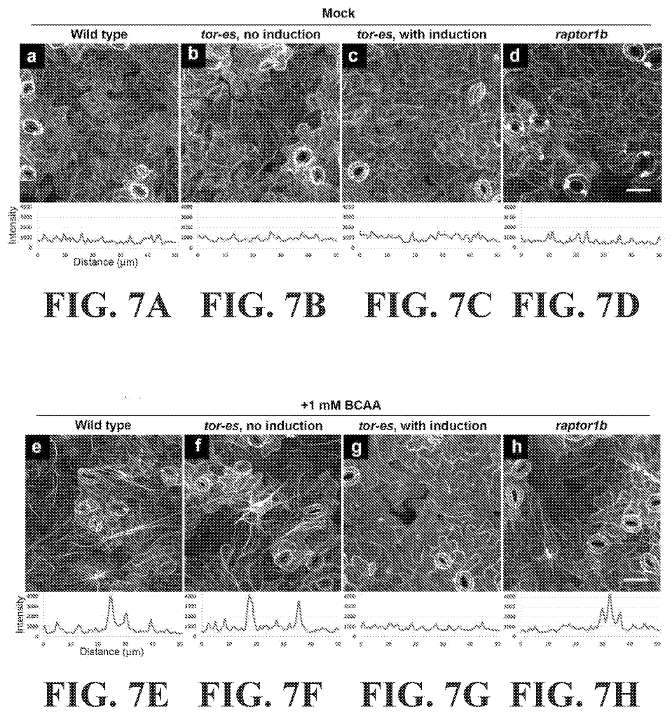

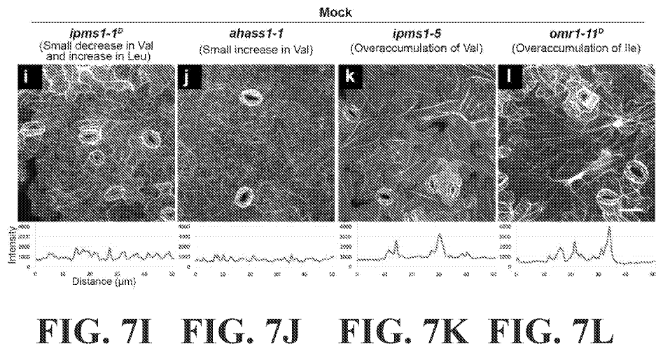

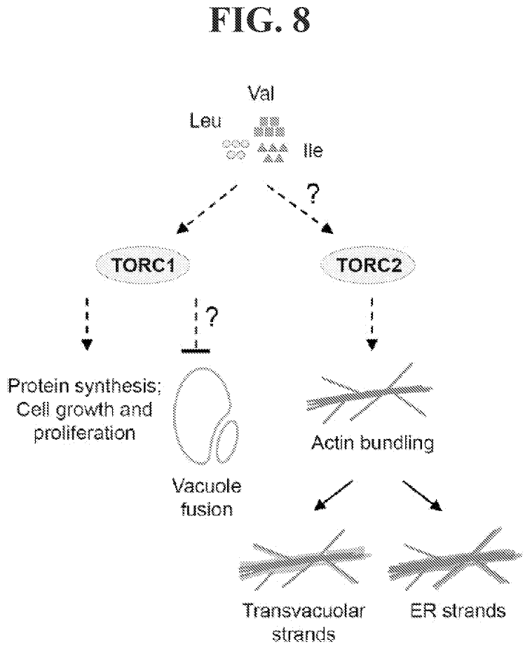

Before induction of TOR silencing, like wild type seedlings, for-es seedlings grown on BCAA-supplemented medium exhibited induced F-actin bundling compared to for-es grown on normal medium (compare FIGS. 7A and 7E with FIGS. 7B and 7F). After induction of TOR silencing, tor-es grown on either medium exhibited similarly low levels of bundling (FIGS. 7C and 7O). These results confirm a functional dependence of TOR signaling and the actin cytoskeleton phenotype due to mis-regulated TOR. By contrast, in raptor1b BCAA feeding led to F-actin bundling (FIG. 7D, 7H). Together, these results indicate that reorganization of F-actin is induced by over-accumulation of BCAAs and is dependent on functional TOR. In addition, these results indicate that the reorganization of F-actin induced by over-accumulation of BCAAs is also an underlying cause of the subcellular phenotype linking BCAA to TOR signaling components other than RAPTOR.

Next, experiments were performed to test the generality of the connection between over-accumulation of BCAAs, morphological alteration of cellular structures, and functional TOR signaling. To do so, a variety of BCAA mutants (Xing and Last. 2017) were used, combined with BCAA feeding. For example, an ipms1-1^{D} mutant was chosen because it exhibits a modest decrease in Val with an increase in Leu. The ahass1-1 mutant exhibits a small increase in Val. The ahass2-7 mutant exhibits decreased Val and Leu. The omr1-11^{D} mutant has a greater than 140-fold increase in Ile compared to WT.

Confocal microscopy analyses of cotyledon epidermal cells revealed that the organization of F-actin in ipms1-1 and ahass1-1 mutants resembled that of wild type (FIGS. 7A, 7I, and 7J). By contrast, enhanced actin bundling was observed following BCAA feeding (1 mM Val, Leu and Ile) and in the ipms1-5 and omr1-11^{D} mutants (FIGS. 7E, 7K, and 7L). Interestingly, the mutants also showed reorganization of F-actin and remodeling of the ER network. Specifically, mutants with moderate increase or decrease in BCAAs showed ER morphology similar to wild type, while wild type grown with BCAA supplementation and mutants that over-accumulate BCAAs showed compromised ER organization with longer and thicker ER strands compared to wild type.

The striking phenotype of enhanced ER strands in omr1-11^{D} was recovered by a 2-hour Torin2 treatment. In addition to bundling of F-actin and enhancement of ER strands, supplementation of BCAAs also induced the formation of prominent trans-vacuole strands.

Together, these results support a general correlation between over-accumulation of BCAAs and distorted actin cytoskeleton and endomembranes.

### REFERENCES

Anderson G H, Veit B, Hanson M R. 2005. The Arabidopsis AtRaptor genes are essential for post-embryonic plant growth. BMC Biology 3:12.
Andrs M, Korabecny J. Jun D, Hodny Z, Bartek J, Kuca K. 2015. Phosphatidylinositol 3-Kinase (PI3K) and phosphatidylinositol 3-kinase-related kinase (PIKK) inhibitors: importance of the morpholine ring. Journal of Medicinal Chemistry 58:41-71.
Angelovici R. Lipka A E, Deason N, Gonzalez-Jorge S, Lin H, Cepela J, Buell R. Gore M A. Dellapenna D. 2013. Genome-wide analysis of branched-chain amino acid levels in Arabidopsis seeds. The Plant Cell 25:4827-4843.
Avila E L. Zouhar J. Agee A E, Carter D G, Chary S N, Raikhel N V. 2003. Tools to study plant organelle biogenesis. Point mutation lines with disrupted vacuoles and high-speed confocal screening of green fluorescent protein-tagged organelles. Plant Physiology 133:1673-1676.
Point mutation lines with disrupted vacuoles and high-speed confocal screening of green fluorescent protein-tagged organelles. Plant Physiology 133:1673-1676.
Benjamin D, Colombi M, Moroni C, Hall M N. 2011. Rapamycin passes the torch: a new generation of mTOR inhibitors. Nature Reviews Drug Discovery 10:868-880.
Boisen S. Hvelplund T, Weisbjerg M R. 2000. Ideal amino acid profiles as a basis for feed protein evaluation. Livestock Production Science 64:239-251.
Brandizzi F, Barlowe C. 2013. Organization of the ER-Golgi interface for membrane traffic control. Nature Reviews Molecular Cell Biology 14:382-392.
Brunn G J, Williams J, Sabers C, Wiederrecht G, Lawrence J C, Abraham R T. 1996. Direct inhibition of the signaling functions of the mammalian target of rapamycin by the phosphoinositide 3-kinase inhibitors, wortmannin and LY294002. The EMBO Journal 15:5256-5267.
Caldana C, Li Y, Leisse A, Zhang Y, Bartholomaeus L, Fernie A R, Willmitzer L. Giavalisco P. 2013. Systemic analysis of inducible target of rapamycin mutants reveal a general metabolic switch controlling growth in Arabidopsis thaliana. The Plant Journal 73:897-909.
Cao P, Renna L, Stefano G, Brandizzi F. 2016. SYP73 anchors the ER to the actin cytoskeleton for maintenance of ER integrity and streaming in Arabidopsis. Current Biology 26:3245-3254.
Chen G-H, Liu M-J, Xiong Y, Sheen J, Wu S-H. 2018. TOR and RPS6 transmit light signals to enhance protein translation in deetiolating Arabidopsis seedlings. PNAS 115:12823-12828.
Chresta C M, Davies B R, Hickson I, Harding T, Cosulich S, Critchlow S E, Vincent J P, Ellston R, Jones D, Sini P, James D, Howard Z. Dudley P. Hughes G, Smith L, Maguire S, Hummersone M, Malagu K, Menear K, Jenkins R, et al. 2010. AZD8055 is a potent, selective, and orally bioavailable ATP-Competitive mammalian target of rapamycin kinase inhibitor with in vitro and in vivo antitumor activity. Cancer Research 70:288-298.
Clough S J. Bent A F. 1998. Floral dip: a simplified method for Agrobacterium-mediated transformation of Arabidopsis thaliana. The Plant Journal 16:735-743.
Cui Y, Cao W, He Y, Zhao Q, Wakazaki M, Zhuang X, Gao J, Zeng Y, Gao C, Ding Y, Wong H Y, Wong W S, Lam H K, Wang P, Ueda T, Rojas-Pierce M,
Toyooka K. Kang B H, Jiang L. 2019. A whole-cell electron tomography model of vacuole biogenesis in Arabidopsis root cells. Nature Plants 5:95-105.
Cutler S R, Ehrhardt D W, Griffitts J S, Somerville C R. 2000. Random GFP::cDNA fusions enable visualization of subcellular structures in cells of Arabidopsis at a high frequency. PNAS 97:3718-3723.
de Kraker J W, Luck K. Textor S. Tokuhisa J G, Gershenzon J. 2007. Two Arabidopsis genes (IPMS1 and IPMS2) encode isopropylmalate synthase, the branchpoint step in the biosynthesis of leucine. Plant Physiology 143: 970-986.
Deeks M J, Cvrckova F, Machesky L M, Mikitova V, Ketelaar T, Zarsky V, Davies B, Hussey P J. 2005. Arabidopsis group ie formins localize to specific cell membrane domains, interact with actin-binding proteins and cause defects in cell expansion upon aberrant expression. New Phytologist 168:529-540.
Deprost D, Truong H N, Robaglia C. Meyer C. 2005. An Arabidopsis homolog of RAPTOR/KOG1 is essential for early embryo development. Biochemical and Biophysical Research Communications 326:844-850.
Deprost D, Yao L, Sormani R. Moreau M. Leterreux G, Nicolai M. Bedu M, Robaglia C, Meyer C. 2007. The Arabidopsis TOR kinase links plant growth, yield, stress resistance and mRNA translation. EMBO Reports 8: 864-870.
Dong Y, Silbermann M, Speiser A, Forieri I, Linster E, Poschet G, Allboje Samami A. Wanatabe M, Sticht C. Teleman A A, Deragon J M, Saito K, Hell R, Wirtz M. 2017. Sulfur availability regulates plant growth via glucose-TOR signaling. Nature Communications 8:1174.
Dunser K, Gupta S, Herger A, Feraru M I, Ringli C, Kleine-Vehn J. 2019. Extracellular matrix sensing by FERONIA and Leucine-Rich repeat extensins controls vacuolar expansion during cellular elongation in Arabidopsis thaliana. The EMBO Journal 38:e100353.
Faso C, Chen Y N, Tamura K, Held M. Zemelis S. Marti L, Saravanan R. Hummel E, Kung L. Miller E, Hawes C, Brandizzi F. 2009. A missense mutation in the Arabidopsis COPII coat protein Sec24A induces the formation of clusters of the endoplasmic reticulum and golgi apparatus. The Plant Cell 21:3655-3671.
Feiguelman G, Fu Y, Yalovsky S. 2018. ROP GTPases Structure-Function and signaling pathways. Plant Physiology 176:57-79.
Field B, Cardon G. Traka M, Botterman J, Vancanneyt G, Mithen R. 2004. Glucosinolate and amino acid biosynthesis in Arabidopsis. Plant Physiology 135:828-839.
Field B. Furniss C. Wilkinson A, Mithen R. 2006. Expression of a Brassica isopropylmalate synthase gene in Arabidopsis perturbs both glucosinolate and amino acid metabolism. Plant Molecular Biology 60:717-727.
French A P, Wilson M H, Kenobi K, Dietrich D. Voß U. Ubeda-Tomas S. Pridmore T P, Wells D M. 2012. Identifying biological landmarks using a novel cell measuring image analysis tool: cell-o-tape. Plant Methods 8:7.
Galili G. Amir R. Fernie A R. 2016. The regulation of essential amino acid synthesis and accumulation in plants. Annual Review of Plant Biology 67:153-178.
Garcia A V, A I-Yousif M, Hirt H. 2012. Role of AGC kinases in plant growth and stress responses. Cellular and Molecular Life Sciences 69:3259-3267.
Gattolin S. Sorieul M, Frigerio L. 2010. Tonoplast intrinsic proteins and vacuolar identity. Biochemical Society Transactions 38:769-773.
Imhof J. Huber F, Reichelt M. Gershenzon J. Wiegreffe C, Lächler K, Binder S. 2014. The small subunit 1 of the Arabidopsis isopropylmalate isomerase is required for normal growth and development and the early stages of glucosinolate formation. PLOS ONE 9:e91071.
Jacinto E, Loewith R, Schmidt A. Lin S, Rüegg M A. Hall A, Hall M N. 2004. Mammalian TOR complex 2 controls the actin cytoskeleton and is rapamycin insensitive. Nature Cell Biology 6:1122-1128.
Ketelaar T, Allwood E G, Anthony R, Voigt B, Menzel D. Hussey P J. 2004. The actin-interacting protein AIPI is essential for actin organization and plant development. Current Biology 14:145-149.
Kim S J, Zemelis-Durfee S, Jensen J K, Wilkerson C G, Keegstra K, Brandizzi F. 2018. In the grass species Brachypodium distachyon, the production of mixed-linkage (1,3;1,4)-b-glucan (MLG) occurs in the golgi apparatus. The Plant Journal: For Cell and Molecular Biology 93:1062-1075.
Kim J, Guan K L. 2019. mTOR as a central hub of nutrient signalling and cell growth. Nature Cell Biology 21:63-71.
Kotoga'ny E, Dudits D. Horvath G V, Ayaydin F. 2010. A rapid and robust assay for detection of S-phase cell cycle progression in plant cells and tissues by using ethynyl deoxyuridine. Plant Methods 6:5.
Li X. Cai W, Liu Y, Li H, Fu L, Liu Z. Xu L, Liu H, Xu T. Xiong Y. 2017. Differential TOR activation and cell proliferation in Arabidopsis root and shoot apexes. PNAS 114:2765-2770.
Liu Q, Wang J, Kang S A, Thoreen C C, Hur W, Ahmed T, Sabatini D M, Gray N S. 2011. Discovery of 9-(6-aminopyridin-3-yl)-1-(3-(trifluoromethyl)phenyl) benzo[h][1,6]naphthyridin-2(1H)-one (Torin2) as a potent, selective, and orally available mammalian target of rapamycin (mTOR) inhibitor for treatment of Cancer. Journal of Medicinal Chemistry 54:1473-1480.
Lafke C, Dunser K, Scheuring D, Kleine-Vehn J. 2015. Auxin regulates SNARE-dependent vacuolar morphology restricting cell size. eLife 4:e05868.
Lu Y J, Day B. 2017. Quantitative evaluation of plant actin cytoskeletal organization during immune signaling. Methods in Molecular Biology 1578:207-221.
Maliga P. 1984. Isolation and characterization of mutants in plant cell culture. Annual Review of Plant Physiology 35:519-542.
Malinovsky F G, Thomsen M F, Nintemann S J, Jagd L M, Bourgine B, Burow M, Kliebenstein D J. 2017. An evolutionarily young defense metabolite influences the root growth of plants via the ancient TOR signaling pathway. eLife 6:e29353.
Marshall R S, Vierstra R D. 2018. Autophagy: the master of bulk and selective recycling. Annual Review of Plant Biology 69:173-208.
Mayer A, Scheglmann D. Dove S. Glatz A, Wickner W, Haas A. 2000. Phosphatidylinositol 4,5-bisphosphate regulates two steps of homotypic vacuole fusion. Molecular Biology of the Cell 11:807-817.
Michaillat L, Baars T L, Mayer A. 2012. Cell-free reconstitution of vacuole membrane fragmentation reveals regulation of vacuole size and number by TORC1. Molecular Biology of the Cell 23:881-895.
Moreau M, Azzopardi M, Clément G, Dobrenel T, Marchive C, Renne C, Martin-Magniette M L, Taconnat L, Renou J P, Robaglia C. Meyer C. 2012. Mutations in the Arabidopsis homolog of LST8/GbL, a partner of the target of rapamycin kinase, impair plant growth, flowering, and metabolic adaptation to long days. The Plant Cell 24:463-481.
Mubeen U, Juppner J, Alpers J. Hincha D K. Giavalisco P. 2018. Target of rapamycin inhibition in Chlamydomonas reinhardtii Triggers de Novo Amino Acid Synthesis by Enhancing Nitrogen Assimilation. The Plant Cell 30:2240-2254.
Nelson B K. Cai X, Nebenfuihr A. 2007. A multicolored set of in vivo organelle markers for co-localization studies in Arabidopsis and other plants. The Plant Journal 51:1126-1136.
Peng C. Uygun S. Shiu S H, Last R L. 2015. The impact of the Branched-Chain ketoacid dehydrogenase complex on amino acid homeostasis in Arabidopsis. Plant Physiology 169:1807-1820.
Pfeiffer A. Janocha D, Dong Y. Medzihradszky A, Schöne S. Daum G, Suzaki T, Forner J, Langenecker T, Rempel E, Schmid M. Wirtz M, Hell R, Lohmann J U. 2016. Integration of light and metabolic signals for stem cell activation at the shoot apical meristem. eLife 5:e17023.
Pu Y, Luo X, Bassham D C. 2017. TOR-Dependent and -Independent pathways regulate autophagy in Arabidopsis thaliana. Frontiers in Plant Science 8:1204.
Ren M, Venglat P, Qiu S, Feng L, Cao Y, Wang E, Xiang D, Wang J, Alexander D, Chalivendra S, Logan D, Mattoo A, Selvaraj G, Datla R. 2012. Target of rapamycin signaling regulates metabolism, growth, and life span in Arabidopsis. The Plant Cell 24:4850-4874.
Renna L, Hanton S L, Stefano G, Bortolotti L, Misra V, Brandizzi F. 2005. Identification and characterization of AtCASP, a plant transmembrane golgi matrix protein. Plant Molecular Biology 58:109-122.
Renna L, Stefano G, Majeran W. Micalella C, Meinnel T, Giglione C, Brandizzi F. 2013. Golgi traffic and integrity depend on N-myristoyl transferase-1 in Arabidopsis. The Plant Cell 25:1756-1773.
Renna L, Stefano G, Slabaugh E, Wormsbaecher C, Sulpizio A, Zienkiewicz K, Brandizzi F. 2018. TGNap1 is required for microtubule-dependent homeostasis of a subpopulation of the plant trans-Golgi network. Nature Communications 9:5313.
Roelants F. Leskoske K. Martinez Marshall M N, Locke M, Thorner J. 2017. The TORC2-Dependent signaling network in the yeast Saccharomyces cerevisiae. Biomolecules 7:66.
Salem M A, Li Y, Wiszniewski A, Giavalisco P. 2017. Regulatory-associated protein of TOR (RAPTOR) alters the hormonal and metabolic composition of Arabidopsis seeds, controlling seed morphology, viability and germination potential. The Plant Journal 92:525-545.
Salem M A. Li Y, Bajdzienko K, Fisahn J, Watanabe M, Hoefgen R, Schöttler M A, Giavalisco P. 2018. RAPTOR controls developmental growth transitions by altering the hormonal and metabolic balance. Plant Physiology 177:565-593.
Sarbassov D D, Ali S M, Kim D H, Guertin D A. Latek R R, Erdjument-Bromage H, Tempst P, Sabatini D M. 2004. Rictor, a Novel Binding Partner of mTOR. Defines a Rapamycin-Insensitive and Raptor-Independent Pathway that Regulates the Cytoskeleton. Current Biology 14:1296-1302.
Saxton R A, Sabatini D M. 2017. mTOR signaling in growth, metabolism, and disease. Cell 168:960-976.
Schaufelberger M, Galbier F, Herger A, de Brito Francisco R, Roffler S, Clement G, Diet A, Hortensteiner S. Wicker T, Ringli C. 2019. Mutations in the Arabidopsis ROL17/isopropylmalate synthase 1 locus alter amino acid content, modify the TOR network, and suppress the root hair cell development mutant Irx1. Journal of Experimental Botany 70:2313-2323.
Schepetilnikov M, Makarian J. Srour O, Geldreich A. Yang Z, Chicher J. Hammann P, Ryabova L A. 2017. GTPase ROP2 binds and promotes activation of target of rapamycin, TOR, in response to auxin. The EMBO Journal 36: 886-903.
Scheuring D, Lofke C. Kruger F, Kittelmann M, Eisa A, Hughes L. Smith R S, Hawes C, Schumacher K, Kleine-Vehn J. 2016. Actin-dependent vacuolar occupancy of the cell determines auxin-induced growth repression. PNAS 113:452-457.
Schindelin J. Arganda-Careras I, Frise E, Kaynig V, Longair M. Pietzsch T, Preibisch S, Rueden C, Saalfeld S, Schmid B, Tinevez J Y, White D J, Hartenstein V. Eliceiri K, Tomancak P, Cardona A. 2012. Fiji: an open-source platform for biological-image analysis. Nature Methods 9:676-682.
Schmidt A, Kunz J, Hall M N. 1996. TOR2 is required for organization of the actin cytoskeleton in yeast. PNAS 93: 13780-13785.
Sheahan M B, Rose R J, McCurdy D W. 2004. Organelle inheritance in plant cell division: the actin cytoskeleton is required for unbiased inheritance of chloroplasts, mitochondria and endoplasmic reticulum in dividing protoplasts. The Plant Journal 37:379-390.
Shi L, Wu Y, Sheen J. 2018. TOR signaling in plants: conservation and innovation. Development 145:dev160887.
Shimomura Y, Murakami T, Nakai N, Nagasaki M, Harris R A. 2004. Exercise promotes BCAA catabolism: effects of BCAA supplementation on skeletal muscle during exercise. The Journal of Nutrition 134:1583S-1587.
Stefano G. Renna L, Moss T, McNew J A, Brandizzi F. 2012. In Arabidopsis, the spatial and dynamic organization of the endoplasmic reticulum and golgi apparatus is influenced by the integrity of the C-terminal domain of RHD3, a non-essential GTPase. The Plant Journal 69:957-966.
Stefano G. Hawes C, Brandizzi F. 2014. ER—the key to the highway. Current Opinion in Plant Biology 22:30-38.
Stefano G. Brandizzi F. 2018. Advances in plant ER architecture and dynamics. Plant Physiology 176:178-186.
Ueda H, Yokota E, Kutsuna N, Shimada T, Tamura K, Shimmen T, Hasezawa S, Dolja V V, Hara-Nishimura 1. 2010. Myosin-dependent endoplasmic reticulum motility and F-actin organization in plant cells. PNAS 107:6894-6899.
Wang J, Cai Y. Miao Y, Lam S K, Jiang L. 2009. Wortmannin induces homotypic fusion of plant prevacuolar compartments. Journal of Experimental Botany 60:3075-3083.
Wang P, Zhao Y, Li Z, Hsu C-C, Liu X, Fu L, Hou Y-J, Du Y, Xie S, Zhang C, Gao J, Cao M, Huang X, Zhu Y, Tang K, Wang X, Tao W A, Xiong Y, Zhu J-K. 2018. Reciprocal regulation of the TOR kinase and ABA receptor balances plant growth and stress response. Molecular Cell 69:100-112.
Weraduwage S M, Kim S-J. Renna L, Anozie F C, Sharkey T D, Brandizzi F. 2016. Pectin methylesterification impacts the relationship between photosynthesis and plant growth in Arabidopsis thaliana. Plant Physiology 171:833-848.
Westrate L M. Lee J E, Prinz W A, Voeltz G K. 2015. Form follows function: the importance of endoplasmic reticulum shape. Annual Review of Biochemistry 84:791-811.
Wu H, Carvalho P, Voeltz G K. 2018. Here, there, and everywhere: the importance of ER membrane contact sites. Science 361:5835.
Xie J, Wang X, Proud C G. 2018. Who does TORC2 talk to? Biochemical Journal 475:1721-1738.
Xing A, Last R L. 2017. A regulatory hierarchy of the Arabidopsis Branched-Chain amino acid metabolic network. The Plant Cell 29:1480-1499.
Xiong Y, McCormack M, Li L. Hall Q, Xiang C, Sheen J. 2013. Glucose-TOR signalling reprograms the transcriptome and activates meristems. Nature 496:181-186.
Xiong F, Zhang R, Meng Z, Deng K, Que Y, Zhuo F, Feng L. Guo S, Datla R, Ren M. 2017. Brassinosteriod insensitive 2 (BIN2) acts as a downstream effector of the target of rapamycin (TOR) signaling pathway to regulate photoautotrophic growth in Arabidopsis. New Phytologist 213:233-249.
Xiong Y, Sheen J. 2012. Rapamycin and glucose-target of rapamycin (TOR) protein signaling in plants. Journal of Biological Chemistry 287:2836-2842.
Yu L. McPhee C K, Zheng L. Mardones G A, Rong Y, Peng J, Mi N, Zhao Y, Liu Z, Wan F, Hailey D W, Oorschot V, Klumperman J, Baehrecke E H. Lenardo M J. 2010. Termination of autophagy and reformation of lysosomes regulated by mTOR. Nature 465:942-946.
Zhang C, Hicks G R, Raikhel N V. 2014. Plant vacuole morphology and vacuolar trafficking. Frontiers in Plant Science 5:476.
Zhang Z, Zhu J Y, Roh J, Marchive C, Kim S K, Meyer C, Sun Y, Wang W. Wang Z Y. 2016. TOR signaling promotes accumulation of BZR1 to balance growth with carbon availability in Arabidopsis. Current Biology 26:1854-1860.
Zhao L, Deng L, Zhang Q, Jing X, Ma M, Yi B, Wen J, Ma C, Tu J, Fu T, Shen J. 2018. Autophagy contributes to sulfonylurea herbicide tolerance via GCN2-independent regulation of amino acid homeostasis. Autophagy 14: 702-714.
Zheng J, Han S W, Rodriguez-Welsh M F, Rojas-Pierce M. 2014. Homotypic vacuole fusion requires VTI11 and is regulated by phosphoinositides. Molecular Plant 7:1026-1040.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements describe some of the elements or features of the invention. The statements provide features that can be claimed in the application and the dependencies of the statements illustrate combinations of features that can be present when included in the claims.

### Statements:

1. A modified plant cell, plant seed, or plant comprising a modified isopropylmalate synthase (IPMS) gene that encodes a modified isopropylmalate synthase (IPMS) protein; or a modified plant cell, plant seed, or plant comprising an expression cassette comprising a promoter operably linked to a nucleic acid segment encoding a modified isopropylmalate synthase (IPMS) protein.
2. The modified plant cell, plant seed, or plant, wherein the modified isopropylmalate synthase protein has isopropylmalate synthase activity or does not have significant isopropylmalate synthase activity.
3. The modified plant cell, plant seed, or plant of statement 1 or 2, wherein the promoter is a tissue specific promoter or a developmentally regulated promoter.
4. The modified plant cell, plant seed, or plant of any of statements 1-3, wherein the promoter is a seed specific promoter.
5. The modified plant cell, plant seed, or plant of any of statements 1-4, wherein the modified isopropylmalate synthase protein has a mutation or modification of its allosteric domain.
6. The modified plant cell, plant seed, or plant of any of statements 1-5, wherein the modified isopropylmalate synthase protein has a mutation or modification located within about 20 amino acids of the C-terminus.
7. The modified plant cell, plant seed, or plant of any of statements 1-6, wherein the modified isopropylmalate synthase protein has a point mutation at a position corresponding to position 606 of SEQ ID NO:2.
8. The modified plant cell, plant seed, or plant of any of statements 1-7, wherein the modified isopropylmalate synthase protein has a replacement of a glycine (G) at a position corresponding to position 606 of SEQ ID NO:2.
9. The modified plant cell, plant seed, or plant of statement 8, wherein the glycine at a position corresponding to position 606 of SEQ ID NO:2 is a glutamic acid (E).
10. The modified plant cell, plant seed, or plant of any of statements 1-9, wherein the plant seeds have increased content of Gln, His, Ile, Leu, Lys, Met, Phe, Thr, Trp, Val, or a combination thereof.
11. The modified plant cell, plant seed, or plant of any of statements 1-10, wherein the plant's vegetative tissues have increased content of Leu, and other amino acids.
12. The modified plant cell, plant seed, or plant of any of statements 1-4, wherein the modified isopropylmalate synthase protein has a mutation or modification of its catalytic domain.
13. The modified plant cell, plant seed, or plant of any of statements 14 or 12, wherein the modified isopropylmalate synthase protein has a mutation within or next to its substrate pocket.
14. The modified plant cell, plant seed, or plant of any of statements 1-4, 12 or 13, wherein the modified isopropylmalate synthase protein has a mutation in its acetyl-CoA binding surface near its substrate pocket.
15. The modified plant cell, plant seed, or plant of any of statements 1-4, 12-14, wherein the modified isopropylmalate synthase protein has as aspartic acid replaced in its catalytic domain.
16. The modified plant cell, plant seed, or plant of any of statements 1-4, 12-15, wherein the modified isopropylmalate synthase protein has as aspartic acid replaced at an amino acid position corresponding to position 228 of SEQ ID NO:2.
17. The modified plant cell, plant seed, or plant of any of statements 1-4, 12-16, wherein the seeds and/or plants have increased content of amino acids comprising valine.
18. The modified plant cell, plant seed, or plant of any of statements 1-17 wherein plants that express the modified isopropylmalate synthase protein have increased amino acid content compared to an average content for one or more amino acids of wild type plants, or parental plants, or plants with a knockout IPMS gene that do not express the modified isopropylmalate synthase protein.
19. The modified plant cell, plant seed, or plant of any of statements 1-18, wherein a plant expressing the modified isopropylmalate synthase protein has at least a 1%, at least a 2%, at least a 3%, at least a 4%, at least a 5%, at least a 10%, at least a 15%, at least a 20%, at least a 25%, at least a 30%, at least a 40%, at least a 50%, at least a 100%, at least a 150%, at least a 200%, at least a 300%, at least a 400%, or at least a 500% increase in the content of one or more amino acids compared to an average content for one or more amino acids of wild type plants, or parental plants, or plants with a knockout IPMS gene that do not express the modified isopropylmalate synthase protein.
20. The modified plant cell, plant seed, or plant of any of statements 1-19, wherein a plant expressing the modified isopropylmalate synthase protein has at least a two-fold, at least a three-fold, at least a four-fold, or at least a five-fold increase in the content of one or more amino acids compared to an average amino acid content of wild type plants, or parental plants, or plants with a knockout IPMS gene that do not express the modified isopropylmalate synthase protein.
21. The modified plant cell, plant seed, or plant of any of statements 1-20, wherein plants that express the modified isopropylmalate synthase protein have increased biomass compared to an average biomass of wild type plants, or parental plants, or plants with a knockout IPMS gene that do not express the modified isopropylmalate synthase protein.
22. The modified plant cell, plant seed, or plant of any of statements 1-21, wherein a plant expressing the modified isopropylmalate synthase protein has at least a 1%, at least a 2%, at least a 3%, at least a 4%, at least a 5%, at least a 10%, at least a 15%, at least a 20%, at least a 25%, at least a 30%, at least a 40%, at least a 50%, at least a 100%, at least a 150%, at least a 200%, at least a 300%, at least a 400%, or at least a 500% increase in biomass compared to an average biomass of wild type plants, or parental plants, or plants with a knockout IPMS gene that do not express the modified isopropylmalate synthase protein.
23. The modified plant cell, plant seed, or plant of any of statements 1-22, wherein the modified isopropylmalate synthase nucleic acid or the modified isopropylmalate synthase protein has at least 70%, at least 80%, at least 85%, at least 90%, at least 95%, at least 96%, at least 97%, at least 98%, at least 99%, or at least 99.5% sequence identity to any of SEQ ID NO:1-38.
24. The modified plant cell, plant seed, or plant of any of statements 1-23, wherein the modified isopropylmalate synthase nucleic acid has at least 70%, at least 80%, at least 85%, at least 90%, at least 95%, at least 96%, at least 97%, at least 98%, at least 99%, or at least 99.5% sequence identity to any of SEQ ID NO:1, 4, 6, 8, 27, 29, 31, 34, or 37.
25. The modified plant cell, plant seed, or plant of any of statements 1-24, wherein the modified isopropylmalate synthase nucleic acid has at least 70%, at least 80%, at least 85%, at least 90%, at least 95%, at least 96%, at least 97%, at least 98%, at least 99%, or at least 99.5% sequence identity to any of SEQ ID NO:4 or 6.
26. The modified plant cell, plant seed, or plant of any of statements 1-25, wherein the modified isopropylmalate synthase protein has at least 70%, at least 80%, at least 85%, at least 90%, at least 95%, at least 96%, at least 97%, at least 98%, at least 99%, or at least 99.5% sequence identity to any of SEQ ID NO:2, 3, 5, 7, 9, 10, 12, 13, 14, 15, 16, 17, 18, 19, 20, 21, 22, 23, 24, 25, 26, 28, 30, 32, 33, 34, 35, 36, or 38.
27. The modified plant cell, plant seed, or plant of any of statements 1-26, which is an agricultural crop species or a forage crop species.
28. The modified plant cell, plant seed, or plant of any of statements 1-27, which is a forage plant (e.g., alfalfa, clover, soybeans, turnips, bromegrass, bluestem, and fescue), starch plant (e.g., canola, potatoes, lupins, sunflower and cottonseed), grain (maize, wheat, barley, oats, rice, sorghum, millet and rye), grass (switchgrass, prairie grass, wheat grass, sudangrass, sorghum, straw-producing plants, miscanthus, switchgrass), sugar producing plants (sugarcane, beets), vegetable plant (e.g., cucumber, lettuce, tomato), Brachypodium, Arabidopsis, bamboo, softwood, or hardwood.
29. A method comprising: selecting a plant cell comprising an endogenous mutation or modification of an isopropylmalate synthase (IPMS) gene, or comprising an expression cassette with an isopropylmalate synthase (IPMS) mutation or modification; generating a plant from the plant cell that expresses a modified isopropylmalate synthase protein; and cultivating the plant.
30. The method of statement 29, further comprising analyzing the amino acid content or biomass of the plant, and selecting one or more plants that have increased amino acid content or increased biomass compared to an average amino acid content or an average biomass of wild type plants, or parental plants, or plants with a knockout IPMS gene that do not express the modified isopropylmalate synthase protein.
31. The method of statement 29 or 30, wherein the mutation or modification is at least one mutation or modification to an endogenous (native) isopropylmalate synthase (IPMS) gene in the plant cell.
32. The method of statement 29 or 30, wherein the expression cassette with an isopropylmalate synthase (IPMS) mutation or modification is an expression cassette comprising a promoter operably linked to a nucleic acid segment encoding the modified isopropylmalate synthase protein.
33. The method of statement 32, wherein the promoter is a tissue specific promoter or a developmentally regulated promoter.
34. The method of statement 32, wherein the promoter is a seed specific promoter.
35. The method of any of statements 29-34, wherein the a modified isopropylmalate synthase protein has isopropylmalate synthase activity or does not have significant isopropylmalate synthase activity.
36. The method of any of statements 29-35, wherein the modified isopropylmalate synthase protein comprises a mutation or modification of its allosteric domain.
37. The method of any of statements 29-36, wherein the modified isopropylmalate synthase protein has a mutation or modification located within about 20 amino acids of the C-terminus.
38. The method of any of statements 29-37, wherein the modified isopropylmalate synthase protein has a point mutation at a position corresponding to position 606 of SEQ ID NO:2.
39. The method of any of statements 29-38, wherein the modified isopropylmalate synthase protein has a replacement of a glycine (G) at a position corresponding to position 606 of SEQ ID NO:2.
40. The method of any of statements 29-39, wherein the glycine at a position corresponding to position 606 of SEQ ID NO:2 is a glutamic acid (E).
41. The method of any of statements 29-40, wherein the plants' seeds have increased content of Gln, His, Ile, Leu, Lys, Met, Phe, Thr, Trp, Val, or a combination thereof.
42. The method of any of statements 29-41, wherein the plants' vegetative tissues have increased content of amino acids comprising Leu, and other amino acids.
43. The method of any of statements 29-41, wherein the plants have increased biomass.
44. The method of any of statements 29-35, wherein the modified isopropylmalate synthase protein comprises a mutation or modification of its catalytic domain.
45. The method of any of statements 29-35 or 44, wherein the modified isopropylmalate synthase protein comprises a mutation or modification of its substrate pocket.
46. The method of any of statements 29-35 or 44-45, wherein the modified isopropylmalate synthase protein comprises a mutation or modification in its acetyl-CoA binding surface near its substrate pocket.
47. The method of any of statements 29-35 or 44-46, wherein the modified isopropylmalate synthase protein comprises a mutation or modification within or next to its substrate pocket and a mutation or modification in its acetyl-CoA binding surface near its substrate pocket.
48. The method of any of statements 29-35 or 44-47, wherein the modified isopropylmalate synthase protein comprises an aspartic acid replacement in its catalytic domain.
49. The method of any of statements 29-35 or 44-48, wherein the modified isopropylmalate synthase protein comprises an aspartic acid replacement at an amino acid position corresponding to position 228 of SEQ ID NO:2.
50. The method of any of statements 29-35 or 44-49, wherein the seeds and/or plants have increased content of amino acids comprising valine.
51. The method of any of statements 29-50, wherein plants that express the modified isopropylmalate synthase protein have increased amino acid content compared to an average content for one or more amino acids of wild type plants, or parental plants, or plants with a knockout IPMS gene that do not express the modified isopropylmalate synthase protein.
52. The method of any of statements 29-51, wherein plants that express the modified isopropylmalate synthase protein have increased content of Gln, His, Ile, Lu, Lys, Met, Phe, Thr, Trp, Val, or a combination thereof.
53. The method of any of statements 29-52, wherein a plant expressing the modified isopropylmalate synthase protein has at least a 1%, at least a 2%, at least a 3%, at least a 4%, at least a 5%, at least a 10%, at least a 15%, at least a 20%, at least a 25%, at least a 30%, at least a 40%, at least a 50%, at least a 100%, at least a 150%, at least a 200%, at least a 300%, at least a 400% e, or at least a 500% increase in the content of one or more amino acids compared to an average content for one or more amino acids of wild type plants, or parental plants, or plants with a knockout IPMS gene that do not express the modified isopropylmalate synthase protein.
54. The method of any of statements 29-53, wherein plants expressing the modified isopropylmalate synthase protein have at least a two-fold, at least a three-fold, at least a four-fold, or at least a five-fold increase in the content of one or more amino acids compared to an average amino acid content of wild type plants, or parental plants, or plants with a knockout IPMS gene that do not express the modified isopropylmalate synthase protein.
55. The method of any of statements 29-54, wherein plants that express a modified isopropylmalate synthase protein have increased biomass compared to an average biomass of wild type plants, or parental plants, or plants with a knockout IPMS gene that do not express the modified isopropylmalate synthase protein.
56. The method of any of statements 29-55, wherein a plant expressing the modified isopropylmalate synthase protein has at least a 1%, at least a 2%, at least a 3%, at least a 4%, at least a 5%, at least a 10%, at least a 15%, at least a 20%, at least a 25%, at least a 30%, at least a 40%, at least a 50%, at least a 100%, at least a 150%, at least a 200%, at least a 300%, at least a 400%, or at least a 500% increase in biomass compared to an average biomass of wild type plants, or parental plants, or plants with a knockout IPMS gene that do not express the modified isopropylmalate synthase protein.
57. The method of any of statements 29-56, wherein the modified isopropylmalate synthase nucleic acid or the modified isopropylmalate synthase protein has at least 70%, at least 80%, at least 85%, at least 90%, at least 95%, at least 96%, at least 97%, at least 98%, at least 99%, or at least 99.5% sequence identity to any of SEQ ID NO:1-38.
58. The method of any of statements 29-57, wherein the modified isopropylmalate synthase nucleic acid or a cDNA copy of an endogenous modified isopropylmalate synthase mRNA has at least 70%, at least 80%, at least 85%, at least 90%, at least 95%, at least 96%, at least 97%, at least 98%, at least 99%, or at least 99.5% sequence identity to any of SEQ ID NO:1, 4, 6, 8, 27, 29, 31, 34, or 37.
59. The method of any of statements 29-58, wherein a cDNA copy of an mRNA encoding the modified isopropylmalate synthase has at least 70%, at least 80%, at least 85%, at least 90%, at least 95%, at least 96%, at least 97%, at least 98%, at least 99%, or at least 99.5% sequence identity to any of SEQ ID NO:4 or 6.
60. The method of any of statements 29-59, wherein the modified isopropylmalate synthase protein has at least 70%, at least 80%, at least 85%, at least 90%, at least 95%, at least 96%, at least 97%, at least 98%, at least 99%, or at least 99.5% sequence identity to any of SEQ ID NO:2, 3, 5, 7, 9, 10, 12, 13, 14, 15, 16, 17, 18, 19, 20, 21, 22, 23, 24, 25, 26, 28, 30, 32, 33, 34, 35, 36, or 38.
61. The method of any of statements 29-60, wherein the modified isopropylmalate synthase protein has at least 70%, at least 80%, at least 85%, at least 90%, at least 95%, at least 96%, at least 97%, at least 98%, at least 99%, or at least 99.5% sequence identity to any of SEQ ID NO: 3, 5, 9, 10, 12, 13, 15, 16, 18, 19, 21, 22, 24, 25, 28, 32, 33, 34, 35, 36, or 38.
62. The method of any of statements 29-61, wherein the plant is an agricultural crop species or a forage crop species.
63. The method of any of statements 29-62, wherein the plant is forage plant (e.g., alfalfa, clover, soybeans, turnips, bromegrass, bluestem, and fescue), starch plant (e.g., canola, potatoes, lupins, sunflower and cottonseed), grain (maize, wheat, barley, oats, rice, sorghum, millet and rye), grass (switchgrass, prairie grass, wheat grass, sudangrass, sorghum, straw-producing plants, miscanthus, switchgrass), sugar producing plants (sugarcane, beets), vegetable plant (e.g., cucumber, lettuce, tomato). Brachypodium, Arabidopsis, bamboo, softwood, or hardwood.
64. The method of any of statements 29-63, further comprising harvesting the selected plant(s), or parts of the selected plant(s).
65. The method of any of statements 29-64, further comprising harvesting seeds from the selected plant(s).
66. The method of any of statements 29-65, further comprising harvesting vegetables or leaves from the selected plant(s).
67. A method comprising (a) cultivating a seed or seedling to produce a mature plant therefrom, wherein the seed or seedling comprises a modified isopropylmalate synthase (IPMS) gene that encodes a modified isopropylmalate synthase (IPMS) protein, or wherein the seed or seedling comprises an expression cassette comprising a promoter operably linked to a nucleic acid segment encoding a modified isopropylmalate synthase (IPMS) protein; and (b) harvesting the mature plant, one or more seeds from the mature plant, or one or more parts of the mature plant.
68. The method of statement 67, wherein the seed or seedling comprises at least one mutation or modification in an endogenous (native) isopropylmalate synthase (IPMS) gene.
69. The method of statement 67 or 68, wherein the seed or seedling comprises an expression cassette comprising a promoter operably linked to a nucleic acid segment encoding a modified isopropylmalate synthase (IPMS) protein.
70. The method of statement 69, wherein the promoter is a tissue specific promoter or a developmentally regulated promoter.
71. The method of statement 69 or 70 wherein the promoter is a seed specific promoter.
72. The method of any of statements 67-71, wherein the modified isopropylmalate synthase protein has isopropylmalate synthase activity or the modified isopropylmalate synthase protein does not have significant isopropylmalate synthase activity.
73. The method of any of statements 67-72, wherein the modified isopropylmalate synthase protein comprises a mutation or modification of its allosteric domain.
74. The method of any of statements 67-73, wherein the modified isopropylmalate synthase protein has a mutation or modification located within about 20 amino acids of the C-terminus.
75. The method of any of statements 67-74, wherein the modified isopropylmalate synthase protein has a point mutation at a position corresponding to position 606 of SEQ ID NO:2.
76. The method of any of statements 67-75, wherein the modified isopropylmalate synthase protein has a replacement of a glycine (G) at a position corresponding to position 606 of SEQ ID NO:2.
77. The method of any of statements 67-76, wherein the glycine at a position corresponding to position 606 of SEQ ID NO:2 is a glutanic acid (E).
78. The method of any of statements 67-77, wherein the plants' seeds have increased content of Gln, His, Ile, Leu, Lys, Met, Phe, Thr, Trp, Val, or a combination thereof.
79. The method of any of statements 67-78, wherein the plants' vegetative tissues have increased content of amino acids comprising Leu, and other amino acids.
80. The method of any of statements 67-79, wherein the plants have increased biomass.
81. The method of any of statements 67-72, wherein the modified isopropylmalate synthase protein comprises a mutation or modification of its catalytic domain.
82. The method of any of statements 67-72, or 81 wherein the modified isopropylmalate synthase protein has a mutation within or next to its substrate pocket.
83. The method of any of statements 67-72, or 81-82, wherein the modified isopropylmalate synthase protein has a mutation in its acetyl-CoA binding surface near its substrate pocket.
84. The method of any of statements 67-72, or 81-83, wherein the modified isopropylmalate synthase protein has as aspartic acid replaced at an amino acid position corresponding to position 228 of SEQ ID NO:2.
85. The method of any of statements 67-72, or 81-84, wherein the seeds and/or plants have increased content of amino acids comprising valine.
86. The method of any of statements 67-85, wherein plants that express the modified isopropylmalate synthase protein have increased amino acid content compared to an average content for one or more amino acids of wild type plants, or parental plants, or plants with a knockout IPMS gene that do not express the modified isopropylmalate synthase protein.
87. The method of any of statements 67-86, wherein plants that express the modified isopropylmalate synthase protein have increased content of Gln, His, Ile, Leu, Lys, Met, Phe, Thr, Trp, Val, or a combination thereof.
88. The method of any of statements 67-87, wherein a plant expressing the modified isopropylmalate synthase protein has at least a 1%, at least a 2%, at least a 3%, at least a 4%, at least a 5%, at least a 10%, at least a 15%, at least a 20%, at least a 25%, at least a 30%, at least a 40%, at least a 50%, at least a 100%, at least a 150%, at least a 200%, at least a 300%, at least a 400%, or at least a 500% increase in the content of one or more amino acids compared to an average content for one or more amino acids of wild type plants, or parental plants, or plants with a knockout IPMS gene that do not express the modified isopropylmalate synthase protein.
89. The method of any of statements 67-88, wherein plants expressing the modified isopropylmalate synthase protein has at least a two-fold, at least a three-fold, at least a four-fold, or at least a five-fold increase in the content of one or more amino acids compared to an average amino acid content of wild type plants, or parental plants, or plants with a knockout IPMS gene that do not express the modified isopropylmalate synthase protein.
90. The method of any of statements 67-89, wherein the increased amino acid content is in the plant's vegetative tissues, seeds, or a combination thereof.
91. The method of any of statements 67-90, wherein plants that express the modified isopropylmalate synthase protein have increased biomass compared to an average biomass of wild type plants, or parental plants, or plants with a knockout IPMS gene that do not express the modified isopropylmalate synthase protein.
92. The method of any of statements 67-91, wherein plants that express the modified isopropylmalate synthase protein have increased biomass compared to an average biomass of wild type plants, or parental plants, or plants with a knockout IPMS gene that do not express the modified isopropylmalate synthase protein.
93. The method of any of statements 67-92, wherein plants expressing the modified isopropylmalate synthase protein has at least a 1%, at least a 2%, at least a 3%, at least a 4%, at least a 5%, at least a 10%, at least a 15%, at least a 20%, at least a 25%, at least a 30%, at least a 40%, at least a 50%, at least a 100%, at least a 150%, at least a 200%, at least a 300%, at least a 400%, or at least a 500% increase in biomass compared to an average biomass of wild type plants, or parental plants, or plants with a knockout IPMS gene that do not express the modified isopropylmalate synthase protein.
94. The method of any of statements 67-93, wherein the modified isopropylmalate synthase nucleic acid or the modified isopropylmalate synthase protein has at least 70%, at least 80%, at least 85%, at least 90%, at least 95%, at least 96%, at least 97%, at least 98%, at least 99%, or at least 99.5% sequence identity to any of SEQ ID NO:1-38.
95. The method of any of statements 67-94, wherein the modified isopropylmalate synthase nucleic acid has at least 70%, at least 80%, at least 85%, at least 90%, at least 95%, at least 96%, at least 97%, at least 98%, at least 99%, or at least 99.5% sequence identity to any of SEQ ID NO:1, 4, 6, 8, 27, 29, 31, 34, or 37.
96. The method of any of statements 67-95, wherein the modified isopropylmalate synthase nucleic acid has at least 70%, at least 80%, at least 85%, at least 90%, at least 95%, at least 96%, at least 97%, at least 98%, at least 99%, or at least 99.5% sequence identity to any of SEQ ID NO:4 or 6.
97. The method of any of statements 67-96, wherein the modified isopropylmalate synthase protein has at least 70%, at least 80%, at least 85%, at least 90%, at least 95%, at least 96%, at least 97%, at least 98%, at least 99%, or at least 99.5% sequence identity to any of SEQ ID NO:2, 3, 5, 7, 9, 10, 12, 13, 14, 15, 16, 17, 18, 19, 20, 21, 22, 23, 24, 25, 26, 28, 30, 32, 33, 34, 35, 36, or 38.
98. The method of any of statements 67-97, wherein the modified isopropylmalate synthase protein has at least 70%, at least 80%, at least 85%, at least 90%, at least 95%, at least 96%, at least 97%, at least 98%, at least 99%, or at least 99.5% sequence identity to any of SEQ ID NO: 3, 5, 9, 10, 12, 13, 15, 16, 18, 19, 21, 22, 24, 25, 28, 32, 33, 34, 35, 36, or 38.
99. The method of any of statements 67-98, which seed or seedling is an agricultural crop species or a forage crop species.
100. The method of any of statements 67-99, which seed or seedling is a forage plant (e.g., alfalfa, clover, soybeans, turnips, bromegrass, bluestem, and fescue), starch plant (e.g., canola, potatoes, lupins, sunflower and cottonseed), grain (maize, wheat, barley, oats, rice, sorghum, millet and rye), grass (switchgrass, prairie grass, wheat grass, sudangrass, sorghum, straw-producing plants, miscanthus, switchgrass), sugar producing plants (sugarcane, beets), vegetable plant (e.g., cucumber, lettuce, tomato), Brachypodium, Arabidopsis, bamboo, softwood, or hardwood.
101. The method of any of statements 67-100, further comprising harvesting seeds from the mature plant.

The specific plants, plant cells, seeds, methods, and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

1. A plant cell, plant seed, or plant comprising at least one of:
a modified or mutant endogenous isopropylmalate synthase (IPMS) gene that encodes the isopropylmalate synthase (IPMS) protein; or
an expression cassette comprising a promoter operably linked to a nucleic acid segment encoding a modified or mutant isopropylmalate synthase protein.

2. (canceled)

3. (canceled)

4. (canceled)

5. (canceled)

6. (canceled)

7. The plant cell, plant seed, or plant of claim 1, wherein the modified isopropylmalate synthase protein does not have significant isopropylmalate synthase activity.

8. The plant cell, plant seed, or plant of claim 1, wherein the modified isopropylmalate synthase protein has at least one of:
a modification within its catalytic domain, a modification within its allosteric domain, or a combination thereof;
an aspartic acid within its catalytic domain that is replaced by another amino acid; or
an aspartic acid at a position corresponding to position 228 of SEQ ID NO:2 that is replaced by another amino acid.

9. (canceled)

10. (canceled)

11. The plant cell, plant seed, or plant of claim 1, wherein the modified isopropylmalate synthase protein has at least one of a glycine within its allosteric domain that is replaced by another amino acid or a glycine at a position corresponding to position 606 of SEQ ID NO:2 that is replaced by another amino acid.

12. (canceled)

13. The plant cell, plant seed, or plant of claim 1, wherein the modified isopropylmalate synthase protein comprises a sequence with at least one amino acid modification to any of SEQ ID NO: 2, 3, 5, 7, 9, 10, 12, 13, 14, 15, 16, 17, 18, 19, 20, 21, 22, 23, 24, 25, 26, 28, 30, 32, 33, 35, 36, or 38.

14. The plant cell, plant seed, or plant of claim 1, wherein the plant or a plant generated from the plant cell or the plant seed has increased amino acid content or wherein the plant or a plant generated from the plant cell or the plant seed has increased content of at least one of Gln, His, Ile, Leu, Lys, Met, Phe, Thr, Trp, Val, or a combination thereof.

15. (canceled)

16. The plant cell, plant seed, or plant of claim 1, which is a forage species, starch species, oil species, grain species, grass species, sugar producing species, vegetable species, canola species, corn species, soybean species, sunflower species, walnut species, or olive species of plant, plant cell or plant seed.

17. The plant cell, plant seed, or plant of claim 1, wherein the modified isopropylmalate synthase (IPMS) protein is generated from a forage species, starch species, oil species, grain species, grass species, sugar producing species, canola, corn, soybean, sunflower, walnut, olive, or vegetable species of isopropylmalate synthase (IPMS).

18. (canceled)

19. (canceled)

20. A method comprising cultivating one or more seeds or seedlings, where the one or more seeds or seedlings have at least one of a modified or mutant endogenous isopropylmalate synthase (IPMS) nucleic acid that encodes a modified isopropylmalate synthase (IPMS) protein or an exogenous expression cassette comprising a promoter operably linked to a nucleic acid segment having at least one mutation or modification in a coding region encoding the modified isopropylmalate synthase (IPMS) protein, to thereby generate one or more modified mature plants, and harvesting at least one of vegetative tissues or seeds from the one or more modified mature plants.

21. (canceled)

22. (canceled)

23. The method of claim 20, wherein the modified isopropylmalate synthase (IPMS) protein does not have significant isopropylmalate synthase activity.

24. The method of claim 20, wherein the modified isopropylmalate synthase protein has at least one of:
a modification within its catalytic domain, a modification within its allosteric domain, or a combination thereof;
an aspartic acid within its catalytic domain that is replaced by another amino acid; or
an aspartic acid at a position corresponding to position 228 of SEQ ID NO:2 that is replaced by another amino acid.

25. (canceled)

26. (canceled)

27. The method of claim 24, wherein the modified isopropylmalate synthase protein has at least one of a glycine within its allosteric domain that is replaced by another amino acid or a glycine at a position corresponding to position 606 of SEQ ID NO:2 that is replaced by another amino acid.

28. (canceled)

29. The method of claim 20, wherein the modified isopropylmalate synthase protein comprises a sequence with at least one amino acid modification to any of SEQ ID NO: 2, 3, 5, 7, 9, 10, 12, 13, 14, 15, 16, 17, 18, 19, 20, 21, 22, 23, 24, 25, 26, 28, 30, 32, 33, 35, 36, or 38.

30. (canceled)

31. (canceled)

32. The method of claim 20, wherein the isopropylmalate synthase (IPMS) nucleic acid, one or more seeds or one or more seedlings are forage species, starch species, oil species, grain species, grass species, sugar producing species, canola species, corn species, soybean species, sunflower species, walnut species, olive species, or vegetable species of isopropylmalate synthase (IPMS) nucleic acid, seeds or seedlings.

33. (canceled)

34. A method comprising: selecting at least one plant cell comprising at least one mutation or modification in an endogenous isopropylmalate synthase (IPMS) gene that expresses a modified isopropylmalate synthase protein, or selecting at least one plant cell comprising an exogenous expression cassette comprising a promoter operably linked to a nucleic acid segment having at least one mutation or modification in a coding region encoding the modified isopropylmalate synthase (IPMS) protein; generating one or more plants from the at least one plant cells so selected, and cultivating the one or more plants to generate one or more mature plants.

35. (canceled)

36. (canceled)

37. The method of claim 34, wherein the one or more mature plants have increased biomass or increased content of one or more amino acid relative to an average amino acid content, or an average biomass of wild type plants, or parental plants, or plants with a knockout IPMS gene that do not express the modified isopropylmalate synthase protein.

38. (canceled)

39. The method of claim 34, wherein the modified isopropylmalate synthase protein has at least one of:
a modification within its catalytic domain, a modification within its allosteric domain, or a combination thereof;
an aspartic acid within its catalytic domain that is replaced by another amino acid; or
an aspartic acid within its catalytic domain that is replaced by another amino acid at a position corresponding to position 228 of SEQ ID NO:2.

40. (canceled)

41. The method of claim 39, wherein the modified isopropylmalate synthase protein has at least one of a glycine within its allosteric domain that is replaced by another amino acid or glycine at position 606 of SEQ ID NO:2 that is replaced by another amino acid.

42. (canceled)

43. The method of claim 34, wherein the modified isopropylmalate synthase protein comprises a sequence with at least one amino acid modification to any of SEQ ID NO: 2, 3, 5, 7, 9, 10, 12, 13, 14, 15, 16, 17, 18, 19, 20, 21, 22, 23, 24, 25, 26, 28, 30, 32, 33, 35, 36, or 38.

44. (canceled)

45. (canceled)

46. The method of claim 34, wherein at least one of the plant cells is a forage species, starch species, oil species, grain species, grass species, sugar producing species, vegetable species, canola species, corn species, soybean species, sunflower species, walnut species, or olive species.

47. (canceled)

48. (canceled)
